(19) United States
(12) Patent Application Publication
(10) Pub. No.: US 2025/0213569 A1
(43) Pub. Date: Jul. 3, 2025

(54) METHODS AND COMPOSITIONS FOR MEASURING AND INHIBITING EXONUCLEASE ACTIVITY

(71) Applicant: New York University, New York, NY (US)

(72) Inventors: Michele PAGANO, New York, NY (US); Gergely RONA, New York, NY (US); Andras ZEKE, Budakeszi (HU); Bearach MIWATANI-MINTER, Houston, TX (US)

(73) Assignee: New York University, New York, NY (US)

(21) Appl. No.: 18/269,245

(22) PCT Filed: Dec. 22, 2021
(86) PCT No.: PCT/US21/64922

§ 371 Date: Jun. 22, 2023

Related U.S. Application Data
(60) Provisional application No. 63/130,174, filed on Dec. 23, 2020.
(60) Provisional application No. 63/236,037, filed on Aug. 23, 2021.

(51) Int. Cl. A61K 31/519; A61K 31/194; A61K 31/37; A61K 31/4375; A61K 31/47; A61K 31/4704; A61K 31/4709; A61K 31/472; A61K 31/498; A61K 31/55; A61K 31/7048; A61K 45/06; A61P 31/14; C12Q 1/34; G01N 21/64

(52) U.S. Cl. CPC A61K 31/519; A61K 31/194; A61K 31/37; A61K 31/4375; A61K 31/47; A61K 31/4704; A61K 31/4709; A61K 31/472; A61K 31/498; A61K 31/55; A61K 31/7048; A61K 45/06; A61P 31/14; C12Q 1/34; G01N 21/6428; G01N 2021/6432; G01N 2021/6439

(57) ABSTRACT

The present disclosure relates to novel methods and compositions for a measuring exonuclease enzyme activity. In particular, the methods entail contacting a sample with a fluorescently-labeled substrate to create a test mixture, incubating the test mixture for a time sufficient for cleavage of the substrate, and measuring the fluorescence signal from the test reaction mixture. In certain aspects, the methods disclosed herein are useful for identifying and/or assessing a modulator of an exonuclease that may be used either alone or in combination with other compounds. The present disclosure also relates to fluorescently-labeled double-stranded nucleic acid compositions useful in the practice of such methods and, still further, to kits for performing the methods of the disclosure. The present disclosure further relates to compounds and methods useful for treating a viral infection.

### SEQUENCE LISTING

The instant application contains a Sequence Listing which has been submitted electronically in ASCII format and is hereby incorporated by reference in its entirety. Said ASCII copy, created on Dec. 20, 2021, is named 243735_000233_SL.txt and is 31,787 bytes in size.

### FIELD OF THE INVENTION

Disclosed herein are novel methods and compositions for measuring exonuclease enzyme activity. In certain aspects, the methods disclosed herein are useful for identifying and/or assessing a modulator of an exonuclease that may be used either alone or in combination with other compounds. The present disclosure also relates to novel nucleic acid compositions useful in the practice of such methods and, still further, to kits for performing the methods of the disclosure. The present disclosure further relates to compounds and methods useful for treating a viral infection.

### BACKGROUND

Coronaviruses (CoVs) are a group of viruses belonging to the Coronaviridae family in the order of Nidovirales that can cause acute to severe respiratory and gastrointestinal tract diseases in humans and other animals. CoVs are enveloped, single-stranded RNA viruses with large ribonucleic acid (RNA) genomes, ranging from approximately 26 to 32 kb. The present COVID-19 or SARS2 (coronavirus disease 2019) pandemic that is wreaking havoc worldwide in public health and economies is caused by severe acute respiratory syndrome coronavirus 2 (SARS-CoV2). SARS-CoV strain caused the SARS outbreak in 2002, and related virus MERS-CoV was responsible for the outbreak of Middle East respiratory syndrome disease in 2012 (Robson et al., 2020).

SARS-CoV2 uses RNA-dependent RNA polymerase (RdRP) to replicate. The low replicative fidelity of RdRP allows for viral adaption to diverse environments at the expense of a high error rate and risk for viral extinction. To offset the low fidelity of RdRP, as with most viruses in the order of Nidovirales, SARS-CoV2 possesses a proofreading RNA repair system that proofreads and removes mismatched nucleotides during genome replication and transcription. Specifically, NSP14 of CoV2 harbors a unique N-terminal 3′ to 5′ exonuclease (ExoN) proofreading domain that excises mismatched nucleotides from the growing 3′ end of the RNA strand, supporting replication fidelity and maintenance of its unusually large genome. X-ray crystallography of SARS-CoV NSP14 complexed with allosteric activator SARS-CoV NSP10, which enhances the activity of NSP14, have revealed key features of the DEDD(h)-type catalytic domain of NSP14, including the Mg²⁺ ion cofactor requisite for catalysis (Bouvet et al., 2012; Yang et al., 2011). High sequence homology between SARS and SARS2 catalytic domains has facilitated detailed modeling of the NSP14/NSP10 exonuclease complex, and its mapping onto published structures (Ferron et al., 2017; Ma et al., 2015; resb.org/structure/5NFY).

Experimental studies suggest that the CoV proofreading RNA repair system is essential for its virulence. Mutations to the catalytic site of the ExoN domain generally leads to lowered viral viability, a hypermutator phenotype and/or complete loss of viral replication, depending on the strain (Becares et al., 2016; Case et al., 2017; Eckerle et al., 2007; Graepel at al., 2017; Graham et al., 2012; Minkskaia et al., 2006; Orgando et al., 2019). Importantly, similarly to SARS-CoV (Orgando et al., 2019), it was recently shown that NSP14 enzymatic exonuclease activity is also essential for the SARS-CoV2 virus (Orgando et al., 2020). While known strains of coronaviruses do not mutate extensively-instead of antigenic shift, they rely on strong interferon system suppression-their resistance to inhibitors is less problematic as compared to other viruses (e.g., influenza viruses). Development of NSP14/NSP10 exonuclease complex inhibitors, while an attractive avenue for future rational therapeutics targeting CoV, has not yet been aggressively pursued, partly due to the lack of robust in vitro screening assays for high sensitivity detection and quantification of NSP14 activity, a tool that could be subsequently used to identify antiviral strategies that inhibit its exonuclease activity. Currently available radioactive exonuclease activity assays require resolution of radioactive RNA products under denaturing gel conditions and are poor candidates for high throughput screening of compounds.

There remains a need for assays for high sensitivity detection and quantification of exonuclease activity for the identification and development of antiviral strategies that inhibit exonuclease activity.

### SUMMARY OF THE INVENTION

In one aspect, provided herein is a fluorescence resonance energy transfer (FRET)-based method for measuring a 3′ to 5′ exonuclease activity in a sample, comprising:
(a) contacting the sample with a fluorescently labeled double-stranded RNA (dsRNA) substrate to create a test reaction mixture, wherein said dsRNA substrate comprises (i) at least one free 3′ OH group, and (ii) a pair of FRET probes comprising a fluorophore and a quencher, wherein one probe is located at the 5′ end of the strand comprising the free 3′ OH group and the other probe is located either at the 5′ end or at the 3′ end of the other strand of said dsRNA substrate, and when the substrate is uncleaved, the quencher quenches the fluorescence signal of the fluorophore;
(b) incubating said test reaction mixture under conditions and for a time sufficient for cleavage of the substrate by the 3′ to 5′ exonuclease, wherein the cleavage of the substrate by the 3′ to 5′ exonuclease causes sufficient separation of the fluorophore and the quencher to reduce quenching of the fluorescence signal of the fluorophore, and
(c) measuring the fluorescence signal emitted from the test reaction mixture.

In some embodiments, the method FRET-based method further comprises comparing the fluorescence signal measured in step (c) to a control fluorescence signal.

In some embodiments, the control fluorescence signal is a predetermined value.

In some embodiments, the control fluorescence signal is the fluorescence signal measured under the same conditions in a control sample comprising the same dsRNA substrate but in the absence of the exonuclease.

In some embodiments, the control fluorescence signal is the fluorescence signal measured under the same conditions in a control sample comprising the same dsRNA substrate and a specific amount of a control nuclease.

In some embodiments, the control nuclease is selected from RNase A, RNase L, PNPase, RNase II, RNase R, Ribonuclease T1, Nuclease BAL-31, and RNase III.

In another aspect, provided herein is a fluorescence resonance energy transfer (FRET)-based method for identifying and/or assessing a modulator of a 3′ to 5′ exonuclease, comprising:
(a) in a test reaction mixture, contacting the exonuclease with a test compound and a fluorescently labeled double-stranded RNA (dsRNA) substrate, wherein said dsRNA substrate comprises (i) at least one free 3′ OH group, and (ii) a pair of FRET probes comprising a fluorophore and a quencher, wherein one probe is located at the 5′ end of the strand comprising the free 3′ OH group and the other probe is located either at the 5′ end or at the 3′ end of the other strand of said dsRNA substrate, and when the substrate is uncleaved, the quencher quenches the fluorescence signal of the fluorophore;
(b) incubating said test reaction mixture under conditions and for a time sufficient for cleavage of the substrate by the exonuclease in the absence of the test compound, wherein the cleavage of the substrate by the exonuclease causes sufficient separation of the fluorophore and the quencher to reduce quenching of the fluorescence signal of the fluorophore;
(c) determining the fluorescence signal emitted from the test reaction mixture;
(d) comparing the fluorescence signal determined in step (c) to a control fluorescence signal, wherein the control fluorescence signal is the fluorescence signal determined under the same conditions in a control sample comprising the same amounts of exonuclease and dsRNA substrate but in the absence of the test compound, and
(e) (i) determining that the test compound is an inhibitor of the exonuclease if the fluorescence signal in the test reaction mixture is lower than in the control reaction mixture, or (ii) determining that the test compound is not an inhibitor of the exonuclease if the fluorescence signal in the test reaction mixture is not lower than in the control reaction mixture, or (iii) determining that the test compound is an activator of the exonuclease if the fluorescence signal in the test reaction mixture is higher than in the control reaction mixture.

In some embodiments, step (a) comprises pre-incubating the exonuclease with the test compound prior to the addition of the dsRNA substrate.

In some embodiments, step (a) comprises adding the test compound after contacting the exonuclease with a dsRNA substrate.

In another aspect, provided herein is a fluorescence resonance energy transfer (FRET)-based method for measuring processivity of a 3′ to 5′ exonuclease, comprising:
(a) contacting the exonuclease with a first fluorescently labeled double-stranded RNA (dsRNA) substrate to create a first reaction mixture, wherein said first dsRNA substrate comprises (i) at least one free 3′ OH group, and (ii) a pair of FRET probes comprising a fluorophore and a quencher, wherein one probe is located at the 5′ end of the strand comprising the free 3′ OH group and the other probe is located either at the 5′ end or at the 3′ end of the other strand of said first dsRNA substrate, and when the substrate is uncleaved, the quencher quenches the fluorescence signal of the fluorophore;
(b) contacting the exonuclease with a second dsRNA substrate to create a second reaction mixture, wherein the second dsRNA substrate differs from the first dsRNA substrate in that it is longer than the first substrate;
(c) incubating said first reaction mixture and said second reaction mixture under conditions and for a time allowing for cleavage of both substrates by the exonuclease, wherein the cleavage of the substrates by the exonuclease causes sufficient separation of the fluorophore and the quencher to reduce quenching of the fluorescence signal of the fluorophore, and
(d) determining the time required for the first reaction mixture and the second reaction mixture to reach the same level of fluorescence signal;
wherein the processivity of the exonuclease is measured as the difference in the length between the first and second substrate divided by the difference in the time required for the first reaction mixture and second reaction mixture to reach the same level of fluorescence signal.

In various embodiments of any of the above methods, the fluorophore is located at the 5′ end of the strand comprising the free 3′ OH group and the quencher is located either at the 5′ end or at the 3′ end of the other strand of said dsRNA substrate. In some embodiments, the quencher is located at the 5′ end of the other strand of the dsRNA substrate. In some embodiments, the quencher is located at the 3′ end of the other strand of the dsRNA substrate.

In various embodiments of any of the above methods, the quencher is located at the 5′ end of the strand comprising the free 3′ OH group and the fluorophore is located either at the 5′ end or at the 3′ end of the other strand of said dsRNA substrate. In some embodiments, the fluorophore is located at the 5′ end of the other strand of the dsRNA substrate. In some embodiments, the fluorophore is located at the 3′ end of the other strand of the dsRNA substrate.

In various embodiments of any of the above methods, the pair of FRET probes are selected from 6FAM-BHQ1, Cy3-BHQ2, TAMRA-BHQ2, TexasRed-BHQ2, and Cy5-BHQ3. In some embodiments, the fluorophore is 6FAM and the quencher is BHQ1. In some embodiments, the fluorophore is TexasRed and the quencher is BHQ2.

In various embodiments of any of the above methods, the at least one free 3′ end of the dsRNA substrate has one or more mismatches. In some embodiments, the at least one free 3′ end of the dsRNA substrate has one to three mismatches. In some embodiments, the one or more mismatches comprise one or more ribonucleotide analogs. In some embodiments, the ribonucleotide analog is selected from Remdesivir, Ribavirin, Favipiravir, N4-Hydroxycytidine (EIDD-1931) or its derivative Molnupiravir, 5-Fluorouracil and Sofosbuvir.

In various embodiments of any of the above methods, the dsRNA substrate has a length between 13 and 20 base pairs.

In various embodiments of any of the above methods, the dsRNA substrate comprises a GC-stretch at the free 3′ end. In some embodiments, the dsRNA substrate comprises a GC-free-stretch adjacent to the fluorophore and/or quencher. In some embodiments, the GC-free-stretch is at least 1 base apart from the fluorophore and/or quencher.

In various embodiments of any of the above methods, the dsRNA substrate comprises a U or A base immediately next to the fluorophore.

In various embodiments of any of the above methods, the dsRNA substrate comprises a single C base in the second or third position from the fluorophore.

In various embodiments of any of the above methods, the dsRNA substrate comprises a 5′ overhang between 1 and 5 base pairs on the other strand.

In various embodiments of any of the above methods, the dsRNA substrate is selected from:

```
5′ FAM-UUGCCGAAUUAAGCGCA-3′ (SEQ ID NO: 1)
                  ||||||||||||||
          3′-CGGCUUAAUUCGCGAAU-BHQ1-5′ (SEQ ID NO: 2)
  5′ FAM-UUGCCGAAUUAAGCGCCA (SEQ ID NO: 3)
                |||||||||||||||||
3′ BHQ1-AACGGCUUAAUUCGCGGAAU (SEQ ID NO: 4)
  5′ FAM-UUUUUUCGGCCCA (SEQ ID NO: 5)
                ||||||||||||
3′ BHQ1-AAAAAAGCCGGGAUAAA (SEQ ID NO: 6)
  5′ FAM-UCUUUUCGGCCCA (SEQ ID NO: 7)
                ||||||||||||
3′ BHQ1-AGAAAAGCCGGGAUAAA (SEQ ID NO: 8)
5′ TxRed-UCUUUUCGGCCCA (SEQ ID NO: 9)
                  ||||||||||||
  3′ BHQ2-AGAAAAGCCGGGAUAAA (SEQ ID NO: 10)
  5′ Cy3-UCUUUUCGGCCCA (SEQ ID NO: 11)
                ||||||||||||
3′ BHQ2-AGAAAAGCCGGGAUAAA (SEQ ID NO: 10)
3′ TAMRA-UCUUUUCGGCCCA (SEQ ID NO: 12)
                  ||||||||||||
  3′ BHQ2-AGAAAAGCCGGGAUAAA (SEQ ID NO: 10)
  5′ Cy5-UCUUUUCGGCCCA (SEQ ID NO: 13)
                ||||||||||||
3′ BHQ3-AGAAAAGCCGGGAUAAA (SEQ ID NO: 14).
```

In various embodiments of any of the above methods, the 3′ to 5′ exonuclease is a proofreading exonuclease. In some embodiments, the proofreading exonuclease is a NSP14 exonuclease or a NSP14/NSP10 exonuclease complex from a virus of the order Nidovirales. In some embodiments, the virus is of the family Coronaviridae. In some embodiments, the virus is a Coronavirus. In some embodiments, the Coronavirus is a SARS-CoV virus, a SARS-CoV2 virus, a MERS-CoV virus, or HCoV-OC43, or a variant thereof. In some embodiments, the Coronavirus is a SARS-CoV2 virus. In some embodiments, the viral infection is a SARS-CoV infection. In some embodiments, the viral infection is a MERS-CoV infection.

In some embodiments, the exonuclease comprises purified NSP14 and NSP10 proteins in 1:1 to 1:5 molar ratio. In some embodiments, the exonuclease comprises purified NSP14 and NSP10 proteins in about 1:3 molar ratio. In some embodiments, the exonuclease is contacted with dsRNA substrate in the presence of Mg²⁺. In some embodiments, the exonuclease is contacted with dsRNA substrate at a temperature between 30° C. to 37° C. for about 1 hour in 50 mM TRIS-HCl, pH=7.5, 2 mM MgCl₂, 2 mM DTT. In some embodiments, the exonuclease is contacted with dsRNA substrate at 37° C. In some embodiments, NSP10 is pre-treated with 0.1 μM to 2 μM ZnCl₂. In some embodiments, NSP10 is pre-treated with 0.2 μM ZnCl₂.

In various embodiments of any of the above methods, the method is performed in a high throughput format.

In another aspect, provided herein is a fluorescently labeled double-stranded RNA (dsRNA) molecule selected from:

```
5′ FAM-UUGCCGAAUUAAGCGCA-3′ (SEQ ID NO: 1)
                  ||||||||||||||
          3′-CGGCUUAAUUCGCGAAU-BHQ1-5′ (SEQ ID NO: 2)
  5′ FAM-UUGCCGAAUUAAGCGCCA (SEQ ID NO: 3)
                |||||||||||||||||
3′ BHQ1-AACGGCUUAAUUCGCGGAAU (SEQ ID NO: 4)
  5′ FAM-UUUUUUCGGCCCA (SEQ ID NO: 5)
                ||||||||||||
3′ BHQ1-AAAAAAGCCGGGAUAAA (SEQ ID NO: 6)
  5′ FAM-UCUUUUCGGCCCA (SEQ ID NO: 7)
                ||||||||||||
3′ BHQ1-AGAAAAGCCGGGAUAAA (SEQ ID NO: 8)
5′ TxRed-UCUUUUCGGCCCA (SEQ ID NO: 9)
                  ||||||||||||
  3′ BHQ2-AGAAAAGCCGGGAUAAA (SEQ ID NO: 10)
  5′ Cy3-UCUUUUCGGCCCA (SEQ ID NO: 11)
                ||||||||||||
3′ BHQ2-AGAAAAGCCGGGAUAAA (SEQ ID NO: 10)
3′ TAMRA-UCUUUUCGGCCCA (SEQ ID NO: 12)
                  ||||||||||||
  3′ BHQ2-AGAAAAGCCGGGAUAAA (SEQ ID NO: 10)
  5′ Cy5-UCUUUUCGGCCCA (SEQ ID NO: 13)
                ||||||||||||
3′ BHQ3-AGAAAAGCCGGGAUAAA (SEQ ID NO: 14).
```

In another aspect, provided herein is a kit comprising one or more fluorescently labeled double-stranded RNA (dsRNA) molecules selected from:

```
5′ FAM-UUGCCGAAUUAAGCGCA-3′ (SEQ ID NO: 1)
                  ||||||||||||||
          3′-CGGCUUAAUUCGCGAAU-BHQ1-5′ (SEQ ID NO: 2)
  5′ FAM-UUGCCGAAUUAAGCGCCA (SEQ ID NO: 3)
                |||||||||||||||||
3′ BHQ1-AACGGCUUAAUUCGCGGAAU (SEQ ID NO: 4)
  5′ FAM-UUUUUUCGGCCCA (SEQ ID NO: 5)
                ||||||||||||
3′ BHQ1-AAAAAAGCCGGGAUAAA (SEQ ID NO: 6)
  5′ FAM-UCUUUUCGGCCCA (SEQ ID NO: 7)
                ||||||||||||
3′ BHQ1-AGAAAAGCCGGGAUAAA (SEQ ID NO: 8)
5′ TxRed-UCUUUUCGGCCCA (SEQ ID NO: 9)
                  ||||||||||||
  3′ BHQ2-AGAAAAGCCGGGAUAAA (SEQ ID NO: 10)
  5′ Cy3-UCUUUUCGGCCCA (SEQ ID NO: 11)
                ||||||||||||
3′ BHQ2-AGAAAAGCCGGGAUAAA (SEQ ID NO: 10)
3′ TAMRA-UCUUUUCGGCCCA (SEQ ID NO: 12)
                  ||||||||||||
  3′ BHQ2-AGAAAAGCCGGGAUAAA (SEQ ID NO: 10)
  5′ Cy5-UCUUUUCGGCCCA (SEQ ID NO: 13)
                ||||||||||||
3′ BHQ3-AGAAAAGCCGGGAUAAA (SEQ ID NO: 14).
```

and optionally instructions for use.

In some embodiments, the kit further comprises a control nuclease selected from RNase A, RNase L, PNPase, RNase II, RNase R, Ribonuclease T1, Nuclease BAL-31, and RNase III. In some embodiments, the kit further comprises a 3′ to 5′ exonuclease. In some embodiments, the 3′ to 5′ exonuclease is a NSP14 exonuclease or a NSP14/NSP10 exonuclease complex from SARS-CoV2 virus. In some embodiments, the kit further comprises a reaction buffer comprising 50 mM TRIS-HCl, pH=7.5, 2 mM MgCl₂, 2 mM DTT.

In another aspect, provided herein is a method of treating a viral infection in a subject comprising administering to the subject a compound capable of inhibiting enzymatic activity of the NSP14-NSP10 complex having the structure of Formula (A):

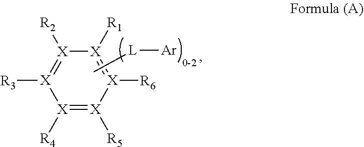

or a pharmaceutically acceptable salt thereof,
wherein X is independently at each occurrence selected from C, O, N, and S;
L is a linker selected independently at each occurrence from a bond, a C₁₋₆ alkyl, C₂₋₆ alkenyl, —CO—, —CO—NH—, —CO—(C₁₋₃ alkyl)-, and —CO—(C₂₋₄ alkenyl)-, wherein the C₁₋₆ alkyl optionally contains 1-2 heteroatoms selected from O, N, and S;
Ar is independently at each occurrence phenyl or a 5- or 6-membered heterocycle comprising from 1 to 3 heteroatoms independently selected from N, O, and S, wherein Ar is optionally substituted with one or more groups R′;
R₁, R₂, R₃, R₄, R₅ and R₆ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In another aspect, provided herein is a method of treating a viral infection in a subject comprising administering to the subject a compound capable of inhibiting enzymatic activity of the NSP14-NSP10 complex having the structure of Formula (B):

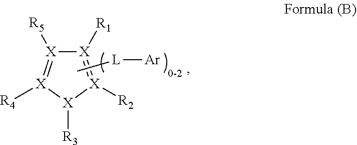

or a pharmaceutically acceptable salt thereof,
wherein X is independently at each occurrence selected from C, O, N, and S;
L is a linker selected independently at each occurrence from a bond, a C₁₋₆ alkyl, C₂₋₆ alkenyl, —CO—, —CO—NH—, —CO—(C₁₋₃ alkyl)-, and —CO—(C₂₋₄ alkenyl)-, wherein the C₁₋₆ alkyl optionally contains 1-2 heteroatoms selected from O, N, and S;
Ar is independently at each occurrence phenyl or a 5- or 6-membered heterocycle comprising from 1 to 3 heteroatoms independently selected from N, O, and S, wherein Ar is optionally substituted with one or more groups R′;
R₁, R₂, R₃, R₄, and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In another aspect, provided herein is a method of treating a viral infection in a subject comprising administering to the subject a compound capable of inhibiting enzymatic activity of the NSP14-NSP10 complex having the structure of Formula (I):

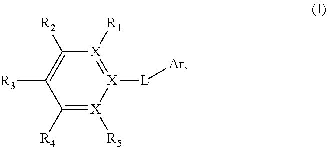

or a pharmaceutically acceptable salt thereof,
wherein X is independently at each occurrence selected from C, O, N and S;
L is a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar is phenyl or a 5- or 6-membered heterocycle comprising from 1 to 3 heteroatoms independently selected from N, O, and S, wherein Ar is optionally substituted with one or more groups R′;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound has the structure of Formula (IA):

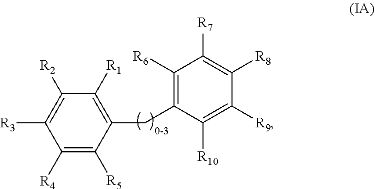

or a pharmaceutically acceptable salt thereof,
wherein R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound having the structure of Formula (IA) is selected from the group consisting of:

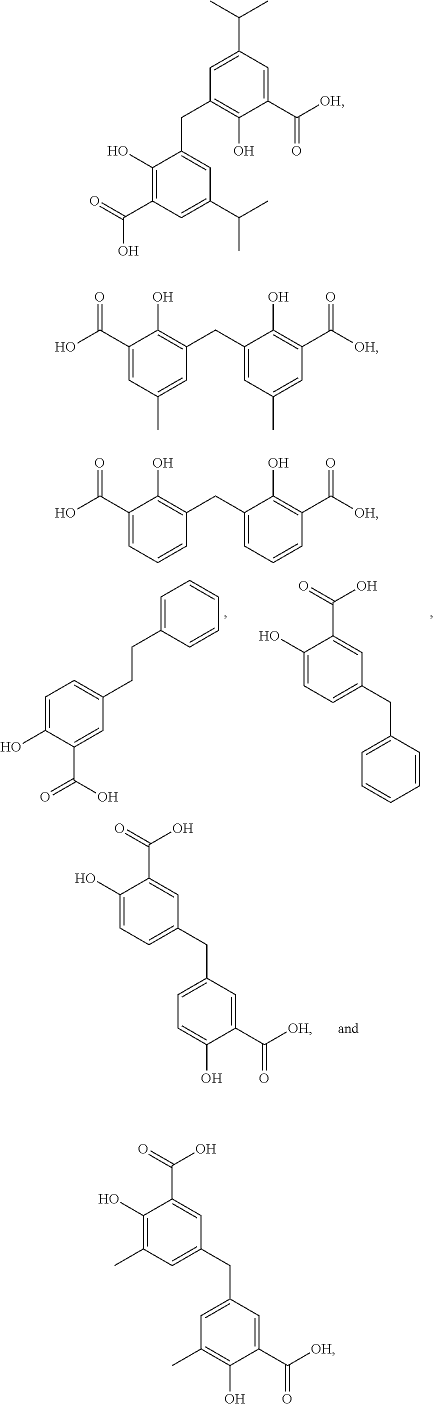

or a pharmaceutically acceptable salt thereof.

In some embodiments, the compound has the structure of Formula (IB):

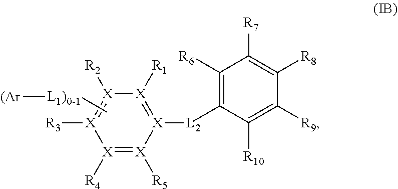

or a pharmaceutically acceptable salt thereof, wherein X is independently at each occurrence selected from C and N;
L₁ and L₂ are independently linkers selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar is phenyl or a 5- or 6-membered heterocycle comprising from 1 to 3 heteroatoms independently selected from N, O, and S, wherein Ar is optionally substituted with one or more groups R′;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound having the structure of Formula (IB) is selected from the group consisting of:

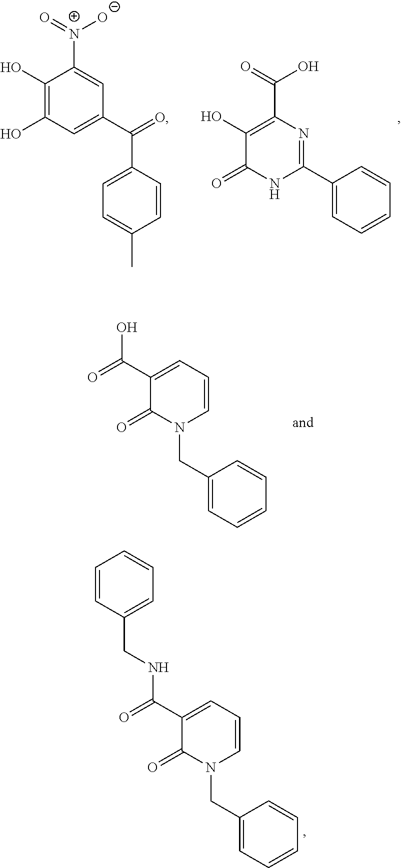 [SMILES: Cc1ccc(C(=O)c2cc(O)c(O)c([N+](=O)[O-])c2)cc1 ; O=C(O)c1nc(-c2ccccc2)[nH]c(=O)c1O ; O=C(O)c1cccn(Cc2ccccc2)c1=O ; O=C(NCc1ccccc1)c1cccn(Cc2ccccc2)c1=O]

or a pharmaceutically acceptable salt thereof.

In some embodiments, the compound has the structure of Formula (IC):

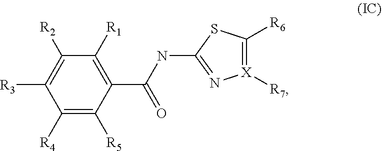

or a pharmaceutically acceptable salt thereof,
wherein X is selected from C and N;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein when X is C at least one of R₁, R₂, R₃, R₄ and R₅ is not H;
R₆ is H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof;
R₇ is absent, H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof; and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound having the structure of Formula (IC) is selected from the group consisting of:

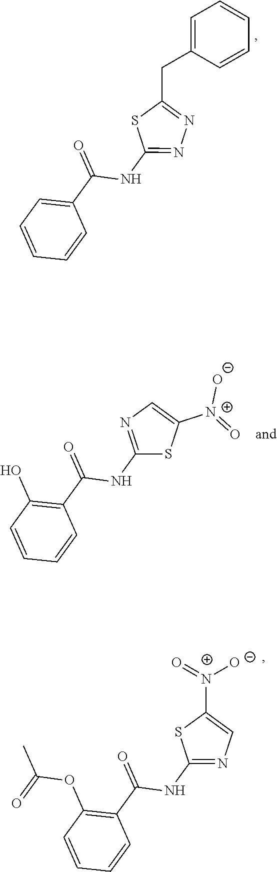 [SMILES: O=C(Nc1nnc(Cc2ccccc2)s1)c1ccccc1 ; O=C(Nc1ncc([N+](=O)[O-])s1)c1ccccc1O ; CC(=O)Oc1ccccc1C(=O)Nc1ncc([N+](=O)[O-])s1]

or a pharmaceutically acceptable salt thereof.

In another aspect, provided herein is a method of treating a viral infection in a subject comprising administering to the subject a compound capable of inhibiting enzymatic activity of the NSP14-NSP10 complex having the structure of Formula (II):

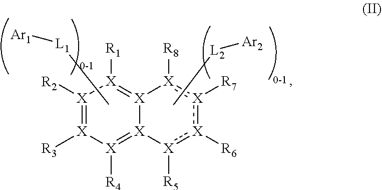

or a pharmaceutically acceptable salt thereof,
wherein X is independently at each occurrence selected from C, O, N, and S;
L₁ and L₂ are independently a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar₁ and Ar₂ are independently a phenyl, a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, R₇ and R₈ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound has the structure of Formula (IIA):

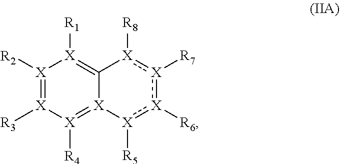

or a pharmaceutically acceptable salt thereof,
wherein X is independently at each occurrence selected from C, O, N, and S;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —F, —Cl, —Br, —I, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form one or more fused rings, and wherein at least two of R₁, R₂, R₃, R₄, R₅, R₆, R₇ and R₈ are not H;
R* is independently selected at each occurrence from hydrogen, C₁₋₁₂ alkyl, C₁₋₁₂ alkenyl, C₆₋₁₂ aryl, and C₁₋₁₂ aralkyl, each of which optionally contains 1-8 heteroatoms selected from halogen, O, N, and S and combinations thereof.

In some embodiments, the compound having structure of Formula (IIA) is selected from the group consisting of:

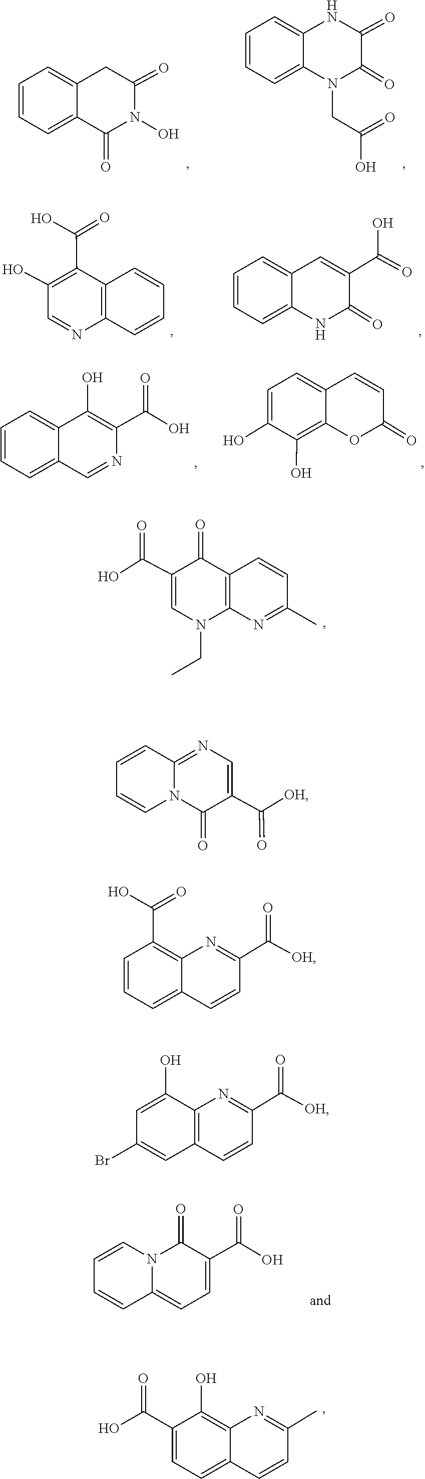

or a pharmaceutically acceptable salt thereof.

In some embodiments, the compound has the structure of Formula (IIB):

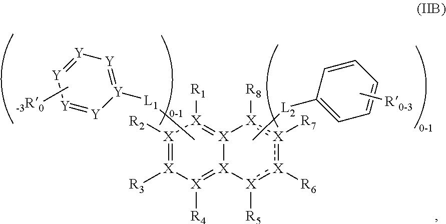

or a pharmaceutically acceptable salt thereof,
wherein X is independently at each occurrence selected from C, O, and N;
Y is independently at each occurrence selected from a bond, C, and N;
L₁ and L₂ are independently a linker selected from a bond and a C₁₋₃ alkyl optionally containing 1-2 heteroatoms selected from O, N, and S;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound having the structure of Formula (IIB) is selected from the group consisting of:

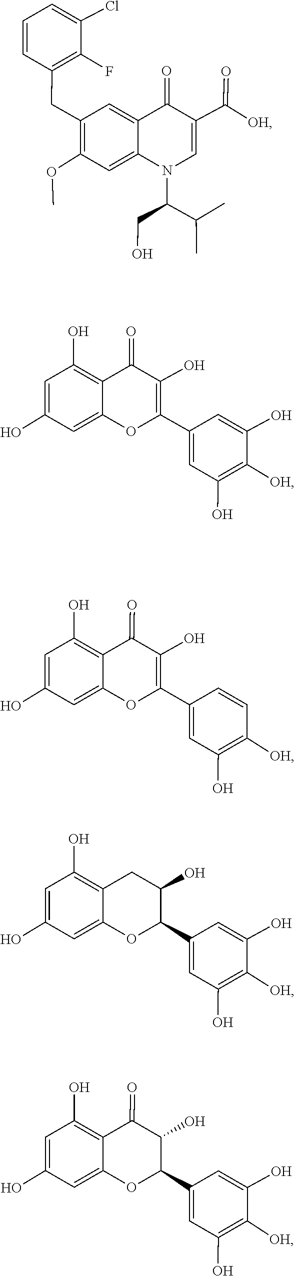 [SMILES: COc1cc2c(cc1Cc1cccc(Cl)c1F)c(=O)c(C(=O)O)cn2[C@H](CO)C(C)C ; O=c1c(O)c(-c2cc(O)c(O)c(O)c2)oc2cc(O)cc(O)c12 ; O=c1c(O)c(-c2ccc(O)c(O)c2)oc2cc(O)cc(O)c12 ; Oc1cc(O)c2c(c1)O[C@H](c1cc(O)c(O)c(O)c1)[C@H](O)C2 ; O=C1c2c(O)cc(O)cc2O[C@H](c2cc(O)c(O)c(O)c2)[C@H]1O]

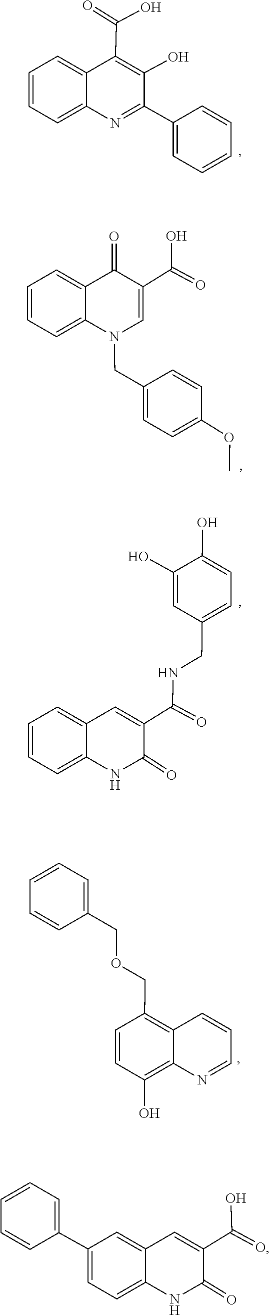 [SMILES: O=C(O)c1c(O)c(-c2ccccc2)nc2ccccc12 ; COc1ccc(Cn2cc(C(=O)O)c(=O)c3ccccc32)cc1 ; O=C(NCc1ccc(O)c(O)c1)c1cc2ccccc2[nH]c1=O ; Oc1ccc(COCc2ccccc2)c2cccnc12 ; O=C(O)c1cc2cc(-c3ccccc3)ccc2[nH]c1=O]

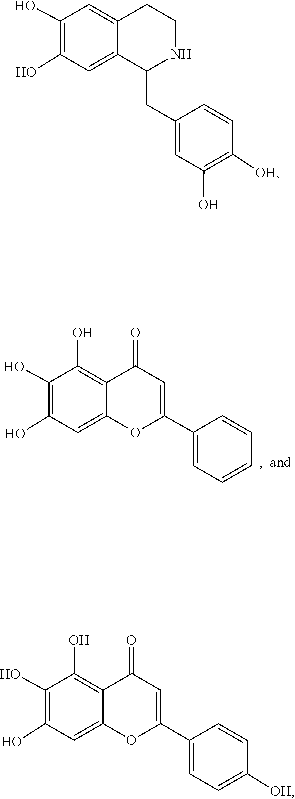 [SMILES: Oc1ccc(CC2NCCc3cc(O)c(O)cc32)cc1O ; O=c1cc(-c2ccccc2)oc2cc(O)c(O)c(O)c12 ; O=c1cc(-c2ccc(O)cc2)oc2cc(O)c(O)c(O)c12]

or a pharmaceutically acceptable salt thereof.

In some embodiments, the compound having the structure of Formula (IIB) is selected from the group consisting of:

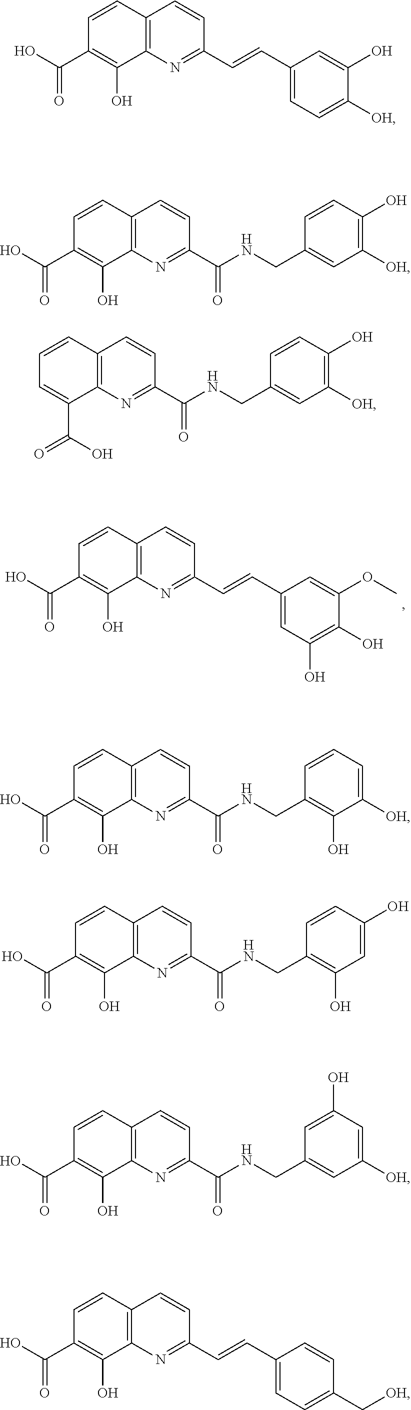

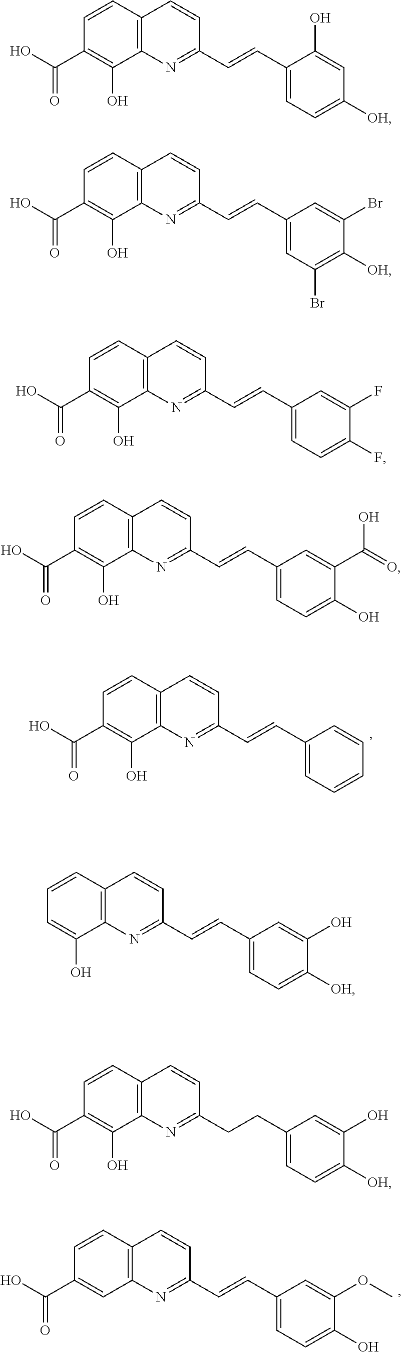

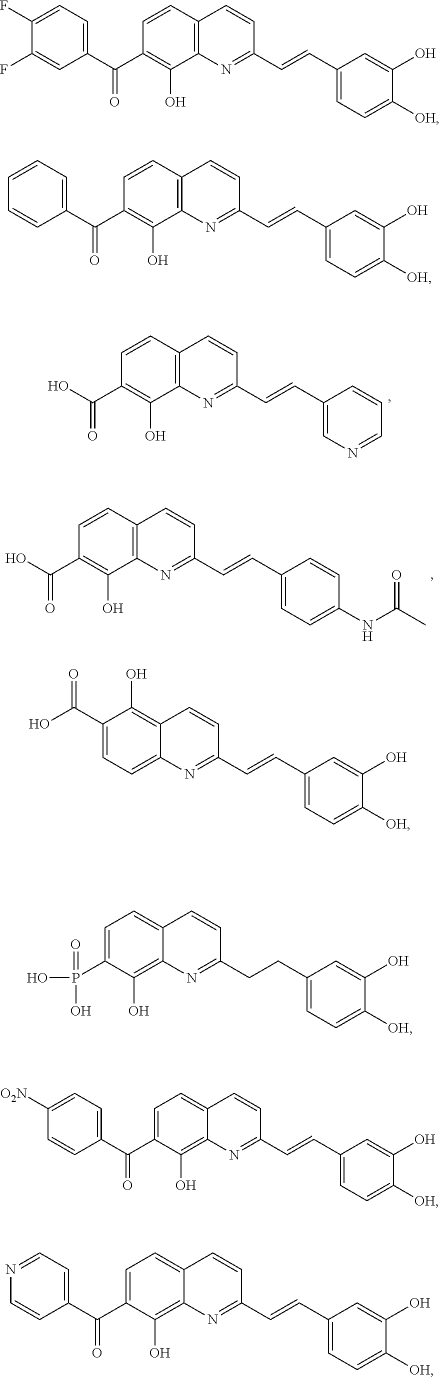

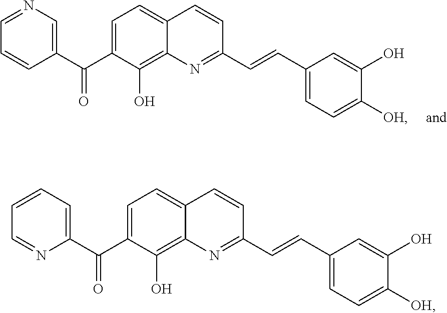 [SMILES: O=C(c1cccnc1)c1ccc2ccc(/C=C/c3ccc(O)c(O)c3)nc2c1O ; O=C(c1ccccn1)c1ccc2ccc(/C=C/c3ccc(O)c(O)c3)nc2c1O]

or a pharmaceutically acceptable salt thereof.

In some embodiments, the compound of Formula (IIB) has the structure:

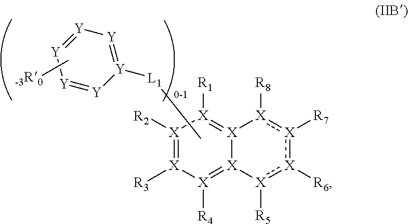

or a pharmaceutically acceptable salt thereof.

In some embodiments, the compound of Formula (IIB′) has the structure:

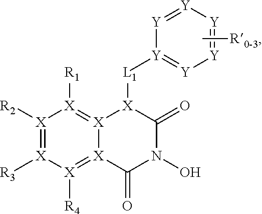

or a pharmaceutically acceptable salt thereof.

In one particular embodiment, the compound of Formula (IIB′) has the structure:

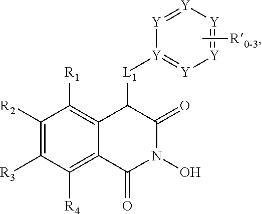

or a pharmaceutically acceptable salt thereof.

In some embodiments, the compound having the structure of Formula (IIB′) is selected from the group consisting of:

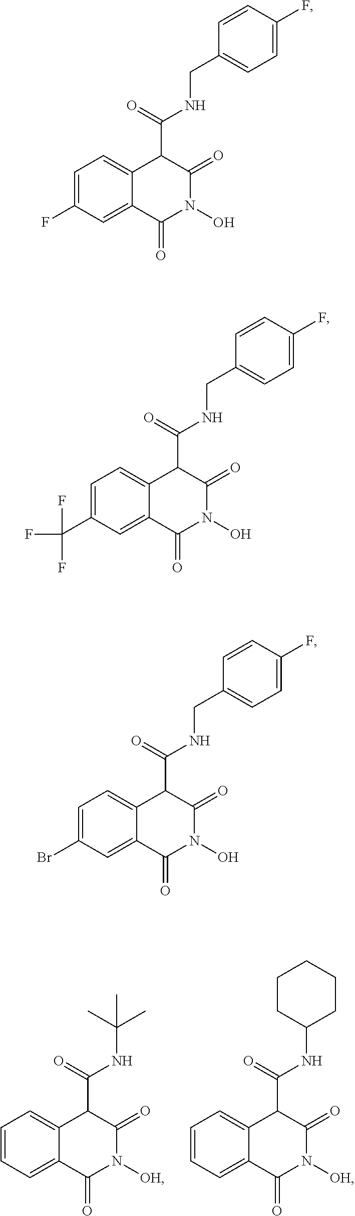 [SMILES: O=C(NCc1ccc(F)cc1)C1C(=O)N(O)C(=O)c2cc(F)ccc21 ; O=C(NCc1ccc(F)cc1)C1C(=O)N(O)C(=O)c2cc(C(F)(F)F)ccc21 ; O=C(NCc1ccc(F)cc1)C1C(=O)N(O)C(=O)c2cc(Br)ccc21 ; CC(C)(C)NC(=O)C1C(=O)N(O)C(=O)c2ccccc21 ; O=C(NC1CCCCC1)C1C(=O)N(O)C(=O)c2ccccc21]

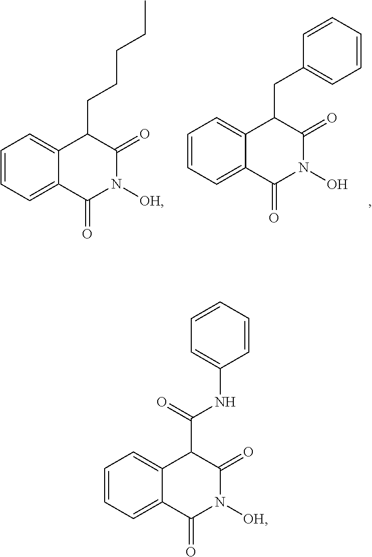

or a pharmaceutically acceptable salt thereof.

In some embodiments, the compound of Formula (IIB) has the structure:

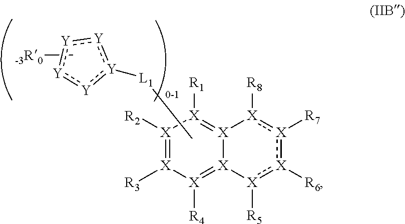

or a pharmaceutically acceptable salt thereof.

In some embodiment, the compound of Formula (IIB″) has the structure:

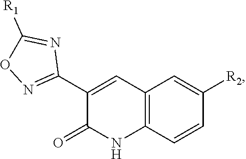

or a pharmaceutically acceptable salt thereof,
wherein R₁ and R₂ are independently H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof.

In some embodiments, the compound having the structure of Formula (IIB″) is selected from the group consisting of:

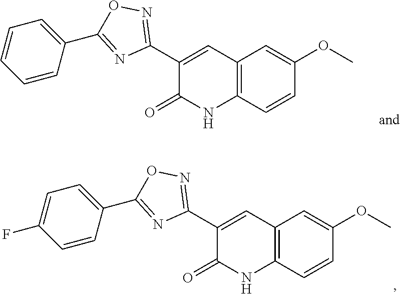 [SMILES: COc1ccc2[nH]c(=O)c(-c3noc(-c4ccccc4)n3)cc2c1 ; COc1ccc2[nH]c(=O)c(-c3noc(-c4ccc(F)cc4)n3)cc2c1]

or a pharmaceutically acceptable salt thereof.

In some embodiments, the compound has the structure of Formula (IIC):

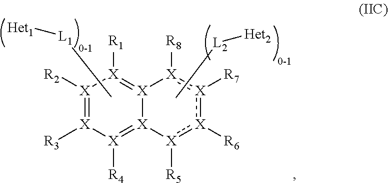

or a pharmaceutically acceptable salt thereof,
wherein X is independently at each occurrence selected from C, O, and N;
L₁ and L₂ are independently a linker selected from a bond and a C₁₋₃ alkyl optionally containing 1-2 heteroatoms selected from O, N, and S;
Het₁ and Het₂ are independently a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound having the structure of Formula (IIC) is selected from the group consisting of:

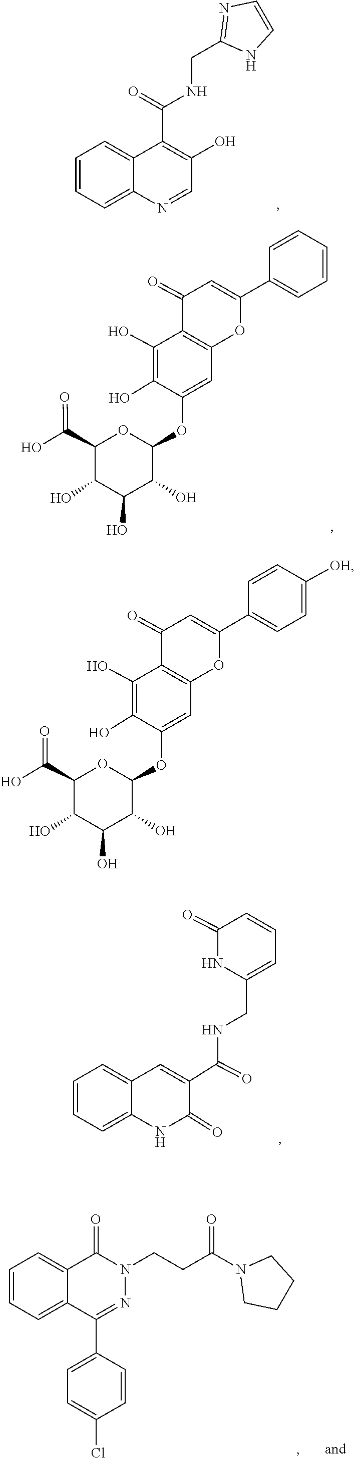 [SMILES: O=C(NCc1ncc[nH]1)c1c(O)cnc2ccccc12 ; O=C(O)[C@H]1O[C@@H](Oc2cc3oc(-c4ccccc4)cc(=O)c3c(O)c2O)[C@H](O)[C@@H](O)[C@@H]1O ; O=C(O)[C@H]1O[C@@H](Oc2cc3oc(-c4ccc(O)cc4)cc(=O)c3c(O)c2O)[C@H](O)[C@@H](O)[C@@H]1O ; O=C(NCc1cccc(=O)[nH]1)c1cc2ccccc2[nH]c1=O ; O=C(CCn1nc(-c2ccc(Cl)cc2)c2ccccc2c1=O)N1CCCC1]

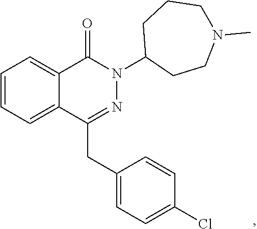 [SMILES: CN1CCCC(n2nc(Cc3ccc(Cl)cc3)c3ccccc3c2=O)CC1]

or a pharmaceutically acceptable salt thereof.

In some embodiments, the compound having the structure of Formula (IIC) is selected from the group consisting of:

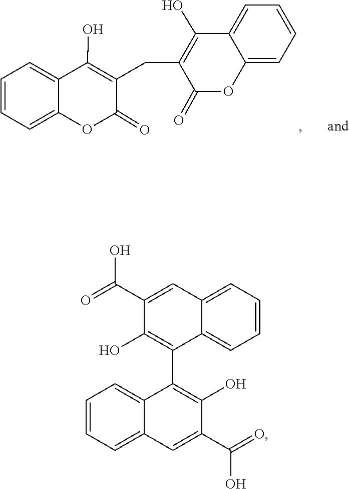 [SMILES: O=c1oc2ccccc2c(O)c1Cc1c(O)c2ccccc2oc1=O ; O=C(O)c1cc2ccccc2c(-c2c(O)c(C(=O)O)cc3ccccc23)c1O]

or a pharmaceutically acceptable salt thereof.

In some embodiments, the adjacent two or more of R₁, R₂, R₃, R₄, R₅, R₆, R₇ and R₈ combine to form one or more fused rings, which may be further substituted with one or more substituents to form a fused polycyclic ring system.

In some embodiments, the compound of formula (II) is selected from the group consisting of:

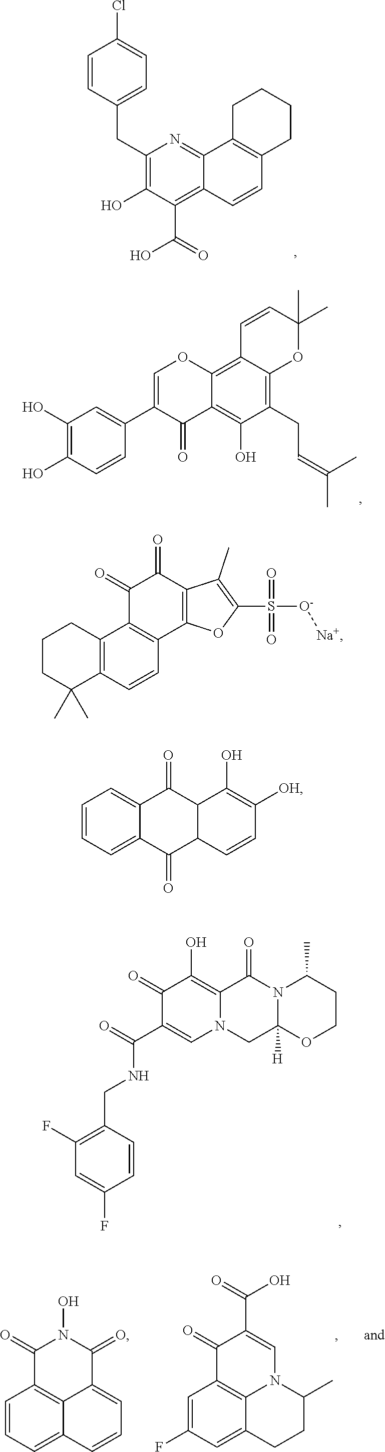

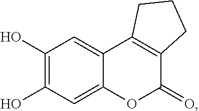 [SMILES: O=c1oc2cc(O)c(O)cc2c2c1CCC2]

or a pharmaceutically acceptable salt thereof.

In some embodiments, the compound having the structure of Formula (II) is selected from the group consisting of PSI-697, Pomiferin, Tanshinone IIa sulfonate, Alizarin, Dolutegravir, Flumequine, and N-hydroxynaphthalimide.

In another aspect, provided herein is a method of treating a viral infection in a subject comprising administering to the subject a compound capable of inhibiting enzymatic activity of the NSP14-NSP10 complex having the structure of Formula (II′):

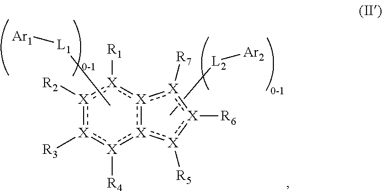

or a pharmaceutically acceptable salt thereof,
wherein X is independently at each occurrence selected from C, O, N, and S;
L₁ and L₂ are independently a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar₁ and Ar₂ are independently a phenyl, a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, and R₇ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In some embodiments, the compound of Formula (II′) is selected from the group consisting of:

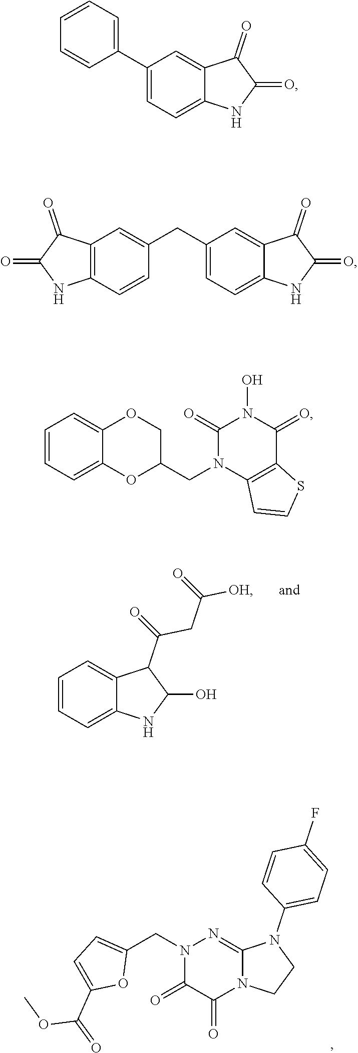 [SMILES: O=C1Nc2ccc(-c3ccccc3)cc2C1=O ; O=C1Nc2ccc(Cc3ccc4c(c3)C(=O)C(=O)N4)cc2C1=O ; O=c1c2sccc2n(CC2COc3ccccc3O2)c(=O)n1O ; O=C(O)CC(=O)C1c2ccccc2NC1O ; COC(=O)c1ccc(Cn2nc3n(c(=O)c2=O)CCN3c2ccc(F)cc2)o1]

or a pharmaceutically acceptable salt thereof.

In some embodiments, (L₁-Ar₁) is absent. In some embodiments, the compound of Formula (I′) has the structure according to Formula (II′A):

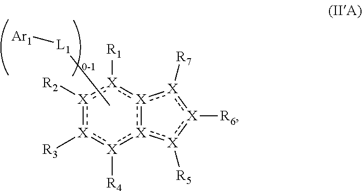

or a pharmaceutically acceptable salt thereof.

In some embodiments, the compound of Formula (II′) has the structure according to Formula (II′B):

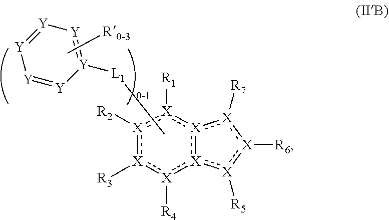

or a pharmaceutically acceptable salt thereof,
wherein Y is independently at each occurrence selected from a bond, C, O, N, and S.

In some embodiments, the compound of Formula (II′) has the structure according to Formula (II′C):

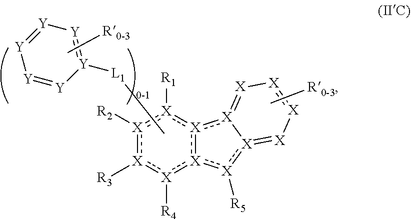

or a pharmaceutically acceptable salt thereof.

In some embodiments, the compound of Formula (II′C) has the structure:

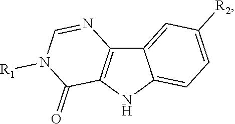

or a pharmaceutically acceptable salt thereof,
wherein R₁ and R₂ are independently H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof.

In some embodiments, the compound having the structure of Formula (II′C) is

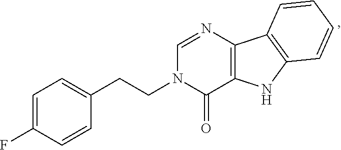 [SMILES: O=c1c2[nH]c3ccccc3c2ncn1CCc1ccc(F)cc1]

or a pharmaceutically acceptable salt thereof.

In some embodiments, (L₂-Ar₂) is absent. In some embodiments, the compound of Formula (II′) has the structure according to Formula (II′D):

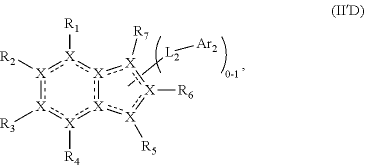

or a pharmaceutically acceptable salt thereof.

In some embodiments, the compound of Formula (II′) has the structure according to Formula (II′E):

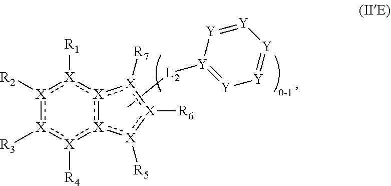

or a pharmaceutically acceptable salt thereof,
wherein Y is independently at each occurrence selected from a bond, C, O, N, and S.

In some embodiments, the adjacent two or more of R₁, R₂, R₃, R₄, R₅, R₆, R₇ and R₈ combine to form one or more fused rings, which may be further substituted with one or more substituents to form a fused polycyclic ring system.

In some embodiments, the compound of Formula (II′) is selected from the group consisting of:

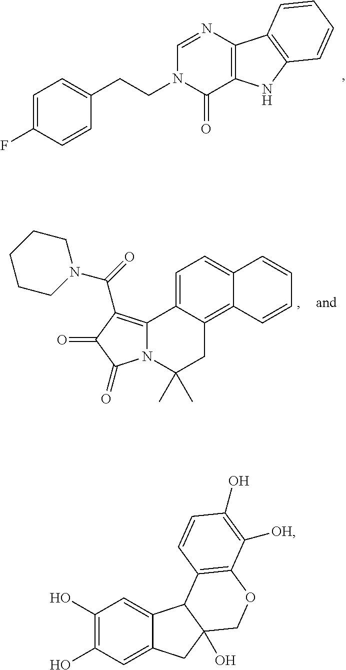 [SMILES: O=c1c2[nH]c3ccccc3c2ncn1CCc1ccc(F)cc1 ; CC1(C)Cc2c(ccc3ccccc23)C2=C(C(=O)N3CCCCC3)C(=O)C(=O)N21 ; Oc1cc2c(cc1O)C1c3ccc(O)c(O)c3OCC1(O)C2]

or a pharmaceutically acceptable salt thereof.

In another aspect, provided herein is a method of treating a viral infection in a subject comprising administering to the subject a compound capable of inhibiting enzymatic activity of the NSP14-NSP10 complex having the structure of Formula (III):

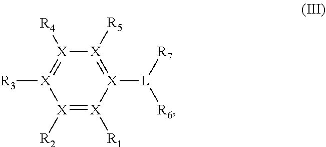

or a pharmaceutically acceptable salt thereof,
wherein X is independently at each occurrence selected from C, O, N, and S;
L is a linker selected from a bond, a C₁₋₁₂ alkyl, C₂₋₁₂ alkenyl, —CO—, —CO—NH—, —CO—(C₁₋₃ alkyl)-, and —CO—(C₂₋₄ alkenyl)-, and combinations thereof, wherein the C₁₋₁₂ alkyl or the C₂₋₁₂ alkenyl optionally contains 1-5 heteroatoms selected from O, N, and S;
R₁, R₂, R₃, R₄, and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring;
R₆ and R₇ are independently at each occurrence H, C₁₋₁₂ alkyl, C₁₋₁₂ alkenyl, C₆₋₁₂ aryl, C₁₋₁₂ aralkyl, C₁-C₁₂ heteroaryl, C₁₋₄ haloalkyl, —F, —Cl, —Br, —I, —OH, ═O, —CO₂H, —NO₂, —N—OH, —HSO₃, —H₂PO₃, —OR*, —(C═O)—R*, —CO₂R*, —CO—NHR*, —SO₂—NHR*, R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In another aspect, provided herein is a method of treating a viral infection in a subject comprising administering to the subject a compound capable of inhibiting enzymatic activity of the NSP14-NSP10 complex having the structure of Formula (IIIA):

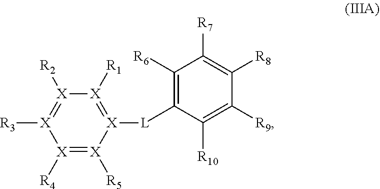

or a pharmaceutically acceptable salt thereof,
wherein X is independently at each occurrence selected from C, N, O, and S;
L is a linker selected from a bond, a C₁₋₁₂ alkyl, C₂₋₁₂ alkenyl, —CO—, —CO—NH—, —CO—(C₁₋₃ alkyl)-, and —CO—(C₂₋₄ alkenyl)-, and combinations thereof, wherein the C₁₋₁₂ alkyl or the C₂₋₁₂ alkenyl optionally contains 1-5 heteroatoms selected from O, N, and S;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least one of R₁, R₂, R₃, R₄ and R₅ is not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

The compound of Formula (IIIA), wherein at least two of R₁, R₂, R₃, R₄ and R₅ are not H.

In another aspect, provided herein is a method of treating a viral infection in a subject comprising administering to the subject a compound capable of inhibiting enzymatic activity of the NSP14-NSP10 complex having the structure of Formula (IIIB):

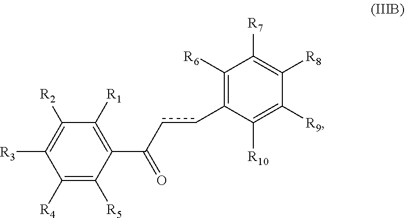

or a pharmaceutically acceptable salt thereof,
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least one of R₁, R₂, R₃, R₄ and R₅ is not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

The compound of Formula (IIIB), wherein at least two of R₁, R₂, R₃, R₄ and R₅ are not H.

In some embodiments, the compound of Formula (IIIB) is selected from the group consisting of:

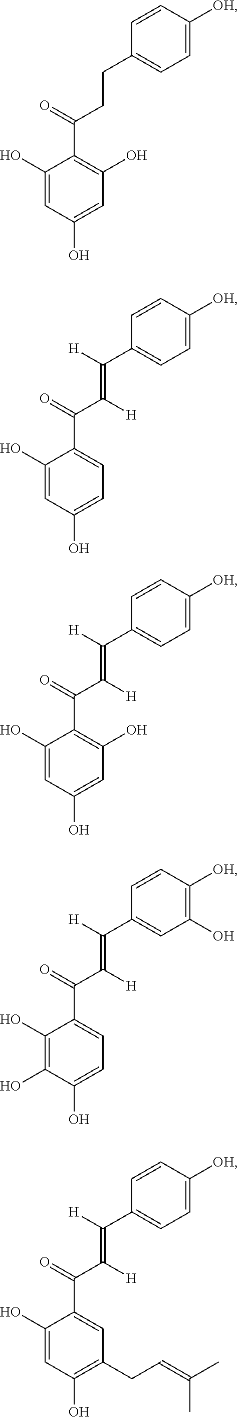 [SMILES: O=C(CCc1ccc(O)cc1)c1c(O)cc(O)cc1O ; O=C(/C=C/c1ccc(O)cc1)c1ccc(O)cc1O ; O=C(/C=C/c1ccc(O)cc1)c1c(O)cc(O)cc1O ; O=C(/C=C/c1ccc(O)c(O)c1)c1ccc(O)c(O)c1O ; CC(C)=CCc1cc(C(=O)/C=C/c2ccc(O)cc2)c(O)cc1O]

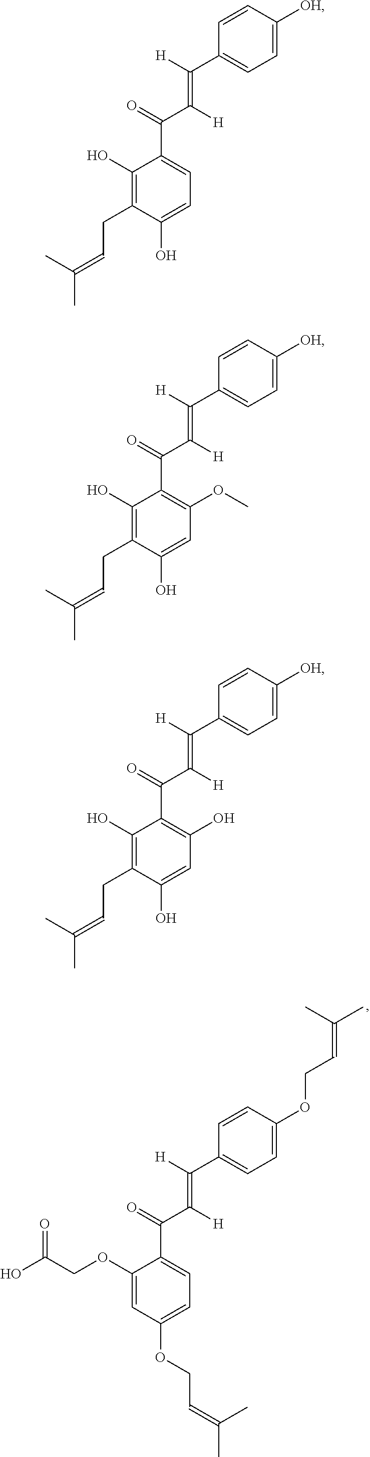 [SMILES: CC(C)=CCc1c(O)ccc(C(=O)/C=C/c2ccc(O)cc2)c1O ; COc1cc(O)c(CC=C(C)C)c(O)c1C(=O)/C=C/c1ccc(O)cc1 ; CC(C)=CCc1c(O)cc(O)c(C(=O)/C=C/c2ccc(O)cc2)c1O ; CC(C)=CCOc1ccc(/C=C/C(=O)c2ccc(OCC=C(C)C)cc2OCC(=O)O)cc1]

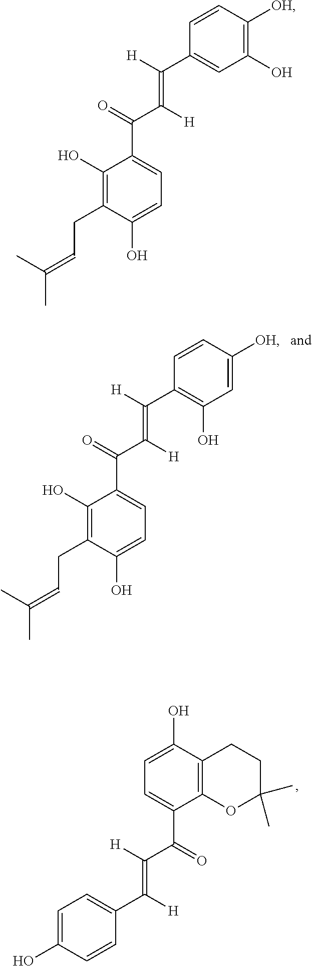 [SMILES: CC(C)=CCc1c(O)ccc(C(=O)/C=C/c2ccc(O)c(O)c2)c1O ; CC(C)=CCc1c(O)ccc(C(=O)/C=C/c2ccc(O)cc2O)c1O ; "and" ; CC1(C)CCc2c(O)ccc(C(=O)/C=C/c3ccc(O)cc3)c2O1]

or a pharmaceutically acceptable salt thereof.

In various embodiments of any of the treatment methods described above, the viral infection is a coronaviral infection. In some embodiments, the viral infection is a SARS-CoV, a SARS-CoV2, a MERS-CoV, a HCoV-229E, a HCoV-NL63, a HCoV-HKU1 or a HCoV-OC43 infection or involves another coronavirus strain of animal or zoonotic origins. In some embodiments, the viral infection is a SARS-CoV2 infection. In some embodiments, the viral infection is a SARS-CoV infection. In some embodiments, the viral infection is a MERS-CoV infection.

In another aspect, provided herein is a method of treating a viral infection in a subject comprising administering to the subject a compound capable of inhibiting enzymatic activity of the NSP14-NSP10 complex having the structure selected from the group presented in Table 2, or a pharmaceutically acceptable salt thereof. In some embodiments, the viral infection is a coronaviral infection. In some embodiments, the viral infection is a SARS-CoV, a SARS-CoV2, a MERS-CoV, a HCoV-229E, a HCoV-NL63, a HCoV-HKU1 or a HCoV-OC43 infection or involves another coronavirus strain of animal or zoonotic origins. In some embodiments, the viral infection is a SARS-CoV2 infection. In some embodiments, the viral infection is a SARS-CoV infection. In some embodiments, the viral infection is a MERS-CoV infection.

In various embodiments of any of the treatment methods described above, the method further comprises administering a ribonucleotide analog to the subject. In some embodiments, the ribonucleotide analog is selected from Remdesivir, Ribavirin, Favipiravir, N₄-Hydroxycytidine (EIDD-1931) or its derivative Molnupiravir, 5-Fluorouracil and Sofosbuvir.

### BRIEF DESCRIPTION OF THE DRAWINGS

FIG. 1 shows coomassie brilliant blue R-250 staining of purified NSP10-His tagged protein and NSP14-His tagged protein expressed in E. coli.

FIG. 2 shows a schematic representation of the structure of the SARS-CoV2 NSP14/NSP10 complex highlighting the DEDD(h)-type catalytic domain of NSP14, including the Mg²⁺ ion cofactor requisite for catalysis.

FIGS. 3A-3B show the design of exemplar 6FAM-BHQ1 reporter-quencher probes and the optimized double-stranded RNA (dsRNA) substrates. The dsRNA substrates were based on both FRET (dynamic or excited state) and static (ground state) quenching mechanisms. A fluorophore reporter 6FAM was attached to the 5′ end of the sense (5′-to-3′) strand. To form the dsRNA substrate, the RNA sense strand was annealed to a complementary antisense strand (3′-to-5′) attached to a non-fluorescent Black Hole Quencher-1 (BHQ1) quencher molecule, which was positioned at either the 5′ or the 3′ end of the antisense strand (FIG. 3A). The designs for optimized oligonucleotide substrates Oligo A-D (SEQ ID NOS 1-8, respectively, in order of appearance) show the consensus structure (FIG. 3B).

FIGS. 4A-4B show NSP14/10 FRET-based activity assay reactions for Oligo A-D. Reactions were visualized and imaged in 200 μl microtubes for side-by-side comparison of fluorescence intensity using a blue light transilluminator (FIG. 4A). Fluorescence intensity (left) was measured and plotted in arbitrary units (AU). Fold change of fluorescence intensity upon RNase treatment (right) was calculated as the amount of fluorescence measured for the RNase treatment group divided by the amount of fluorescence measured for the reaction buffer alone group for each dsRNA substrate (FIG. 4B).

FIGS. 5A-5B show NSP14/10 FRET-based activity assay reactions for Oligo A and Oligo B. Images of fluorescent signal emitted from the reactions across conditions (FIG. 5A). Fluorescence intensity plotted in arbitrary units (AU) for Oligo A and Oligo B across different reaction conditions (FIG. 5B).

FIGS. 6A-6B demonstrate Mg²⁺ chelator-based (EDTA) inhibition of NSP14/NSP10 complex activity, as detected by FRET-based activity assay. Images of fluorescent signal emitted from the reactions across conditions (FIG. 6A). Fluorescence intensity of the reactions quantified across three EDTA concentrations (5 mM, 10 mM, and 50 mM EDTA), revealed inhibition of the NSP14/NSP10 complex exonuclease activity within the 0-5 mM range (FIG. 6A). Subsequent testing of finer concentration gradations at <5 mM EDTA (across a total of six different concentrations: 0.16 mM, 0.32 mM, 0.63 mM, 1.25 mM, 2.5 mM and 5 mM EDTA) showed titration of free Mg²⁺ from the reaction mixture, and thus complete inhibition of complex activity on Oligo B, at approximately 2 mM EDTA (FIG. 6B).

FIGS. 7A-7B show NSP10-mediated facilitation of NSP14 exonuclease activity. Oligo D was diluted in reaction buffer containing increasing molar excess ratios of NSP14:NSP10 (1:1-1:5 molar ratio, range). Images of fluorescent signal emitted from the reactions (FIG. 7A). A plot of fluorescence intensity relative to the NSP14 reaction condition across increasing molar excess ratios of NSP14:NSP10 (FIG. 7B).

FIGS. 8A-8B show evaluation of background fluorescence from common contaminants. A panel of four purified recombinant proteins (8-Oxoguanine DNA Glycosylase [OGG1]; BTB Domain and CNC Homolog 1 [Bach1]; Ubiquitin conjugating enzyme UbcH3; bovine serum albumin [BSA]) were tested in parallel with unbound NSP14, unbound NSP10, and NSP14/NSP10 complex by FRET-based activity assay. Images of fluorescent signal emitted from the reactions (FIG. 8A). A plot of fluorescence intensity relative to the reaction mixture reaction alone (buffer) condition across the reaction conditions (buffer, NSP14, NSP10, NSP14/10, OGG1, Bach1, UbcH3, BSA) shows that background contamination was negligible by comparison to that observed for the NSP14/NSP10 complex (FIG. 8B).

FIG. 9 shows a graph comparing the kinetic profile of NSP14/NSP10 complex activity on dsRNA substrate designs Oligo A-D, as detected by FRET-based activity assay. The NSP14/NSP10 complex digested each of Oligo A-D in a time dependent manner, with the reactions generally reaching completion between approximately 30 to 40 minutes (approximately 1,800 seconds to 2500 seconds). Given that Oligo B was longer than Oligo C (or Oligo D) by 5 base pairs (bps), Oligo B required approximately 3,000 (approximately 50 minutes) seconds to reach reaction completion, whereas Oligo C required approximately 2000 seconds (approximately 33 minutes). Together, these findings indicate that the rate of digestion of the NSP14/10 complex was approximately 0.3 bp/second.

FIG. 10 shows a panel of graphs of kinetic profiles of unbound NSP14, unbound NSP10, and NSP14/NSP10 complex activity on dsRNA substrate designs Oligo A-D, as detected by FRET-based activity assay. The signal-to-noise ratio was determined as the fold change in fluorescence signal intensity (an index of complex activity) measured for the NSP14/10 complex condition relative to that of NSP10 alone condition. For each Oligo A-D, NSP14/NSP10 fluorescence intensity was more than an order of magnitude higher than that of NSP10 alone (1.32-fold to 4.32-fold, range).

FIGS. 11A-11B show plots of dose-response curves for 2-Hydroxyisoquinoline-1,3(2H,4H)-dione. The dose-response curve for 2-Hydroxyisoquinoline-1,3(2H,4H)-dione shows half maximal inhibitory concentration (IC₅₀) as 397 μM (FIG. 11A). The dose-response curves for 2-Hydroxyisoquinoline-1,3(2H,4H)-dione inhibition of the NSP14/10 complex, unbound NSP10, and Dimethyl Sulfoxide (DMSO) control indicates highly specific 2-Hydroxyisoquinoline-1,3(2H,4H)-dione inhibition of NSP14/NSP10 complex exonuclease activity (FIG. 11B). IC₅₀ values were measured in the linear reaction range, and less than 60% of the total substrate had been consumed at readout. FIG. 11C shows a plot of the fluorescent intensity signal during the time course of NSP14/NSP10 inhibition across increasing concentrations of 2-Hydroxyisoquinoline-1,3(2H,4H)-dione (mM), as detected by FRET-based activity assay.

FIG. 11D shows a plot of dose-response curves for 5,5′-Methylenedisalicylic acid. The dose-response curve for 5,5′-Methylenedisalicylic acid shows half maximal inhibitory concentration (IC₅₀) as 202 μM. IC₅₀ values were measured in the linear reaction range, and less than 60% of the total substrate had been consumed at readout. FIG. 11E shows a plot of the fluorescent intensity signal during the time course of NSP14/NSP10 inhibition across increasing concentrations of 5,5′-Methylenedisalicylic acid (mM), as detected by FRET-based activity assay.

FIG. 11F shows a plot of dose-response curves for 3-Hydroxy-N-[(1H-imidazol-2-yl)methyl]quinoline-4-carboxamide. The dose-response curve for 3-Hydroxy-N-[(1H-imidazol-2-yl)methyl]quinoline-4-carboxamide shows half maximal inhibitory concentration (IC₅₀) as 211 M. IC₅₀ values were measured in the linear reaction range, and less than 60% of the total substrate had been consumed at readout. FIG. 11G shows a plot of the fluorescent intensity signal during the time course of NSP14/NSP10 inhibition across increasing concentrations of 3-Hydroxy-N-[(1H-imidazol-2-yl)methyl]quinoline-4-carboxamide (mM), as detected by FRET-based activity assay.

FIG. 11H shows a plot of dose-response curves for Phloretin. The dose-response curve for Phloretin shows half maximal inhibitory concentration (IC₅₀) as 335 μM. IC₅₀ values were measured in the linear reaction range, and less than 60% of the total substrate had been consumed at readout. FIG. 11I shows a plot of the fluorescent intensity signal during the time course of NSP14/NSP10 inhibition across increasing concentrations of Phloretin (mM), as detected by FRET-based activity assay.

FIG. 11J shows a plot of dose-response curves for Dicoumarol. The dose-response curve for Dicoumarol shows half maximal inhibitory concentration (IC₅₀) above 500 μM. FIG. 11K shows a plot of the fluorescent intensity signal during the time course of NSP14/NSP10 inhibition across increasing concentrations of Dicoumarol (mM), as detected by FRET-based activity assay.

FIG. 12 shows the results from a viability assay. In brief, A549, human lung cells, were treated for 48 hours with the indicated compounds. Viability was measured with alamarBlue assay following the recommendation of the manufacturer.

FIG. 13 shows a schematic representation of the Thermofluor (thermal shift) assay used to interrogate the thermal stability of the NSP14/NSP10 complex in the presence or absence of 2-Hydroxyisoquinoline-1,3(2H,4H)-dione.

FIGS. 14A-14B show the thermal stability of a control protein tested in the presence or absence of its cognate ligand, as determined by Thermofluor (thermal shift) assay. Derivatized melting curves for a control protein in the presence or absence of its cognate ligand (FIG. 14A). Derivatized melting curves for the reaction buffer plus DMSO and reaction buffer plus Hydroxyisoqunoline-1,3(2H,4H)-dione (FIG. 14B).

FIGS. 15A-15B show the thermal stability of the NSP14/NSP10 complex in the presence or absence of 2-Hydroxyisoquinoline-1,3(2H,4H)-dione (NHID #13), as determined by Thermofluor (thermal shift) assay. The derivatized melting curve for the NSP14/10 complex plus DMSO, and the NSP14/10 complex in the presence of Hydroxyisoqunoline-1,3(2H,4H)-dione (FIG. 15A). Raw melting curve data for the NSP14/10 complex plus DMSO, and the NSP14/10 complex in the presence of Hydroxyisoqunoline-1,3(2H,4H)-dione (FIG. 15B).

FIG. 16 shows the FRET signal obtained with digestion of Oligo D by either RNase A or the NSP14/10 complex followed by subsequent addition of Hydroxyisoquinoline-1,3(2H,4H)-dione. Reactions were visualized and imaged in 200 μl microtubes for side-by-side comparison of fluorescence intensity using a blue light transilluminator (left). Fluorescence intensity was measured and plotted (right) in arbitrary units (AU).

FIG. 17 shows a comparison of the double-stranded RNA (dsRNA) substrate designs comprising 6FAM-BHQ1 and TexasRed-BHQ2 reporter-quencher probes (SEQ ID NOS 1-10, respectively, in order of appearance). For the 6FAM-BHQ1 FRET probes, a fluorophore reporter 6FAM was attached to the 5′ end of the sense (5′-to-3′) strand. The RNA sense strand was annealed to a complementary antisense strand (3′-to-5′) attached to a non-fluorescent Black Hole Quencher-1 (BHQ1) quencher molecule, which was positioned at either the 5′ or the 3′ end of the antisense strand. For the TexasRed-BHQ2 FRET pair, a fluorophore reporter TexasRed was attached to the 5′ end of the sense (5′-to-3′) strand. The RNA sense strand was annealed to a complementary antisense strand (3′-to-5′) attached to a non-fluorescent Black Hole Quencher-2 (BHQ2) quencher molecule, which was positioned at the 3′ end of the antisense strand. The dsRNA substrate for Oligo E was identical to that of Oligo D. Optimized oligonucleotide substrates Oligo A-E show various aspects of the consensus structure.

FIGS. 18A-18B show NSP14/10 FRET-based activity assay reactions for Oligo A-E. Reactions were visualized and imaged in 200 μl microtubes for side-by-side comparison of fluorescence intensity using a blue light transilluminator (FIG. 18A). Fluorescence intensity (left) was measured and plotted in arbitrary units (AU). Fold change of fluorescence intensity upon RNase treatment (right) was calculated as the amount of fluorescence measured for the RNase treatment group divided by the amount of fluorescence measured for the reaction buffer alone group for each dsRNA substrate (FIG. 18B).

FIG. 19 shows a graph comparing the kinetic profile of NSP14/NSP10 complex activity on dsRNA substrate designs Oligo A-E, as detected by FRET-based activity assay.

FIG. 20 shows a graph of the individual kinetic profiles for the activity of unbound NSP10, NSP14/NSP10 complex alone, and NSP14/NSP10 complex plus Hydroxyisoquinoline-1,3(2H,4H)-dione (#13; 500 μM) using Oligo E.

FIG. 21 shows a gel electrophoresis assay. Degradation of Oligo B was observed as faster migrating bands versus inhibition of the NSP14/10 complex (Oligo B digestion), which resulted in a slower migration pattern. Inhibition of NSP14/10 by Hydroxyisoquinoline-1,3(2H,4H)-dione (#13) was greater at higher (500 μM) versus lower (100 μM) concentrations.

FIG. 22 depicts binding to the catalytic site with a simple monocyclic core ring (FIG. 22A, 3,3′ Methylenedisalicylic acid), with a polycyclic core ring (FIG. 22B, Tetrahydropapaveroline), or using alternative binding modes (FIG. 22C, Xanthohumol).

FIG. 23A shows a plot of dose-response curves for 2-Oxo-6-phenyl-1H-quinoline-3-carboxylic acid. The dose-response curve for 2-Oxo-6-phenyl-1H-quinoline-3-carboxylic acid shows half maximal inhibitory concentration (IC₅₀) of 185 μM. IC₅₀ values were measured in the linear reaction range, and less than 60% of the total substrate had been consumed at readout.

FIG. 23B shows a plot of the fluorescent intensity signal during the time course of NSP14/NSP10 inhibition across increasing concentrations of 2-Oxo-6-phenyl-1H-quinoline-3-carboxylic acid (μM), as detected by FRET-based activity assay.

FIG. 24A shows a plot of dose-response curves for Tetrahydropapaveroline. The dose-response curve for Tetrahydropapaveroline shows half maximal inhibitory concentration (IC₅₀) of 12 μM. IC₅₀ values were measured in the linear reaction range, and less than 60% of the total substrate had been consumed at readout. FIG. 24B shows a plot of the fluorescent intensity signal during the time course of NSP14/NSP10 inhibition across increasing concentrations of Tetrahydropapaveroline (μM), as detected by FRET-based activity assay.

FIG. 25A shows a plot of dose-response curves for Bavachalcone. The dose-response curve for Bavachalcone shows half maximal inhibitory concentration (IC₅₀) of 21.62 μM. IC₅₀ values were measured in the linear reaction range, and less than 60% of the total substrate had been consumed at readout. FIG. 25B shows a plot of the fluorescent intensity signal during the time course of NSP14/NSP10 inhibition across increasing concentrations of Bavachalcone (μM), as detected by FRET-based activity assay.

FIG. 26A shows a plot of dose-response curves for Xanthohumol. The dose-response curve for Xanthohumol shows half maximal inhibitory concentration (IC₅₀) of 16 μM. IC₅₀ values were measured in the linear reaction range, and less than 60% of the total substrate had been consumed at readout. FIG. 26B shows a plot of the fluorescent intensity signal during the time course of NSP14/NSP10 inhibition across increasing concentrations of Xanthohumol (μM), as detected by FRET-based activity assay.

FIG. 27A shows a plot of dose-response curves for 2-(4-Chlorobenzyl)-3-hydroxy-7,8,9,10-tetrahydrobenzo(H)quinoline-4-carboxylic acid (PSI-697). The dose-response curve for PSI-697 shows half maximal inhibitory concentration (IC₅₀) of 36.04 μM. IC₅₀ values were measured in the linear reaction range, and less than 60% of the total substrate had been consumed at readout. FIG. 27B shows a plot of the fluorescent intensity signal during the time course of NSP14/NSP10 inhibition across increasing concentrations of PSI-697 (μM), as detected by FRET-based activity assay.

FIG. 28A shows a plot of dose-response curves for Tanshinone IIA sulfonate. The dose-response curve for Tanshinone IIA sulfonate shows half maximal inhibitory concentration (IC₅₀) of 1.98 μM. IC₅₀ values were measured in the linear reaction range, and less than 60% of the total substrate had been consumed at readout. FIG. 28B shows a plot of the fluorescent intensity signal during the time course of NSP14/NSP10 inhibition across increasing concentrations of Tanshinone IIA sulfonate (μM), as detected by FRET-based activity assay.

FIG. 29A shows a plot of dose-response curves for Scutellarein. The dose-response curve for Scutellarein shows half maximal inhibitory concentration (IC₅₀) of 96.25 μM. IC₅₀ values were measured in the linear reaction range, and less than 60% of the total substrate had been consumed at readout. FIG. 29B shows a plot of the fluorescent intensity signal during the time course of NSP14/NSP10 inhibition across increasing concentrations of Scutellarein (μM), as detected by FRET-based activity assay.

FIG. 30A shows a plot of dose-response curves for Isobavachalcone. The dose-response curve for Isobavachalcone shows half maximal inhibitory concentration (IC₅₀) of 16.6 μM. IC₅₀ values were measured in the linear reaction range, and less than 60% of the total substrate had been consumed at readout. FIG. 30B shows a plot of the fluorescent intensity signal during the time course of NSP14/NSP10 inhibition across increasing concentrations of Isobavachalcone (μM), as detected by FRET-based activity assay.

FIG. 31A shows a plot of dose-response curves for 3-[(3-Carboxy-2-hydroxy-5-propan-2-ylphenyl)methyl]-2-hydroxy-5-propan-2-ylbenzoic acid. The dose-response curve for 3-[(3-Carboxy-2-hydroxy-5-propan-2-ylphenyl)methyl]-2-hydroxy-5-propan-2-ylbenzoic acid shows half maximal inhibitory concentration (IC₅₀) of 86.33 μM. IC₅₀ values were measured in the linear reaction range, and less than 60% of the total substrate had been consumed at readout.

FIG. 31B shows a plot of the fluorescent intensity signal during the time course of NSP14/NSP10 inhibition across increasing concentrations of 3-[(3-Carboxy-2-hydroxy-5-propan-2-ylphenyl)methyl]-2-hydroxy-5-propan-2-ylbenzoic acid (μM), as detected by FRET-based activity assay.

FIG. 32A shows a plot of dose-response curves for Sofalcone. The dose-response curve for Sofalcone shows half maximal inhibitory concentration (IC₅₀) of 28.79 μM. IC₅₀ values were measured in the linear reaction range, and less than 60% of the total substrate had been consumed at readout. FIG. 32B shows a plot of the fluorescent intensity signal during the time course of NSP14/NSP10 inhibition across increasing concentrations of Sofalcone (μM), as detected by FRET-based activity assay.

FIG. 33A shows a plot of dose-response curves for Pomiferin. The dose-response curve for Pomiferin shows half maximal inhibitory concentration (IC₅₀) of 5.9 μM. IC₅₀ values were measured in the linear reaction range, and less than 60% of the total substrate had been consumed at readout. FIG. 33B shows a plot of the fluorescent intensity signal during the time course of NSP14/NSP10 inhibition across increasing concentrations of Pomiferin (μM), as detected by FRET-based activity assay.

FIG. 34A shows a plot of dose-response curves for Desmethylxanthohumol. The dose-response curve for Desmethylxanthohumol shows half maximal inhibitory concentration (IC₅₀) of 215.7 μM. IC₅₀ values were measured in the linear reaction range, and less than 60% of the total substrate had been consumed at readout. FIG. 34B shows a plot of the fluorescent intensity signal during the time course of NSP14/NSP10 inhibition across increasing concentrations of Desmethylxanthohumol (μM), as detected by FRET-based activity assay.

FIG. 35A shows a plot of dose-response curves for Corylifol B. The dose-response curve for Corylifol B shows half maximal inhibitory concentration (IC₅₀) of 145.6 μM. IC₅₀ values were measured in the linear reaction range, and less than 60% of the total substrate had been consumed at readout. FIG. 35B shows a plot of the fluorescent intensity signal during the time course of NSP14/NSP10 inhibition across increasing concentrations of Corylifol B (μM), as detected by FRET-based activity assay.

FIG. 36A shows a plot of dose-response curves for Isodorsmanin A. The dose-response curve for Isodorsmanin A shows half maximal inhibitory concentration (IC₅₀) of 26.48 μM. IC₅₀ values were measured in the linear reaction range, and less than 60% of the total substrate had been consumed at readout. FIG. 36B shows a plot of the fluorescent intensity signal during the time course of NSP14/NSP10 inhibition across increasing concentrations of Isodorsmanin A (μM), as detected by FRET-based activity assay.

FIG. 37A shows a plot of dose-response curves for 2-(3,4-dihydroxystyryl)-8-hydroxyquinoline-7-carboxylic acid (KH161). The dose-response curve for KH161 shows half maximal inhibitory concentration (IC₅₀) of 34 μM. IC₅₀ values were measured in the linear reaction range, and less than 60% of the total substrate had been consumed at readout. FIG. 37B shows a plot of the fluorescent intensity signal during the time course of NSP14/NSP10 inhibition across increasing concentrations of KH161 (mM), as detected by FRET-based activity assay.

FIG. 38A shows a plot of dose-response curves for 2-(3,4-dihydroxybenzylcarbamoyl)-8-hydroxyquinoline-7-carboxylic acid (TOF452). The dose-response curve for TOF452 shows half maximal inhibitory concentration (IC₅₀) of 551 μM. IC₅₀ values were measured in the linear reaction range, and less than 60% of the total substrate had been consumed at readout. FIG. 38B shows a plot of the fluorescent intensity signal during the time course of NSP14/NSP10 inhibition across increasing concentrations of TOF452 (mM), as detected by FRET-based activity assay.

FIG. 39A shows a plot of dose-response curves for 2-((3-methoxy-4,5-dihydroxy)styryl)-8-hydroxyquinoline-7-carboxylic acid (FZ41). The dose-response curve for FZ41 shows half maximal inhibitory concentration (IC₅₀) of 164 μM. IC₅₀ values were measured in the linear reaction range, and less than 60% of the total substrate had been consumed at readout. FIG. 39B shows a plot of the fluorescent intensity signal during the time course of NSP14/NSP10 inhibition across increasing concentrations of FZ41 (mM), as detected by FRET-based activity assay.

FIG. 40A shows a plot of dose-response curves for 2-(2,3-dihydroxybenzylcarbamoyl)-8-hydroxyquinoline-7-carboxylic acid (TOF438). The dose-response curve for TOF438 shows half maximal inhibitory concentration (IC₅₀) of 110 μM. IC₅₀ values were measured in the linear reaction range, and less than 60% of the total substrate had been consumed at readout. FIG. 40B shows a plot of the fluorescent intensity signal during the time course of NSP14/NSP10 inhibition across increasing concentrations of TOF438 (mM), as detected by FRET-based activity assay.

FIG. 41A shows a plot of dose-response curves for 2-(3,5-dihydroxybenzylcarbamoyl)-8-hydroxyquinoline-7-carboxylic acid (TOF540). The dose-response curve for TOF540 shows half maximal inhibitory concentration (IC₅₀) of 232 μM. IC₅₀ values were measured in the linear reaction range, and less than 60% of the total substrate had been consumed at readout. FIG. 41B shows a plot of the fluorescent intensity signal during the time course of NSP14/NSP10 inhibition across increasing concentrations of TOF540 (mM), as detected by FRET-based activity assay.

FIG. 42A shows a plot of dose-response curves for 2-(3,5-dibromo-4-hydroxystyryl)-8-hydroxyquinoline-7-carboxylic acid (FZ112). The dose-response curve for FZ112 shows half maximal inhibitory concentration (IC₅₀) of 26.68 μM. IC₅₀ values were measured in the linear reaction range, and less than 60% of the total substrate had been consumed at readout. FIG. 42B shows a plot of the fluorescent intensity signal during the time course of NSP14/NSP10 inhibition across increasing concentrations of FZ112 (μM), as detected by FRET-based activity assay.

FIG. 43A shows a plot of dose-response curves for 2-(3,4-difluorostyryl)-8-hydroxyquinoline-7-carboxylic acid (BI033). The dose-response curve for BI033 shows half maximal inhibitory concentration (IC₅₀) of 53.22 μM. IC₅₀ values were measured in the linear reaction range, and less than 60% of the total substrate had been consumed at readout. FIG. 43B shows a plot of the fluorescent intensity signal during the time course of NSP14/NSP10 inhibition across increasing concentrations of BI033 (μM), as detected by FRET-based activity assay.

FIG. 44A shows a plot of dose-response curves for 2-(3-carboxy-4-hydroxystyryl)-8-hydroxyquinoline-7-carboxylic acid (KHD304). The dose-response curve KHD304 shows half maximal inhibitory concentration (IC₅₀) of 24.6 μM. IC₅₀ values were measured in the linear reaction range, and less than 60% of the total substrate had been consumed at readout. FIG. 44B shows a plot of the fluorescent intensity signal during the time course of NSP14/NSP10 inhibition across increasing concentrations of KHD304 (μM), as detected by FRET-based activity assay.

FIG. 45A shows a plot of dose-response curves 2-(3,4-dihydroxystyryl)-8-hydroxyquinoline (KH153). The dose-response curve for KH153 shows half maximal inhibitory concentration (IC₅₀) of 33.31 μM. IC₅₀ values were measured in the linear reaction range, and less than 60% of the total substrate had been consumed at readout. FIG. 45B shows a plot of the fluorescent intensity signal during the time course of NSP14/NSP10 inhibition across increasing concentrations of KH153 (μM), as detected by FRET-based activity assay.

FIG. 46A shows a plot of dose-response curves for 2-(3,4-dihydroxyphenethyl)-8-hydroxyquinoline-7-carboxylic acid (KHD342). The dose-response curve for KHD342 shows half maximal inhibitory concentration (IC₅₀) of 67.12 μM. IC₅₀ values were measured in the linear reaction range, and less than 60% of the total substrate had been consumed at readout.

FIG. 46B shows a plot of the fluorescent intensity signal during the time course of NSP14/NSP10 inhibition across increasing concentrations of KHD342 (μM), as detected by FRET-based activity assay.

FIG. 47A shows a plot of dose-response curves for 7-(3,4-difluorobenzoyl)-2-(3,4-dihydroxyvinyl)-8-hydroxyquinoline (MBN91). The dose-response curve for MBN91 shows half maximal inhibitory concentration (IC₅₀) of 7.65 μM. IC₅₀ values were measured in the linear reaction range, and less than 60% of the total substrate had been consumed at readout. FIG. 47B shows a plot of the fluorescent intensity signal during the time course of NSP14/NSP10 inhibition across increasing concentrations of MBN91 (μM), as detected by FRET-based activity assay.

FIG. 48A shows a plot of dose-response curves for 7-benzoyl-2-(3,4-dihydroxyvinyl)-8-hydroxyquinoline (MBN120). The dose-response curve for MBN120 shows half maximal inhibitory concentration (IC₅₀) of 2.70 μM. IC₅₀ values were measured in the linear reaction range, and less than 60% of the total substrate had been consumed at readout. FIG. 48B shows a plot of the fluorescent intensity signal during the time course of NSP14/NSP10 inhibition across increasing concentrations of MBN120 (μM), as detected by FRET-based activity assay.

FIG. 49A shows a plot of dose-response curves for 2-(3,4-dihydroxystyryl)-5-hydroxyquinoline-6-carboxylic acid (BI050). The dose-response curve for BIO50 shows half maximal inhibitory concentration (IC₅₀) of 30.66 μM. IC₅₀ values were measured in the linear reaction range, and less than 60% of the total substrate had been consumed at readout. FIG. 49B shows a plot of the fluorescent intensity signal during the time course of NSP14/NSP10 inhibition across increasing concentrations of BIO50 (μM), as detected by FRET-based activity assay.

FIG. 50A shows a plot of dose-response curves for 2-(3,4-dihydroxyphenethyl)-8-hydroxyquinoline-7-phosphonic acid (MBN68). The dose-response curve for MBN68 shows half maximal inhibitory concentration (IC₅₀) of 28.60 μM. IC₅₀ values were measured in the linear reaction range, and less than 60% of the total substrate had been consumed at readout. FIG. 50B shows a plot of the fluorescent intensity signal during the time course of NSP14/NSP10 inhibition across increasing concentrations of MBN68 (μM), as detected by FRET-based activity assay.

FIG. 51 shows coomassie brilliant blue R-250 staining of purified SARS-CoV and MERS-CoV NSP10-His tagged protein and NSP14-His tagged protein expressed in E. coli.

FIG. 52 shows comparative graphs of kinetic profiles of SARS-CoV2, SARS-CoV and MERS-Cov NSP14/NSP10 complex, NSP14/NSP10 complex+EDTA and unbound NSP10 activity on dsRNA substrate Oligo D, as detected by FRET-based activity assay with appropriate controls.

FIGS. 53A-53B show the thermal stability of the NSP14/NSP10 complex in the presence or absence of 7-trifluoromethyl-N-(4-fluorobenzyl)-2-hydroxy-1,3-dioxo-4H-isoquinoline-4-carboxamide (VS59), Isobavachalcone and Solfacone, as determined by Thermofluor (thermal shift) assay. The derivatized melting curve for the NSP14/10 complex plus DMSO, and the NSP14/10 complex in the presence of 7-trifluoromethyl-N-(4-fluorobenzyl)-2-hydroxy-1,3-dioxo-4H-isoquinoline-4-carboxamide (VS59), Isobavachalcone and Solfacone (FIG. 53A). Raw melting curve data for the NSP14/10 complex plus DMSO, and the NSP14/10 complex in the presence of 7-trifluoromethyl-N-(4-fluorobenzyl)-2-hydroxy-1,3-dioxo-4H-isoquinoline-4-carboxamide (VS59), Isobavachalcone and Solfacone (FIG. 53B).

FIG. 54A shows the FRET signal obtained with digestion of Oligo D by RNase A followed by subsequent addition of #96, #112, #54, #60, #77, #68, #69 and #78 from Table 2. Fluorescence intensity was measured and plotted in arbitrary units (AU). Autofluorescence of compounds #96, #112, #54, #60, #77, #68, #69 and #78 are shown in FIG. 54B at the indicated wavelength at 250 uM final concentration. Graphs were normalized to DMSO. Error bars represent SD from one experiment using technical triplicates.

FIG. 55 shows a gel electrophoresis assay. Degradation of Oligo B was observed as faster migrating bands versus inhibition of the NSP14/10 complex (Oligo B digestion), which resulted in a slower migration pattern. Inhibition of NSP14/10 by compounds #96, #112, #54, #60, #77, #68, #69 and #78 from Table 2 is shown.

FIG. 56 shows representative graphs of the antiviral activity (full symbols) and cytotoxicity (empty symbols) of compounds #112, #77 and #68 from Table 2 in A549-ACE2 cells infected with SARS-CoV2. Compounds were applied at the following concentrations: #112: 60 uM, #77: 15 uM, #68: 25 uM. Remdesivir concentrations are indicated on the X-axis in uM. Error bars represents SEM. IF images of representative wells show anti-N staining (red) and DAPI signal (blue) at indicated drug concentrations. Graph shows the EC50 values from three independent experiments using technical triplicates. Error bars represents SEM.

FIG. 57 shows representative graphs of the antiviral activity (full symbols) and cytotoxicity (empty symbols) of compounds #112, #77 and #68 from Table 2 in HCM3 cells infected with HCoV-OC43. Compounds were applied at the following concentrations: #112: 60 uM, #77: 15 uM, #68: 25 uM. Remdesivir concentrations are indicated on the X-axis in uM. Error bars represents SEM. IF images of representative wells show anti-N staining (red) and DAPI signal (blue) at indicated drug concentrations. Graph shows the EC50 values from three independent experiments using technical triplicates. Error bars represents SEM.

### DETAILED DESCRIPTION

A description of example embodiments follows.

Disclosed herein are novel compositions and methods for a fluorescence-quenching based assay of nuclease enzyme activity. The present disclosure further relates to novel nucleic acid compositions that are substrates for the nuclease enzymes such that action of an exonuclease enzyme on said substrates results in a measurable change in the substrates using the method of the disclosure. Substrates specific for certain exonuclease enzyme activities can be used either alone or in combination. In some aspects, the disclosure provides for compositions comprising a fluorescently labeled double-stranded RNA (dsRNA) substrate that can serve as a reagent to detect many different nuclease enzymes. The nucleic acid compositions of the invention contain pendant groups that allow for fluorescence-quenching detection methods using fluorescence resonance energy transfer (FRET)-based approaches. In some aspects, a fluorescence reporter group and a fluorescence quencher group are physically connected by a chemical linkage that is cleaved during the course of the assay. Substrate cleavage leads to the physical separation of the reporter-quencher pair, which results in loss of quenching effect and a concomitant rise in fluorescence signal by the reporter group. In certain aspects, the methods disclosed herein are useful for identifying and/or assessing a modulator of an exonuclease that may be used either alone or in combination with other compounds. The present disclosure further encompasses kits for performing the methods of the invention and instructions for use. Methods disclosed herein may be performed in a high throughput format. The high throughput format may be employed in an industrial setting.

In another aspect, the present disclosure provides compounds capable of inhibiting enzymatic activity of an exonuclease (e.g., NSP14 or NSP14-NSP10 complex). In another aspect, the present disclosure provides methods of inhibiting enzymatic activity of an exonuclease (e.g., NSP14 or NSP14-NSP10 complex) with the inhibitory compounds of the present disclosure.

The methods and compositions disclosed herein may be useful to act as a template for pharmaceutical development, and/or for the design of medically useful inhibitor molecules.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

### Definitions

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The terminology used herein is for the purpose of describing particular embodiments only and is not intended to be limiting. As used herein, the indefinite articles “a”, “an” and “the” should be understood to include plural reference unless the context clearly indicates otherwise.

The term “nuclease” refers to any of various enzymes that catalyzes the breakdown of nucleic acids by cleaving bonds between adjacent nucleotides. In some embodiments, the nuclease referred to herein is an RNA nuclease.

The term “exonuclease” refers to any of various enzymes that catalyzes the cleavage of one or more nucleotides from the 3′ or 5′ ends of a nucleic acid. In some embodiments, the exonuclease referred to herein is an RNA exonuclease. As a non-limiting example, the exonuclease may be a NSP14 exonuclease. As another non-limiting example, the exonuclease may be a NSP14/NSP10 exonuclease complex.

The term “3′ to 5′ exonuclease” refers to any of various enzymes that catalyzes the cleavage of one or more nucleotides from the 3′ end a nucleic acid. As a non-limiting example, the 3′ to 5′ exonuclease may be a NSP14 exonuclease. As another non-limiting example, the exonuclease may be a NSP14/NSP10 3′ to 5′ exonuclease complex.

The term “fluorescence signal” refers to any measurable light that is emitted from a fluorophore.

The term “fluorophore” or “fluorophore reporter” or “reporter” refers to any of various fluorescent compounds that can absorb light at a wavelength and then emit light at a wavelength. In certain embodiments, the fluorescent reporter group is fluorescein, tetrachlorofluorescein, hexachlorofluorescein, rhodamine, tetramethylrhodamine, a Cy dye, Texas Red, a Bodipy dye, or an Alexa dye. Non-limiting examples of a fluorophore are 6FAM, TexasRed, TAMRA, Cy3, and Cy5.

The term “FRET probe” refers to a fluorophore or a quencher.

The term “FRET probe pair” or “pair of FRET probes” refers to any pair of a fluorophore and/or a quencher. As a non-limiting example, the FRET probe pair may be 6FAM-BHQ1, Cy3-BHQ2, TAMRA-BHQ2, TexasRed-BHQ2, and Cy5-BHQ3.

The term “GC-stretch” refers to a chain of nucleotides comprising any of a number of guanine (G) and/or cytosine (C) nucleotides.

The term “modulator”, as used herein, refers to a compound that modulates the activity of a nuclease (e.g., NSP14 or NSP14/NSP10 exonuclease). The modulator may act as an activator, an inhibitor, or both.

The term “processivity” refers the ability of enzyme (e.g., a nuclease) to catalyze a reaction continuously without dissociating from its substrate.

The term “proofreading exonuclease” refers to an exonuclease that may recognize incorrectly paired nucleotides, including ribonucleotides and deoxyribonucleotides, or variants or analogues thereof. For example, the incorrectly paired nucleotides may include nucleotide analogues such as Remdesivir, Ribavirin, Favipiravir, N₄-Hydroxycytidine (EIDD-1931) or its derivative Molnupiravir, 5-Fluorouracil and Sofosbuvir, and modified nucleotides (e.g., oxidized or deaminated nucleotides). In certain embodiments, the proofreading exonuclease may be an RNA exonuclease. As a non-limiting example, the proofreading exonuclease may be a NSP14 exonuclease. As another non-limiting example, the proofreading exonuclease may be a NSP14/NSP10 exonuclease complex.

The term “quencher” refers to any of various compounds that quenches the fluorescent signal of a fluorophore. In some embodiments, the fluorescence-quenching group is a nitrogen-substituted xanthene compound, a substituted 4-(phenyldiazenyl)phenylamine compound, or a substituted 4-(phenyldiazenyl)naphthylamine compound. In certain embodiments, the fluorescence-quenching group is Black-Hole Quenchers (BHQ) 1, 2, or 3 (e.g., available from Biosearch Technologies, Inc.). In certain embodiments, the fluorescence-quenching group is 4-(4′-dimethylaminophenylazo)benzoic acid), N,N′-dimethyl-N,N′-diphenyl-4-((5-t-butoxycarbonylaminopentyl) aminocarbonyl) piperidinylsulfonerhodamine (e.g., sold as QSY-7™ by Molecular Probes, Eugene, Oreg.), 4′,5′-dinitrofluorescein, pipecolic acid amide (e.g., sold as QSY-33™ by Molecular Probes, Eugene, Oreg.) 4-[4-nitrophenyldiazinyl]phenylamine, or 4-[4-nitrophenyldiazinyl]naphthylamine (e.g., sold by Epoch Biosciences, Bothell, Wash.).

The term “ribonucleotide analog” refers to a compound having similar structure and/or function to a ribonucleotide. Non-limiting examples of a ribonucleotide analog are Remdesivir, Ribavirin, Favipiravir, N₄-Hydroxycytidine (EIDD-1931) or its derivative Molnupiravir, 5-Fluorouracil, Sofosbuvir, and analogs and derivatives thereof.

As used herein, the term “alkyl” is given its ordinary meaning in the art and can include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁₋₂₀ for straight chain, C₂₋₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, a cycloalkyl group is a cyclopropyl, a cyclobutyl, a cyclopentyl, or a cyclohexyl group. In some embodiments, an alkyl group can be a lower alkyl group, wherein a lower alkyl group comprises 1-4 carbon atoms (e.g., C₁₋₄ for straight chain lower alkyls). When used in the context of a divalent alkyl group, it is to be understood that “alkyl” refers to an alkylene group.

As used herein, the term “alkenyl” refers to an alkyl group, as defined herein, including straight-chain alkenyl groups, branched-chain alkenyl groups, and cycloalkenyl groups having one or more double bonds. In certain embodiments, a straight chain or branched chain alkenyl has about 1-20 carbon atoms in its backbone (e.g., C₂₋₂₀ for straight chain, C₃₋₂₀ for branched chain), and alternatively, about 2-10 carbon atoms, or about 2 to 6 carbon atoms. In some embodiments, an alkenyl group has 1, 2, 3, 4, 5, or 6 double bonds. In some embodiments, a cycloalkenyl ring has from about 3-10 carbon atoms in the ring structure where such rings are monocyclic or bicyclic, and alternatively about 5, 6 or 7 carbons in the ring structure, and 1, 2, or 3 double bonds. In some embodiments, a cycloalkenyl group is a cyclopropenyl, a cyclobutenyl, a cyclobutadienyl, a cyclopentenyl, a cyclopentadienyl, a cyclohexenyl, or a cyclohexadienyl group. In some embodiments, an alkenyl group can be a lower alkenyl group, wherein a lower alkenyl group comprises 2-4 carbon atoms (e.g., C₂₋₄ for straight chain lower alkenyls). In one embodiment, a cycloalkenyl group has six carbon atoms and one double bond.

As used herein, the term “alkynyl” refers to an alkyl group, as defined herein, including straight-chain alkynyl groups, branched-chain alkynyl groups, and cycloalkynyl groups having one or more triple bonds. In certain embodiments, a straight chain or branched chain alkynyl has about 2-20 carbon atoms in its backbone (e.g., C₂₋₂₀ for straight chain, C₃₋₂₀ for branched chain), and alternatively, about 2-10 carbon atoms, or about 2 to 6 carbon atoms. In some embodiments, an alkynyl group has 1, 2, 3, 4, 5, or 6 triple bonds. In some embodiments, a cycloalkynyl ring has from about 6-12 carbon atoms in the ring structure where such rings are monocyclic or bicyclic, and alternatively about 8, 9, or 10 carbons in the ring structure, and 1, 2, or 3 triple bonds. In some embodiments, an alkynyl group can be a lower alkynyl group, wherein a lower alkynyl group comprises 2-4 carbon atoms (e.g., C₂₋₄ for straight chain lower alkynyls).

The term “heteroalkyl” is given its ordinary meaning in the art and refers to alkyl groups as described herein in which one or more carbon atoms is replaced with a heteroatom (e.g., oxygen, nitrogen, sulfur, and the like). In some embodiments, a heteroalkyl group can have one or more of methylene groups replaced with —O—, —S—, or —NH—, in which the hydrogen of —NH— is optionally substituted. Examples of heteroalkyl groups include, but are not limited to, alkoxy, poly(ethylene glycol)-, alkyl-substituted amino, tetrahydrofuranyl, piperidinyl, morpholinyl, etc.

The term “haloalkyl” is given its ordinary meaning in the art and refers to alkyl groups as described herein in which one or more hydrogen atoms is replaced by a halogen atom, i.e., F, Cl, Br, or I. In some embodiments a haloalkyl group can be a perfluoroalkyl group, i.e., a group where all hydrogen atoms are replaced with fluoride atoms. In some embodiments a haloalkyl group can be a halomethyl group, i.e., a C₁ group with 1, 2, or 3 halogen atoms, e.g., —CF₃, —CF₂H, —CH₂F, —CH₂Cl, —CH₂Br; —CH₂I. In some embodiments a haloalkyl group can be, e.g., —CF₃, —CF₂H, —CH₂F, —CH₂Cl, —CH₂Br; —CH₂I, —CH₂CF₃, CH₂CH₂F, —CH₂CH₂Br, —CH₂CH₂Cl, —CH₂CH₂I, etc.

The term “haloalkoxy” is given its ordinary meaning in the art and refers to alkoxy groups as described herein, i.e. alkyl groups bonded to an oxygen atom, in which one or more hydrogen atoms is replaced by a halogen atom, i.e., F, Cl, Br, or I. In some embodiments a haloalkoxy group can be a perfluoroalkoxy group, i.e., a group where all hydrogen atoms are replaced with fluoride atoms. In some embodiments a haloalkoxy group can be, e.g., —OCF₃, —OCF₂H, —OCH₂F, —OCH₂Cl, —OCH₂Br; —OCH₂I, —OCH₂CF₃, —OCH₂CH₂F, —OCH₂CH₂Br, —OCH₂CH₂Cl, —OCH₂CH₂I, etc.

The term “aryl” used alone or as part of a larger moiety as in “aralkyl,” “aralkoxy,” “aryloxy” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” can be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which can bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The term “aralkyl” refers to alkyl groups as described herein in which one or more hydrogen atoms is substituted by aryl groups, where the radical or point of attachment is on the alkyl group. The alkyl part of an aralkyl group is optionally substituted as described in the term “alkyl” above. The aryl part of the aralkyl group is optionally substituted as described in the term “alkyl” above.

The term “alkylaryl” refers to aryl groups as described herein in which one or more hydrogen atoms is substituted by alkyl groups, where the radical or point of attachment is on the aryl group. The aryl part of the alkylaryl group is optionally substituted as described in the term “aryl” above. The alkyl part of an alkylaryl group is optionally substituted as described in the term “alkyl” above.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quatemized form of a basic nitrogen. Heteroaryl groups include, without limitation, pyridine, quinoline, isoquinoline, quinolizine, pyrido[1,2-a]pyrazine, 1,8-naphthyridine, purine, chromene, indole, phenanthrene, benzo[H]quinoline, anthraquinone, and phenanthrol[1,2-b]furan groups. In some embodiments, a heteroaryl is a heterobiaryl group, such as bipyridyl and the like. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group can be monocyclic, bicyclic, tricyclic, tetracyclic, and/or otherwise polycyclic. The term “heteroaryl” can be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is saturated, partially unsaturated, or aromatic, and having, in addition to carbon atoms, one or more, e.g., one to four, heteroatoms, as defined above. As used herein, the term “heterocycle” encompasses heteroaryl groups, as defined above. In one embodiment, a heterocycle can be a saturated, partially unsaturated, or aromatic, 5-7 membered monocyclic moiety comprising from 1 to 3 nitrogen atoms, e.g., a pyrrole, an imidazole, a pyrazole, a pyrazole, a triazole, a piperidine, a piperazine, a pyridazine, a pyridine, 2H-pyridine, a pyridone, a pyrimidine, or a pyrazine, including monovalent or divalent radicals thereof. When used in reference to a ring atom of a heterocycle, the term “nitrogen” includes a substituted nitrogen. In one embodiment, a heterocycle can be a saturated, partially unsaturated, or aromatic, 5-7 membered monocyclic moiety comprising from 1 to 3 oxygen atoms, e.g., a tetrahydrofuran (i.e., oxolane), a furan, a dihydrofuran, a dioxolane, a tetrahydropyran (i.e., oxane), a pyran, a dihydropyran, a dioxane, a dioxine, a trioxane, an oxepane, or an oxepine, including monovalent or divalent radicals thereof. In one embodiment, a heterocycle can be thiophene, oxazole, thiazole, or morpholine, including monovalent or divalent radicals thereof.

A heterocyclic ring can be attached, e.g., to its pendant group, at any heteroatom or carbon atom that results in a stable structure. Examples of such saturated or partially unsaturated heterocyclic radicals include, without limitation, tetrahydropyranyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as phenyl, indolinyl, 3H-indolyl, chromanyl, phenanthridinyl, or tetrahydroquinolinyl. A heterocyclyl group can be monocyclic, bicyclic, tricyclic, tetracyclic, and/or otherwise polycyclic. The term “heterocyclylalkyl” refers to an alkyl group substituted by a heterocyclyl.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

The term “heteroatom” means one or more of oxygen, sulfur, nitrogen, phosphorus, or silicon, including any oxidized form of nitrogen, sulfur, phosphorus, or silicon; the quaternized form of any basic nitrogen. In some embodiments, a heteroatom can be a substitutable nitrogen of a heterocyclic ring.

The term “unsaturated,” as used herein, means that a moiety has one or more units of unsaturation.

The term “halogen” means F, Cl, Br, or I atom, and/or its radical or substituent, namely —F, —Cl, —Br, or —I.

As described herein, in certain embodiments, certain compounds of the disclosure can be indicated to comprise “optionally substituted” moieties. When indicated, in general, the term “substituted,” whether preceded by the term “optionally” or not, means that one or more hydrogens of the designated moiety are replaced with a suitable substituent. Unless otherwise indicated, an “optionally substituted” group can have a suitable substituent at each substitutable position of the group, and when more than one position in any given structure can be substituted with more than one substituent selected from a specified group, the substituent can be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

As used herein, a substituent, e.g., —B, can be represented as a

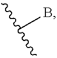

where [symbol] denotes a point of attachment.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

The present application also includes pharmaceutically acceptable salts of the compounds described herein. The “pharmaceutically acceptable salts” include a subset of the “salts” described above which are conventional non-toxic salts of the parent compound formed, for example, from non-toxic inorganic or organic acids. Lists of suitable salts are found in Remington's Pharmaceutical Sciences, 17th ed., Mack Publishing Company, Easton, Pa., 1985, p. 1418 and Berge, S M et al, Journal of Pharmaceutical Science, 1977, 66, 1, 1-19. By way of an example, in an embodiment of the disclosure pharmaceutically acceptable salts can comprise a suitable anion selected from F⁻, Cl⁻, Br⁻, I⁻, OH⁻, ⁻BF₄, CF₃SO₃⁻, monobasic sulfate, dibasic sulfate, monobasic phosphate, dibasic phosphate, or tribasic phosphate, NO₃⁻, PF₆⁻, NO₂⁻, carboxylate, CₑF_{f}SO₃⁻, (where e=2-10 and f=2e+1), acetate, aspartate, benzenesulfonate, benzoate, besylate, bicarbonate, bitartrate, camsylate, carbonate, citrate, decanoate, edetate, esylate, fumarate, gluceptate, gluconate, glutamate, glycolate, glycollyalarsanilate, hexanoate, hydrabamine, hydroxynaphthoate, isthionate, lactate, lactobionate, malate, maleate, mandelate, mesylate, methylbromide, methylnitrate, mucate, napsylate, octanoate, oleate, oxalate, palmitate, pamoate, pantothenate, polygalacturonate, propionate, salicylate, stearate, subacetate, succinate, tartrate, teoclate, tosylate, or triethiiodide. By way of another example, in an embodiment of the disclosure pharmaceutically acceptable salts can comprise a suitable cation selected from aluminum, arginine, benzathine, calcium, chloroprocaine, choline, diethanolamine, ethanolamine, ethylenediamine, lysine, magnesium, histidine, lithium, meglumine, potassium, procaine, sodium, triethylamince, or zinc. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### FRET—Based Methods

In one aspect is provided a fluorescence resonance energy transfer (FRET)-based method for determining measuring a 3′ to 5′ exonuclease activity in a sample, comprising: (a) contacting the sample with a fluorescently labeled double-stranded RNA (dsRNA) substrate to create a test reaction mixture, wherein said dsRNA substrate comprises (i) at least one free 3′ OH group, and (ii) a pair of FRET pair probes comprising a fluorophore and a quencher, wherein the one fluorophore probe is located at the 5′ end of the strand comprising the free 3′ OH group and the quencher probe is located either at the 5′ end or at the 3′ end of the other strand of said dsRNA substrate, and when the substrate is uncleaved, the quencher quenches the fluorescence signal of the fluorophore; (b) incubating said test reaction mixture under conditions and for a time sufficient for cleavage of the substrate by the 3′ to 5′ exonuclease, wherein the cleavage of the substrate by the 3′ to 5′ exonuclease causes sufficient separation of the fluorophore and the quencher to reduce quenching of the fluorescence signal of the fluorophore, and (c) measuring the fluorescence signal emitted from the test reaction mixture. In some embodiments, the method may further comprise comparing the fluorescence signal determined measured in step (c) to a control fluorescence signal. In some embodiments, the control fluorescence signal may be a predetermined value. In some embodiments, the control fluorescence signal may be the fluorescence signal measured under the same conditions in a control sample comprising the same dsRNA substrate but in the absence of the exonuclease. In some embodiments, the control fluorescence signal may be the fluorescence signal measured determined under the same conditions in a control sample comprising the same dsRNA substrate and a specific amount of a control nuclease. In some embodiments, the control nuclease may be selected from RNase A, RNase L, PNPase, RNase II, RNase R, Ribonuclease T1, Nuclease BAL-31, and RNase III.

In another aspect is provided a fluorescence resonance energy transfer (FRET)-based method for identifying and/or assessing a modulator of a 3′ to 5′ exonuclease, comprising: (a) in a test reaction mixture, contacting the exonuclease with a test compound and a fluorescently labeled double-stranded RNA (dsRNA) substrate, wherein said dsRNA substrate comprises (i) at least one free 3′ OH group, and (ii) a pair of FRET pair probes comprising a fluorophore and a quencher, wherein the fluorophore one probe is located at the 5′ end of the strand comprising the free 3′ OH group and the quencher other probe is located either at the 5′ end or at the 3′ end of the other strand of said dsRNA substrate, and when the substrate is uncleaved, the quencher quenches the fluorescence signal of the fluorophore; (b) incubating said test reaction mixture under conditions and for a time sufficient for cleavage of the substrate by the exonuclease in the absence of the test compound, wherein the cleavage of the substrate by the exonuclease causes sufficient separation of the fluorophore and the quencher to reduce quenching of the fluorescence signal of the fluorophore; (c) determining the fluorescence signal emitted from the test reaction mixture; (d) comparing the fluorescence signal determined in step (c) to a control fluorescence signal, wherein the control fluorescence signal is the fluorescence signal determined under the same conditions in a control sample comprising the same amounts of exonuclease and dsRNA substrate but in the absence of the test compound, and (e) (i) determining that the test compound is an inhibitor of the exonuclease if the fluorescence signal in the test reaction mixture is lower than in the control reaction mixture, or (ii) determining that the test compound is not an inhibitor of the exonuclease if the fluorescence signal in the test reaction mixture is not lower than in the control reaction mixture, or (iii) determining that the test compound is an activator of the exonuclease if the fluorescence signal in the test reaction mixture is higher than in the control reaction mixture. In some embodiments, step (a) may comprise pre-incubating the exonuclease with the test compound prior to the addition of the dsRNA substrate. In some embodiments, step (a) may comprise adding the test compound after contacting the exonuclease with a dsRNA substrate.

In another aspect is provided a fluorescence resonance energy transfer (FRET)-based method for measuring processivity of a 3′ to 5′ exonuclease, comprising: (a) contacting the exonuclease with a first fluorescently labeled double-stranded RNA (dsRNA) substrate to create a first reaction mixture, wherein said first dsRNA substrate comprises (i) at least one free 3′ OH group, and (ii) a pair of FRET probes pair comprising a fluorophore and a quencher, wherein one probe the fluorophore is located at the 5′ end of the strand comprising the free 3′ OH group and the quencher other probe is located either at the 5′ end or at the 3′ end of the other strand of said first dsRNA substrate, and when the substrate is uncleaved, the quencher quenches the fluorescence signal of the fluorophore; (b) contacting the exonuclease with a second dsRNA substrate to create a second reaction mixture, wherein the second dsRNA substrate differs from the first dsRNA substrate in that it is longer than the first substrate; (c) incubating said first reaction mixture and said second reaction mixture under conditions and for a time allowing for cleavage of both substrates by the exonuclease, wherein the cleavage of the substrates by the exonuclease causes sufficient separation of the fluorophore and the quencher to reduce quenching of the fluorescence signal of the fluorophore, and (d) determining the time required for the first reaction mixture and the second reaction mixture to reach the same level of fluorescence signal; wherein the processivity of the exonuclease is measured as the difference in the length between the first and second substrate divided by the difference in the time required for the first reaction mixture and second reaction mixture to reach the same level of fluorescence signal.

In some embodiments, the fluorescence resonance energy transfer (FRET)-based method for measuring processivity of a 3′ to 5′ exonuclease activity may be performed in a high throughput format.

Double Stranded RNA (dsRNA) Substrates

In some embodiments, the fluorescence resonance energy transfer (FRET)-based methods for measuring a 3′ to 5′ exonuclease activity in a sample disclosed herein comprises contacting the sample with a fluorescently labeled double-stranded RNA (dsRNA) substrate to create a test reaction mixture.

In some embodiments, the dsRNA substrate comprises at least one free 3′ OH group.

In some embodiments, the dsRNA substrate comprises a pair of FRET probes comprising a fluorophore and a quencher.

In some embodiments, one probe is located at the 5′ end of the strand comprising the free 3′ OH group and the other probe is located either at the 5′ end or at the 3′ end of the other strand of said dsRNA substrate, and when the substrate is uncleaved, the quencher quenches the fluorescence signal of the fluorophore.

In some embodiments, incubating the test reaction mixture comprising the dsRNA substrates comprising the FRET probes under conditions and for a time sufficient for cleavage of the substrate by the 3′ to 5′ exonuclease causes sufficient separation of the fluorophore and the quencher to reduce quenching of the fluorescence signal of the fluorophore. In some embodiments, the fluorescence signal emitted from the test reaction mixture may be measured.

In some embodiments, the fluorophore may be located at the 5′ end of the strand comprising the free 3′ OH group and the quencher is located either at the 5′ end or at the 3′ end of the other strand of said dsRNA substrate.

In some embodiments, the quencher is located at the 5′ end of the other strand of the dsRNA substrate.

In some embodiments, the quencher may be located at the 3′ end of the other strand of the dsRNA substrate.

In some embodiments, the quencher may be located at the 5′ end of the strand comprising the free 3′ OH group and the fluorophore is located either at the 5′ end or at the 3′ end of the other strand of said dsRNA substrate.

In some embodiments, the fluorophore may be located at the 5′ end of the other strand of the dsRNA substrate.

In some embodiments, the fluorophore may be located at the 3′ end of the other strand of the dsRNA substrate.

In some embodiments, the fluorophore may be selected from 6FAM, TexasRed, TAMRA, Cy3, and Cy5.

In some embodiments, the fluorophore may be 6FAM.

In some embodiments, the fluorophore may be TexasRed.

In some embodiments, the quencher may be selected from BHQ1, BHQ2, and BHQ3.

In some embodiments, the quencher may be BHQ1.

In some embodiments, the quencher may be BHQ2.

In some embodiments, the pair of FRET probes may be selected from 6FAM-BHQ1, Cy3-BHQ2, TAMRA-BHQ2, TexasRed-BHQ2, and Cy5-BHQ3.

In some embodiments, the fluorophore is 6FAM and the quencher is BHQ1

In some embodiments, the fluorophore is TexasRed and the quencher is BHQ2.

In some embodiments, the at least one free 3′ end of the dsRNA substrate has one or more mismatches. In some embodiments, the at least one free 3′ end of the dsRNA substrate has one to three mismatches. In some embodiments, the one or more mismatches comprise one or more ribonucleotide analogs. Non-limiting examples of ribonucleotide analogs include Remdesivir, Ribavirin, Favipiravir, N₄-Hydroxycytidine (EIDD-1931) or its derivative Molnupiravir, 5-Fluorouracil and Sofosbuvir.

In some embodiments the dsRNA substrate has a length between 1 and 100 base pairs. In some embodiments the dsRNA substrate has a length between 10 and 80 base pairs. In some embodiments the dsRNA substrate has a length between 10 and 60 base pairs. In some embodiments the dsRNA substrate has a length between 10 and 40 base pairs. In some embodiments the dsRNA substrate has a length between 10 and 20. In some embodiments the length of the dsRNA substrate is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 base pairs.

In some embodiments the dsRNA substrate has a length between 10 and 20. In some embodiments the length of the dsRNA substrate is 10, 11, 12, 13, 14, 15, 16, 17, 18, 19, or 20 base pairs.

In some embodiments the dsRNA substrate has a length between 13 and 20 base pairs. In some embodiments the length of the dsRNA substrate is 13, 14, 15, 16, 17, 18, 19 or 20 base pairs.

In some embodiments the dsRNA substrate has a length of at least 13 base pairs.

In some embodiments the dsRNA substrate has a length of at least 14 base pairs.

In some embodiments the dsRNA substrate has a length of at least 15 base pairs.

In some embodiments the dsRNA substrate has a length of at least 16 base pairs.

In some embodiments the dsRNA substrate has a length of at least 17 base pairs.

In some embodiments the dsRNA substrate has a length of at least 18 base pairs.

In some embodiments the dsRNA substrate has a length of at least 19 base pairs.

In some embodiments the dsRNA substrate has a length of at least 20 base pairs.

In some embodiments, dsRNA substrate comprises a GC-stretch at the free 3′ end.

In some embodiments, the dsRNA substrate comprises a GC-free-stretch adjacent to the fluorophore and/or quencher. In some embodiments, the dsRNA substrate comprises a GC-free-stretch that is not adjacent to the fluorophore and/or quencher.

In some embodiments, the GC-free-stretch is at least 10 bases apart from the fluorophore and/or quencher. In some embodiments, the GC-free-stretch is 1, 2, 3, 4, 5, 6, 7, 8, 9, or 10 bases apart from the fluorophore and/or quencher. In some embodiments, the GC-free-stretch is at least 1 base apart from the fluorophore and/or the quencher.

In some embodiments, the dsRNA substrate comprises a U, A, C, or G base immediately next to the fluorophore. In some embodiments, the dsRNA substrate comprises a U base immediately next to the fluorophore. In some embodiments, the dsRNA substrate comprises a A base immediately next to the fluorophore. In some embodiments, the dsRNA substrate comprises a U or A base immediately next to the fluorophore. In some embodiments, the dsRNA substrate comprises a C base immediately next to the fluorophore. In some embodiments, the dsRNA substrate comprises a G base immediately next to the fluorophore.

In some embodiments, the dsRNA substrate comprises a single U, A, C, or G base in the second or third position from the fluorophore. In some embodiments, the dsRNA substrate comprises a single U base in the second or third position from the fluorophore. In some embodiments, the dsRNA substrate comprises a single A base in the second or third position from the fluorophore. In some embodiments, the dsRNA substrate comprises a single C base in the second or third position from the fluorophore. In some embodiments, the dsRNA substrate comprises a single G base in the second or third position from the fluorophore.

In some embodiments, the dsRNA substrate comprises a 5′ overhang between 1 and 5 base pairs. In some embodiments, the dsRNA substrate comprises a 5′ overhang of 1, 2, 3, 4, or 5 base pairs. In some embodiments, the dsRNA substrate comprises a 5′ overhang of 1 base pair. In some embodiments, the dsRNA substrate comprises a 5′ overhang of 2 base pairs. In some embodiments, the dsRNA substrate comprises a 5′ overhang of 3 base pairs. In some embodiments, the dsRNA substrate comprises a 5′ overhang of 4 base pairs. In some embodiments, the dsRNA substrate comprises a 5′ overhang of 5 base pairs.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 1.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 2.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 3.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 4.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 5.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 6.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 7.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 8.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 10.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 11.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 12.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 13.

In some embodiments, the dsRNA substrate comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 14.

In some embodiments, the dsRNA substrate may be selected from:

```
5′ FAM-UUGCCGAAUUAAGCGCA-3′ (SEQ ID NO: 1)
                  ||||||||||||||
          3′-CGGCUUAAUUCGCGAAU-BHQ1-5′ (SEQ ID NO: 2)
  5′ FAM-UUGCCGAAUUAAGCGCCA (SEQ ID NO: 3)
                |||||||||||||||||
3′ BHQ1-AACGGCUUAAUUCGCGGAAU (SEQ ID NO: 4)
  5′ FAM-UUUUUUCGGCCCA (SEQ ID NO: 5)
                ||||||||||||
3′ BHQ1-AAAAAAGCCGGGAUAAA (SEQ ID NO: 6)
  5′ FAM-UCUUUUCGGCCCA (SEQ ID NO: 7)
                ||||||||||||
3′ BHQ1-AGAAAAGCCGGGAUAAA (SEQ ID NO: 8)
5′ TxRed-UCUUUUCGGCCCA (SEQ ID NO: 9)
                  ||||||||||||
  3′ BHQ2-AGAAAAGCCGGGAUAAA (SEQ ID NO: 10)
  5′ Cy3-UCUUUUCGGCCCA (SEQ ID NO: 11)
                ||||||||||||
3′ BHQ2-AGAAAAGCCGGGAUAAA (SEQ ID NO: 10)
3′ TAMRA-UCUUUUCGGCCCA (SEQ ID NO: 12)
                  ||||||||||||
  3′ BHQ2-AGAAAAGCCGGGAUAAA (SEQ ID NO: 10)
  5′ Cy5-UCUUUUCGGCCCA (SEQ ID NO: 13)
                ||||||||||||
3′ BHQ3-AGAAAAGCCGGGAUAAA (SEQ ID NO: 14).
```

In some embodiments, the dsRNA substrate may be

```
5′ FAM-UUGCCGAAUUAAGCGCA-3′ (SEQ ID NO: 1)
                  ||||||||||||||
          3′-CGGCUUAAUUCGCGAAU-BHQ1-5′ (SEQ ID NO: 2)
```

In some embodiments, the dsRNA substrate may be:

```
  5′ FAM-UUGCCGAAUUAAGCGCCA (SEQ ID NO: 3)
                |||||||||||||||||
3′ BHQ1-AACGGCUUAAUUCGCGGAAU (SEQ ID NO: 4)
```

In some embodiments, the dsRNA substrate may be:

```
  5′ FAM-UUUUUUCGGCCCA (SEQ ID NO: 5)
                ||||||||||||
3′ BHQ1-AAAAAAGCCGGGAUAAA (SEQ ID NO: 6)
```

In some embodiments, the dsRNA substrate may be:

```
  5′ FAM-UCUUUUCGGCCCA (SEQ ID NO: 7)
                ||||||||||||
3′ BHQ1-AGAAAAGCCGGGAUAAA (SEQ ID NO: 8)
```

In some embodiments, the dsRNA substrate may be:

```
5′ TxRed-UCUUUUCGGCCCA (SEQ ID NO: 9)
                  ||||||||||||
  3′ BHQ2-AGAAAAGCCGGGAUAAA (SEQ ID NO: 10)
```

In some embodiments, the dsRNA substrate may be:

```
5′ TxRed-UCUUUUCGGCCCA (SEQ ID NO: 9)
                  ||||||||||||
  3′ BHQ2-AGAAAAGCCGGGAUAAA (SEQ ID NO: 10)
```

In some embodiments, the dsRNA substrate may be:

```
  5′ Cy3-UCUUUUCGGCCCA (SEQ ID NO: 11)
                ||||||||||||
3′ BHQ2-AGAAAAGCCGGGAUAAA (SEQ ID NO: 10)
```

In some embodiments, the dsRNA substrate may be:

```
  5′ Cy5-UCUUUUCGGCCCA (SEQ ID NO: 13)
                ||||||||||||
3′ BHQ3-AGAAAAGCCGGGAUAAA (SEQ ID NO: 14)
```

### FRET Probes

In some embodiments, the fluorescence resonance energy transfer (FRET)-based methods for measuring a 3′ to 5′ exonuclease activity in a sample disclosed herein comprise contacting the sample with a fluorescently labeled double-stranded RNA (dsRNA) substrate to create a test reaction mixture, wherein said dsRNA substrate comprises (i) at least one free 3′ OH group, and (ii) a pair of FRET probes.

In some embodiments, the dsRNA substrate comprises a pair of FRET probes comprising a fluorophore and a quencher.

In certain embodiments, one of the pair of FRET probes is located at the 5′ end of the strand comprising the free 3′ OH group and the other of the pair of FRET probes is located either at the 5′ end or at the 3′ end of the other strand of said dsRNA substrate, and when the substrate is uncleaved, the quencher quenches the fluorescence signal of the fluorophore.

In some embodiments, incubating the test reaction mixture comprising the dsRNA substrates comprising the FRET probes under conditions and for a time sufficient for cleavage of the substrate by the 3′ to 5′ exonuclease causes sufficient separation of the fluorophore and the quencher to reduce quenching of the fluorescence signal of the fluorophore. In some embodiments, the fluorescence signal emitted from the test reaction mixture may be measured.

In some embodiments, the fluorophore may be located at the 5′ end of the strand comprising the free 3′ OH group and the quencher is located either at the 5′ end or at the 3′ end of the other strand of said dsRNA substrate. In some embodiments, the quencher is located at the 5′ end of the other strand of the dsRNA substrate. In some embodiments, the quencher may be located at the 3′ end of the other strand of the dsRNA substrate.

In some embodiments, the quencher may be located at the 5′ end of the strand comprising the free 3′ OH group and the fluorophore is located either at the 5′ end or at the 3′ end of the other strand of said dsRNA substrate.

In some embodiments, the fluorophore may be located at the 5′ end of the other strand of the dsRNA substrate.

In some embodiments, the fluorophore may be located at the 3′ end of the other strand of the dsRNA substrate.

In some embodiments, the fluorophore is absent and is not attached to the dsRNA substrate. In some embodiments, the quencher is absent and is not attached to the dsRNA substrate. In some embodiments, either the fluorophore or the quencher is absent and is not attached to the dsRNA substrate. In some embodiments, both the fluorophore and the quencher are absent and neither is attached to the dsRNA substrate.

In some embodiments, the fluorophore may be selected from 6FAM, TexasRed, TAMRA, Cy3, and Cy5.

In some embodiments, the fluorophore may be 6FAM.

In some embodiments, the fluorophore may be TexasRed.

In some embodiments, the quencher may be selected from BHQ1, BHQ2, and BHQ3.

In some embodiments, the quencher may be BHQ1.

In some embodiments, the quencher may be BHQ2.

In some embodiments, the pair of FRET probes may be selected from 6FAM-BHQ1, Cy3-BHQ2, TAMRA-BHQ2, TexasRed-BHQ2, and Cy5-BHQ3.

In some embodiments, the fluorophore is 6FAM and the quencher is BHQ1

In some embodiments, the fluorophore is TexasRed and the quencher is BHQ2.

### Nucleases

In some embodiments, the fluorescence resonance energy transfer (FRET)-based methods disclosed herein may be used for measuring nuclease activity. A non-limiting example of a nuclease is a exonuclease.

In some embodiments, the FRET-based methods disclosed herein may be used for measuring 3′ to 5′ nuclease activity.

In some embodiments, the FRET-based methods disclosed herein may be used for measuring 5′ to 3′ nuclease activity.

In some embodiments, the FRET-based methods disclosed herein may be used for measuring 3′ to 5′ exonuclease activity.

In some embodiments, the FRET-based methods disclosed herein may be used for measuring 5′ to 3′ exonuclease activity.

In some embodiments, FRET-based methods disclosed herein may be for measuring a 3′ to 5′ exonuclease activity.

In some embodiments, FRET-based methods disclosed herein may be used for measuring a 3′ to 5′ exonuclease activity of a proofreading exonuclease. Non-limiting examples of a proofreading exonuclease are a NSP14 exonuclease or a NSP14/NSP10 exonuclease complex.

In some embodiments, the NSP14 exonuclease is complexed with, or bound to, the NSP10 to form a NSP14/NSP10 exonuclease complex. In some embodiments, the NSP14 exonuclease is not complexed with, or bound to, the NSP10 and a NSP14/NSP10 exonuclease complex is not formed. Description and SEQ ID NOs of exemplary nucleotide and amino acid sequences for exemplary NSP4 exonucleases (SARS-CoV-2 Replicase polyprotein lab 5926-6452, Accession no. YP_009725309.1, SARS-CoV Replicase polyprotein lab, Accession no. NP_828871.1, MERS-CoV Replicase polyprotein lab, Accession no. YP_009047225.1) and NSP10 exonucleases (SARS-CoV-2 Replicase polyprotein 1a 4254-4392, Accession no. YP_009725306.1, SARS-CoV Replicase polyprotein lab, Accession no. YP_009944375.1, MERS-CoV Replicase polyprotein lab, Accession no. YP_009047222.1) are provided in Table 1.

TABLE 1
Exemplary nucleotide and amino acid sequences for NSP14 and NSP10

| Description | Sequence | SEQ ID NO: |
|---|---|---|
| NSP14 | ATGGCCGAAAATGTCACGGGTCTGTTCAAAGATT | 15 |
| nucleotide | GTTCCAAAGTTATCACGGGTCTGCATCCGACGCA | |
| sequence | AGCACCGACGCACCTGTCTGTCGATACCAAATTT | |
| (SARS-CoV2) | AAAACGGAAGGCCTGTGCGTGGATATTCCGGGTA | |
| | TCCCGAAAGACATGACGTATCGTCGCCTGATTAG | |
| | CATGATGGGCTTCAAAATGAACTATCAAGTGAAT | |
| | GGTTACCCGAACATGTTTATTACCCGTGAAGAAG | |
| | CCATCCGTCATGTGCGCGCATGGATCGGCTTCGA | |
| | TGTTGAAGGTTGTCACGCAACGCGCGAAGCTGTT | |
| | GGCACCAATCTGCCGCTGCAGCTGGGCTTTAGCA | |
| | CGGGTGTGAACCTGGTTGCGGTGCCGACCGGTTA | |
| | TGTTGATACCCCGAACAATACGGACTTCAGTCGC | |
| | GTGTCCGCAAAACCGCCGCCGGGTGATCAGTTTA | |
| | AACATCTGATTCCGCTGATGTACAAAGGTCTGCC | |
| | GTGGAATGTGGTTCGTATTAAAATCGTTCAAATG | |
| | CTGTCAGATACCCTGAAAAACCTGTCGGACCGCG | |
| | TCGTGTTCGTTCTGTGGGCACACGGCTTTGAACTG | |
| | ACGTCTATGAAATATTTCGTCAAAATCGGTCCGG | |
| | AACGCACCTGCTGTCTGTGCGATCGTCGCGCGAC | |
| | CTGTTTTTCAACGGCGTCGGACACCTACGCCTGCT | |
| | GGCATCACAGTATTGGCTTCGATTATGTCTACAAT | |
| | CCGTTTATGATCGACGTGCAGCAATGGGGCTTCA | |
| | CCGGTAATCTGCAGTCGAACCATGATCTGTATTG | |
| | CCAAGTCCATGGTAACGCGCACGTGGCCAGCTGT | |
| | GACGCCATTATGACGCGTTGCCTGGCAGTGCACG | |
| | AATGTTTTGTTAAACGCGTCGATTGGACCATTGA | |
| | ATATCCGATTATCGGCGACGAACTGAAAATCAAT | |
| | GCGGCCTGTCGTAAAGTGCAGCACATGGTTGTCA | |
| | AAGCAGCTCTGCTGGCCGATAAATTTCCGGTTCT | |
| | GCACGACATTGGTAACCCGAAAGCTATCAAATGC | |
| | GTGCCGCAGGCGGATGTTGAATGGAAATTCTATG | |
| | ATGCCCAACCGTGTTCTGACAAAGCATACAAAAT | |
| | CGAAGAACTGTTTTATAGTTACGCTACGCATTCCG | |
| | ATAAATTCACCGACGGCGTTTGCCTGTTTTGGAAC | |
| | TGTAATGTCGATCGCTATCCGGCCAATAGTATCGT | |
| | TTGCCGTTTTGACACCCGCGTCCTGTCCAACCTGA | |
| | ATCTGCCGGGTTGTGATGGCGGTTCACTGTACGTC | |
| | AATAAACATGCATTTCACACCCCGGCTTTCGACA | |
| | AATCCGCGTTTGTTAACCTGAAACAGCTGCCGTTT | |
| | TTCTATTACAGCGATTCTCCGTGCGAAAGCCATG | |
| | GCAAACAAGTGGTTTCTGATATTGACTATGTGCC | |
| | GCTGAAAAGTGCGACCTGCATCACGCGTTGTAAC | |
| | CTGGGCGGTGCTGTTTGTCGTCATCACGCGAATG | |
| | AATATCGCCTGTACCTGGATGCTTACAACATGAT | |
| | GATTAGCGCGGGTTTCTCTCTGTGGGTGTATAAAC | |
| | AGTTTGATACCTACAACCTGTGGAATACGTTTACC | |
| | CGTCTGCAAGGCGGCGGTTCTGGCGGCGGCTCCC | |
| | ACCACCACCATCACCATTAA | |
| NSP14 | MAENVTGLFKDCSKVITGLHPTQAPTHLSVDTKFKT | 16 |
| amino acid | EGLCVDIPGIPKDMTYRRLISMMGFKMNYQVNGYP | |
| sequence | NMFITREEAIRHVRAWIGFDVEGCHATREAVGTNLP | |
| (SARS-CoV2) | LQLGFSTGVNLVAVPTGYVDTPNNTDFSRVSAKPPP | |
| | GDQFKHLIPLMYKGLPWNVVRIKIVQMLSDTLKNL | |
| | SDRVVFVLWAHGFELTSMKYFVKIGPERTCCLCDR | |
| | RATCFSTASDTYACWHHSIGFDYVYNPFMIDVQQW | |
| | GFTGNLQSNHDLYCQVHGNAHVASCDAIMTRCLA | |
| | VHECFVKRVDWTIEYPIIGDELKINAACRKVQHMV | |
| | VKAALLADKFPVLHDIGNPKAIKCVPQADVEWKFY | |
| | DAQPCSDKAYKIEELFYSYATHSDKFTDGVCLFWN | |
| | CNVDRYPANSIVCRFDTRVLSNLNLPGCDGGSLYV | |
| | NKHAFHTPAFDKSAFVNLKQLPFFYYSDSPCESHGK | |
| | QVVSDIDYVPLKSATCITRCNLGGAVCRHHANEYR | |
| | LYLDAYNMMISAGFSLWVYKQFDTYNLWNTFTRL | |
| | QGGGSGGGSHHHHHH | |
| NSP10 | ATGGCTGGTAATGCTACGGAAGTCCCGGCAAATA | 17 |
| nucleotide | GTACGGTGCTGTCGTTCTGTGCGTTCGCTGTTGAT | |
| sequence | GCGGCAAAAGCCTACAAAGATTATCTGGCCAGTG | |
| (SARS-CoV2) | GCGGTCAGCCGATTACGAACTGCGTTAAAATGCT | |
| | GTGTACCCATACCGGTACCGGTCAGGCCATCACC | |
| | GTCACCCCGGAAGCCAATATGGACCAAGAATCTT | |
| | TTGGCGGTGCATCTTGCTGTCTGTATTGCCGTTGT | |
| | CATATTGATCACCCGAACCCGAAAGGCTTCTGCG | |
| | ACCTGAAGGGTAAATACGTCCAGATCCCGACCAC | |
| | GTGTGCTAACGATCCGGTGGGCTTTACCCTGAAA | |
| | AATACGGTGTGCACCGTTTGTGGCATGTGGAAAG | |
| | GCTACGGTTGCAGCTGTGACCAACTGCGCGAACC | |
| | GATGCTGCAAGGCGGCGGCTCAGGCGGCGGCTCA | |
| | CACCACCACCACCATCATTAA | |
| NSP10 | MAGNATEVPANSTVLSFCAFAVDAAKAYKDYLAS | 18 |
| amino acid | GGQPITNCVKMLCTHTGTGQAITVTPEANMDQESF | |
| sequence | GGASCCLYCRCHIDHPNPKGFCDLKGKYVQIPTTCA | |
| (SARS-CoV2) | NDPVGFTLKNTVCTVCGMWKGYGCSCDQLREPML | |
| | QGGGSGGGSHHHHHH | |
| NSP14 | ATGGCTGAAAATGTAACAGGACTATTTAAAGACT | 20 |
| nucleotide | GTTCCAAAATCATCACCGGCCTGCATCCGACCCA | |
| sequence | GGCACCGACTCACCTGTCCGTGGATATCAAATTT | |
| (SARS-CoV) | AAAACCGAGGGTCTGTGCGTGGATATTCCGGGTA | |
| | TTCCGAAAGATATGACCTACCGTCGTCTGATTTCT | |
| | ATGATGGGTTTCAAGATGAATTATCAGGTTAATG | |
| | GTTACCCGAATATGTTCATCACGCGTGAAGAGGC | |
| | AATTCGTCATGTTCGTGCTTGGATTGGATTCGACG | |
| | TTGAAGGCTGCCATGCTACCAGAGATGCTGTGGG | |
| | TACAAACCTTCCACTGCAACTGGGTTTCTCGACCG | |
| | GGGTGAACTTGGTTGCTGTCCCAACGGGCTACGT | |
| | GGACACTGAGAACAACACCGAGTTCACCCGAGTT | |
| | AATGCGAAACCGCCTCCGGGCGACCAGTTTAAAC | |
| | ACCTGATCCCGTTGATGTATAAAGGTCTGCCGTG | |
| | GAATGTTGTGCGTATTAAGATCGTACAAATGCTG | |
| | TCCGACACCCTGAAGGGTCTTAGCGACCGCGTTG | |
| | TCTTTGTGCTGTGGGCACATGGCTTCGAATTGACC | |
| | AGCATGAAATACTTCGTGAAAATCGGTCCGGAGC | |
| | GTACCTGCTGTCTGTGTGATAAGCGCGCGACGTG | |
| | CTTTAGCACCTCTAGCGACACTTACGCCTGTTGGA | |
| | ACCACAGCGTAGGCTTCGACTACGTGTATAACCC | |
| | ATTTATGATCGATGTGCAGCAGTGGGGTTTCACC | |
| | GGTAACCTGCAAAGCAACCACGATCAGCATTGTC | |
| | AGGTTCACGGCAACGCGCATGTTGCCAGCTGTGA | |
| | TGCGATCATGACTCGTTGCCTGGCGGTCCACGAG | |
| | TGCTTTGTTAAACGTGTTGACTGGTCCGTCGAGTA | |
| | CCCGATTATCGGCGACGAACTGCGTGTGAACAGC | |
| | GCGTGCCGTAAAGTCCAGCACATGGTTGTGAAGT | |
| | CCGCGCTCCTGGCCGATAAGTTCCCGGTTCTGCAC | |
| | GACATCGGCAACCCGAAAGCAATCAAGTGCGTAC | |
| | CGCAGGCGGAAGTTGAGTGGAAATTTTACGATGC | |
| | GCAGCCGTGCAGCGACAAGGCGTACAAGATTGA | |
| | AGAGTTGTTCTACAGTTATGCTACGCATCACGAT | |
| | AAGTTTACCGATGGTGTGTGCTTGTTTTGGAACTG | |
| | CAACGTCGACCGTTATCCGGCGAATGCCATTGTTT | |
| | GTCGCTTTGATACCCGCGTGTTGTCTAATTTGAAT | |
| | TTACCCGGGTGCGACGGCGGTTCGCTGTATGTGA | |
| | ACAAACATGCGTTCCATACCCCGGCATTCGATAA | |
| | GTCAGCCTTCACCAATCTCAAGCAACTGCCGTTTT | |
| | TTTATTATAGCGACAGCCCGTGCGAAAGCCATGG | |
| | TAAACAAGTCGTTAGCGACATCGACTACGTGCCG | |
| | TTGAAGAGCGCGACATGCATCACCCGTTGTAACT | |
| | TGGGCGGTGCCGTGTGCCGCCATCACGCGAATGA | |
| | ATACCGCCAGTATCTGGATGCGTATAACATGATG | |
| | ATTAGCGCGGGTTTCTCTCTGTGGATTTATAAGCA | |
| | ATTCGACACGTACAACTTATGGAACACCTTTACC | |
| | CGCCTGCAAGGTGGCGGCTCTGGTGGCGGCTCCC | |
| | ACCACCACCACCACCACTAA | |
| NSP14 | MAENVTGLFKDCSKIITGLHPTQAPTHLSVDIKFKTE | 21 |
| amino acid | GLCVDIPGIPKDMTYRRLISMMGFKMNYQVNGYPN | |
| sequence | MFITREEAIRHVRAWIGFDVEGCHATRDAVGTNLPL | |
| (SARS-CoV) | QLGFSTGVNLVAVPTGYVDTENNTEFTRVNAKPPP | |
| | GDQFKHLIPLMYKGLPWNVVRIKIVQMLSDTLKGL | |
| | SDRVVFVLWAHGFELTSMKYFVKIGPERTCCLCDK | |
| | RATCFSTSSDTYACWNHSVGFDYVYNPFMIDVQQ | |
| | WGFTGNLQSNHDQHCQVHGNAHVASCDAIMTRCL | |
| | AVHECFVKRVDWSVEYPIIGDELRVNSACRKVQHM | |
| | VVKSALLADKFPVLHDIGNPKAIKCVPQAEVEWKF | |
| | YDAQPCSDKAYKIEELFYSYATHHDKFTDGVCLFW | |
| | NCNVDRYPANAIVCRFDTRVLSNLNLPGCDGGSLY | |
| | VNKHAFHTPAFDKSAFTNLKQLPFFYYSDSPCESHG | |
| | KQVVSDIDYVPLKSATCITRONLGGAVCRHHANEY | |
| | RQYLDAYNMMISAGFSLWIYKQFDTYNLWNTFTRL | |
| | QGGGSGGGSHHHHHH | |
| NSP10 | ATGGCAGGAAATGCGACAGAAGTACCCGCTAAC | 22 |
| nucleotide | AGCACCGTCCTGAGCTTCTGCGCGTTTGCAGTTGA | |
| sequence | TCCAGCGAAGGCGTATAAAGATTACTTGGCTTCC | |
| (SARS-CoV) | GGCGGCCAACCGATTACCAATTGCGTGAAGATGC | |
| | TGTGTACCCATACTGGTACAGGCCAAGCGATCAC | |
| | GGTTACCCCGGAAGCCAACATGGATCAAGAGTCG | |
| | TTCGGCGGCGCATCTTGTTGCCTGTACTGCCGTTG | |
| | TCATATCGACCATCCGAATCCGAAAGGCTTTTGC | |
| | GACTTAAAGGGTAAATACGTGCAGATTCCGACCA | |
| | CGTGCGCTAACGACCCGGTTGGTTTCACCTTGCGC | |
| | AACACCGTATGCACCGTGTGCGGTATGTGGAAAG | |
| | GTTATGGTTGTAGCTGCGACCAGCTGCGTGAGCC | |
| | GCTGATGCAGGGTGGCGGTTCCGGTGGTGGCAGC | |
| | CATCACCACCACCACCACTAA | |
| NSP10 | MAGNATEVPANSTVLSFCAFAVDPAKAYKDYLAS | 23 |
| amino acid | GGQPITNCVKMLCTHTGTGQAITVTPEANMDQESF | |
| sequence | GGASCCLYCRCHIDHPNPKGFCDLKGKYVQIPTTCA | |
| (SARS-CoV) | NDPVGFTLRNTVCTVCGMWKGYGCSCDQLREPLM | |
| | QGGGSGGGSHHHHHH | |
| NSP14 | ATGTCACAAATAGTAACAGGACTATTTAAAGATT | 24 |
| nucleotide | GTAGCCGTGAGACCAGCGGCTTGTCCCCGGCGTA | |
| sequence | CGCACCGACCTATGTGAGCGTTGATGATAAATAC | |
| (MERS-CoV) | AAGACCTCTGATGAACTTTGCGTGAACTTGAATTT | |
| | ACCGGCTAATGTCCCGTACAGTCGTGTTATTAGCC | |
| | GCATGGGCTTCAAATTAGACGCTACGGTTCCGGG | |
| | CTACCCGAAACTATTCATCACCCGGGAGGAAGCG | |
| | GTTCGTCAGGTTCGTAGCTGGATCGGTTTCGATGT | |
| | GGAAGGTGCCCACGCCTCGCGCAACGCCTGCGGT | |
| | ACAAATGTTCCGCTGCAGCTGGGTTTCAGCACCG | |
| | GTGTTAATTTTGTTGTCCAACCAGTGGGCGTGGTC | |
| | GACACCGAGTGGGGTAACATGCTGACGGGTATCG | |
| | CCGCTCGCCCACCCCCGGGTGAACAGTTTAAGCA | |
| | CCTGGTTCCGCTGATGCATAAAGGTGCGGCGTGG | |
| | CCTATTGTGCGCCGTCGTATTGTACAAATGTTGTC | |
| | GGACACGCTGGATAAGTTGTCCGACTACTGTACC | |
| | TTCGTGTGCTGGGCACACGGCTTCGAGCTGACCT | |
| | CTGCGTCCTACTTCTGCAAAATCGGCAAAGAGCA | |
| | GAAATGTTGCATGTGTAACCGTCGCGCTGCTGCG | |
| | TACAGCAGCCCGCTGCAAAGCTATGCGTGCTGGA | |
| | CCCACTCTTGCGGTTACGATTATGTCTATAACCCG | |
| | TTTTTTGTCGACGTTCAACAGTGGGGTTACGTAGG | |
| | CAACCTGGCGACTAACCACGATCGTTATTGCAGC | |
| | GTGCACCAGGGTGCGCACGTGGCCTCAAATGATG | |
| | CGATCATGACCCGTTGCCTGGCGATTCATAGCTG | |
| | CTTTATCGAGCGCGTGGACTGGGATATCGAATAC | |
| | CCGTATATCAGCCATGAAAAAAAACTGAACAGCT | |
| | GCTGCCGTATTGTCGAACGCAATGTGGTACGTGC | |
| | AGCGCTGCTGGCGGGTAGCTTTGACAAGGTCTAC | |
| | GATATCGGTAACCCGAAGGGCATCCCGATTGTTG | |
| | ACGATCCGGTTGTGGACTGGCATTATTTTGACGCC | |
| | CAGCCGTTGACCCGCAAAGTGCAACAACTGTTCT | |
| | ACACTGAGGATATGGCAAGCCGTTTTGCGGATGG | |
| | TCTCTGCCTGTTCTGGAATTGTAACGTTCCGAAGT | |
| | ACCCAAATAACGCAATTGTTTGCCGTTTTGACACT | |
| | CGCGTTCACAGCGAATTCAACCTGCCGGGGTGCG | |
| | ACGGCGGTTCGCTGTATGTGAATAAGCATGCGTT | |
| | TCATACCCCGGCGTACGACGTGTCTGCGTTTCGTG | |
| | ATCTGAAGCCGCTCCCGTTTTTCTATTACTCCACG | |
| | ACCCCGTGTGAAGTTCATGGTAACGGTTCTATGA | |
| | TTGAGGACATCGACTACGTGCCGCTTAAGAGCGC | |
| | GGTTTGCATTACCGCATGCAACTTAGGAGGCGCG | |
| | GTGTGTCGTAAGCACGCAACCGAGTATCGTGAGT | |
| | ATATGGAAGCGTACAACTTGGTTTCCGCTTCCGG | |
| | CTTCCGCCTGTGGTGTTACAAGACGTTCGACATCT | |
| | ATAACCTGTGGTCCACCTTCACCAAAGTTCAGGG | |
| | CGGCGGCTCGGGTGGCGGCAGCCATCACCACCAT | |
| | CACCACTAA | |
| NSP14 | MSQIVTGLFKDCSRETSGLSPAYAPTYVSVDDKYKT | 25 |
| amino acid | SDELCVNLNLPANVPYSRVISRMGFKLDATVPGYPK | |
| sequence | LFITREEAVRQVRSWIGFDVEGAHASRNACGTNVPL | |
| (MERS-CoV) | QLGFSTGVNFVVQPVGVVDTEWGNMLTGIAARPPP | |
| | GEQFKHLVPLMHKGAAWPIVRRRIVQMLSDTLDKL | |
| | SDYCTFVCWAHGFELTSASYFCKIGKEQKCCMCNR | |
| | RAAAYSSPLQSYACWTHSCGYDYVYNPFFVDVQQ | |
| | WGYVGNLATNHDRYCSVHQGAHVASNDAIMTRCL | |
| | AIHSCFIERVDWDIEYPYISHEKKLNSCCRIVERNVV | |
| | RAALLAGSFDKVYDIGNPKGIPIVDDPVVDWHYFD | |
| | AQPLTRKVQQLFYTEDMASRFADGLCLFWNCNVP | |
| | KYPNNAIVCRFDTRVHSEFNLPGCDGGSLYVNKHA | |
| | FHTPAYDVSAFRDLKPLPFFYYSTTPCEVHGNGSMI | |
| | EDIDYVPLKSAVCITACNLGGAVCRKHATEYREYM | |
| | EAYNLVSASGFRLWCYKTFDIYNLWSTFTKVQGGG | |
| | SGGGSHHHHHH | |
| NSP10 | ATGGCTGGATCAAATACAGAATTTGCAAGTAACA | 26 |
| nucleotide | GCTCGGTGCTTTCTTTGGTTAACTTCACCGTAGAT | |
| sequence | CCGCAAAAAGCGTATCTGGACTTCGTGAACGCCG | |
| (MERS-CoV) | GTGGTGCTCCGCTGACCAATTGCGTGAAGATGCT | |
| | GACGCCAAAGACCGGCACCGGTATTGCGATCAGC | |
| | GTGAAGCCGGAAAGCACCGCTGACCAAGAGACG | |
| | TACGGCGGCGCGAGCGTCTGCCTGTACTGCCGTG | |
| | CACATATCGAGCATCCGGATGTTAGCGGGGTGTG | |
| | CAAATACAAAGGTAAATTTGTCCAGATCCCGGCG | |
| | CAGTGTGTTCGTGATCCTGTTGGTTTTTGCCTGAG | |
| | CAATACTCCGTGTAATGTTTGCCAGTATTGGATTG | |
| | GTTATGGTTGTAACTGCGACTCTTTGCGCCAGGCG | |
| | GCACTGCCGCAAGGTGGCGGCTCCGGCGGTGGCT | |
| | CCCATCACCACCACCACCACTAA | |
| NSP10 | MAGSNTEFASNSSVLSLVNFTVDPQKAYLDFVNAG | 27 |
| amino acid | GAPLTNCVKMLTPKTGTGIAISVKPESTADQETYGG | |
| sequence | ASVCLYCRAHIEHPDVSGVCKYKGKFVQIPAQCVR | |
| (MERS-CoV) | DPVGFCLSNTPCNVCQYWIGYGCNCDSLRQAALPQ | |
| | GGGSGGGSHHHHHH | |

In some embodiments, the NSP14 exonuclease comprises an amino acid sequence of SEQ ID NO: 16 as encoded by a nucleotide sequence of SEQ ID NO: 15. In some embodiments, the nucleotide sequence is a DNA sequence. In some embodiments, the DNA sequence is complementary DNA (cDNA). In some embodiments, the cDNA sequence is codon-optimized. In some embodiments, the cDNA sequence is E. coli codon-optimized. In some embodiments, the cDNA sequence is not codon-optimized.

In some embodiments, the NSP14 exonuclease comprises an amino sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 16. In some embodiments, the NSP14 exonuclease amino acid sequence is encoded by a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 15.

In some embodiments, the NSP10 comprises an amino acid sequence of SEQ ID NO: 18 as encoded by a nucleotide sequences of SEQ ID NO: 17. In some embodiments, the nucleotide sequence is a DNA sequence. In some embodiments, the DNA sequence is complementary DNA (cDNA). In some embodiments, the cDNA sequence is codon-optimized. In some embodiments, the cDNA sequence is E. coli codon-optimized. In some embodiments, the cDNA sequence is not codon-optimized.

In some embodiments, the NSP10 comprises an amino sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18. In some embodiments, the NSP10 amino acid sequence is encoded by a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 17.

In some embodiments, the NSP14 exonuclease comprises an amino acid sequence of SEQ ID NO: 21 as encoded by a nucleotide sequence of SEQ ID NO: 20. In some embodiments, the nucleotide sequence is a DNA sequence. In some embodiments, the DNA sequence is complementary DNA (cDNA). In some embodiments, the cDNA sequence is codon-optimized. In some embodiments, the cDNA sequence is E. coli codon-optimized. In some embodiments, the cDNA sequence is not codon-optimized.

In some embodiments, the NSP14 exonuclease comprises an amino sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21. In some embodiments, the NSP14 exonuclease amino acid sequence is encoded by a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20.

In some embodiments, the NSP10 comprises an amino acid sequence of SEQ ID NO: 23 as encoded by a nucleotide sequences of SEQ ID NO: 22. In some embodiments, the nucleotide sequence is a DNA sequence. In some embodiments, the DNA sequence is complementary DNA (cDNA). In some embodiments, the cDNA sequence is codon-optimized. In some embodiments, the cDNA sequence is E. coli codon-optimized. In some embodiments, the cDNA sequence is not codon-optimized.

In some embodiments, the NSP10 comprises an amino sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 23. In some embodiments, the NSP10 amino acid sequence is encoded by a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 22.

In some embodiments, the NSP14 exonuclease comprises an amino acid sequence of SEQ ID NO: 25 as encoded by a nucleotide sequence of SEQ ID NO: 24. In some embodiments, the nucleotide sequence is a DNA sequence. In some embodiments, the DNA sequence is complementary DNA (cDNA). In some embodiments, the cDNA sequence is codon-optimized. In some embodiments, the cDNA sequence is E. coli codon-optimized. In some embodiments, the cDNA sequence is not codon-optimized.

In some embodiments, the NSP14 exonuclease comprises an amino sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 25. In some embodiments, the NSP14 exonuclease amino acid sequence is encoded by a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 24.

In some embodiments, the NSP10 comprises an amino acid sequence of SEQ ID NO: 27 as encoded by a nucleotide sequences of SEQ ID NO: 26. In some embodiments, the nucleotide sequence is a DNA sequence. In some embodiments, the DNA sequence is complementary DNA (cDNA). In some embodiments, the cDNA sequence is codon-optimized. In some embodiments, the cDNA sequence is E. coli codon-optimized. In some embodiments, the cDNA sequence is not codon-optimized.

In some embodiments, the NSP10 comprises an amino sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 27. In some embodiments, the NSP10 amino acid sequence is encoded by a nucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 26.

In some embodiments, FRET-based methods disclosed herein may be used for measuring a 3′ to 5′ exonuclease activity of a NSP14 exonuclease.

In some embodiments, FRET-based methods disclosed herein may be used for measuring a 3′ to 5′ exonuclease activity of a NSP14/NSP10 exonuclease complex.

In some embodiments, the NSP14 exonuclease or NSP14/NSP10 exonuclease complex may be from a virus of the order Nidovirales. In some embodiments, the NSP14 exonuclease or NSP14/NSP10 exonuclease complex may be from a virus of the family Coronaviridae. In some embodiments, the virus is a Coronavirus (CoV). As a non-limiting example, the Coronavirus may be SARS-CoV, SARS-CoV2, MERS-CoV, HCoV-OC43, HCoV-229E, or any related animal or zoonotic Coronaviridae strain. In some embodiments, the Coronavirus virus is a SARS-CoV2 virus. In some embodiments, the Coronavirus virus is a SARS-CoV virus. In some embodiments, the Coronavirus virus is MERS-CoV virus.

In some embodiments, the exonuclease comprises purified NSP14 and NSP10 proteins in 1:1 to 1:50 molar ratio. In some embodiments, the exonuclease comprises purified NSP14 and NSP10 proteins in a 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or 1:50 molar ration. In some embodiments, the exonuclease comprises purified NSP14 and NSP10 proteins in 1:1 to 1:5 molar ratio. In some embodiments the exonuclease comprises purified NSP14 and NSP10 proteins in 1:1 molar ratio. In some embodiments the exonuclease comprises purified NSP14 and NSP10 proteins in 1:2 molar ratio. In some embodiments the exonuclease comprises purified NSP14 and NSP10 proteins in 1:3 molar ratio. In some embodiments the exonuclease comprises purified NSP14 and NSP10 proteins in 1:4 molar ratio. In some embodiments the exonuclease comprises purified NSP14 and NSP10 proteins in 1:5 molar ratio.

In some embodiments, the FRET-based methods for measuring a 3′ to 5′ exonuclease activity in a sample disclosed herein comprise contacting the sample with a fluorescently labeled double-stranded RNA (dsRNA) substrate. In some embodiments, the exonuclease is contacted with dsRNA substrate in the presence of Mg²⁺. In some embodiments, the exonuclease is contacted with dsRNA substrate in the absence of Mg²⁺.

In some embodiments, the exonuclease is contacted with dsRNA substrate at a temperature between 1° C. to 50° C. In some embodiments, the exonuclease is contacted with dsRNA substrate at a temperature of 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the exonuclease is contacted with dsRNA substrate at a temperature of about 4° C. In some embodiments, the exonuclease is contacted with dsRNA substrate at a temperature of about 37° C.

In some embodiments, the exonuclease is contacted with dsRNA substrate at a temperature between 30° C. to 37° C. In some embodiments, the exonuclease is contacted with dsRNA substrate at a temperature of about 30° C. In some embodiments, the exonuclease is contacted with dsRNA substrate at a temperature of about 31° C. In some embodiments, the exonuclease is contacted with dsRNA substrate at a temperature of about 32° C. In some embodiments, the exonuclease is contacted with dsRNA substrate at a temperature of about 33° C. In some embodiments, the exonuclease is contacted with dsRNA substrate at a temperature of about 34° C. In some embodiments, the exonuclease is contacted with dsRNA substrate at a temperature of about 35° C. In some embodiments, the exonuclease is contacted with dsRNA substrate at a temperature of about 36° C. In some embodiments, the exonuclease is contacted with dsRNA substrate at a temperature of about 37° C.

In some embodiments, the exonuclease is contacted with dsRNA substrate for at least about 1 min to at least about 240 min. In some embodiments, the exonuclease is contacted with dsRNA substrate for at least about 1 min, 2 min, 5 min, 10 min, 20 min, 30 min, 40 min, 50 min, 60 min, 70 min, 80 min, 90 min, 100 min, 110 min, 120 min, 130 min, 140 min, 150 min, 160 min, 170 min, 180 min, 190 min, 200 min, 210 min, 220 min, 230 min, or 240 min. In some embodiments, the exonuclease is contacted with dsRNA substrate for about 30 min. In some embodiments, the exonuclease is contacted with dsRNA substrate for about 40 min. In some embodiments, the exonuclease is contacted with dsRNA substrate for about 50 min. In some embodiments, the exonuclease is contacted with dsRNA substrate for about 60 min.

In some embodiments, the exonuclease is contacted with dsRNA substrate for about 1 hour to about 5 hours. In some embodiments, the exonuclease is contacted with dsRNA substrate for about 1 hour. In some embodiments, the exonuclease is contacted with dsRNA substrate for about 2 hours. In some embodiments, the exonuclease is contacted with dsRNA substrate for about 3 hours. In some embodiments, the exonuclease is contacted with dsRNA substrate for about 4 hours. In some embodiments, the exonuclease is contacted with dsRNA substrate for about 5 hours.

In some embodiments, the exonuclease is contacted with dsRNA substrate in a solution. As a non-limiting example, the solution may comprise TRIS-HCl, MgCl₂, and DTT. As a non-limiting example, the solution comprises TRIS-HCl. As a non-limiting example, the solution comprises MgCl₂. As a non-limiting example, the solution comprises DTT.

In some embodiments, the solution comprises TRIS-HCl at a concentration of at least about 1 mM to 200 mM. As a non-limiting example, the solution may comprises TRIS-HCl at a concentration of about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM or 200 mM. In some embodiments, the solution may comprise TRIS-HCL at a concentration of about 50 mM.

In some embodiments, the solution may comprise TRIS-HCl at a pH of least about a pH of 4 to at least about a pH of 12. As a non-limiting example, the solution may comprise TRIS-HCl at a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9 or 12.0. In some embodiments, the solution may comprise TRIS-HCl at a pH of 7.5.

In some embodiments, the solution may comprise MgCl₂ at a concentration of at least about 0.1 to at least about 5 mM. As a non-limiting example, the solution may comprise MgCl₂ at a concentration of 0.1 mM, 0.2 mM, 0.3 mM, 0.4 mM, 0.5 mM, 1 mM, 1.5 mM, 2 mM, 2.5 mM, 3 mM, 3.5 mM, 4 mM, 4.5 mM, or 5 mM. In some embodiments, the solution may comprise MgCl₂ at a concentration of 2 mM.

In some embodiments, the solution may comprise DTT at a concentration of at least about 0.1 mM to at least about 5 mM. As a non-limiting example, the solution may comprise DTT at a concentration of 0.1 mM, 0.2 mM, 0.3 mM, 0.4 mM, 0.5 mM, 1 mM, 1.5 mM, 2 mM, 2.5 mM, 3 mM, 3.5 mM, 4 mM, 4.5 mM, or 5 mM DTT. In some embodiments, the solution may comprise DTT at a concentration of 2 mM.

In some embodiments, the exonuclease is contacted with dsRNA substrate at a temperature between 30° C. to 37° C.

In some embodiments, the exonuclease is contacted with dsRNA substrate at a temperature between 30° C. to 37° C. for about 1 hour.

In some embodiments, the exonuclease is contacted with dsRNA substrate at a temperature between 30° C. to 37° C. for about 1 hour in a solution. In some embodiments, the solution may comprise in 50 mM TRIS-HCl, pH=7.5, 2 mM MgCl₂, 2 mM DTT.

In some embodiments, the exonuclease is contacted with dsRNA substrate at a temperature between 30° C. to 37° C. for about 1 hour in 50 mM TRIS-HCl, pH=7.5, 2 mM MgCl₂, 2 mM DTT.

In some embodiments, the NSP10 may be pre-treated with ZnCl₂. In some embodiments, the NSP10 may be pre-treated with ZnCl₂ at a concentration of at least about 0.1 M to at least about 4 μM. As a non-limiting example, the NSP10 may be pre-treated with ZnCl₂ at a concentration of 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 2.1 μM, 2.2 μM, 2.3 μM, 2.4 μM, 2.5 μM, 2.6 μM, 2.7 μM, 2.8 μM, 2.9 μM, 3.0 μM, 3.1 μM, 3.2 μM, 3.3 μM, 3.4 μM, 3.5 μM, 3.6 μM, 3.7 μM, 3.8 μM, 3.9 M or 4.0 μM. In some embodiments, the NSP10 may be pre-treated with ZnCl₂ at a concentration of at least about 0.1 μM to at least about 2 μM. In some embodiments, the NSP10 may be pre-treated with ZnCl₂ at a concentration of 0.2 μM.

### Test Compounds

In one aspect is provided a fluorescence resonance energy transfer (FRET)-based method for identifying and/or assessing a modulator of a 3′ to 5′ exonuclease, comprising: (a) in a test reaction mixture, contacting the exonuclease with a test compound and a fluorescently labeled double-stranded RNA (dsRNA) substrate, wherein said dsRNA substrate comprises (i) at least one free 3′ OH group, and (ii) a pair of FRET pair probes comprising a fluorophore and a quencher, wherein the fluorophore one probe is located at the 5′ end of the strand comprising the free 3′ OH group and the quencher other probe is located either at the 5′ end or at the 3′ end of the other strand of said dsRNA substrate, and when the substrate is uncleaved, the quencher quenches the fluorescence signal of the fluorophore; (b) incubating said test reaction mixture under conditions and for a time sufficient for cleavage of the substrate by the exonuclease in the absence of the test compound, wherein the cleavage of the substrate by the exonuclease causes sufficient separation of the fluorophore and the quencher to reduce quenching of the fluorescence signal of the fluorophore; (c) determining the fluorescence signal emitted from the test reaction mixture; (d) comparing the fluorescence signal determined in step (c) to a control fluorescence signal, wherein the control fluorescence signal is the fluorescence signal determined under the same conditions in a control sample comprising the same amounts of exonuclease and dsRNA substrate but in the absence of the test compound, and (e) (i) determining that the test compound is an inhibitor of the exonuclease if the fluorescence signal in the test reaction mixture is lower than in the control reaction mixture, or (ii) determining that the test compound is not an inhibitor of the exonuclease if the fluorescence signal in the test reaction mixture is not lower than in the control reaction mixture, or (iii) determining that the test compound is an activator of the exonuclease if the fluorescence signal in the test reaction mixture is higher than in the control reaction mixture. In some embodiments, step (a) may comprise pre-incubating the exonuclease with the test compound prior to the addition of the dsRNA substrate. In some embodiments, step (a) may comprise adding the test compound after contacting the exonuclease with a dsRNA substrate.

In one aspect is provided a fluorescence resonance energy transfer (FRET)-based method for identifying and/or assessing a modulator of a 3′ to 5′ exonuclease. Description and inhibitory activity of exemplary modulators of 3′ to 5′ exonucleases are provided below and in Table 2.

### Description of Structure-Activity Relationships (SAR) and the Underlying Design Principles of Inhibitory Compounds

In one aspect, the present disclosure provides compounds capable of inhibiting enzymatic activity of the NSP14-NSP10 complex. In another aspect, the present disclosure provides methods of inhibiting enzymatic activity of the NSP14-NSP10 complex with the inhibitory compounds of the present disclosure.

It is contemplated that there are multiple binding sites and multiple binding poses that can inhibit the exonuclease (ExoN) enzymatic activity of the NSP14-NSP10 complex. In accordance with these observations, there are multiple scaffolds and diverse structures that can realize this inhibitory action.

It has been surprisingly discovered that the following structural motifs, as described in detail below, may possess inhibitory activity of the NSP14-NSP10 complex. Without wishing to be bound by theory, it is contemplated that the inhibitory compounds of the present disclosure belong to the following classes: Formula (I) main site binding simple-ring core compounds, Formula (II) main site binding polycyclic core compounds and Formula (III) compounds that bind in alternative mode(s). For every clade, exemplary non-limiting embodiments are also presented, illustrating the SAR principles outlined in the description of each group.

In various embodiments, compounds acting as inhibitors might be either of a completely natural, semi-synthetic or synthetic source. In one embodiment, inhibitory compounds are naturally occurring. In another embodiment, inhibitory compounds are synthetic. In yet another embodiment, inhibitory compounds are semi-synthetic, i.e. they possess naturally occurring moieties, but have undergone some synthetic modifications.

In some embodiments, the inhibitory compounds may already possess an industrial or medicinal use that is unrelated to NSP14-NSP10 inhibition. In other embodiments, the inhibitory compounds may not have any prior identified use to date.

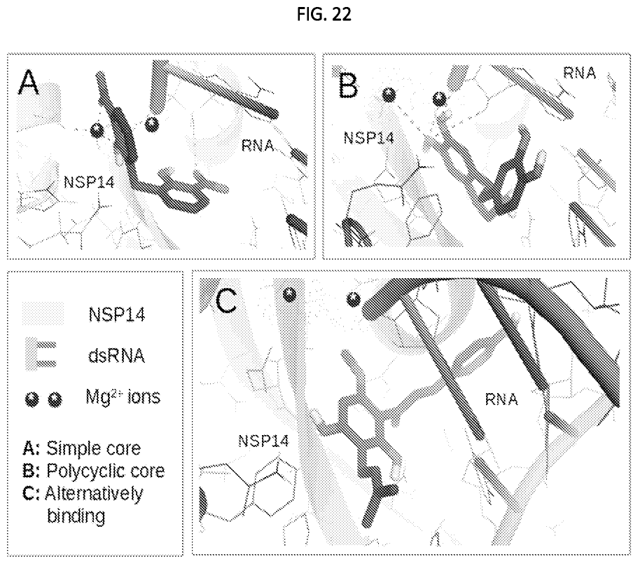

Non-limiting examples for the modelling of binding and computer-assisted design of inhibitory compounds are depicted in FIG. 22. This Figure depicts binding to the catalytic site with a simple monocyclic core ring (FIG. 22A, 3,3′ Methylenedisalicylic acid), with a polycyclic core ring (FIG. 22B, Tetrahydropapaveroline), or using alternative binding modes (FIG. 22C, Xanthohumol).

### Compounds of Formulas (A), (B), and (I)

In one aspect, the present disclosure provides compounds capable of inhibiting enzymatic activity of the NSP14-NSP10 complex having the structure of Formula (A):

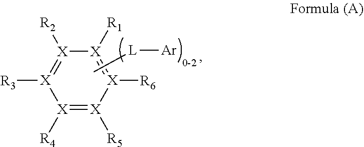

or a pharmaceutically acceptable salt thereof,
wherein X is independently at each occurrence selected from C, O, N, and S;
L is a linker selected independently at each occurrence from a bond, a C₁₋₆ alkyl, C₂₋₆ alkenyl, —CO—, —CO—NH—, —CO—(C₁₋₃ alkyl)-, and —CO—(C₂₋₄ alkenyl)-, wherein the C₁₋₆ alkyl optionally contains 1-2 heteroatoms selected from O, N, and S;
Ar is independently at each occurrence phenyl or a 5- or 6-membered heterocycle comprising from 1 to 3 heteroatoms independently selected from N, O, and S, wherein Ar is optionally substituted with one or more groups R′;
R₁, R₂, R₃, R₄, R₅ and R₆ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In another aspect, the present disclosure provides compounds capable of inhibiting enzymatic activity of the NSP14-NSP10 complex having the structure of Formula (B):

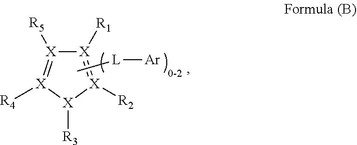

or a pharmaceutically acceptable salt thereof,
wherein X is independently at each occurrence selected from C, O, N, and S;
L is a linker selected independently at each occurrence from a bond, a C₁₋₆ alkyl, C₂₋₆ alkenyl, —CO—, —CO—NH—, —CO—(C₁₋₃ alkyl)-, and —CO—(C₂₋₄ alkenyl)-, wherein the C₁₋₆ alkyl optionally contains 1-2 heteroatoms selected from O, N, and S;
Ar is independently at each occurrence phenyl or a 5- or 6-membered heterocycle comprising from 1 to 3 heteroatoms independently selected from N, O, and S, wherein Ar is optionally substituted with one or more groups R′;
R₁, R₂, R₃, R₄, and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In another aspect, the present disclosure provides compounds capable of inhibiting enzymatic activity of the NSP14-NSP10 complex having the structure of Formula (I):

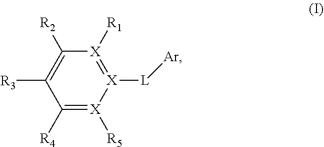

or a pharmaceutically acceptable salt thereof,
wherein X is independently at each occurrence selected from C, O, N, and S;
L is a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar is phenyl or a 5- or 6-membered heterocycle comprising from 1 to 3 heteroatoms independently selected from N, O, and S, wherein Ar is optionally substituted with one or more groups R′;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In one embodiment, the compounds capable of inhibiting enzymatic activity of the NSP14-NSP10 complex have the structure of Formula (IA):

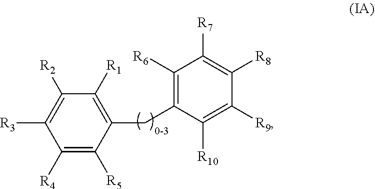

or a pharmaceutically acceptable salt thereof,
wherein R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In one particular embodiment, the compounds capable of inhibiting enzymatic activity of the NSP14-NSP10 complex have the structure of Formula (IA) are:

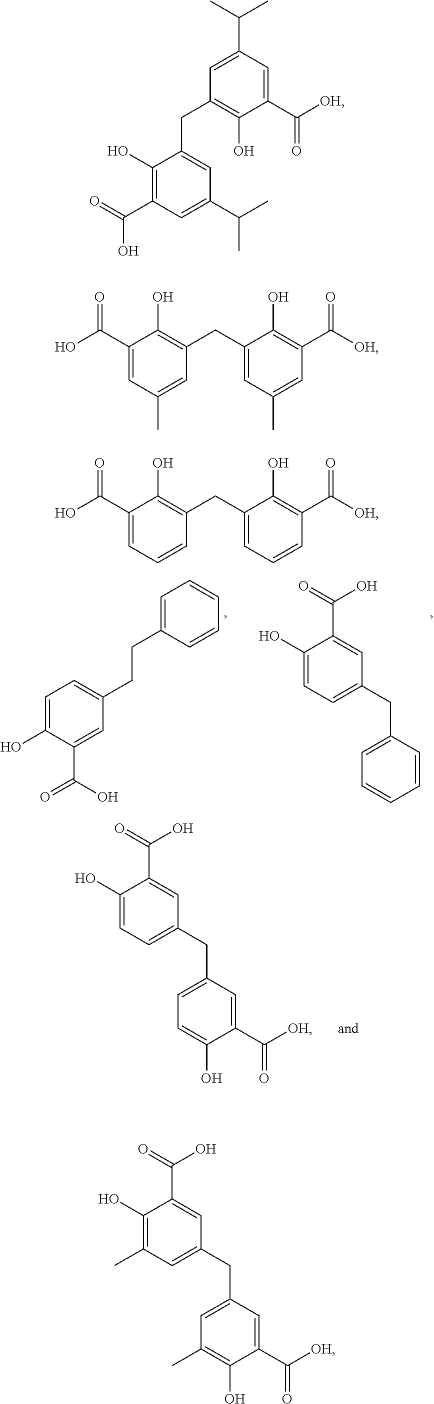

or a pharmaceutically acceptable salt thereof.

In one embodiment, the compounds capable of inhibiting enzymatic activity of the NSP14-NSP10 complex have the structure of Formula (IB):

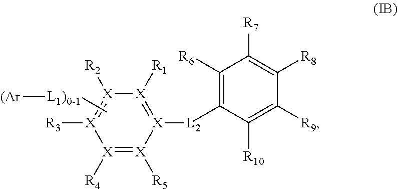

or a pharmaceutically acceptable salt thereof,
wherein X is independently at each occurrence selected from C and N;
L₁ and L₂ are independently linkers selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar is phenyl or a 5- or 6-membered heterocycle comprising from 1 to 3 heteroatoms independently selected from N, O, and S, wherein Ar is optionally substituted with one or more groups R′;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In one particular embodiment, the compounds capable of inhibiting enzymatic activity of the NSP14-NSP10 complex have the structure of Formula (IB) are:

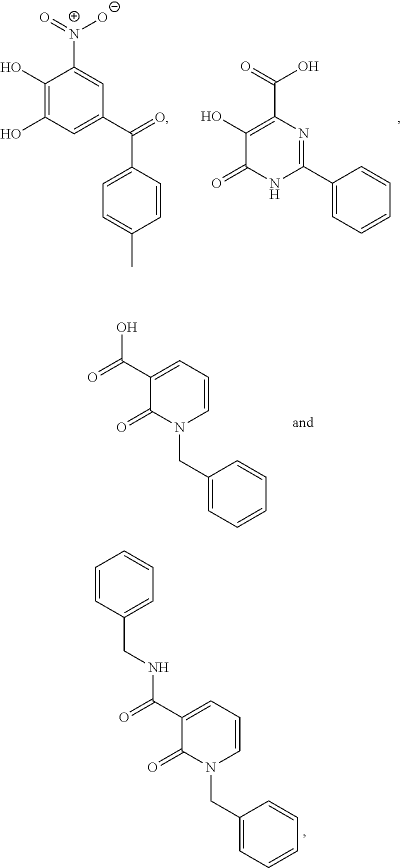

or a pharmaceutically acceptable salt thereof.

In one embodiment, the compounds capable of inhibiting enzymatic activity of the NSP14-NSP10 complex have the structure of Formula (IC):

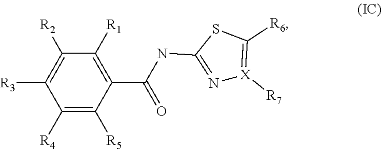

or a pharmaceutically acceptable salt thereof,
wherein X is selected from C and N;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein when X is C at least one of R₁, R₂, R₃, R₄ and R₅ is not H;
R₆ is H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof;
R₇ is absent, H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof; and
R* is independently selected at each occurrence from hydrogen or C₁-C₁₂ hydrocarbons each of which optionally contains 1-8 heteroatoms selected from halogen, O, N, and S and combinations thereof.

In one particular embodiment, the compounds capable of inhibiting enzymatic activity of the NSP14-NSP10 complex have the structure of Formula (IC) are:

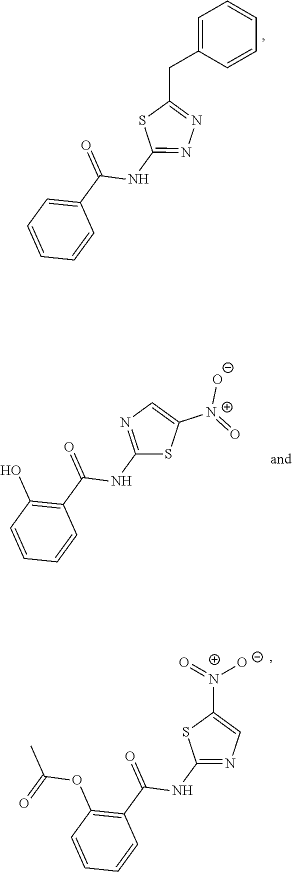

or a pharmaceutically acceptable salt thereof.

### Compounds of Formulas (II) and (II′)

In another aspect, the present disclosure provides compounds capable of inhibiting enzymatic activity of the NSP14-NSP10 complex having the structure of Formula (II):

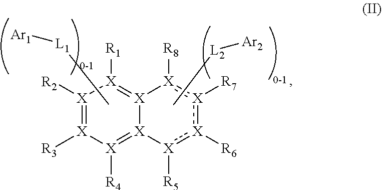

or a pharmaceutically acceptable salt thereof,
wherein X is independently at each occurrence selected from C, O, N, and S;
L₁ and L₂ are independently a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar₁ and Ar₂ are independently a phenyl, a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, R₇ and R₈ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and

R* is independently selected at each occurrence from hydrogen or C₁-C₁₂ hydrocarbons each of which optionally contains 1-8 heteroatoms selected from halogen, O, N, and S and combinations thereof. In one embodiment, the compounds capable of inhibiting enzymatic activity of the NSP14-NSP10 complex have the structure of Formula (IIA):

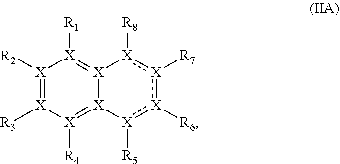

or a pharmaceutically acceptable salt thereof,
wherein X is independently at each occurrence selected from C, O, N, and S;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —F, —Cl, —Br, —I, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form one or more fused rings, and wherein at least two of R₁, R₂, R₃, R₄, R₅, R₆, R₇ and R₈ are not H;
R* is independently selected at each occurrence from hydrogen, C₁₋₁₂ alkyl, C₁₋₁₂ alkenyl, C₆₋₁₂ aryl, and C₁₋₁₂ aralkyl, each of which optionally contains 1-8 heteroatoms selected from halogen, O, N, and S and combinations thereof.

In one particular embodiment, the compounds capable of inhibiting enzymatic activity of the NSP14-NSP10 complex have the structure of Formula (IIA) are:

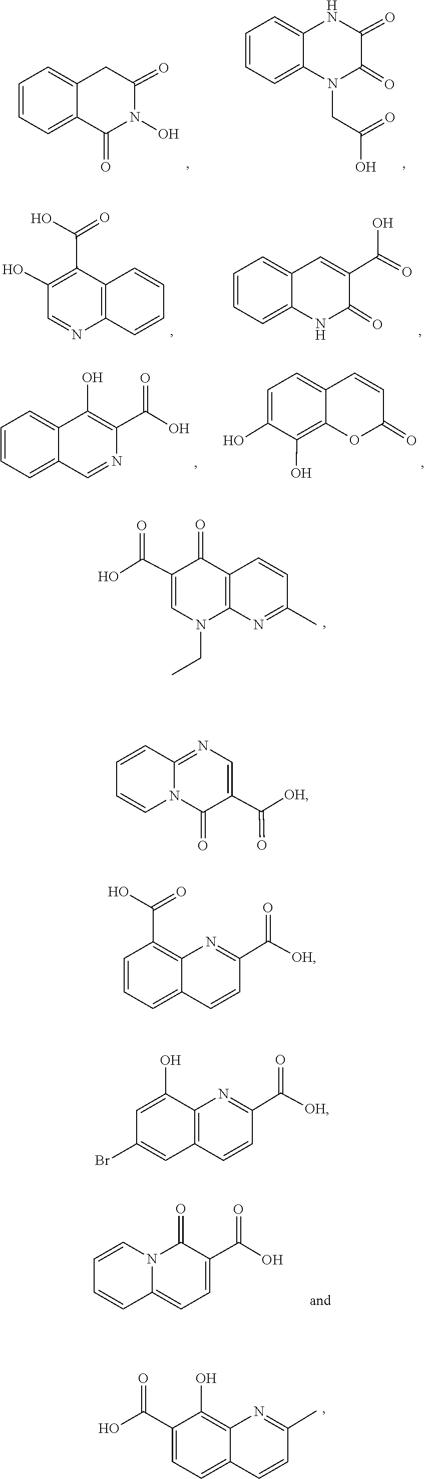

or a pharmaceutically acceptable salt thereof.

In one embodiment, the compounds capable of inhibiting enzymatic activity of the NSP14-NSP10 complex have the structure of Formula (IIB):

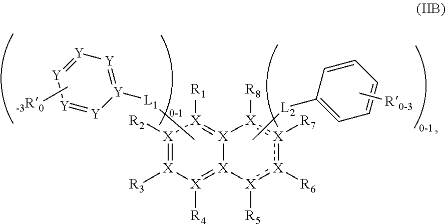

or a pharmaceutically acceptable salt thereof,
wherein X is independently at each occurrence selected from C, O, and N;
wherein Y is independently at each occurrence selected from a bond, C, O, and N;
L₁ and L₂ are independently a linker selected from a bond and a C₁₋₃ alkyl optionally containing 1-2 heteroatoms selected from O, N, and S;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In one particular embodiment, the compounds capable of inhibiting enzymatic activity of the NSP14-NSP10 complex having the structure of Formula (IIB) are:

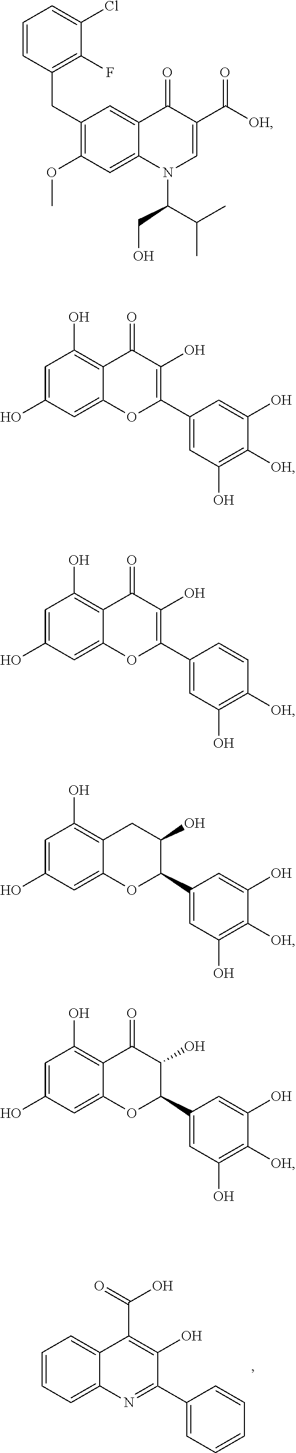 [SMILES: COc1cc2c(cc1Cc1cccc(Cl)c1F)c(=O)c(C(=O)O)cn2[C@H](CO)C(C)C ; O=c1c(O)c(-c2cc(O)c(O)c(O)c2)oc2cc(O)cc(O)c12 ; O=c1c(O)c(-c2ccc(O)c(O)c2)oc2cc(O)cc(O)c12 ; Oc1cc(O)c2c(c1)O[C@H](c1cc(O)c(O)c(O)c1)[C@H](O)C2 ; O=C1c2c(O)cc(O)cc2O[C@H](c2cc(O)c(O)c(O)c2)[C@H]1O ; O=C(O)c1c(O)c(-c2ccccc2)nc2ccccc12]

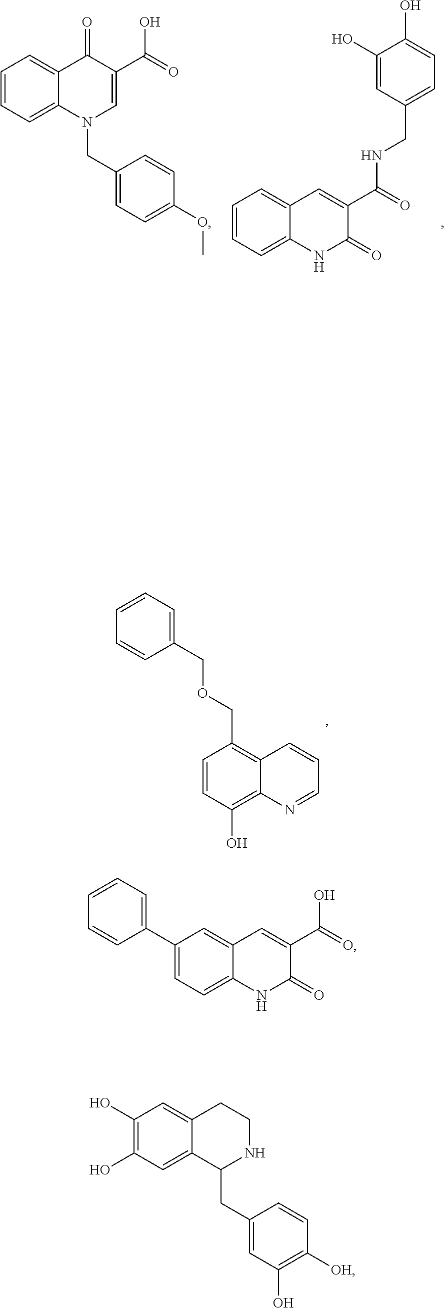

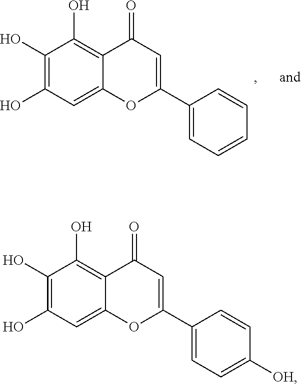 [SMILES: O=c1cc(-c2ccccc2)oc2cc(O)c(O)c(O)c12 ; O=c1cc(-c2ccc(O)cc2)oc2cc(O)c(O)c(O)c12]

or a pharmaceutically acceptable salt thereof.

In another embodiment, the compounds capable of inhibiting enzymatic activity of the NSP14-NSP10 complex having the structure of Formula (IIB) are:

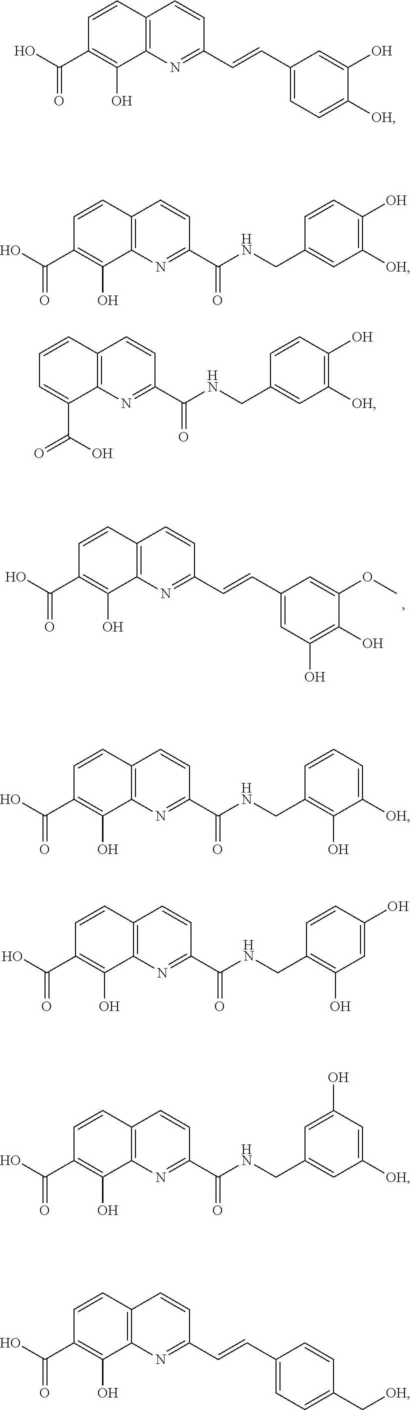

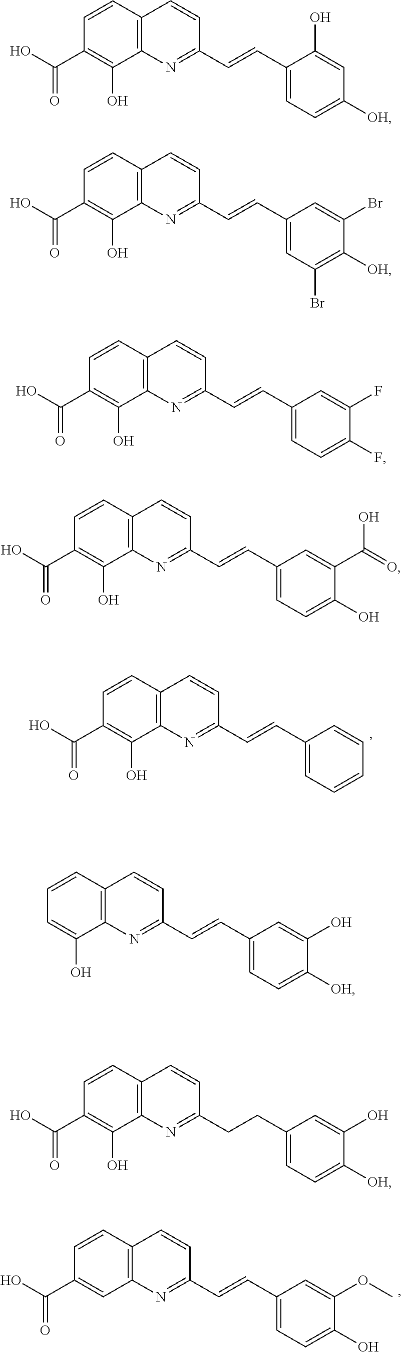

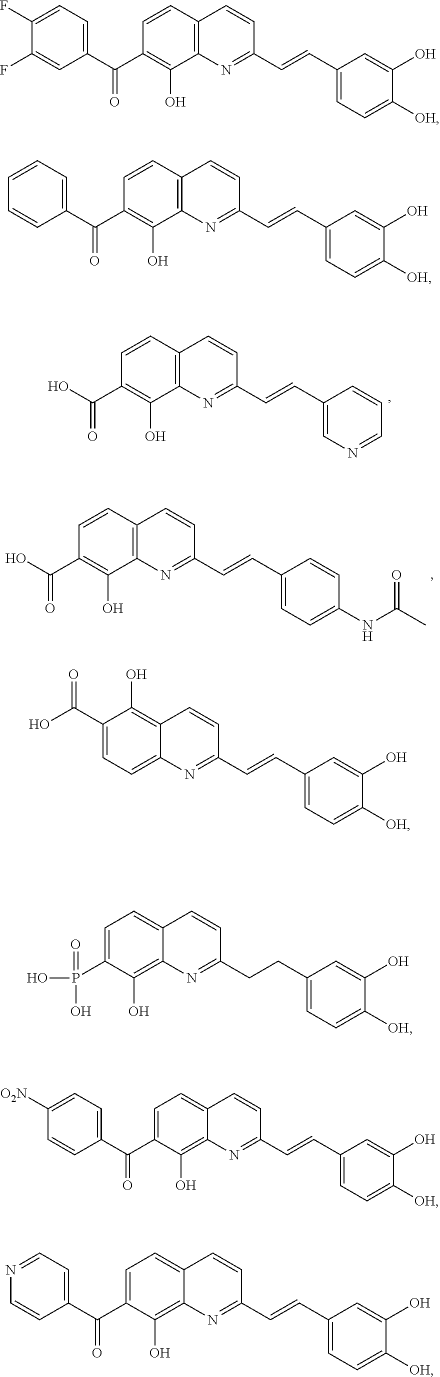

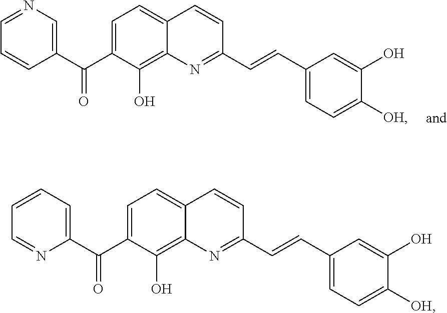 [SMILES: O=C(c1cccnc1)c1ccc2ccc(/C=C/c3ccc(O)c(O)c3)nc2c1O ; O=C(c1ccccn1)c1ccc2ccc(/C=C/c3ccc(O)c(O)c3)nc2c1O]

or a pharmaceutically acceptable salt thereof.

In one particular embodiment, the compounds capable of inhibiting enzymatic activity of the NSP14-NSP10 complex are those described in FR2839646, PCT/FR2003/001487, US2005/0261336, U.S. Pat. No. 7,479,497, US2008/0161350, EP1507532, WO2003/096965, JP2005531554, AU2003251047, CA2489102, U.S. Pat. Nos. 6,670,377, and 7,064,133, the contents of all of which are incorporated by reference herein in their entireties.

In some embodiments, the compound of Formula (IIB) has the structure:

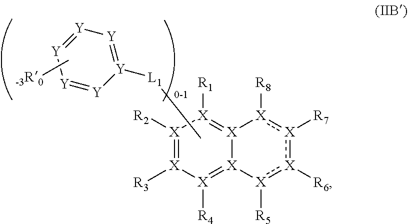

or a pharmaceutically acceptable salt thereof.

In some embodiments, the compound of Formula (IIB′) has the structure:

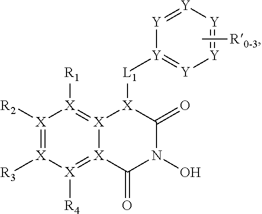

or a pharmaceutically acceptable salt thereof.

In one particular embodiment, the compound of Formula (IIB′) has the structure:

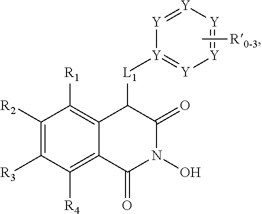

or a pharmaceutically acceptable salt thereof.

In some embodiments, the compound having the structure of Formula (IIB′) is selected from the group consisting of:

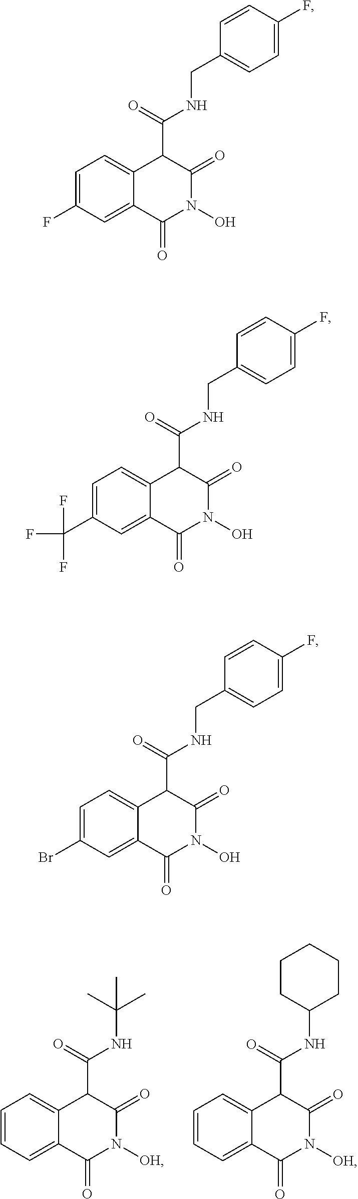 [SMILES: O=C(NCc1ccc(F)cc1)C1C(=O)N(O)C(=O)c2cc(F)ccc21 ; O=C(NCc1ccc(F)cc1)C1C(=O)N(O)C(=O)c2cc(C(F)(F)F)ccc21 ; O=C(NCc1ccc(F)cc1)C1C(=O)N(O)C(=O)c2cc(Br)ccc21 ; CC(C)(C)NC(=O)C1C(=O)N(O)C(=O)c2ccccc21 ; O=C(NC1CCCCC1)C1C(=O)N(O)C(=O)c2ccccc21]

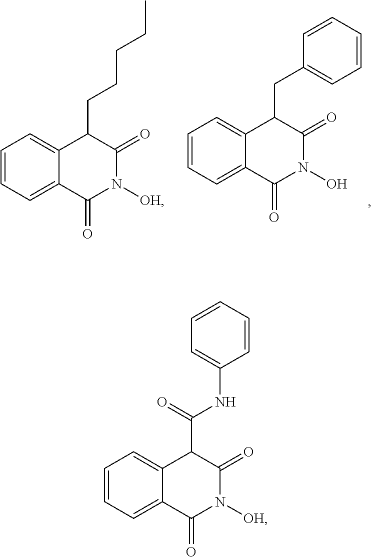

or a pharmaceutically acceptable salt thereof.

In some embodiments, the compound of Formula (IIB) has the structure:

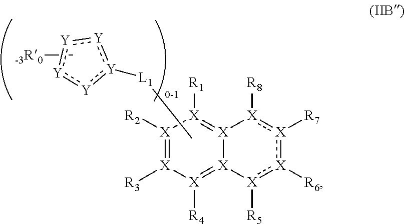

or a pharmaceutically acceptable salt thereof.

In some embodiment, the compound of Formula (IIB″) has the structure:

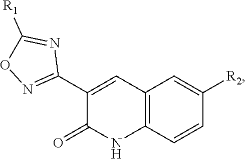

or a pharmaceutically acceptable salt thereof,
wherein R₁ and R₂ are independently H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof.

In some embodiments, R₁ is phenyl, optionally substituted with one or more of —F; —Cl; —Br; —I; —OH. In some embodiments, R₁ is unsubstituted phenyl. In some embodiments, R₁ is phenyl substituted with —F.

In some embodiments, R₂ is C₁₋₁₂ alkyl optionally containing an O. In some embodiments, R₂ is OCH₃.

In some embodiments, the compound having the structure of Formula (IIB″) is selected from the group consisting of:

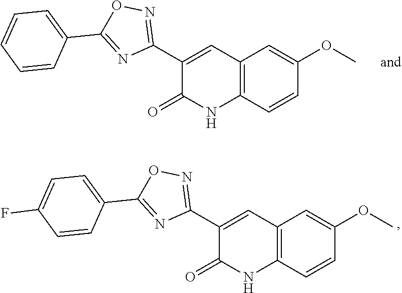 [SMILES: COc1ccc2[nH]c(=O)c(-c3noc(-c4ccccc4)n3)cc2c1 ; COc1ccc2[nH]c(=O)c(-c3noc(-c4ccc(F)cc4)n3)cc2c1]

or a pharmaceutically acceptable salt thereof.

In one embodiment, the compounds capable of inhibiting enzymatic activity of the NSP14-NSP10 complex have the structure of Formula (IIC):

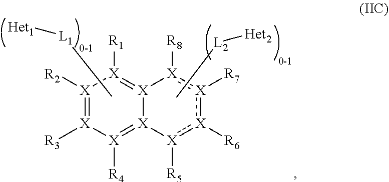

or a pharmaceutically acceptable salt thereof,
wherein X is independently at each occurrence selected from C, O, and N;
L₁ and L₂ are independently a linker selected from a bond and a C₁₋₃ alkyl optionally containing 1-2 heteroatoms selected from O, N, and S; Het₁ and Het₂ are independently a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In one particular embodiment, the compounds capable of inhibiting enzymatic activity of the NSP14-NSP10 complex having the structure of Formula (IIC) are:

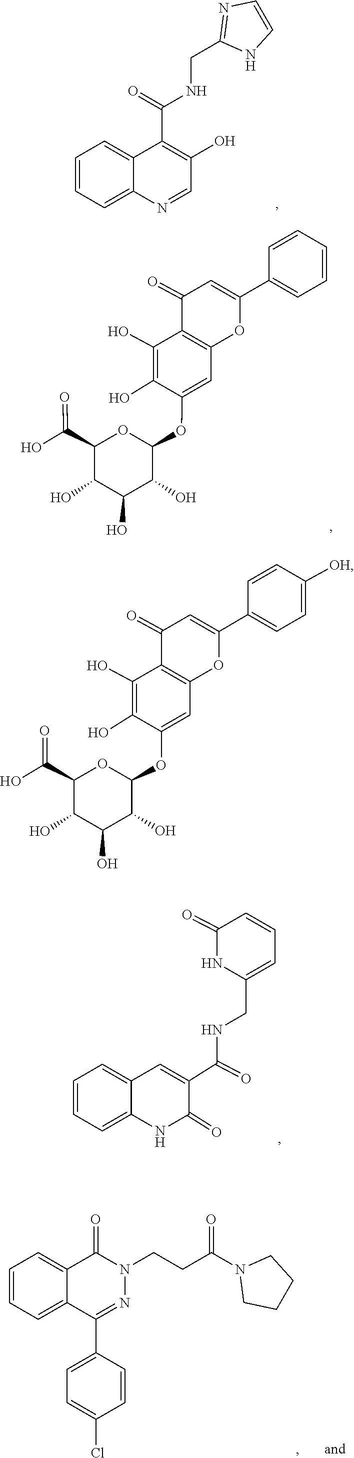 [SMILES: O=C(NCc1ncc[nH]1)c1c(O)cnc2ccccc12 ; O=C(O)[C@H]1O[C@@H](Oc2cc3oc(-c4ccccc4)cc(=O)c3c(O)c2O)[C@H](O)[C@@H](O)[C@@H]1O ; O=C(O)[C@H]1O[C@@H](Oc2cc3oc(-c4ccc(O)cc4)cc(=O)c3c(O)c2O)[C@H](O)[C@@H](O)[C@@H]1O ; O=C(NCc1cccc(=O)[nH]1)c1cc2ccccc2[nH]c1=O ; O=C(CCn1nc(-c2ccc(Cl)cc2)c2ccccc2c1=O)N1CCCC1]

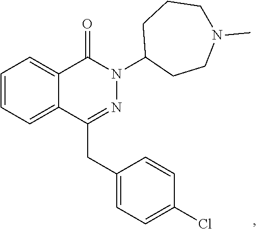 [SMILES: CN1CCCC(n2nc(Cc3ccc(Cl)cc3)c3ccccc3c2=O)CC1]

or a pharmaceutically acceptable salt thereof.

In another particular embodiment, the compounds capable of inhibiting enzymatic activity of the NSP14-NSP10 complex having the structure of Formula (IIC) are:

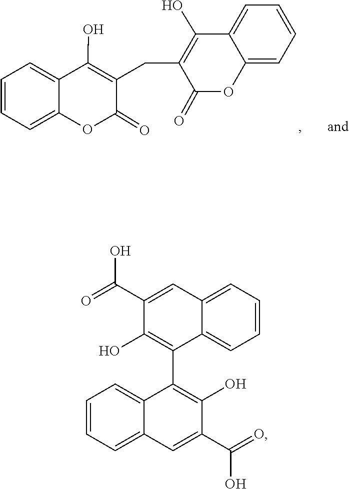 [SMILES: O=c1oc2ccccc2c(O)c1Cc1c(O)c2ccccc2oc1=O ; O=C(O)c1cc2ccccc2c(-c2c(O)c(C(=O)O)cc3ccccc23)c1O]

or a pharmaceutically acceptable salt thereof.

In one embodiment of compounds of Formula (II), adjacent two or more of R₁, R₂, R₃, R₄, R₅, R₆, R₇ and R₈ combine to form one or more fused rings, which may be further substituted with one or more substituents to form a fused polycyclic ring system. In some particular embodiments, the compounds of Formula (II) are:

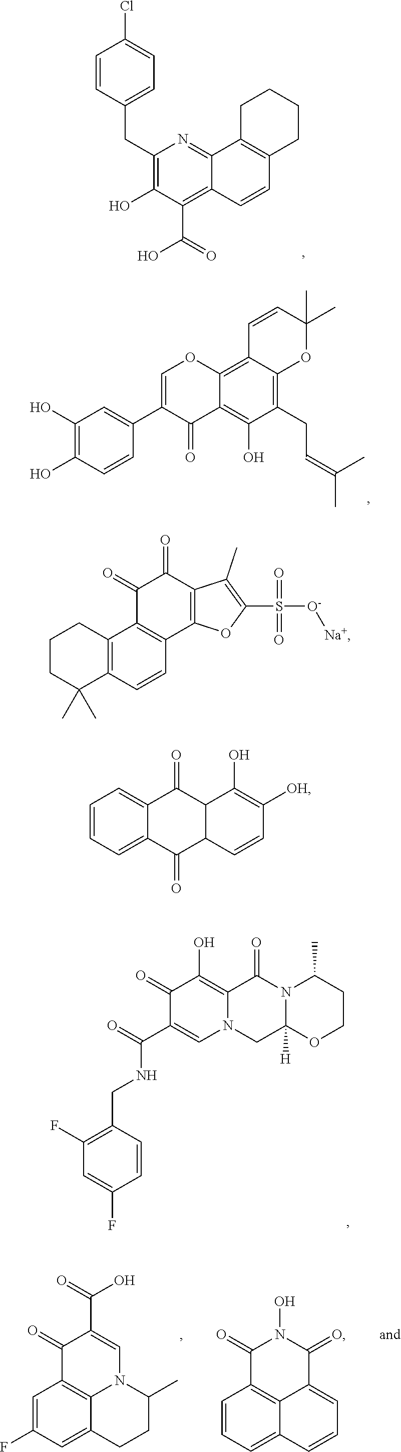

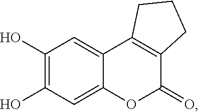 [SMILES: O=c1oc2cc(O)c(O)cc2c2c1CCC2]

or a pharmaceutically acceptable salt thereof.

In one embodiment, the compounds of Formula (II) have an anthracene

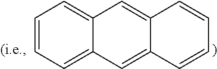 [SMILES: c1ccc2cc3ccccc3cc2c1]

core, wherein one or more carbons may be optionally replaced with a N, O, or S, and wherein one or more carbons may be optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring.

In another embodiment, the compounds of Formula (II) have a phenanthrene

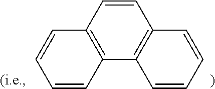 [SMILES: c1ccc2c(c1)ccc1ccccc12]

core, wherein one or more carbons may be optionally replaced with a N, O, or S, and wherein one or more carbons may be optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring.

In one embodiment, the compounds of Formula (II) are selected from the group consisting of PSI-697, Pomiferin, Tanshinone IIa sulfonate, Alizarin, Dolutegravir, Flumequine, and N-hydroxynaphthalimide.

In another aspect, the present disclosure provides compounds capable of inhibiting enzymatic activity of the NSP14-NSP10 complex having the structure of Formula (II′):

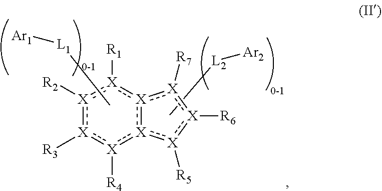

or a pharmaceutically acceptable salt thereof,
wherein X is independently at each occurrence selected from C, O, N, and S;
L₁ and L₂ are independently a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar₁ and Ar₂ are independently a phenyl, a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, and R₇ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

In one particular embodiment of Formula (II′), the inhibitory compounds may have the structure selected from:

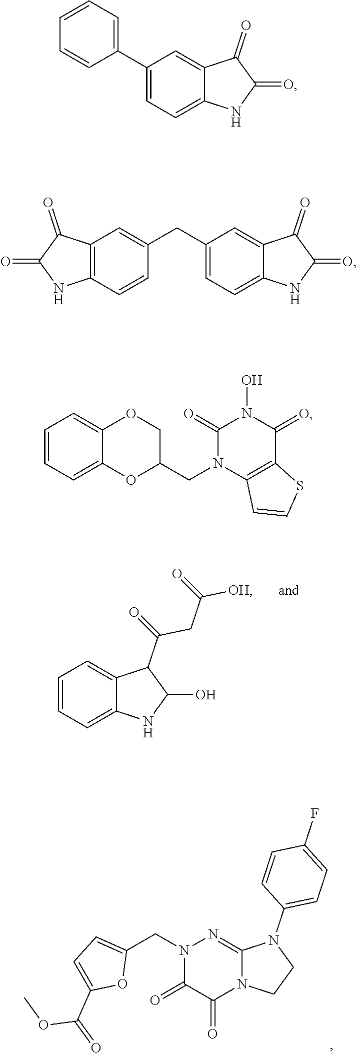 [SMILES: O=C1Nc2ccc(-c3ccccc3)cc2C1=O ; O=C1Nc2ccc(Cc3ccc4c(c3)C(=O)C(=O)N4)cc2C1=O ; O=c1c2sccc2n(CC2COc3ccccc3O2)c(=O)n1O ; O=C(O)CC(=O)C1c2ccccc2NC1O ; COC(=O)c1ccc(Cn2nc3n(c(=O)c2=O)CCN3c2ccc(F)cc2)o1]

or a pharmaceutically acceptable salt thereof.

In addition to the formulas above, other, different scaffolds are also capable of Exonuclease (ExoN) inhibition.

Without wishing to be bound by theory, it is postulated that one possible binding site of divergent scaffolds involves the compound tightly sandwiching between the RNA substrate and the protein surface (that we herein term the “under RNA pose”), or compounds that use their central, non-aromatic linker segment for metal ion coordination.

In some embodiments, (L₁-Ar₁) is absent. In some embodiments, the compound of Formula (II′) has the structure according to Formula (II′A):

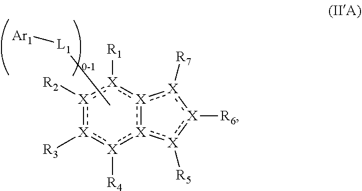

or a pharmaceutically acceptable salt thereof.

In some embodiments, the compound of Formula (II′) has the structure according to Formula (II′B):

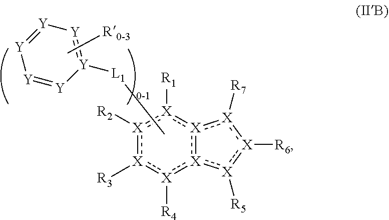

or a pharmaceutically acceptable salt thereof,
wherein Y is independently at each occurrence selected from a bond, C, O, N, and S.

In some embodiments, the compound of Formula (II′) has the structure according to Formula (II′C):

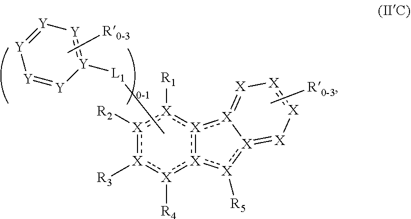

or a pharmaceutically acceptable salt thereof.

In some embodiments, the compound of Formula (II′C) has the structure:

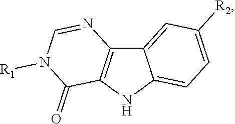

or a pharmaceutically acceptable salt thereof,
wherein R₁ and R₂ are independently H, C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof.

In some embodiments, the compound having the structure of Formula (II′C) is

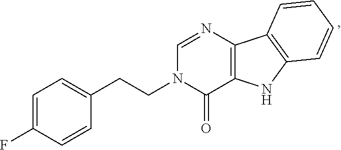 [SMILES: O=c1c2[nH]c3ccccc3c2ncn1CCc1ccc(F)cc1]

or a pharmaceutically acceptable salt thereof.

In some embodiments, (L₂-Ar₂) is absent. In some embodiments, the compound of Formula (II′) has the structure according to Formula (II′D):

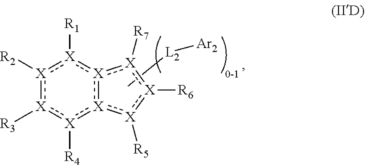

or a pharmaceutically acceptable salt thereof.

In some embodiments, the compound of Formula (II′) has the structure according to Formula (II′E):

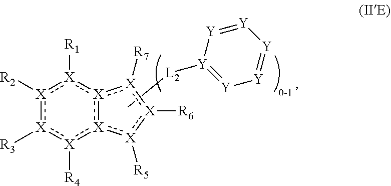

or a pharmaceutically acceptable salt thereof,
wherein Y is independently at each occurrence selected from a bond, C, O, N, and S.

In some embodiments, the adjacent two or more of R₁, R₂, R₃, R₄, R₅, R₆, R₇ and R₈ combine to form one or more fused rings, which may be further substituted with one or more substituents to form a fused polycyclic ring system.

In some embodiments, the compound of Formula (II′) is selected from the group consisting of:

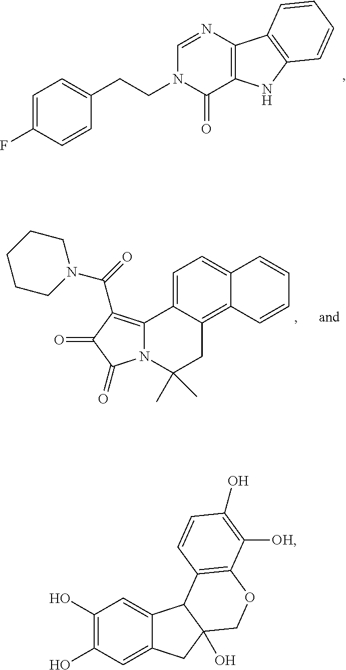 [SMILES: O=c1c2[nH]c3ccccc3c2ncn1CCc1ccc(F)cc1 ; CC1(C)Cc2c(ccc3ccccc23)C2=C(C(=O)N3CCCCC3)C(=O)C(=O)N21 ; Oc1cc2c(cc1O)C1c3ccc(O)c(O)c3OCC1(O)C2]

or a pharmaceutically acceptable salt thereof.

In one alternative aspect, the present disclosure provides compounds capable of inhibiting enzymatic activity of the NSP14-NSP10 complex having the structure of Formula (III):

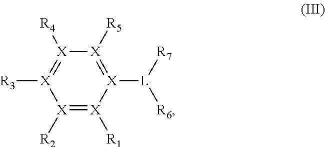

or a pharmaceutically acceptable salt thereof,
wherein X is independently at each occurrence selected from C, O, N, and S;
L is a linker selected from a bond, a C₁₋₁₂ alkyl, C₂₋₁₂ alkenyl, —CO—, —CO—NH—, —CO—(C₁₋₃ alkyl)-, and —CO—(C₂₋₄ alkenyl)-, and combinations thereof, wherein the C₁₋₁₂ alkyl or the C₂₋₁₂ alkenyl optionally contains 1-5 heteroatoms selected from O, N, and S;
R₁, R₂, R₃, R₄, and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring;
R₆ and R₇ are independently at each occurrence H, C₁₋₁₂ alkyl, C₁₋₁₂ alkenyl, C₆₋₁₂ aryl, C₁₋₁₂ aralkyl, C₁-C₁₂ heteroaryl, C₁₋₄ haloalkyl, —F, —Cl, —Br, —I, —OH, ═O, —CO₂H, —NO₂, —N—OH, —HSO₃, —H₂PO₃, —OR*, —(C═O)—R*, —CO₂R*, —CO—NHR*, —SO₂—NHR*, R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof. In another alternative aspect, the present disclosure provides compounds capable of inhibiting enzymatic activity of the NSP14-NSP10 complex having two connected rings connected with a linear or branched linker of length ranging from 1 to 6 atoms. The linker may contain double bonds as well as heteroatoms. The linker may also include aliphatic, aromatic, or heteroatom-containing rings. At least one of the rings is aromatic.

In one embodiment, the compounds capable of inhibiting enzymatic activity of the NSP14-NSP10 complex may have the structure of Formula (IIIA):

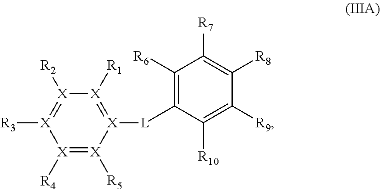

or a pharmaceutically acceptable salt thereof,
wherein X is independently at each occurrence selected from C, N, O, and S;
L is a linker selected from a bond, a C₁₋₁₂ alkyl, C₂₋₁₂ alkenyl, —CO—, —CO—NH—, —CO—(C₁₋₃ alkyl)-, and —CO—(C₂₋₄ alkenyl)-, and combinations thereof, wherein the C₁₋₁₂ alkyl or the C₂₋₁₂ alkenyl optionally contains 1-5 heteroatoms selected from O, N, and S;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least one of R₁, R₂, R₃, R₄ and R₅ is not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof. One can design compounds binding this way with either monocyclic or polycyclic rings.

In one embodiment, the compounds capable of inhibiting enzymatic activity of the NSP14-NSP10 complex may have the structure of Formula (IIIB):

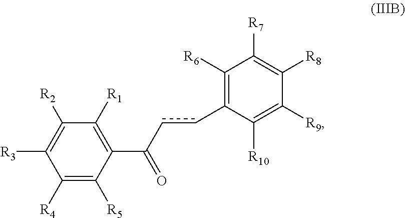

or a pharmaceutically acceptable salt thereof,
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least one of R₁, R₂, R₃, R₄ and R₅ is not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

The main ring should carry two types of substituent(s) for enhanced binding (not excluding other types of substituents):
(A) One or more substituent(s) capable of metal ion coordination (preferably Mg²⁺). Such groups include, but are not limited to: Hydroxyl (—OH), Oxo (═O), Carboxylic acid (—COOH), Nitro (—NO₂), N-hydroxyl (N—OH), Sulphonic acid (—HSO₃), Phosphonic acid (—H₂PO₃), Ether (—O—), Ester (—COO—), Amide (—CONH—), Sulphonamide (—SO₂—NH—), etc.
(B) One or more hydrophobic substituent(s), with either aliphatic, double bond containing or aromatic realizations (or a combination thereof). Such groups include, but are not limited to: Methyl-, Ethyl-, Phenyl-, Benzyl-, Isoprenyl-, etc.

The linker can also participate in metal ion binding in particular embodiments.

In specific embodiments, one of more of the rings can also be polycyclic, partly or entirely nonaromatic.

One can also design compounds binding in the same way that lack the main or the side ring.

Due to the similarity of rules for main site binding (I and II), and for alternatively binding compounds (III), the same molecule that has been designed to bind in a given orientation, might in reality associate with the enzyme in one or more other binding modes.

In some embodiments, the inhibitory compounds of Formula (IIIB) are:

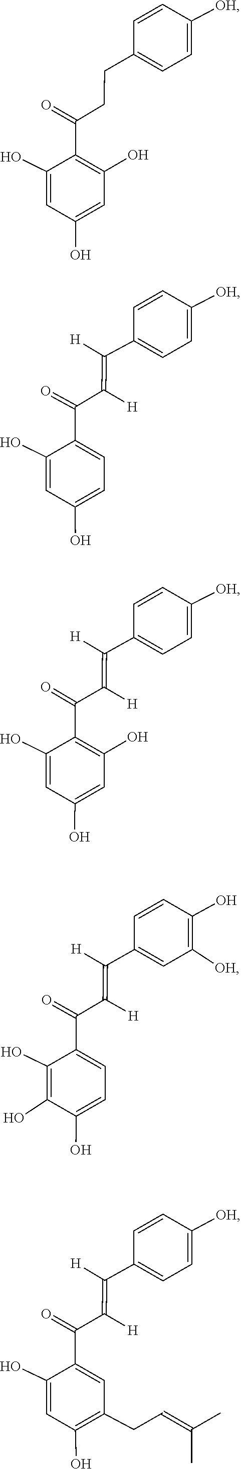 [SMILES: O=C(CCc1ccc(O)cc1)c1c(O)cc(O)cc1O ; O=C(/C=C/c1ccc(O)cc1)c1ccc(O)cc1O ; O=C(/C=C/c1ccc(O)cc1)c1c(O)cc(O)cc1O ; O=C(/C=C/c1ccc(O)c(O)c1)c1ccc(O)c(O)c1O ; CC(C)=CCc1cc(C(=O)/C=C/c2ccc(O)cc2)c(O)cc1O]

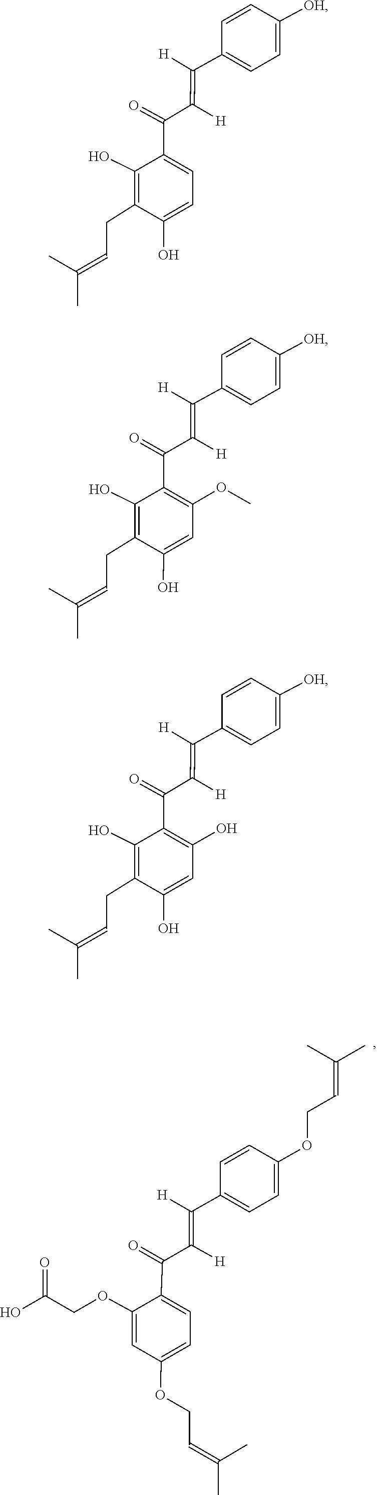 [SMILES: CC(C)=CCc1c(O)ccc(C(=O)/C=C/c2ccc(O)cc2)c1O ; COc1cc(O)c(CC=C(C)C)c(O)c1C(=O)/C=C/c1ccc(O)cc1 ; CC(C)=CCc1c(O)cc(O)c(C(=O)/C=C/c2ccc(O)cc2)c1O ; CC(C)=CCOc1ccc(/C=C/C(=O)c2ccc(OCC=C(C)C)cc2OCC(=O)O)cc1]

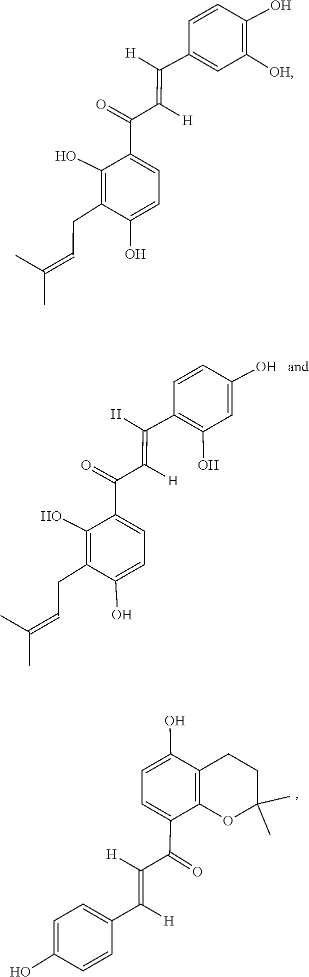

or a pharmaceutically acceptable salt thereof. In one particular embodiment, the inhibitory compounds may be naturally occurring chalcones, dihydrochalcones, prenyl-chalcones, and derivatives or chemical analogues thereof (both natural and synthetic). The main ring of these compounds may contain both metal ion binding groups (exemplified by —OH) and hydrophobic groups (exemplified by prenyl groups) for enhanced binding.

The linker between the main and the ancillary ring (of length n=3 in the above non-limiting examples) can either be aliphatic (as in dihydrochalcones, like phloretin) or contain double bonds (as in true chalcones, like isoliquiritigenin).

TABLE 2
Exemplary modulators of 3′ to 5′ exonucleases

| | | | Inhibitory | SARS- |
|---|---|---|---|---|
| # | | | activity | CoV2 |
| ID | Name | Compound Structures | present | IC₅₀ |
| 1 | 2,3,4- Trihydroxy- benzoic acid | 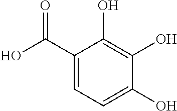 [SMILES: O=C(O)c1ccc(O)c(O)c1O] | − | |
| 2 | 2,4-Dioxo-4- phenylbutanoic acid | 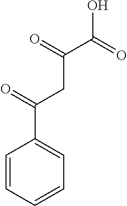 [SMILES: O=C(O)C(=O)CC(=O)c1ccccc1] | − | |
| 3 | Daphnetin | 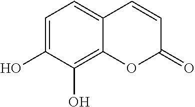 [SMILES: O=c1ccc2ccc(O)c(O)c2o1] | − | |
| 4 | Propyl gallate | 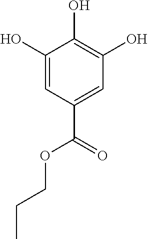 | − | |
| 5 | Elvitegravir | 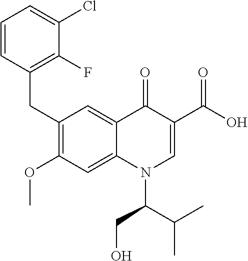 [SMILES: COc1cc2c(cc1Cc1cccc(Cl)c1F)c(=O)c(C(=O)O)cn2[C@H](CO)C(C)C] | − | |
| 6 | Raltegravir | 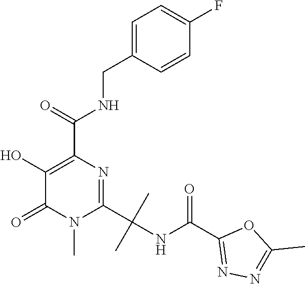 [SMILES: Cc1nnc(C(=O)NC(C)(C)c2nc(C(=O)NCc3ccc(F)cc3)c(O)c(=O)n2C)o1] | − | |
| 7 | Myricetin | 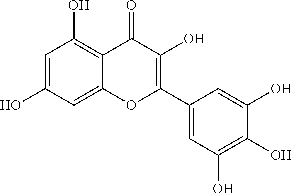 [SMILES: O=c1c(O)c(-c2cc(O)c(O)c(O)c2)oc2cc(O)cc(O)c12] | − | |
| 8 | Quercetin | 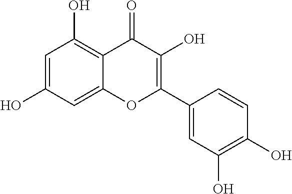 [SMILES: O=c1c(O)c(-c2ccc(O)c(O)c2)oc2cc(O)cc(O)c12] | − | |
| 9 | (-)- Epigallocatechin | 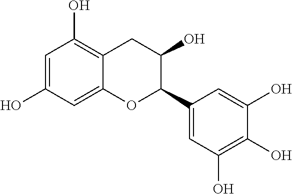 [SMILES: Oc1cc(O)c2c(c1)O[C@H](c1cc(O)c(O)c(O)c1)[C@H](O)C2] | − | |
| 10 | Benserazide hydrochloride | 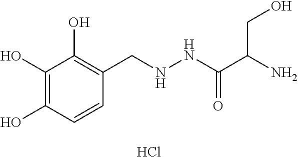 [SMILES: NC(CO)C(=O)NNCc1ccc(O)c(O)c1O ; Cl] | − | |
| 11 | Dihydromyricetin | 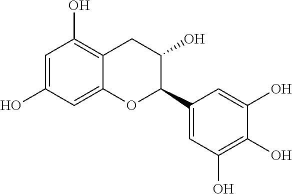 [SMILES: Oc1cc(O)c2c(c1)O[C@H](c1cc(O)c(O)c(O)c1)[C@@H](O)C2] | − | |
| 12 | Tolcapone | 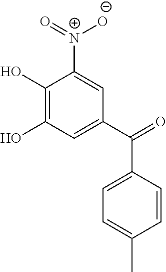 [SMILES: Cc1ccc(C(=O)c2cc(O)c(O)c([N+](=O)[O-])c2)cc1] | − | |
| 13 | 2- Hydroxy- isoquinoline- 1,3(2H,4H)- dione | 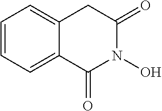 [SMILES: O=C1Cc2ccccc2C(=O)N1O] | +++ | ~400 uM |
| 14 | 5,6-Dihydroxy- 2- phenylpyrimidine- 4-carboxylic acid | 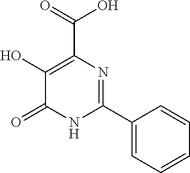 [SMILES: O=C(O)c1nc(-c2ccccc2)[nH]c(=O)c1O] | − | |
| 15 | 2-(2,3-dioxo- 1,2,3,4- tetrahydro- quinoxalin- 1-yl)acetic acid | 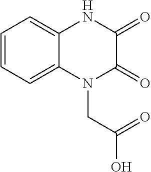 [SMILES: O=C(O)Cn1c(=O)c(=O)[nH]c2ccccc21] | − | |
| 16 | Dolutegravir | 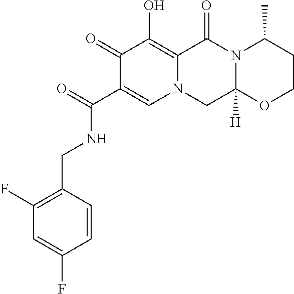 [SMILES: C[C@@H]1CCO[C@H]2Cn3cc(C(=O)NCc4ccc(F)cc4F)c(=O)c(O)c3C(=O)N12] | + | |
| 17 | 3- hydroxyquinoline- 4-carboxylic acid | 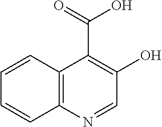 [SMILES: O=C(O)c1c(O)cnc2ccccc12] | − | |
| 18 | 2-oxo- 1,2,5,6,7,8- hexahydro- quinoline- 3-carboxylic acid | 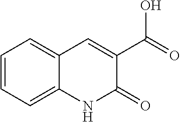 [SMILES: O=C(O)c1cc2ccccc2[nH]c1=O] | − | |
| 19 | N- Hydroxy- naphthalimide | 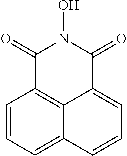 [SMILES: O=C1c2cccc3cccc(c23)C(=O)N1O] | − | |
| 20 | 1-((2,3- Dihydrobenzo[b] [1,4]dioxin-2- yl)methyl)-3- hydroxythieno [3,2-d]pyrimidine- 2,4(1H,3H)- dione (LNT1) | 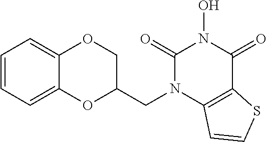 [SMILES: O=c1c2sccc2n(CC2COc3ccccc3O2)c(=O)n1O] | − | |
| 21 | 4- hydroxy- isoquinoline- 3- carboxylic acid | 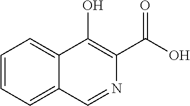 [SMILES: O=C(O)c1ncc2ccccc2c1O] | − | |
| 22 | 4,5,6-trimethyl- 2-oxo-1,2- dihydro-3- pyridinecarboxylic acid | 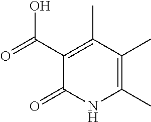 | − | |
| 23 | Dicoumarol | 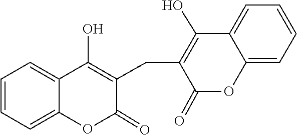 [SMILES: O=c1oc2ccccc2c(O)c1Cc1c(O)c2ccccc2oc1=O] | ++ | >500 uM |
| 24 | Flumequine | 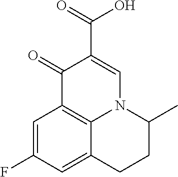 [SMILES: CC1CCc2cc(F)cc3c(=O)c(C(=O)O)cn1c23] | − | |
| 25 | Nalidixic acid (sodium salt) | 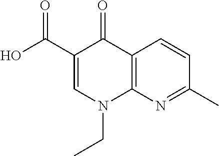 [SMILES: CCn1cc(C(=O)O)c(=O)c2ccc(C)nc21] | − | |
| 26 | 3-Hydroxy-3-(2- oxo-1,2- dihydro-indol-3- ylidene)- propionic acid | 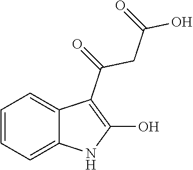 [SMILES: O=C(O)CC(=O)c1c(O)[nH]c2ccccc12] | − | |
| 27 | N-(5-Benzyl- 1,3,4-thiadiazol- 2-yl)benzamide | 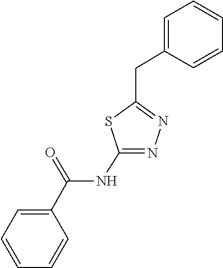 [SMILES: O=C(Nc1nnc(Cc2ccccc2)s1)c1ccccc1] | − | |
| 28 | 1-Benzyl-2-oxo- 1,2- dihydropyridine- 3-carboxylic acid | 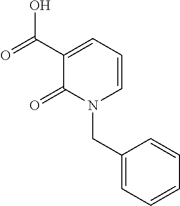 [SMILES: O=C(O)c1cccn(Cc2ccccc2)c1=O] | − | |
| 29 | 2,3-Cresotic acid, 5,5′- methylenedi- | 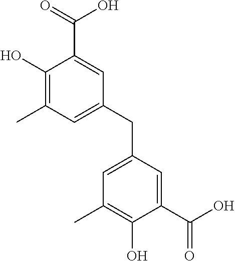 [SMILES: Cc1cc(Cc2cc(C)c(O)c(C(=O)O)c2)cc(C(=O)O)c1O] | + | |
| 30 | 5,5′- Methyl- enedisalicylic acid | 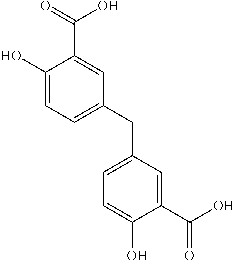 [SMILES: O=C(O)c1cc(Cc2ccc(O)c(C(=O)O)c2)ccc1O] | +++ | 200 uM |
| 31 | 3-Hydroxy-N- [(1H-imidazol-2- yl)methyl] quinoline-4- carboxamide | 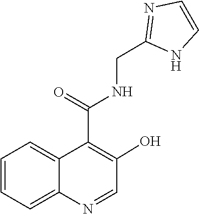 [SMILES: O=C(NCc1ncc[nH]1)c1c(O)cnc2ccccc12] | +++ | 210 uM |
| 32 | 4-Oxo-4H- pyrido[1,2- a]pyrimidine-3- carboxylic acid | 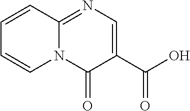 [SMILES: O=C(O)c1cnc2ccccn2c1=O] | − | |
| 33 | 3-hydroxy-2- phenyl-4- quinoline- carboxylic acid (Oxicinchophen) | 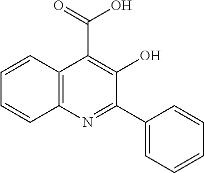 [SMILES: O=C(O)c1c(O)c(-c2ccccc2)nc2ccccc12] | +++ | |
| 34 | 1-(4- methoxybenzyl)- 4-oxo-1,4- dihydroquinoline- 3-carboxylic acid (BQCA) | 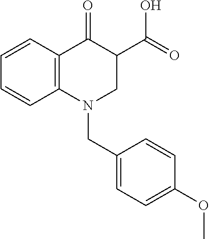 [SMILES: COc1ccc(CN2CC(C(=O)O)C(=O)c3ccccc32)cc1] | − | |
| 35 | N,1-Dibenzyl-2- oxo-1,2- dihydropyridine- 3-carboxamide | 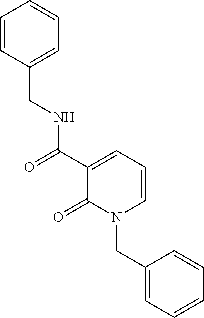 [SMILES: O=C(NCc1ccccc1)c1cccn(Cc2ccccc2)c1=O] | ++ | |
| 36 | Azelastine | 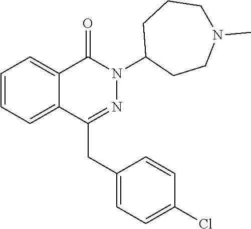 [SMILES: CN1CCCC(n2nc(Cc3ccc(Cl)cc3)c3ccccc3c2=O)CC1] | ++ | |
| 37 | 4-(4- Chlorophenyl)- 2-[3-oxo-3- (pyrrolidin-1- yl)propyl] phthalazin- 1(2H)-one | 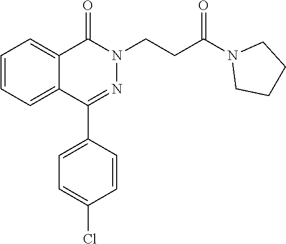 [SMILES: O=C(CCn1nc(-c2ccc(Cl)cc2)c2ccccc2c1=O)N1CCCC1] | − | |
| 38 | Phloretin | 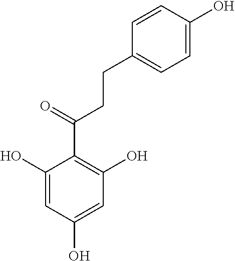 [SMILES: O=C(CCc1ccc(O)cc1)c1c(O)cc(O)cc1O] | +++ | 335 uM |
| 39 | 3,3′- Methyl- enedisalicylic acid | 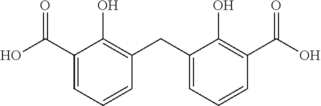 [SMILES: O=C(O)c1cccc(Cc2cccc(C(=O)O)c2O)c1O] | ++++ | 91 uM |
| 40 | 5-Benzyl-2- hydroxybenzoic acid | 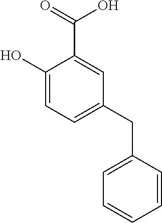 [SMILES: O=C(O)c1cc(Cc2ccccc2)ccc1O] | ++ | |
| 41 | 2-Hydroxy-5-(2- phenylethyl) benzoic acid | 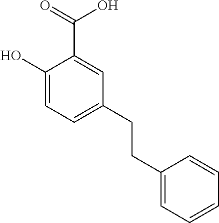 [SMILES: O=C(O)c1cc(CCc2ccccc2)ccc1O] | ++ | |
| 42 | N-[(3,4- Dihydroxyphenyl) methyl]-2-oxo- 1H-quinoline-3- carboxamide | 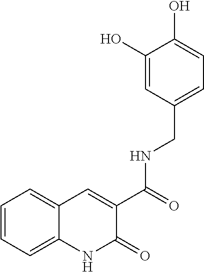 [SMILES: O=C(NCc1ccc(O)c(O)c1)c1cc2ccccc2[nH]c1=O] | +++ | |
| 43 | 5,5′- methylenebis (1H-indole-2,3- dione) | 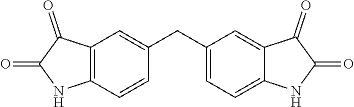 [SMILES: O=C1Nc2ccc(Cc3ccc4c(c3)C(=O)C(=O)N4)cc2C1=O] | ++ | |
| 44 | 5-Phenylisatin | 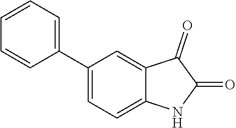 [SMILES: O=C1Nc2ccc(-c3ccccc3)cc2C1=O] | +++ | |
| 45 | 5-[(8- Hydroxyquinolin- 5- yl)methyl] quinolin- 8-ol | 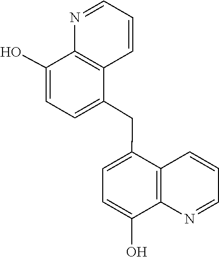 [SMILES: Oc1ccc(Cc2ccc(O)c3ncccc23)c2cccnc12] | − | |
| 46 | 5- [(Benzyloxy) methyl]quinolin-8- ol | 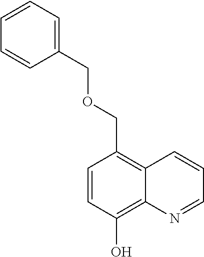 [SMILES: Oc1ccc(COCc2ccccc2)c2cccnc12] | − | |
| 47 | Alizarin | 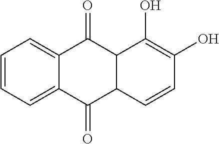 [SMILES: O=C1c2ccccc2C(=O)C2C(O)=C(O)C=CC12] | +++ | |
| 48 | Isoliquiritigenin | 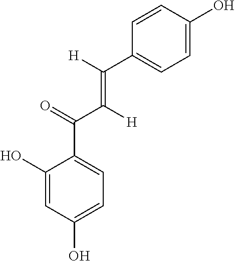 [SMILES: O=C(/C=C/c1ccc(O)cc1)c1ccc(O)cc1O] | +++ | |
| 49 | 2-Oxo-N-[(6- oxo-1H-pyridin- 2-yl)methyl]- 1H-quinoline-3- carboxamide | 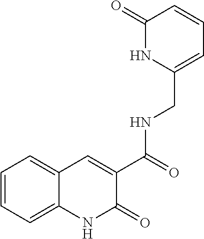 [SMILES: O=C(NCc1cccc(=O)[nH]1)c1cc2ccccc2[nH]c1=O] | − | |
| 50 | 2,8- Quinoline- dicarboxylic acid (#2- MB-IX-181) | 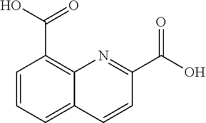 [SMILES: O=C(O)c1ccc2cccc(C(=O)O)c2n1] | + | |
| 51 | 6-Bromo-8- hydroxyquinoline- 3 carboxylic acid (#4-STL-1- 277) | 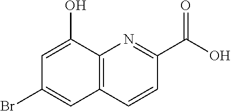 [SMILES: O=C(O)c1ccc2cc(Br)cc(O)c2n1] | + | |
| 52 | 4-Oxo-4H- quinolizine-3- carboxylic acid (#3-STL-1-279) | 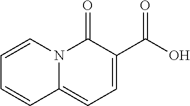 [SMILES: O=C(O)c1ccc2ccccn2c1=O] | + | |
| 53 | 2-Oxo-6-phenyl- 1H-quinoline-3- carboxylic acid | 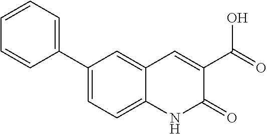 [SMILES: O=C(O)c1cc2cc(-c3ccccc3)ccc2[nH]c1=O] | +++ | 185 uM |
| 54 | Tetra- hydropapaveroline | 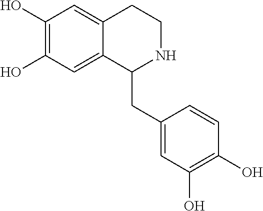 [SMILES: Oc1ccc(CC2NCCc3cc(O)c(O)cc32)cc1O] | ++++ | 10.06 uM |
| 55 | Baicalein | 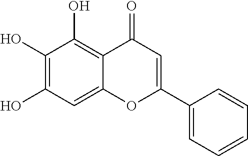 [SMILES: O=c1cc(-c2ccccc2)oc2cc(O)c(O)c(O)c12] | +++ | |
| 56 | Baicalin | 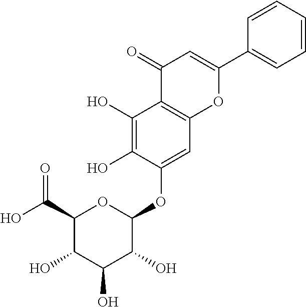 [SMILES: O=C(O)[C@H]1O[C@@H](Oc2cc3oc(-c4ccccc4)cc(=O)c3c(O)c2O)[C@H](O)[C@@H](O)[C@@H]1O] | − | |
| 57 | Bavachalcone | 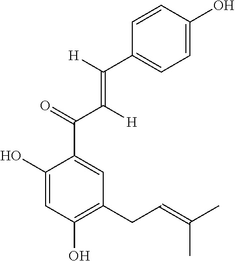 [SMILES: CC(C)=CCc1cc(C(=O)/C=C/c2ccc(O)cc2)c(O)cc1O] | ++++ | 21.62 uM |
| 58 | Xanthohumol | 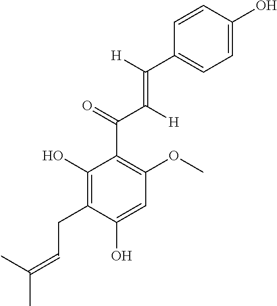 [SMILES: COc1cc(O)c(CC=C(C)C)c(O)c1C(=O)/C=C/c1ccc(O)cc1] | ++++ | 16 uM |
| 59 | PSI-697 | 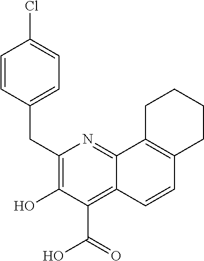 [SMILES: O=C(O)c1c(O)c(Cc2ccc(Cl)cc2)nc2c3c(ccc12)CCCC3] | ++++ | 36.04 uM |
| 60 | Tanshinone IIA sulfonate | 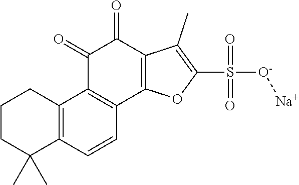 | +++++ | 2.20 ± 1.10 uM |
| 61 | (sodium) Scutellarin | 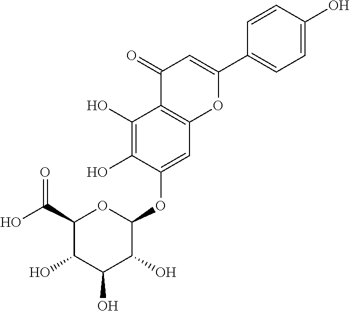 [SMILES: O=C(O)[C@H]1O[C@@H](Oc2cc3oc(-c4ccc(O)cc4)cc(=O)c3c(O)c2O)[C@H](O)[C@@H](O)[C@@H]1O] | − | |
| 62 | Scutellarein | 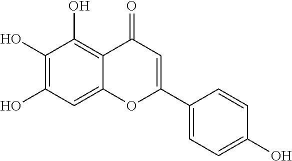 [SMILES: O=c1cc(-c2ccc(O)cc2)oc2cc(O)c(O)c(O)c12] | ++++ | 96.25 uM |
| 63 | 2,2′-Dihydroxy- 1,1′-binaphthyl- 3,3′-dicarboxylic acid | 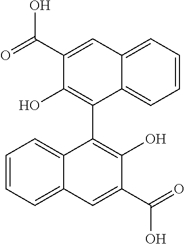 [SMILES: O=C(O)c1cc2ccccc2c(-c2c(O)c(C(=O)O)cc3ccccc23)c1O] | − | |
| 64 | 3-[(3-Carboxy- 2-hydroxy-5- methylphenyl)m ethyl]-2- hydroxy-5- methylbenzoic acid (Methyl- MDSA) | 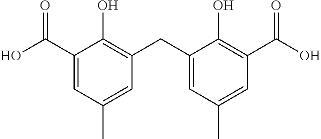 [SMILES: Cc1cc(Cc2cc(C)cc(C(=O)O)c2O)c(O)c(C(=O)O)c1] | − | |
| 65 | 3-[(3-Carboxy- 2-hydroxy-5- propan-2- ylphenyl)methyl]- 2-hydroxy-5- propan-2- ylbenzoic acid (Isopropyl- MDSA) | 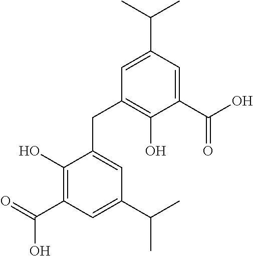 [SMILES: CC(C)c1cc(Cc2cc(C(C)C)cc(C(=O)O)c2O)c(O)c(C(=O)O)c1] | ++++ | 86.33 uM |
| 66 | Tizoxanide | 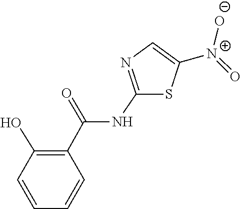 [SMILES: O=C(Nc1ncc([N+](=O)[O-])s1)c1ccccc1O] | + | |
| 67 | Nitazoxanide | 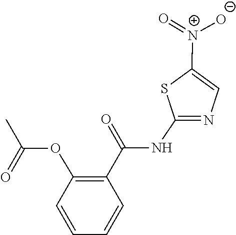 [SMILES: CC(=O)Oc1ccccc1C(=O)Nc1ncc([N+](=O)[O-])s1] | − | |
| 68 | Sofalcone | 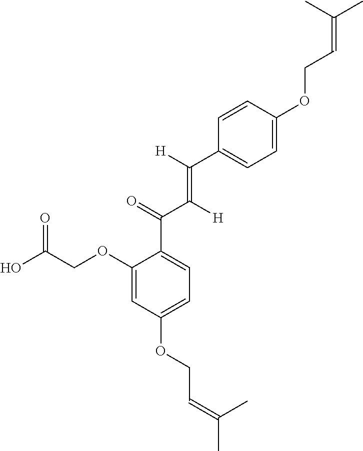 [SMILES: CC(C)=CCOc1ccc(/C=C/C(=O)c2ccc(OCC=C(C)C)cc2OCC(=O)O)cc1] | ++++ | 21.99 ± 6.02 uM |
| 69 | Pomiferin | 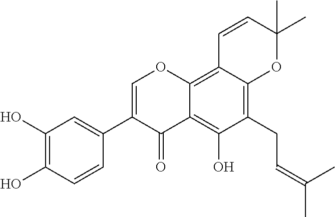 [SMILES: CC(C)=CCc1c2c(c3occ(-c4ccc(O)c(O)c4)c(=O)c3c1O)C=CC(C)(C)O2] | ++++ | 4.12 ± 3.25 uM |
| 70 | Medicagenic acid | 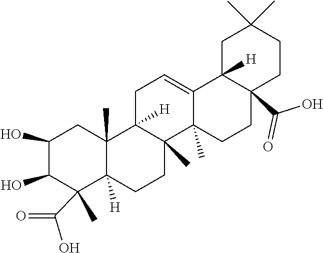 | − | |
| 71 | Desmethyl- xanthohumol | 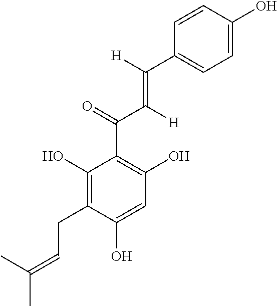 [SMILES: CC(C)=CCc1c(O)cc(O)c(C(=O)/C=C/c2ccc(O)cc2)c1O] | +++/ ++++ | 215.7 uM |
| 72 | Corylifol B | 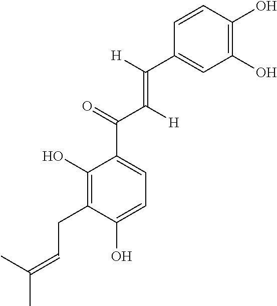 [SMILES: CC(C)=CCc1c(O)ccc(C(=O)/C=C/c2ccc(O)c(O)c2)c1O] | +++/ ++++ | 145.6 uM |
| 73 | Naringenin chalcone | 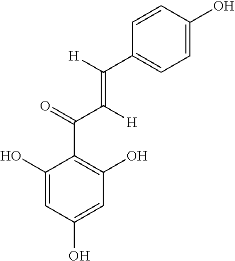 [SMILES: O=C(/C=C/c1ccc(O)cc1)c1c(O)cc(O)cc1O] | +++ | |
| 74 | Morachalcone A | 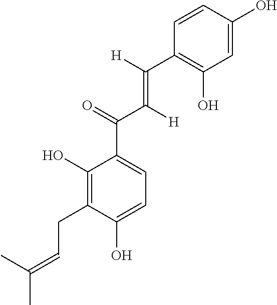 [SMILES: CC(C)=CCc1c(O)ccc(C(=O)/C=C/c2ccc(O)cc2O)c1O] | +++ | |
| 75 | Isodorsmanin A | 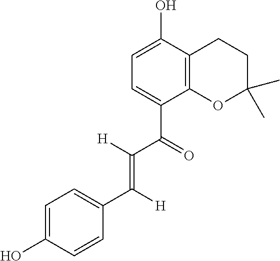 [SMILES: CC1(C)CCc2c(O)ccc(C(=O)/C=C/c3ccc(O)cc3)c2O1] | ++++ | 26.48 uM |
| 76 | Okanin | 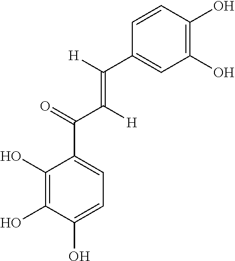 [SMILES: O=C(/C=C/c1ccc(O)c(O)c1)c1ccc(O)c(O)c1O] | ++ | |
| 77 | Isobavachalcone | 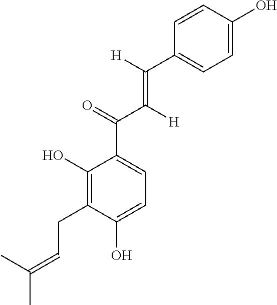 [SMILES: CC(C)=CCc1c(O)ccc(C(=O)/C=C/c2ccc(O)cc2)c1O] | ++++ | 17.43 ± 1.39 uM |
| 78 | 2-methyl-8- hydroxyquinoline- 7-carboxylic acid | 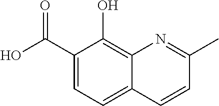 | − | |
| 79 | 2-(3,4- dihydroxystyryl)- 8- hydroxyquinoline- 7-carboxylic acid (KH161) | 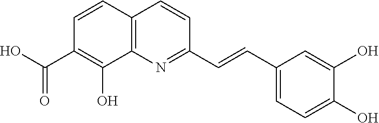 [SMILES: O=C(O)c1ccc2ccc(/C=C/c3ccc(O)c(O)c3)nc2c1O] | ++++ | 34 uM |
| 80 | 2-(3,4- dihydroxybenzyl carbamoyl)-8- hydroxyquinoline- 7-carboxylic acid (TOF452) | 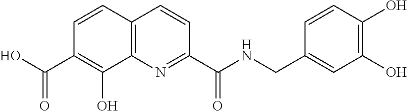 [SMILES: O=C(NCc1ccc(O)c(O)c1)c1ccc2ccc(C(=O)O)c(O)c2n1] | +++ | 551 uM |
| 81 | 2-(3,4- dihydroxybenzyl carbamoyl)- quinoline-8- carboxylic acid (MD20) | 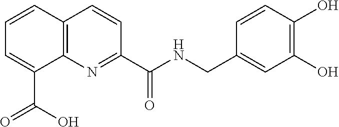 [SMILES: O=C(NCc1ccc(O)c(O)c1)c1ccc2cccc(C(=O)O)c2n1] | ++ | |
| 82 | 2-((3-methoxy- 4,5-dihydroxy) styryl)-8- hydroxyquinoline- 7-carboxylic acid (FZ41) | 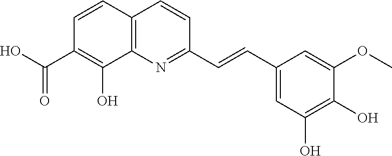 [SMILES: COc1cc(/C=C/c2ccc3ccc(C(=O)O)c(O)c3n2)cc(O)c1O] | ++++ | 164 uM |
| 83 | 2-(2,3- dihydroxybenzyl carbamoyl)-8- hydroxyquinoline- 7-carboxylic acid (TOF438) | 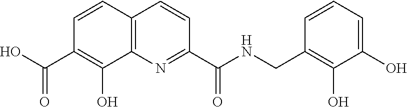 [SMILES: O=C(NCc1cccc(O)c1O)c1ccc2ccc(C(=O)O)c(O)c2n1] | ++++ | 110 uM |
| 84 | 2-(2,4- dihydroxybenzyl carbamoyl)-8- hydroxyquinoline- 7-carboxylic acid (TOF456) | 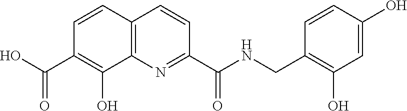 [SMILES: O=C(NCc1ccc(O)cc1O)c1ccc2ccc(C(=O)O)c(O)c2n1] | ++ | |
| 85 | 2-(3,5- dihydroxybenzyl carbamoyl)-8- hydroxyquinoline- 7-carboxylic acid (TOF540) | 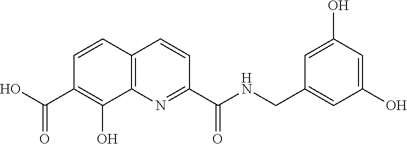 [SMILES: O=C(NCc1cc(O)cc(O)c1)c1ccc2ccc(C(=O)O)c(O)c2n1] | +++ | 232 uM |
| 86 | 2-(4- hydroxymethyl- styryl)- 8- hydroxyquinoline- 7-carboxylic acid (BIO23) | 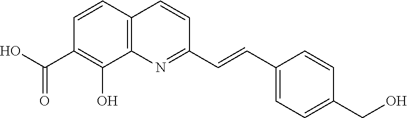 [SMILES: O=C(O)c1ccc2ccc(/C=C/c3ccc(CO)cc3)nc2c1O] | ++++ | |
| 87 | 2-(2,4- dihydroxystyryl)- 8- hydroxyquinoline- 7-carboxylic acid (KH278) | 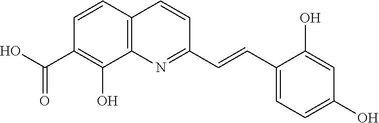 [SMILES: O=C(O)c1ccc2ccc(/C=C/c3ccc(O)cc3O)nc2c1O] | ++++ | |
| 88 | 2-(3,5-dibromo- 4-hydroxy styryl)-8- hydroxyquinoline- 7-carboxylic acid (FZ112) | 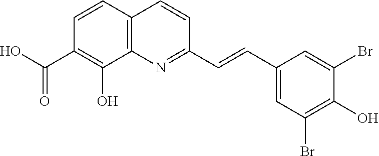 [SMILES: O=C(O)c1ccc2ccc(/C=C/c3cc(Br)c(O)c(Br)c3)nc2c1O] | ++++ | 26.68 uM |
| 89 | 2-(3,4- difluorostyryl)- 8- hydroxyquinoline- 7-carboxylic acid (BIO33) | 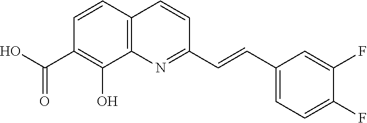 [SMILES: O=C(O)c1ccc2ccc(/C=C/c3ccc(F)c(F)c3)nc2c1O] | ++++ | 53.22 uM |
| 90 | 2-(3-carboxy-4- hydroxystyryl)- 8- hydroxyquinoline- 7-carboxylic acid (KHD304) | 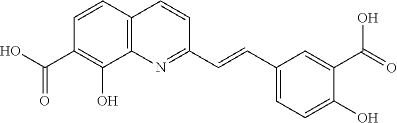 [SMILES: O=C(O)c1cc(/C=C/c2ccc3ccc(C(=O)O)c(O)c3n2)ccc1O] | ++++ | 24.6 uM |
| 91 | 2-styryl-8- hydroxyquinoline- 7-carboxylic acid (KHD302) | 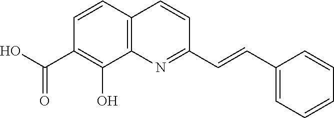 [SMILES: O=C(O)c1ccc2ccc(/C=C/c3ccccc3)nc2c1O] | ++++ | |
| 92 | 2-(3,4- dihydroxystyryl)- 8- hydroxyquinoline (KH153) | 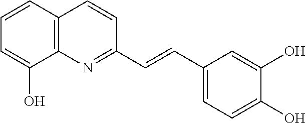 [SMILES: Oc1ccc(/C=C/c2ccc3cccc(O)c3n2)cc1O] | ++++ | 33.31 uM |
| 93 | 7-(3,4- difluorobenzoyl)- 2-(3,4- dihydroxyvinyl)- 8- hydroxyquinoline (MBN91) | 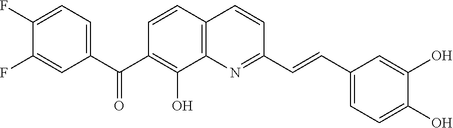 [SMILES: O=C(c1ccc(F)c(F)c1)c1ccc2ccc(/C=C/c3ccc(O)c(O)c3)nc2c1O] | +++++ | 7.65 uM |
| 94 | 2-(3,4- dihydroxy- phenethyl)- 8- hydroxyquinoline- 7-carboxylic acid (KHD342) | 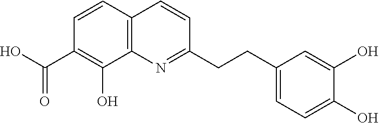 [SMILES: O=C(O)c1ccc2ccc(CCc3ccc(O)c(O)c3)nc2c1O] | ++++ | 67.12 uM |
| 95 | 2-(3-methoxy-4- hydroxystyryl)- 8- hydroxyquinoline- 7-carboxylic acid (FZ117) | 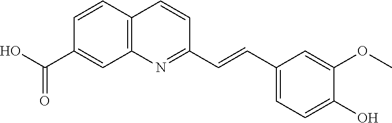 [SMILES: COc1cc(/C=C/c2ccc3ccc(C(=O)O)cc3n2)ccc1O] | +++ | |
| 96 | 7-benzoyl-2- (3,4- dihydroxyvinyl)- 8- hydroxyquinoline (MBN120) | 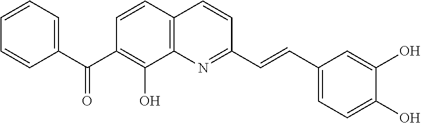 [SMILES: O=C(c1ccccc1)c1ccc2ccc(/C=C/c3ccc(O)c(O)c3)nc2c1O] | +++++ | 6.65 + 3.40 uM |
| 97 | 2-(2-(3- pyridinyl) ethenyl)-8- hydroxyquinoline- 7-carboxylic acid (FZ222) | 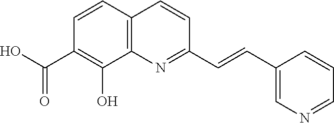 [SMILES: O=C(O)c1ccc2ccc(/C=C/c3cccnc3)nc2c1O] | ++ | |
| 98 | 2-(4- (acetylamino) styryl)-8- hydroxyquinoline- 7-carboxylic acid (FZ43) | 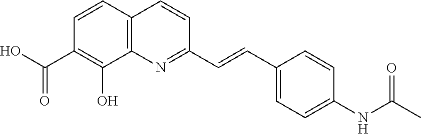 [SMILES: CC(=O)Nc1ccc(/C=C/c2ccc3ccc(C(=O)O)c(O)c3n2)cc1] | +++ | |
| 99 | 2-(3,4- dihydroxystyryl)- 5- hydroxyquinoline- 6-carboxylic acid (BIO50) | 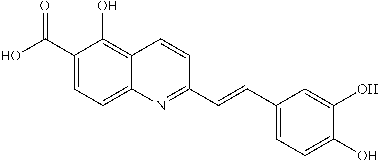 [SMILES: O=C(O)c1ccc2nc(/C=C/c3ccc(O)c(O)c3)ccc2c1O] | ++++ | 30.66 uM |
| 100 | 2-(3,4- dihydroxy- phenethyl)-8- hydroxyquinoline- 7-phosphonic acid (MBN68) | 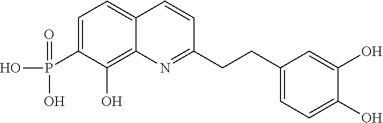 [SMILES: O=P(O)(O)c1ccc2ccc(CCc3ccc(O)c(O)c3)nc2c1O] | ++++ | 28.6 uM |
| 101 | [2-[2-(3,4- Dihydroxyphenyl) vinyl]-8- hydroxyquinolin- 7-yl]-(4- nitrophenyl) methanone (MBN87) | 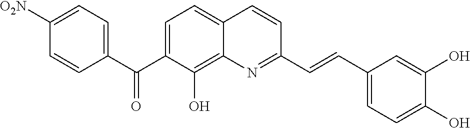 [SMILES: O=C(c1ccc([N+](=O)[O-])cc1)c1ccc2ccc(/C=C/c3ccc(O)c(O)c3)nc2c1O] | +++++ | 5.2 uM |
| 102 | [2-[2-(3,4- Dihydroxyphenyl) vinyl]-8- hydroxyquinolin- 7-yl]-pyridin-2- yl-methanone (MBN131) | 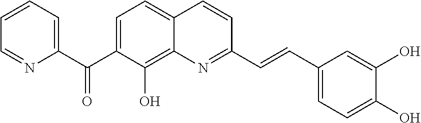 [SMILES: O=C(c1ccccn1)c1ccc2ccc(/C=C/c3ccc(O)c(O)c3)nc2c1O] | +++++ | 8.4 uM |
| 103 | [2-[2-(3,4- Dihydroxyphenyl) vinyl]-8- hydroxyquinolin- 7-yl]-pyridin-4- yl-methanone (MBN132) | 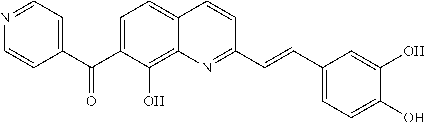 [SMILES: O=C(c1ccncc1)c1ccc2ccc(/C=C/c3ccc(O)c(O)c3)nc2c1O] | ++++ | 57.17 uM |
| 104 | Methyl 5-((8-(4- fluorophenyl)- 3,4-dioxo- 3,4,7,8- tetrahydroimidazo [2,1-c] [1,2,4]triazin- 2(6H)- yl)methyl)furan- 2-carboxylate | [symbol] | + | ~1455 uM |
| 105 | 3-[2-(4- Fluorophenyl) ethyl]-3,5- dihydro-4H- pyrimido [5,4- b]indol-4-one | [symbol] | +++ | 217 uM |
| 106 | 11,11-Dimethyl- 3-(morpholin-4- ylcarbonyl)- 10,11- dihydronaphtho [1,2-g]indolizine- 1,2-dione | [symbol] | + | 1074 uM |
| 107 | 7,8-Dihydroxy- 2,3- dihydrocyclopenta [c]chromen- 4(1H)-one | 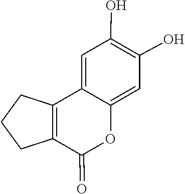 [SMILES: O=c1oc2cc(O)c(O)cc2c2c1CCC2] | + | |
| 108 | 7,11b- Dihydroindeno [2,1-c]chromene- 3,4,6a,9,10(6H)- pentol (Hematoxylin) | 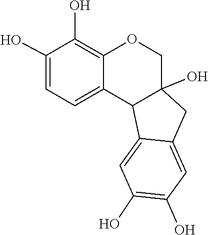 [SMILES: Oc1cc2c(cc1O)C1c3ccc(O)c(O)c3OCC1(O)C2] | +++ | 400 uM |
| 109 | 3-(5-(4- Fluorophenyl)- 1,2,4-oxadiazol- 3-yl)-6- methoxyquinolin- 2-ol | 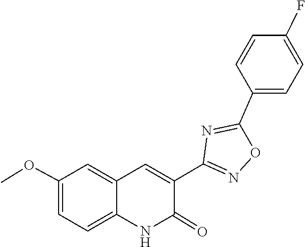 [SMILES: COc1ccc2[nH]c(=O)c(-c3noc(-c4ccc(F)cc4)n3)cc2c1] | ++++ | 79.86 uM |
| 110 | 6-Methoxy-3-(5- phenyl-1,2,4- oxadiazol-3- yl)quinolin-2-ol | 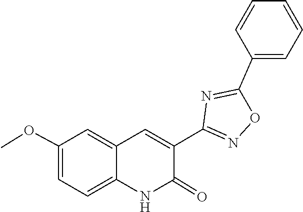 [SMILES: COc1ccc2[nH]c(=O)c(-c3noc(-c4ccccc4)n3)cc2c1] | ++++ | 113.6 uM |
| 111 | 7-fluoro-N-(4- fluorobenzyl)- 2-hydroxy-1,3- dioxo-4H- isoquinoline-4- carboxamide (VS56) | 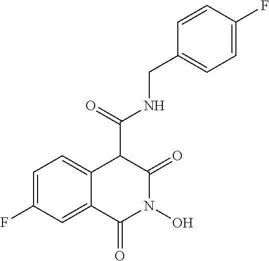 [SMILES: O=C(NCc1ccc(F)cc1)C1C(=O)N(O)C(=O)c2cc(F)ccc21] | ++++ | 41.4 uM |
| 112 | 7- trifluoromethyl- N-(4- fluorobenzyl)-2- hydroxy-1,3- dioxo-4H- isoquinoline-4- carboxamide (VS59) | 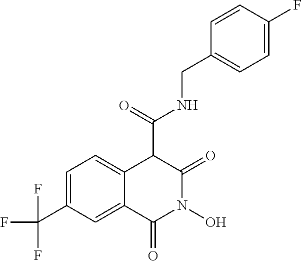 [SMILES: O=C(NCc1ccc(F)cc1)C1C(=O)N(O)C(=O)c2cc(C(F)(F)F)ccc21] | ++++ | 19.43 ± 8.37uM |
| 113 | 7-bromo-N-(4- fluorobenzyl)- 2-hydroxy-1,3- dioxo-4H- isoquinoline-4- carboxamide (VS54) | 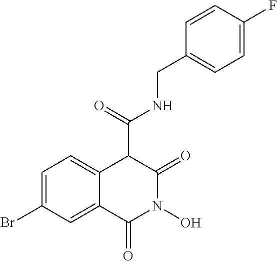 [SMILES: O=C(NCc1ccc(F)cc1)C1C(=O)N(O)C(=O)c2cc(Br)ccc21] | ++++ | 39.75 uM |
| 114 | N-(tert-butyl)-2- hydroxy-1,3- dioxo-4H- isoquinoline-4- carboxamide (AV01) | 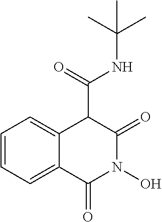 [SMILES: CC(C)(C)NC(=O)C1C(=O)N(O)C(=O)c2ccccc21] | ++ | |
| 115 | N-cyclohexyl-2- hydroxy-1,3- dioxo-4H- isoquinoline-4- carboxamide (MB200) | 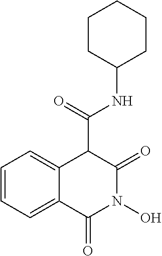 [SMILES: O=C(NC1CCCCC1)C1C(=O)N(O)C(=O)c2ccccc21] | +++ | 146 uM |
| 116 | 4-pentyl-2- hydroxy-1,3- dioxo-4H- isoquinoline (MB09) | 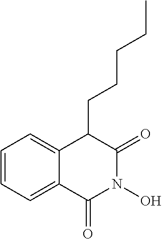 [SMILES: CCCCCC1C(=O)N(O)C(=O)c2ccccc21] | + | |
| 117 | 4-benzyl-2- hydroxy-1,3- dioxo-4H- isoquinoline (MB05) | 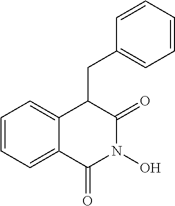 [SMILES: O=C1c2ccccc2C(Cc2ccccc2)C(=O)N1O] | +++ | 160 uM |
| 118 | N-phenyl-2- hydroxy-1,3- dioxo-4H- isoquinoline-4- carboxamide (MB105) | 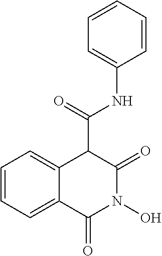 [SMILES: O=C(Nc1ccccc1)C1C(=O)N(O)C(=O)c2ccccc21] | ++++ | 40.4 uM |
| 119 | [2-[2-(3,4- Dihydroxyphenyl) vinyl]-8- hydroxyquinolin- 7-yl]-pyridin-3- yl-methanone (MD96) | 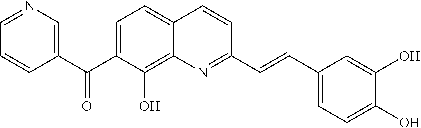 [SMILES: O=C(c1cccnc1)c1ccc2ccc(/C=C/c3ccc(O)c(O)c3)nc2c1O] | +++++ | 7.04 uM |

| | SARS- | MERS- | | |
|---|---|---|---|---|
| # | CoV | CoV | Solubility | Toxicity |
| ID | IC₅₀ | IC₅₀ | issue | at IC₅₀ |
| 1 | | | | |
| 2 | | | | |
| 3 | | | | |
| 4 | | | | |
| 5 | | | | |
| 6 | | | | |
| 7 | | | | |
| 8 | | | | |
| 9 | | | | |
| 10 | | | | |
| 11 | | | | |
| 12 | | | | |
| 13 | | | No | Yes |
| 14 | | | | |
| 15 | | | | |
| 16 | | | | |
| 17 | | | | |
| 18 | | | | |
| 19 | | | | |
| 20 | | | | |
| 21 | | | | |
| 22 | | | | |
| 23 | | | | |
| 24 | | | | |
| 25 | | | | |
| 26 | | | | |
| 27 | | | Some | |
| 28 | | | | |
| 29 | | | | |
| 30 | | | No | No |
| 31 | | | No | No |
| 32 | | | | |
| 33 | | | | |
| 34 | | | | |
| 35 | | | | |
| 36 | | | | |
| 37 | | | | |
| 38 | | | | |
| 39 | | | | |
| 40 | | | | |
| 41 | | | | |
| 42 | | | | |
| 43 | | | | |
| 44 | | | | |
| 45 | | | | |
| 46 | | | | |
| 47 | | | | |
| 48 | | | | |
| 49 | | | | |
| 50 | | | | |
| 51 | | | | |
| 52 | | | | |
| 53 | | | some | no |
| 54 | 1.22 uM | 10.23 uM | no | no |
| 55 | | | | |
| 56 | | | | |
| 57 | | | some | yes |
| 58 | | | no | yes |
| 59 | | | no | yes |
| 60 | 1.29 uM | 2.92 uM | no | no |
| 61 | | | | |
| 62 | | | no | no |
| 63 | | | | |
| 64 | | | | |
| 65 | | | no | no |
| 66 | | | | |
| 67 | | | | |
| 68 | 10.08 uM | 53.26 uM | No | Some |
| 69 | 0.25 uM | 1.29 uM | Some | Yes |
| 70 | | | | |
| 71 | | | No | Yes |
| 72 | | | | |
| 73 | | | No | Yes |
| 74 | | | No | Yes |
| 75 | | | No | Yes |
| 76 | | | No | Yes |
| 77 | 14.95 uM | 21.78 uM | no | some |
| 78 | | | No | No |
| 79 | | | No | No |
| 80 | | | | |
| 81 | | | | |
| 82 | | | | |
| 83 | | | | |
| 84 | | | | |
| 85 | | | | |
| 86 | | | | |
| 87 | | | | |
| 88 | | | Yes | No |
| 89 | | | Yes | Yes |
| 90 | | | No | No |
| 91 | | | | |
| 92 | | | Yes | Yes |
| 93 | | | Yes | Yes |
| 94 | | | No | No |
| 95 | | | Some | No |
| 96 | 0.71 uM | 3.95 uM | | |
| 97 | | | | |
| 98 | | | | |
| 99 | | | No | Yes |
| 100 | | | Some | No |
| 101 | | | No | |
| 102 | | | No | |
| 103 | | | No | |
| 104 | | | No | |
| 105 | | | No | |
| 106 | | | No | |
| 107 | | | No | |
| 108 | | | No | |
| 109 | | | No | |
| 110 | | | No | |
| 111 | | | No | |
| 112 | 4.22 uM | 4.39 uM | No | |
| 113 | | | No | |
| 114 | | | No | |
| 115 | | | No | |
| 116 | | | No | |
| 117 | | | No | |
| 118 | | | No | |
| 119 | | | No | |

[symbol]

indicates data missing or illegible when filed

or a pharmaceutically acceptable salt thereof.

In another aspect, the present disclosure provides a method of treating a viral infection in a subject comprising administering to the subject a compound capable of inhibiting enzymatic activity of the NSP14-NSP10 complex having the structure of any of the Formulas above, e.g., Formula (I) or Formula (II), or a pharmaceutically acceptable salt thereof.

In one embodiment, the viral infection is a coronaviral infection, i.e., the virus is a Coronavirus.

In one embodiment, the viral infection is a SARS-CoV2, a SARS-CoV, a MERS-CoV, a HCoV-229E, a HCoV-NL63, a HCoV-HKU1 or a HCoV-OC43 infection, i.e., the virus is a SARS-CoV2, a SARS-CoV, a MERS-CoV, a HCoV-229E, a HCoV-NL63, a HCoV-HKU1 or a HCoV-OC43 virus, or a variant thereof.

In one embodiment, the viral infection is a SARS-CoV2 infection, i.e., the virus is a SARS-CoV2 virus, or a variant thereof.

In one embodiment, the viral infection is a SARS-CoV infection, i.e., the virus is a SARS-CoV virus, or a variant thereof.

In one embodiment, the viral infection is a MERS-CoV infection, i.e., the virus is a MERS-CoV virus, or a variant thereof.

In one embodiment, the present disclosure provides a method of treating a SARS-CoV2 infection in a subject comprising administering to the subject a compound capable of inhibiting enzymatic activity of the NSP14-NSP10 complex having the structure of any of the Formulas above, e.g., Formula (I) or Formula (II), or a pharmaceutically acceptable salt thereof.

In various embodiments, the methods of treating a viral infection (e.g., SARS-CoV2) described herein further comprises administering a ribonucleotide analog to the subject. In some embodiments, the ribonucleotide analog is selected from Remdesivir, Ribavirin, Favipiravir, N₄-Hydroxycytidine (EIDD-1931) or its derivative Molnupiravir, 5-Fluorouracil, and Sofosbuvir. In some embodiments, the ribonucleotide analog may be an active metabolite of the ribonucleotide analog, such as GS-441524-MP (Remdesivir active metabolite).

Pertinent to present day drug discovery efforts, the ExoN domain of the NSP14/NSP10 exonuclease complex has rendered available antiviral compounds ineffective in the treatment of CoV, as many such compounds, e.g., ribonucleoside/ribonucleotide analogs including Remdesivir and wide-spectrum Ribavarin, are unable to effectively evade its catalytic proofreading activity (Ferron et al., 2018; Shannon et al., 2020). It is comtemplated that ribonucleotide analog efficacy may be enhanced with mutant NSP14, and ExoN inhibitors could boost efficacy of ribonucleotide analogs (e.g., Remdesivir, Ribavirin, Favipiravir, N₄-Hydroxycytidine (EIDD-1931) or its derivative Molnupiravir, 5-Fluorouracil, and Sofosbuvir) when used in combination.

In some embodiments, the compound capable of inhibiting enzymatic activity of the NSP14-NSP10 complex and the ribonucleotide analog are administered concurrently. In some embodiments, the compound capable of inhibiting enzymatic activity of the NSP14-NSP10 complex is administered before the ribonucleotide analog. In some embodiments, the compound capable of inhibiting enzymatic activity of the NSP14-NSP10 complex is administered after the ribonucleotide analog.

### Kits

In another aspect is provided a kit comprising one or more fluorescently labeled double-stranded RNA (dsRNA) molecules selected from:

```
5′ FAM-UUGCCGAAUUAAGCGCA-3′ (SEQ ID NO: 1)
                  ||||||||||||||
          3′-CGGCUUAAUUCGCGAAU-BHQ1-5′ (SEQ ID NO: 2)
  5′ FAM-UUGCCGAAUUAAGCGCCA (SEQ ID NO: 3)
                |||||||||||||||||
3′ BHQ1-AACGGCUUAAUUCGCGGAAU (SEQ ID NO: 4)
  5′ FAM-UUUUUUCGGCCCA (SEQ ID NO: 5)
                ||||||||||||
3′ BHQ1-AAAAAAGCCGGGAUAAA (SEQ ID NO: 6)
  5′ FAM-UCUUUUCGGCCCA (SEQ ID NO: 7)
                ||||||||||||
3′ BHQ1-AGAAAAGCCGGGAUAAA (SEQ ID NO: 8)
5′ TxRed-UCUUUUCGGCCCA (SEQ ID NO: 9)
                  ||||||||||||
  3′ BHQ2-AGAAAAGCCGGGAUAAA (SEQ ID NO: 10)
  5′ Cy3-UCUUUUCGGCCCA (SEQ ID NO: 11)
                ||||||||||||
3′ BHQ2-AGAAAAGCCGGGAUAAA (SEQ ID NO: 10)
3′ TAMRA-UCUUUUCGGCCCA (SEQ ID NO: 12)
                  ||||||||||||
  3′ BHQ2-AGAAAAGCCGGGAUAAA (SEQ ID NO: 10)
  5′ Cy5-UCUUUUCGGCCCA (SEQ ID NO: 13)
                ||||||||||||
3′ BHQ3-AGAAAAGCCGGGAUAAA (SEQ ID NO: 14).
```

and optionally instructions for use.

In some embodiments, the kit further may comprise a control nuclease selected from RNase A, RNase L, PNPase, RNase II, RNase R, Ribonuclease T1, Nuclease BAL-31, and RNase III. In some embodiments, the kit further comprises a 3′ to 5′ exonuclease. In some embodiments, the 3′ to 5′ exonuclease may comprise a NSP14 exonuclease or a NSP14/NSP10 exonuclease complex from SARS-CoV2 virus. In some embodiments, the kit may further comprise a reaction buffer comprising 50 mM TRIS-HCl, pH=7.5, 2 mM MgCl₂, 2 mM DTT.

### EXAMPLES

The following examples are provided to further describe some of the embodiments disclosed herein. The examples are intended to illustrate, not to limit, the disclosed embodiments.

### Example 1. Expression and Purification of His-Tagged NSP14 and NSP10 of SARS-CoV, SARS-CoV2 and MERS-CoV Proteins

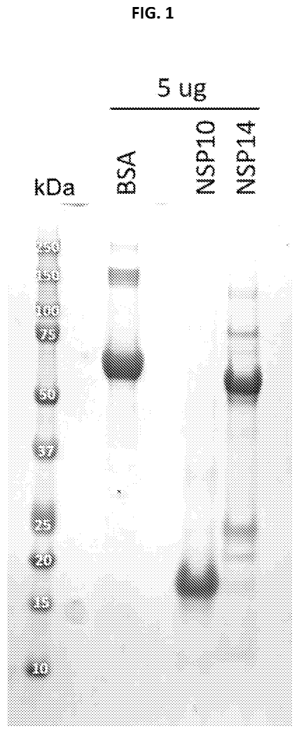

E. coli codon-optimized NSP14 and NSP10 of SARS-CoV2 cDNA sequences were individually engineered into pET30a+ plasmid vectors (Ndel-HindIII restriction sites) to produce recombinant proteins carrying a single C-terminal His-tag. Expression in E. coli and purification of recombinant His-tagged proteins was performed. Purified recombinant NSP10-His tagged protein (3.8 mg/ml, 236 M) and NSP14-His tagged protein (0.49 mg/ml, 8 M) were resolved via sodium dodecyl sulphate-polyacrylamide gel electrophoresis (5 g/well; 50 mM Tris-HCl, 150 mM NaCl, 10% Glycerol, 2 mM DTT; pH 8.0). Bands for NSP10-His tagged protein (16.3 kDa) and NSP14-His tagged proteins (61.6 kDa) were detected and visualized using coomassie brilliant blue R-250 are shown in FIG. 1.

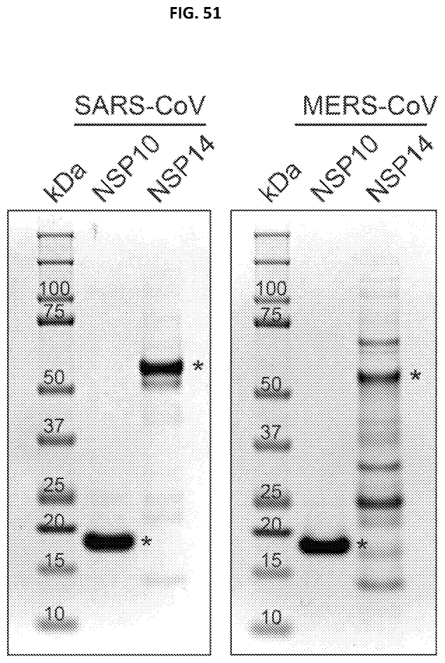

E. coli codon-optimized NSP14 and NSP10 of SARS-CoV and MERS-CoV cDNA sequences were individually engineered into pET30a+ plasmid vectors (Ndel-HindIII restriction sites) to produce recombinant proteins carrying a single C-terminal His-tag. Expression in E. coli and purification of recombinant His-tagged proteins was performed. Purified recombinant NSP10-His tagged protein and NSP14-His tagged protein were resolved via sodium dodecyl sulphate-polyacrylamide gel electrophoresis (5 g/well; 50 mM Tris-HCl, 150 mM NaCl, 10% Glycerol, 2 mM DTT; pH 8.0). Bands for NSP10-His tagged protein (˜16 kDa) and NSP14-His tagged proteins (˜60 kDa) were detected and visualized using coomassie brilliant blue R-250 are shown in FIG. 51.

Description and SEQ ID NOs of exemplary nucleotide and amino acid sequences for NSP14 and NSP10 of SARS-CoV2, SARS-CoV and MERS-CoV are provided above in Table 1 (see above).

### Example 2. Substrate Designs for FRET-Based Testing of SARS-CoV2 NSP14/10 Exonuclease Activity

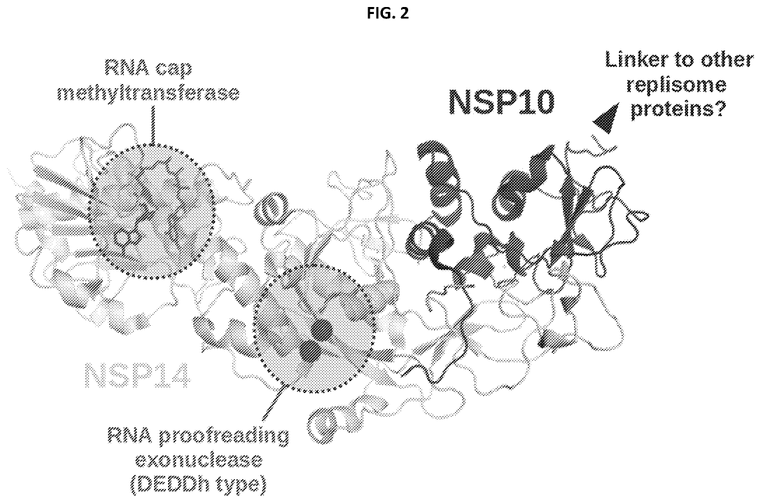

The low replicative fidelity of RNA virus RNA-dependent RNA-polymerase (RdRP) allows for viral adaption to diverse environments at the expense of a high error rate and risk for viral extinction. NSP14 harbors a unique N-terminal 3′ to 5′ exonuclease (ExoN) proofreading domain that excises mismatched nucleotides from the growing 3′end of the RNA strand, supporting replication fidelity and maintenance of the atypically large CoV genome. X-ray crystallography of SARS-CoV NSP14 complexed with allosteric activator SARS-CoV NSP10, which enhances the activity of NSP14, have revealed key features of the DEDD(h)-type catalytic domain of NSP14, including dual Mg²⁺ ion cofactors requisite for catalysis (Bouvet et al., 2012; Yang et al., 2011). High sequence homology between SARS-CoV and SARS-CoV2 catalytic domains has informed accurate modeling of the NSP14-NSP10 complex, and its mapping onto published structures (Ferron et al., 2017; Ma et al., 2015; https://resb.org/structure/5NFY). A schematic representation of the structure of the SARS-CoV-2 NSP14/NSP10 complex highlighting the catalytic center of NSP14 DEDDh nuclease is shown in FIG. 2.

Pertinent to present day drug discovery efforts, the ExoN domain of the NSP14/NSP10 complex has rendered available antiviral compounds ineffective in the treatment of SARS-CoV and SARS-CoV2, as many such compounds, e.g., nucleoside analogs including Remdesivir, are unable to effectively evade its catalytic proofreading activity. Further, development of NSP14/NSP10 inhibitors, while a promising avenue for future rational therapeutics targeting CoV, has not yet been aggressively pursued, partly due to the lack of robust in vitro screening assays for high sensitivity detection and quantification of NSP14 activity, a tool that can be subsequently used to identify antiviral strategies that inhibit its exonuclease activity. Currently available radioactive exonuclease activity assays require resolution of radioactive RNA products under denaturing gel conditions and are poor candidates for high throughput screening of compounds. A primary goal of the present Example was to design novel fluorescence resonance energy transfer (FRET) double-stranded oligonucleotide (RNA) pairs for highly sensitive and effective interrogation of SARS-CoV2 NSP14/NSP10 complex catalytic integrity.

i. Selection and Location of Reporter-Quencher Probes

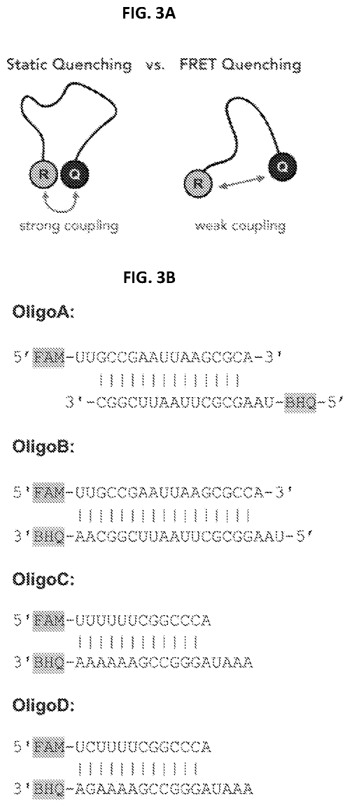

The double-stranded RNA (dsRNA) substrate designs and methods of their use disclosed herein were based both on FRET (dynamic or excited state) and static (ground state) quenching mechanisms, as exemplified in FIG. 3A. A fluorophore reporter 6FAM was attached to the 5′ end of the sense (5′-to-3′) strand of each dsRNA substrate design. As a single derivative isomer of Fluorescein, 6FAM fluorescent dye is an attractive reporter as it can be acquired with excitation/emission of approximately 495/520 nm with a Fluorescein isothiocyanate (FITC) or green fluorescent protein (GFP) filter common to most fluorescence detection equipment. To form the dsRNA substrate, the RNA sense strand was annealed to a complementary antisense strand (3′-to-5′) attached to a non-fluorescent Black Hole Quencher-1 (BHQ1) quencher molecule, which was positioned at either the 5′ or the 3′ end of the antisense strand. BHQ1 is a dark quencher molecule exhibiting strong absorption from 480-580 nm, a wavelength spectrally optimal for 6FAM. Thus, the proximity of BHQ1 to 6FAM in the intact dsRNA substrate provided for quenching of the reporter fluorescent signal. As dsRNA substrate designs of the present invention were optimized for their enzymatic breakdown via NSP14/NSP10 complex activity, spatial separation of the 6FAM from the BHQ1 during such a reaction resulted in enhanced fluorescent signal from the reporter molecule with increasing distance from quencher molecule. That a biologically relevant hit elicited an enhanced rather than a diminished signal substantially reduced the likelihood that changes in enzymatic activity detected using the assay were the result of unwanted artifact e.g., from compromised reagents. While 6FAM-BHQ1, with emitted wavelength detected in the green channel, was selected for use in the present study, several alternatives quencher-reporter probes were identified for future evaluation such as, but not limited to: Cy3-BHQ2 and TAMARA-BHQ2 (emitted wavelength detected in the red channel); TexasRed-BHQ2 (emitted wavelength detected in the near far-red channel); and, Cy5-BHQ3 (emitted wavelength detected in the far-red channel).

ii. Design of Substrates for Testing NSP14/NSP10 Exonuclease Activity

Double-stranded oligonucleotide substrates for measuring NSP14/NSP10 exonuclease activity were designed to both meet the demands intrinsic to the NSP14/NSP10 complex, as well as to maximize reporter-quencher probe (e.g., 6FAM-BHQ1) performance. Previous studies by others using radiolabeled dsRNA oligos (see e.g., Ferron et al., 2017) have shown that the SARS-CoV NSP14-NSP10 complex requires a free, accessible 3′ end to hydrolyze dsRNA; possesses limited processivity from the 3′ end in support of substrates that are reasonable in length (e.g., between 13 and 20 base pairs); and, demonstrates a propensity toward binding 3′ mismatches. While longer substrates with higher melting temperatures are associated with low background signal and enhanced signal-to-noise ratio, the NSP14/10 complex requires more time to hydrolyze an increasing number of bases, which may prolong or even prevent reaction completion. A single G or C base positioned within proximal (2-3 nucleotides) to the fluorophore reporter 6FAM were also incorporated into substrate designs, thereby providing anchor points for annealed substrates associated with enhanced quenching stability in the absence of digestion. A longer GC-stretch at the free 3′ was used to maximise the oligo destabilizing effect of the few nucleotides removed by the NSP14-10 complex. Otherwise, a GC-free-stretch was connecting the two ends of the oligo. To minimize fluorescence quenching caused by a neighboring base, and thus maximize assay sensitivity, uracil (U) was also placed directly adjacent to the 6FAM reporter. A detailed description of the selection and location of the 6FAM-BHQ1 reporter-quencher probes of the substrates of the invention, as well as additional alternative reporter-quencher probes for use in the same, is provided above.

In accordance with the above-summarized guidelines, the consensus structure for the oligonucleotide substrates was designed to meet several criteria, including but not limited to: 1) a dsRNA structure; 2) at least one free 3′ OH group; 3) a pair of FRET probes comprising a fluorophore and quencher with one probe located at the 5′ end of the strand comprising the free 3′ OH group and the other probe as located either at the 5′ end or at the 3′ end of the other strand of the dsRNA substrate; and optionally 4) a reasonable length (e.g., 13-20 base pairs); 5) a GC-stretch at the free 3′ end, but a GC-free-stretch adjacent to the fluorophore and/or quencher; 6) a U or A base immediately next to the fluorophore and/or a single C base in the second or third position from the fluorophore; and, 7) a 5′ overhang between 1 and 5 base pairs on the other strand. The designs for exemplar optimized oligonucleotide substrates A-D, and discussed in further detail below, are presented in FIG. 3B.

Briefly, the oligonucleotide substrates were optimized as follows: (1) Oligo A was capable of FRET quenching. While the background starting signal when the dsRNA was properly annealed was higher for Oligo A than for other substrate designs, this design was beneficial in that it had two NSP14/10 accessible 3′ ends (one being a mismatch), therefore the exonuclease enzyme was able to start hydrolyzing on either end of the substrate. This resulted in faster destabilization of the oligonucleotide, leading to the separation of the FRET pairs at the opposite ends. (2) Oligos B-D were capable of both FRET and static quenching, providing a much lower background signal when the dsRNA was properly annealed as compared to Oligo A. However, these substrates had only one free 3′ end (with a mismatch) to start the reaction. Therefore, the NSP14/10 enzyme complex needed to be processive enough to hydrolyze a sufficient number of bases from the single 3′ end to destabilize the oligonucleotide to separate the FRET pair thereby gaining fluorescence. (3) Oligo B was longer than Oligo C and Oligo D and had a higher melting temperature. This resulted in low background signal and high signal-to-noise ratio. However, as the NSP14/10 exonuclease required more time to hydrolyze enough bases, the reaction was more slowly completed than with Oligo C or D. Ultimately, NSP14/10 was not processive enough to drive the reaction to completion as compared to RNase A (see Example 4). The precise sequence was carefully chosen to maximize performance. The free 3′ end of the sequence contained a GC rich segment. When this GC rich segment was removed by NSP14/10 nuclease complex this oligonucleotide was more readily melting at 37° C. The single G or C base close to the 6FAM dye provided an anchor point for the annealed oligonucleotide for enhanced quenching stability when not digested. Uracil adjacent to the 6FAM reporter was used to minimize fluorescence quenching caused by the neighboring base (to maximize assay sensitivity). (4) Oligo C and Oligo D differed by a single base pair. Oligo D had a slightly better signal-to-noise ratio due to its higher melting temperature (see Example 2 and Example 3) even at 37° C. Oligo C was an earlier iteration but Oligo D is superior to it. Both Oligo C and Oligo D were shorter than Oligo B, and thus have a slightly higher background signal and reduced signal-to-noise ratio compared to Oligo B. However, both of these oligonucleotide designs had faster reaction times than Oligo B, and NSP14/10 was processive enough to completely digest these away. The free 3′ end of the sequence contained a GC rich segment. When this GC rich segment was removed by the NSP14/10 exonuclease complex, the oligonucleotide easily melted at 37° C. Oligo D had a single cytosine second to 6FAM, which stabilized the oligonucleotide at 37° C. when the oligo is intact, resulting in lower background signal compared to Oligo C. Uracil adjacent to 6FAM was used to minimize fluorescence quenching caused by the neighboring base (to maximize assay sensitivity). The above-discussed signal-to-noise, processivity, and reaction time findings, as well as corresponding data, are presented in greater detail in the below Examples (see e.g., Example 2 and Example 4).

iii. Preparation of Substrates

Double-stranded oligonucleotide substrates for measuring NSP14/NSP10 exonuclease activity were prepared the following way: sense and antisense single stranded RNA oligos were mixed together at 50 M final concentration in nuclease-free water. Reaction mixture was heated to 95° C. for 5 minutes and was gradually cooled down to 12° C. at a ramp rate of 4° C./min. The resulting annealed 50 μM double stranded RNA oligo was kept on ice and directly used in the activity assay.

iv. Evaluation of Substrate Designs

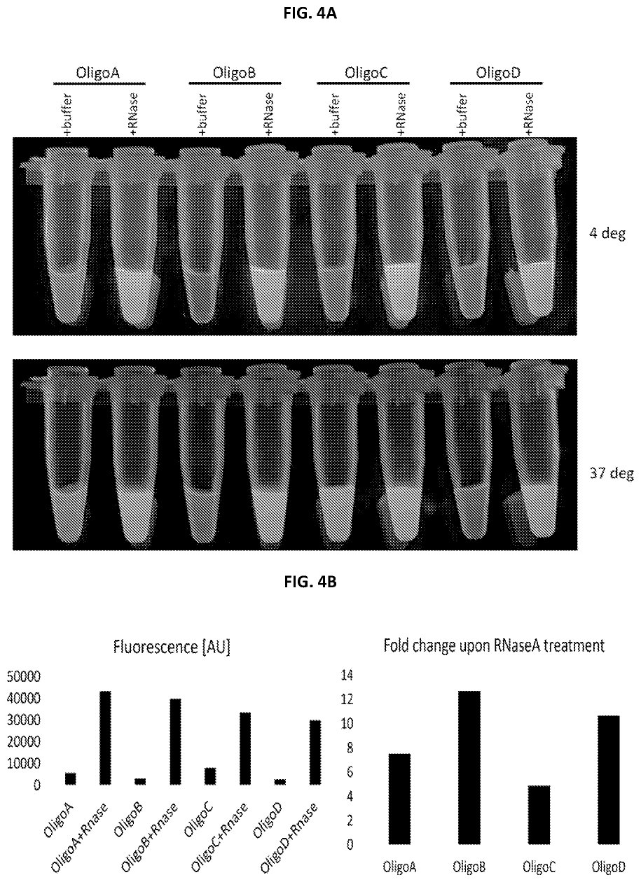

Annealed dsRNA substrates Oligo A-D (at a final concentration of 1 μM) were individually diluted in either: (1) reaction buffer (50 mM Tris-HCl, 2 mM MgCl₂, 2 mM DTT, pH 7.5) alone, to gauge background fluorescence, a property dependent on the quenching efficiency at a given temperature; or, (2) reaction buffer treated with RNase A (100 g/ml), to provide an index (positive control) of the theoretical maximum green fluorescent signal upon completion of a reaction (i.e., effective separation of the FRET probe pair). Non-limiting examples of additional positive controls contemplated for use included RNase L, RNase R, PNPase, Ribonuclease T1 and Nuclease BAL-31. Following dilution, samples were incubated at 4° C. and at 37° C. for 1 h, and reactions for Oligo A-D visualized and imaged in 200 μl microtubes for side-by-side comparison of fluorescence intensity using a blue light transilluminator. Images of the reactions are displayed in FIG. 4A.

For quantification of fluorescence intensity, reactions for Oligos A-D, prepared and incubated at 37° C. for 1 h as described above, were transferred to individual wells of a black, flat-well, low binding 96-well microplate. The microplate was then placed in a plate reader with excitation set at 490 nm (+/−9 nm) and emission detected at 520 nm (+/−20 nm). Fluorescence was measured and plotted in arbitrary units (AU), as shown in FIG. 4B (left). Fold change of fluorescence intensity upon RNase treatment was calculated as the amount of fluorescence measured for the RNase treatment group (AU) divided by the amount of fluorescence measured for the reaction buffer alone group (AU) for each dsRNA substrate, as presented in FIG. 4B (right).

### Example 3. Testing and Validation of dsRNA Substrate Designs for the FRET-Based Activity Assay

A FRET-based activity assay was used to test dsRNA substrate designs for high-throughput and optimized detection of NSP14/NSP10 complex exonuclease activity. For initial trials, annealed dsRNA substrates Oligo A and B (at a final concentration of 1 M each) were individually diluted to satisfy the following reaction conditions: (1) reaction buffer (50 mM Tris-HCl, 2 mM MgCl₂, 2 mM DTT, pH 7.5) alone (no enzyme); (2) reaction buffer treated with RNase A (100 g/ml); (3) reaction buffer additionally containing unbound NSP14 (200 nM); (4) reaction buffer additionally containing unbound NSP10 (600 nM); or, (5) reaction buffer additionally containing 200 nM NSP14 and 600 nMNSP10 mixture (later on defined just as NSP14/NSP10 complex). To enhance complex activity, the NSP10 stock solution (30 μM) was first pre-treated (incubation on ice for 10 minutes) with 0.2 M ZnCl₂ prior to addition to the reaction mixture.

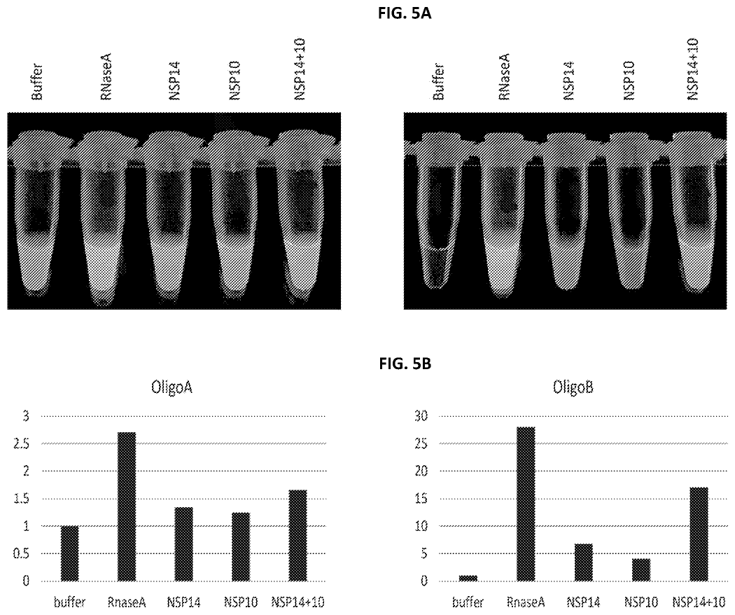

Following dilution, samples were incubated at 37° C. for 1 h, and reactions were visualized and imaged as described above. Results are displayed in FIG. 5A.

Fluorescence intensity of the reactions was quantified as described above. Fluorescence intensity plotted in arbitrary units (AU) for Oligo A and Oligo B across different reaction conditions is presented in FIG. 5B. For both Oligo A and Oligo B substrate designs, unbound NSP14 alone exhibited some exonuclease activity relative to the reaction buffer control, in agreement with the literature (e.g., see Bouvet et al., 2012, PMID: 22635272) however greater separation of probe pairs (higher fluorescence intensity) was observed for the NSP14/NSP10 mixture.

i. Validation of Sensitivity of NSP14/10 Exonuclease Activity to EDTA

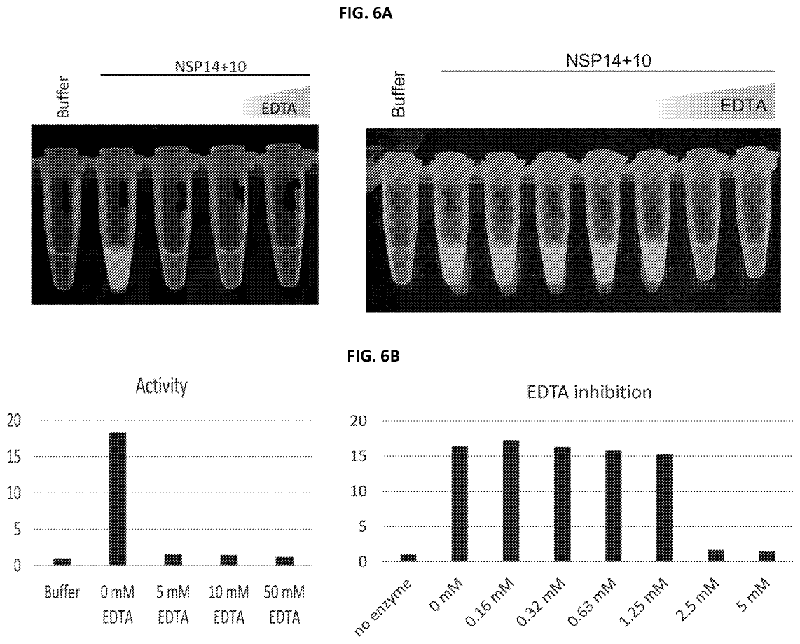

NSP14/NSP10 complex exonuclease activity requires Mg²⁺ ions to facilitate removal of mis-incorporated nucleotides. The present study was designed to validate the sensitivity of NSP14/NSP10 complex exonuclease activity to metal-chelating agent Ethylenediaminetetraacetic acid (EDTA). Annealed dsRNA substrate Oligo B (1 μM) and NSP14/NSP10 complex stock were diluted in reaction buffer (50 mM Tris-HCL, 2 mM MgCl₂, 2 mM DTT, pH 7.5) as described above containing increasing concentrations of EDTA (5-50 mM, range) for chelation of Mg²⁺ from the reaction buffer. Reaction buffer alone (no enzyme) served as a negative control. Samples were incubated at 37° C. for 1 h prior to visualization and imaging of fluorescent signal as described above, as presented in FIG. 6A. Fluorescence intensity of the reactions was quantified as described above. A broad range of EDTA concentrations (5-50 mM) was screened across a total of three concentrations (5 mM, 10 mM, and 50 mM EDTA), revealing inhibition of the NSP14/NSP10 complex exonuclease activity within the 0-5 mM EDTA range.

As described above, subsequent testing of finer concentration gradations at <5 mM EDTA (across a total of six different concentrations: 0.16 mM, 0.32 mM, 0.63 mM, 1.25 mM, 2.5 mM and 5 mM EDTA) showed titration of free Mg²⁺ from the reaction mixture, and thus complete inhibition of complex activity on Oligo B, at approximately 2 mM EDTA. Results are displayed in FIG. 6B. This study demonstrates feasibility of Mg²⁺ chelator-based inhibition of the NSP14/NSP10 exonuclease complex using the FRET-based activity assay.

ii. Validation of Facilitation of NSP14 Exonuclease Activity by NSP10

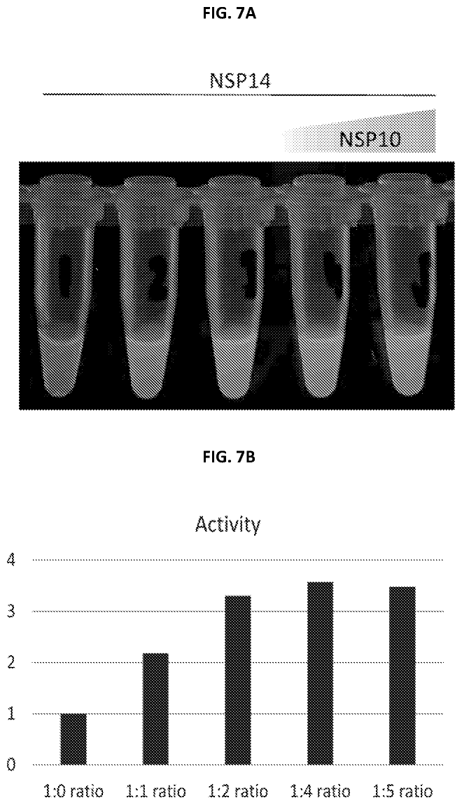

NSP10 is an allosteric activator of NSP14, conferring structural stability to the NSP14 exonuclease domain to support enzymatic integrity. The present study was designed to validate NSP10-mediated facilitation of NSP14 exonuclease activity. Annealed dsRNA substrate Oligo D (1 μM) was diluted in reaction buffer (50 mM Tris-HCl, 2 mM MgCl₂, 2 mM DTT, pH 7.5) containing increasing molar excess ratios of NSP14:NSP10 (1:1-1:5 molar ratio, range; 200 nM NSP14 with 200, 400, 600, 800 or 1000 nM NSP10 to achieve 1:1-1:5 molar ratios). As a control NSP14 without any NSP10 was used. Following dilution, samples were incubated at 37° C. for 1 h, and fluorescent signal emitted from reactions was visualized and imaged as described above. Results are displayed in FIG. 7A.

Fluorescence intensity of the reactions was quantified as described above. A description of fluorescence intensity relative to the NSP14 reaction condition across increasing molar excess ratios of NSP14:NSP10 is presented in FIG. 7B. Unbound NSP14 exhibited only very weak activity, negligible by comparison to that observed for the NSP14/NSP10 complex. NSP10 facilitated NSP14 activity to 1:4 molar excess of NSP10. To limit background nuclease impurities, a 1:3 NSP14:NSP10 molar ratio was selected for use in subsequent assays.

iii. Measurement of Nuclease Contaminants

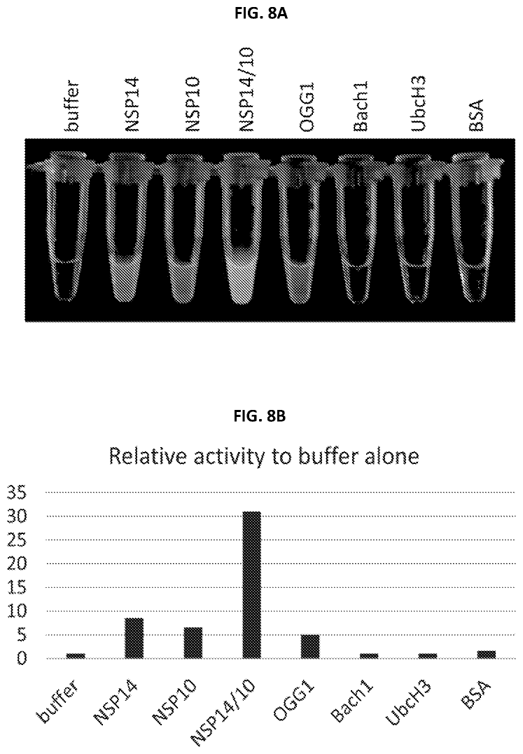

To measure background fluorescence from common contaminants, a panel of four unrelated (i.e., non-exonuclease), purified recombinant proteins (8-Oxoguanine DNA Glycosylase [OGG1]; BTB Domain And CNC Homolog 1 [Bach1]; Ubiquitin conjugating enzyme UbcH3; bovine serum albumin [BSA]) were tested in parallel with unbound NSP14, unbound NSP10, and NSP14/NSP10 complex by FRET-based activity assay, using methods similar to those described above. Annealed dsRNA substrate Oligo D (1 μM) was diluted to satisfy the following reaction conditions: (1) reaction buffer (50 mM Tris-HCl, 2 mM MgCl₂, 2 mM DTT, pH 7.5) alone (no enzyme); (2) reaction buffer additionally containing unbound NSP14 (200 nM); (3) reaction buffer additionally containing unbound NSP10 (600 nM); (4) reaction buffer additionally containing 200 nM NSP14 and 600 nM NSP10; (5) reaction buffer additionally containing OGG1 (800 nM); (6) reaction buffer additionally containing Bach1 (800 nM); (7) reaction buffer additionally containing UbcH3 (800 nM); or, (8) reaction buffer additionally containing BSA (800 nM). Samples were incubated at 37° C. for 1 h prior to visualization and imaging of fluorescent signal as described above. Results are displayed in FIG. 8A.

Fluorescence intensity of the reactions was quantified as described above. A description of fluorescence intensity relative to the reaction mixture reaction alone (buffer) condition across the reaction conditions is presented in FIG. 8B. Signal from background contamination was negligible by comparison to that observed for the NSP14/NSP10 complex. Additional negative control experiments in which reactions were treated with DNase I were performed, similarly revealing no increase in background fluorescent signal (data not shown).

### Example 4. Enzymatic Kinetic Profiling of Substrate Designs

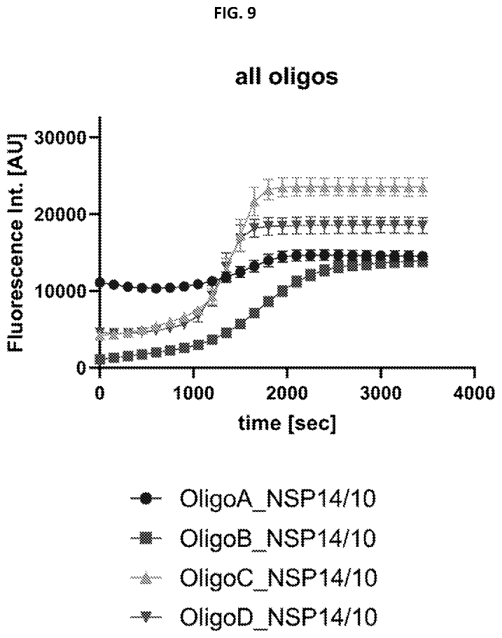

For kinetic profiling of NSP14/NSP10 complex activity on dsRNA substrate designs, the FRET-based activity assay as described above was used to quantify the time course of fluorescence intensity to reaction completion across Oligo A-D oligonucleotides, assessed in parallel. As the length of the probe was a key determinant of the amount of time required to reach reaction completion, combined testing of probes of different lengths (base pairs) in a single assay provided an estimate of the processivity of the NSP14/NSP10 complex. Annealed double-stranded RNA substrate designs Oligo A-D (1 μM) were individually diluted in reaction buffer (50 mM Tris-HCl, 2 mM MgCl₂, 2 mM DTT, pH 7.5) containing NSP14 (200 nM) plus NSP10 (600 nM). To enhance complex activity, the NSP10 stock solution (30 μM) was first pre-treated with 0.2 μM ZnCl₂ prior to addition to the reaction buffer. Reaction fluorescence intensity (arbitrary units, AU) was quantified at 37° C. for 1 h total time. As shown in FIG. 9, the NSP14/NSP10 complex digested each of Oligo A-D in a time dependent manner, with the reactions generally reaching completion between approximately 30 to 40 minutes (approximately 1,800 seconds to 2500 seconds). Given that Oligo B was longer by 5 base pairs (bps) than Oligo C (or Oligo D), Oligo B required approximately 3,000 (approximately 50 minutes) seconds to reach reaction completion, whereas Oligo C required approximately 2000 seconds (approximately 33 minutes). Together, these findings indicate that the rate of digestion by the NSP14/10 complex was approximately 0.3 bp/second, an estimate in line with the current literature (see e.g., Bouvet et al, 2012).

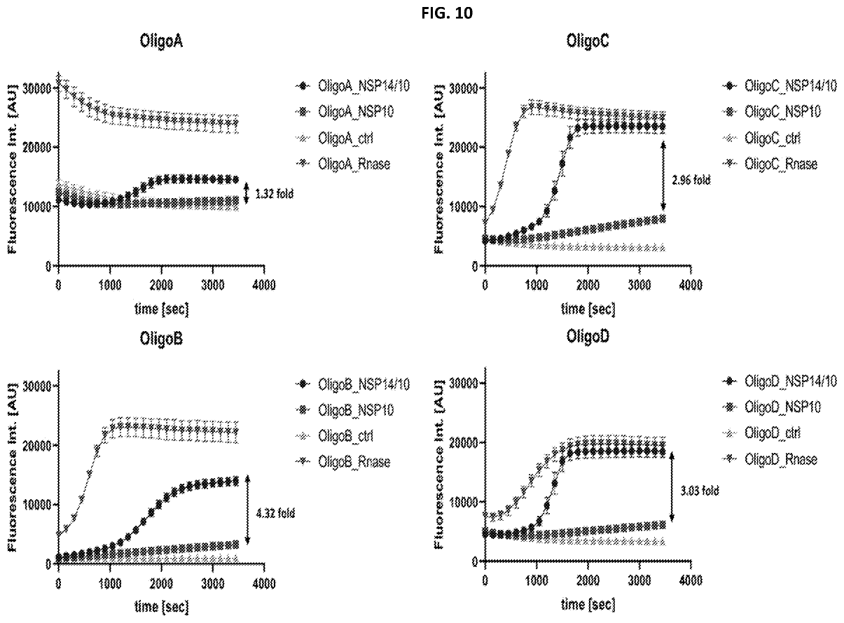

Individual kinetic profiles of unbound NSP14, unbound NSP10, and NSP14/NSP10 complex activity on dsRNA substrate designs Oligo A-D were generated, as displayed in FIG. 10. Annealed double-stranded RNA substrate designs Oligo A-D (1 μM) were individually diluted in reaction buffer (50 mM Tris-HCL, 2 mM MgCl₂, 2 mM DTT, pH 7.5) containing either unbound NSP14 (200 nM); unbound NSP10 (600 nM); or, NSP14 (200 nM) plus NSP10 (600 nM) to form the NSP14/NSP10 complex. Oligos A-D were diluted in reaction buffer alone, or in reaction buffer treated with RNase A as positive and negative controls, respectively. Following a reaction time of 1 h, the signal-to-noise ratio was determined as the fold change in fluorescence signal intensity (an index of complex activity) measured for the NSP14/10 complex condition relative that of NSP10 alone condition, the latter likely representative of background signal resulting from nuclease contamination by purified protein(s). For each Oligo A-D, NSP14/NSP10 fluorescence intensity was more than an order of magnitude higher than that of NSP10 alone (1.32-fold to 4.32-fold, range). The highest signal-to-noise ratio, and hence greatest sensitivity (i.e., the highest fold change fluorescence intensity) was observed for Oligo B (4.32-fold). Oligo C and Oligo D each proceeded to reaction completion, reaching fluorescent intensity levels similar to the positive control RNase A condition, while exhibiting signal-to-noise ratios similar to that of Oligo B (2.96-fold and 3.03-fold, respectively). Notably, unlike with NSP14/10, as above, the time course of digestion by RNase A was unaffected by the length of the substrate, and thus was no different between Oligo B versus Oligo C designs. Digestion by RNase A is fast, but still needs a few seconds to digest Oligos B-D to an extent that the oligo gets destabilized and the FRET quenching stops. However, with Oligo A, since the fluorophore and the quencher are on opposite end even a single cut by RNase A is enough to disable the FRET quenching. Therefore, the reaction is immediate, therefore even a 0 second it shows maximal level of signal intensity.

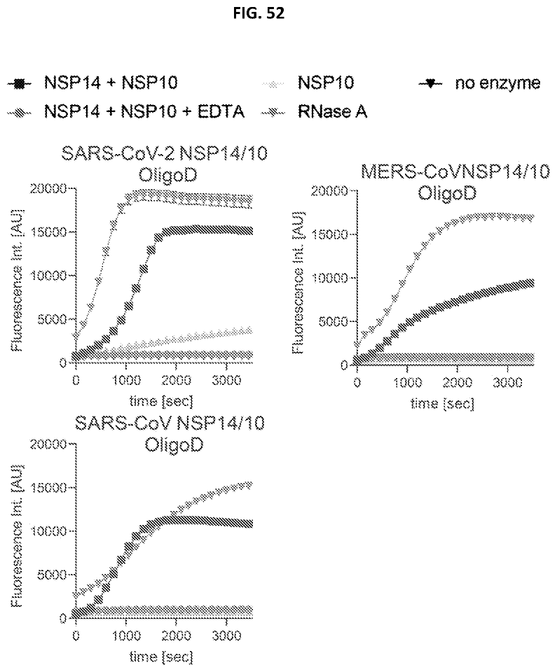

For comparison of kinetic profiling of SARS-CoV2, SARS-CoV and MERS-CoV NSP14/NSP10 complex activity on dsRNA Oligo D, the FRET-based activity assay, as described above, was used to quantify the time course of fluorescence intensity until reaction completion. Annealed double-stranded RNA substrate designs Oligo D (1 μM) were individually diluted in reaction buffer (50 mM Tris-HCl, 2 mM MgCl₂, 2 mM DTT, pH 7.5). This reaction buffer contained NSP14 (200 nM) plus NSP10 (600 nM) for MERS-CoV and NSP14 (40 nM) plus NSP10 (120 nM) for SARS-CoV. To enhance complex activity, the NSP10 stock solution (30 μM) was first pre-treated with 0.2 μM ZnCl₂ prior to addition to the reaction buffer. Reaction fluorescence intensity (arbitrary units, AU) was quantified at 37° C. for 1 h total time. Data on FIG. 52 shows that the FRET-based activity assay is capable of measuring NSP14/NSP10 complex activity of SARS-CoV2, SARS-CoV and MERS-CoV. Oligo D was diluted in reaction buffer alone, or in reaction buffer treated with RNase A as positive and negative controls, respectively. Signal from NSP10 alone condition, is likely representative of background signal resulting from nuclease contamination by purified protein(s). NSP14/NSP10 complex in the presence of 2.5 mM EDTA do not show exonuclease activity, showing specificity of the assay.

### Example 5. In Vitro Inhibition of SARS NSP14/NSP10 Exonuclease Activity

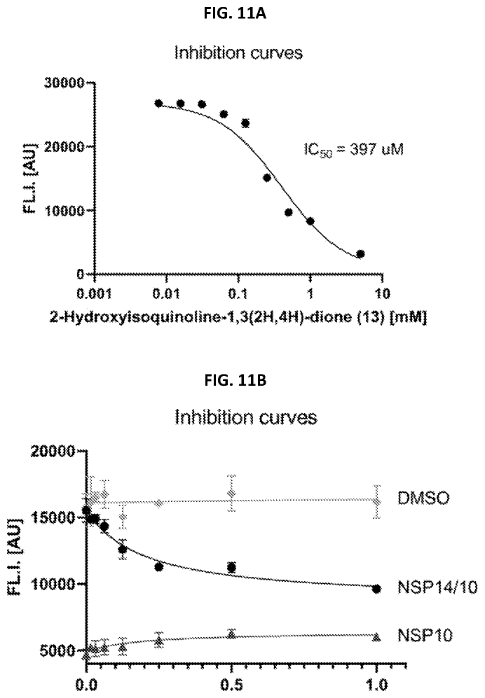

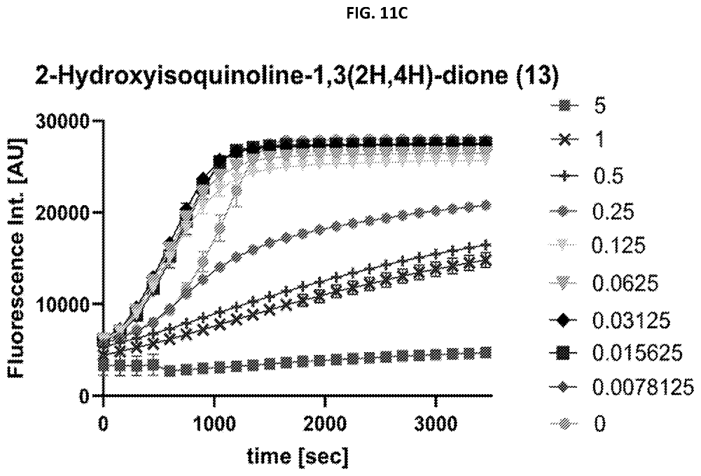

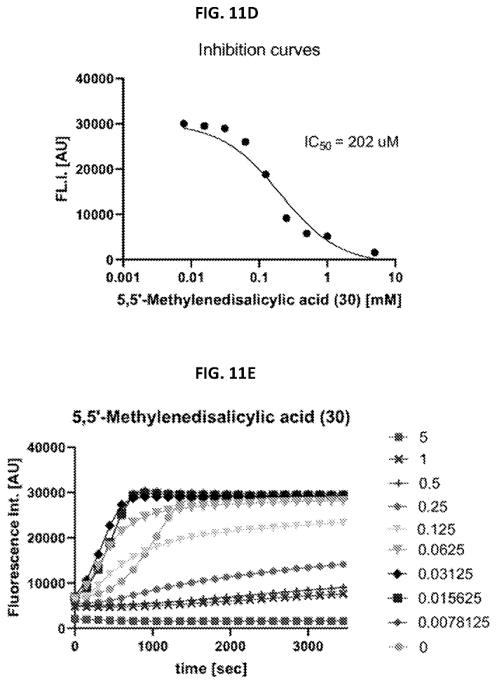

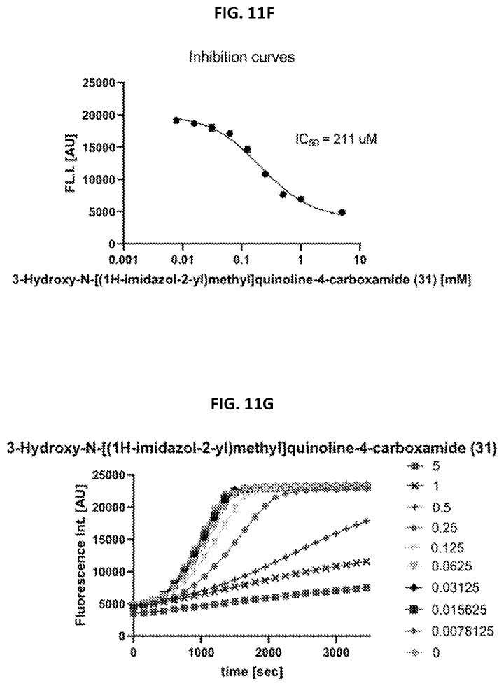

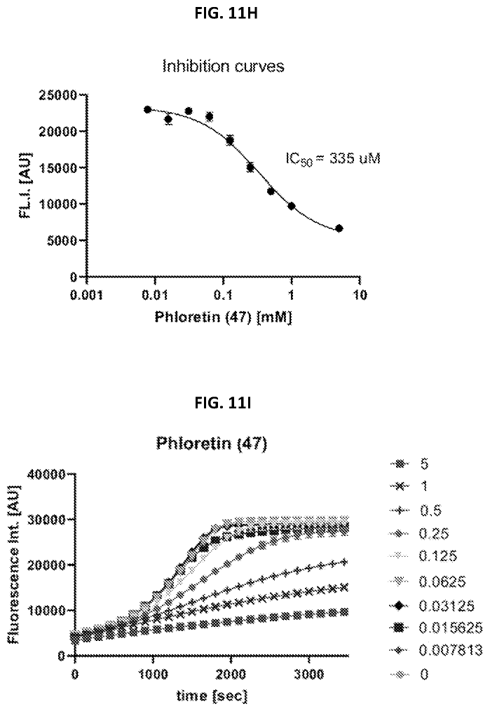

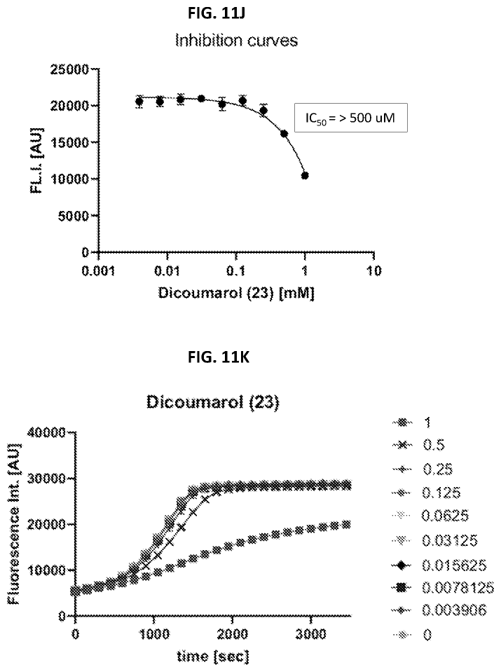

Commercially available 2-Hydroxyisoquinoline-1,3(2H,4H)-dione (CAS No: 6890-08-0), a well-known scaffold backbone used to develop human immunodeficiency virus (HIV) integrase activity, was selected for proof-of-principle in vitro efficacy studies of SARS-CoV2 NSP14/NSP10 exonuclease inhibition by FRET-based activity assay. The FRET-based activity assay was performed as described above. A description of a dose-response curve for 2-Hydroxyisoquinoline-1,3(2H,4H)-dione is presented in FIG. 11A. Dose response curves were used to determine the half maximal inhibitory concentration (IC₅₀) value of the select compounds against NSP14/NSP10 (aka inhibition of NSP14/NSP10 exonuclease activity by 50%). Kinetic activity profiles up to 1 hour of the NSP14/NSP10 complex on a dsRNA substrate (using Oligo D) were generated. These profiles were generated in the absence or presence of the select compound using in a defined range of concentration (between 0 mM and 1 M). The readouts for the IC₅₀ values were measured in the linear reaction range, where less than 60% of the total substrate had been consumed at the time of the readout (between 400-1000 seconds reaction time). The half maximal inhibitory concentration (IC₅₀) value for 2-Hydroxyisoquinoline-1,3(2H,4H)-dione (aka inhibition of NSP14/NSP10 exonuclease activity by 50%) was determined as 397 μM. Activity dose-response curves for 2-Hydroxyisoquinoline-1,3(2H,4H)-dione inhibition of the NSP14/10 complex, unbound NSP10, and Dimethyl Sulfoxide (DMSO) control are shown in FIG. 11B. Hydroxyisoquinoline-1,3(2H,4H)-dione did not inhibit the non-specific background of NSP10, indicative of highly specific 2-Hydroxyisoquinoline-1,3(2H,4H)-dione inhibition of NSP14/NSP10 complex exonuclease activity. DMSO, the vehicle control, was applied at the same amount as used in the 2-Hydroxyisoquinoline-1,3(2H,4H)-dione does response curve. DMSO on its own did not inhibit the NSP14/NSP10 complex at any given concentration. The fluorescent intensity signal during the time course of the reaction across increasing concentrations (mM) of 2-Hydroxyisoquinoline-1,3(2H,4H)-dione is shown in FIG. 11C.

5,5′-Methylenedisalicylic acid was also tested for in vitro inhibition of SARS-CoV2 NSP14/NSP10 exonuclease by FRET-based activity assay. The FRET-based activity assay was performed as described above. A description of a dose-response curve for 5,5′-Methylenedisalicylic acid is presented in FIG. 11D. The half maximal inhibitory concentration (IC₅₀) value for 5,5′-Methylenedisalicylic acid of NSP14/NSP10 exonuclease activity was determined as 202 μM. The fluorescent intensity signal during the time course of the reaction across increasing concentrations (mM) of 5,5′-Methylenedisalicylic acid is shown in FIG. 11E.

3-Hydroxy-N-[(1H-imidazol-2-yl)methyl]quinoline-4-carboxamide was also tested for in vitro inhibition of SARS-CoV2 NSP14/NSP10 exonuclease by FRET-based activity assay. The FRET-based activity assay was performed as described above. A description of a dose-response curve for 3-Hydroxy-N-[(1H-imidazol-2-yl)methyl]quinoline-4-carboxamide is presented in FIG. 11F. The half maximal inhibitory concentration (IC₅₀) value for 3-Hydroxy-N-[(1H-imidazol-2-yl)methyl]quinoline-4-carboxamide of NSP14/NSP10 exonuclease activity was determined as 211 μM. The fluorescent intensity signal during the time course of the reaction across increasing concentrations (mM) of 3-Hydroxy-N-[(1H-imidazol-2-yl)methyl]quinoline-4-carboxamide is shown in FIG. 11G.

Phloretin was also tested for in vitro inhibition of SARS-CoV2 NSP14/NSP10 exonuclease by FRET-based activity assay. The FRET-based activity assay was performed as described above. A description of a dose-response curve for Phloretin is presented in FIG. 11H. The half maximal inhibitory concentration (IC₅₀) value for Phloretin of NSP14/NSP10 exonuclease activity was determined as 335 μM. The fluorescent intensity signal during the time course of the reaction across increasing concentrations (mM) of Phloretin is shown in FIG. 11I.

Dicoumarol was also tested for in vitro inhibition of SARS-CoV2 NSP14/NSP10 exonuclease by FRET-based activity assay. The FRET-based activity assay was performed as described above. A description of a dose-response curve for Dicoumarol is presented in FIG. 11J. The half maximal inhibitory concentration (IC₅₀) value for Dicoumarol of NSP14/NSP10 exonuclease activity was determined as above 500 μM. The fluorescent intensity signal during the time course of the reaction across increasing concentrations (mM) of Dicoumarol is shown in FIG. 11K.

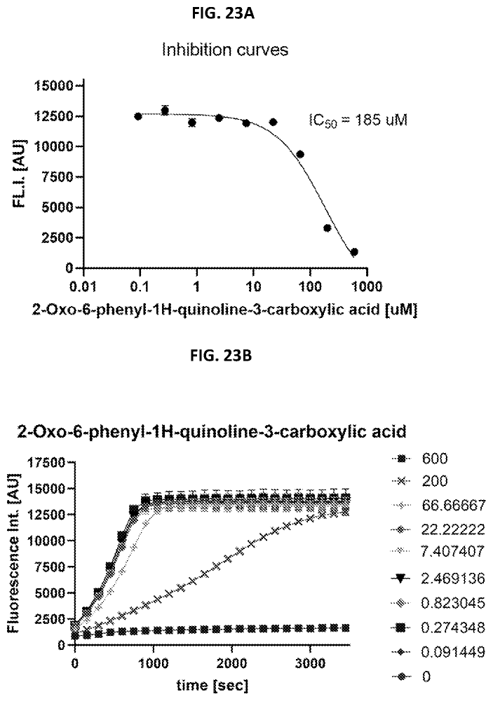

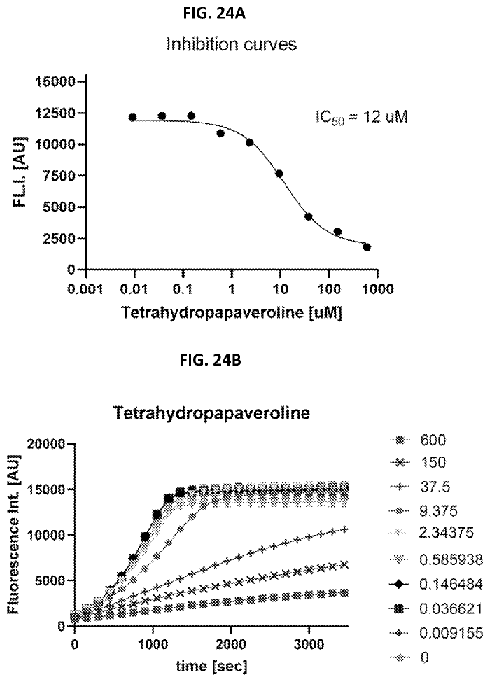

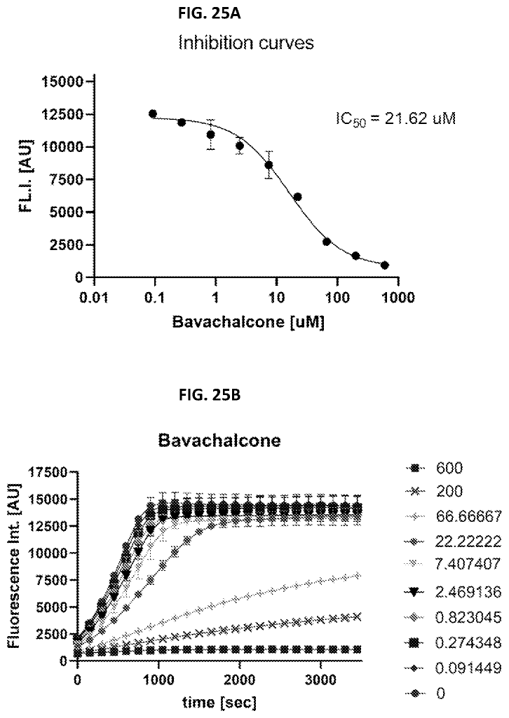

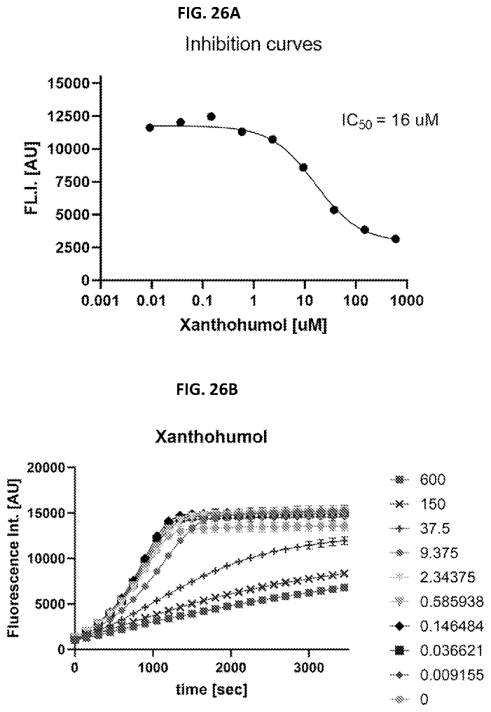

2-Oxo-6-phenyl-1H-quinoline-3-carboxylic acid was also tested for in vitro inhibition of SARS-CoV2 NSP14/NSP10 exonuclease by FRET-based activity assay. The FRET-based activity assay was performed as described above. A description of a dose-response curve for 2-Oxo-6-phenyl-1H-quinoline-3-carboxylic acid is presented in FIG. 23A. The half maximal inhibitory concentration (IC₅₀) value for 2-Oxo-6-phenyl-1H-quinoline-3-carboxylic acid of NSP14/NSP10 exonuclease activity was determined as 185 μM. The fluorescent intensity signal during the time course of the reaction across increasing concentrations (μM) of 2-Oxo-6-phenyl-1H-quinoline-3-carboxylic acid is shown in FIG. 23B.

Tetrahydropapaveroline was also tested for in vitro inhibition of SARS-CoV2 NSP14/NSP10 exonuclease by FRET-based activity assay. The FRET-based activity assay was performed as described above. A description of a dose-response curve for Tetrahydropapaveroline is presented in FIG. 24A. The half maximal inhibitory concentration (IC₅₀) value for Tetrahydropapaveroline of NSP14/NSP10 exonuclease activity was determined as 12 μM. The fluorescent intensity signal during the time course of the reaction across increasing concentrations (μM) of Tetrahydropapaveroline is shown in FIG. 24B.

Bavachalcone was also tested for in vitro inhibition of SARS-CoV2 NSP14/NSP10 exonuclease by FRET-based activity assay. The FRET-based activity assay was performed as described above. A description of a dose-response curve for Bavachalcone is presented in FIG. 25A. The half maximal inhibitory concentration (IC₅₀) value for Bavachalcone of NSP14/NSP10 exonuclease activity was determined as 21.62 μM. The fluorescent intensity signal during the time course of the reaction across increasing concentrations (μM) of Bavachalcone is shown in FIG. 25B.

Xanthohumol was also tested for in vitro inhibition of SARS-CoV2 NSP14/NSP10 exonuclease by FRET-based activity assay. The FRET-based activity assay was performed as described above. A description of a dose-response curve for Xanthohumol is presented in FIG. 26A. The half maximal inhibitory concentration (IC₅₀) value for Xanthohumol of NSP14/NSP10 exonuclease activity was determined as 16 μM. The fluorescent intensity signal during the time course of the reaction across increasing concentrations (μM) of Xanthohumol is shown in FIG. 26B.

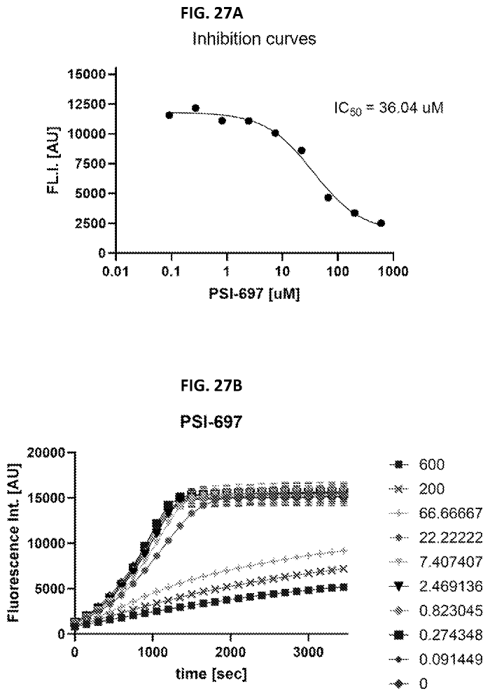

2-(4-Chlorobenzyl)-3-hydroxy-7,8,9,10-tetrahydrobenzo(H)quinoline-4-carboxylic acid (PSI-697) was also tested for in vitro inhibition of SARS-CoV2 NSP14/NSP10 exonuclease by FRET-based activity assay. The FRET-based activity assay was performed as described above. A description of a dose-response curve for PSI-697 is presented in FIG. 27A. The half maximal inhibitory concentration (IC₅₀) value for PSI-697 of NSP14/NSP10 exonuclease activity was determined as 36.04 μM. The fluorescent intensity signal during the time course of the reaction across increasing concentrations (μM) of PSI-697 is shown in FIG. 27B.

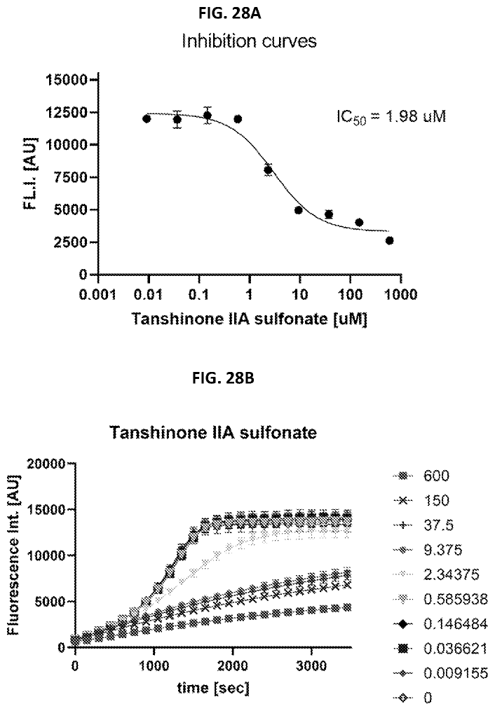

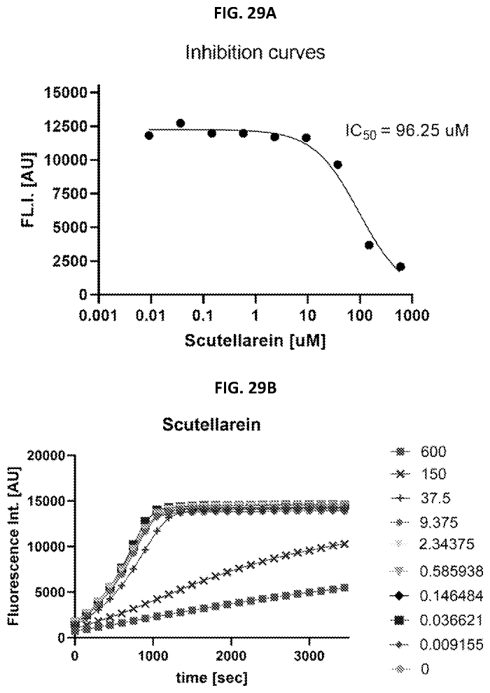

Tanshinone IIA sulfonate was also tested for in vitro inhibition of SARS-CoV2 NSP14/NSP10 exonuclease by FRET-based activity assay. The FRET-based activity assay was performed as described above. A description of a dose-response curve for Tanshinone IIA sulfonate is presented in FIG. 28A. The half maximal inhibitory concentration (IC₅₀) value for Tanshinone IIA sulfonate of NSP14/NSP10 exonuclease activity was determined as 1.98 μM. The fluorescent intensity signal during the time course of the reaction across increasing concentrations (μM) of Tanshinone IIA sulfonate is shown in FIG. 28B.

Scutellarein was also tested for in vitro inhibition of SARS-CoV2 NSP14/NSP10 exonuclease by FRET-based activity assay. The FRET-based activity assay was performed as described above. A description of a dose-response curve for Scutellarein is presented in FIG. 29A. The half maximal inhibitory concentration (IC₅₀) value for Scutellarein of NSP14/NSP10 exonuclease activity was determined as 96.25 μM. The fluorescent intensity signal during the time course of the reaction across increasing concentrations (μM) of Scutellarein is shown in FIG. 29B.

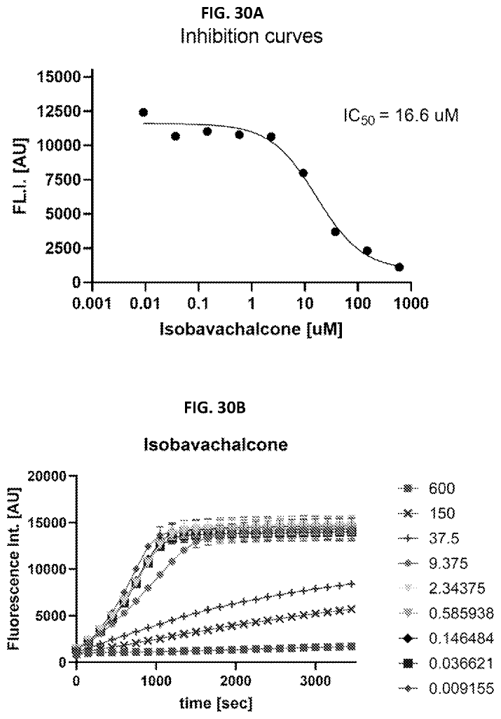

Isobavachalcone was also tested for in vitro inhibition of SARS-CoV2 NSP14/NSP10 exonuclease by FRET-based activity assay. The FRET-based activity assay was performed as described above. A description of a dose-response curve for Isobavachalcone is presented in FIG. 30A. The half maximal inhibitory concentration (IC₅₀) value for Isobavachalcone of NSP14/NSP10 exonuclease activity was determined as 16.6 μM. The fluorescent intensity signal during the time course of the reaction across increasing concentrations (μM) of Isobavachalcone is shown in FIG. 30B.

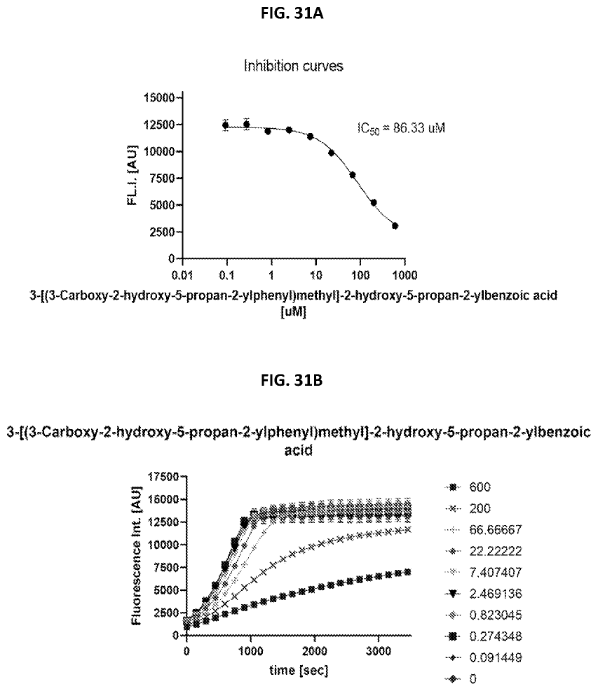

3-[(3-Carboxy-2-hydroxy-5-propan-2-ylphenyl)methyl]-2-hydroxy-5-propan-2-ylbenzoic acid was also tested for in vitro inhibition of SARS-CoV2 NSP14/NSP10 exonuclease by FRET-based activity assay. The FRET-based activity assay was performed as described above. A description of a dose-response curve for 3-[(3-Carboxy-2-hydroxy-5-propan-2-ylphenyl)methyl]-2-hydroxy-5-propan-2-ylbenzoic acid is presented in FIG. 31A. The half maximal inhibitory concentration (IC₅₀) value for 3-[(3-Carboxy-2-hydroxy-5-propan-2-ylphenyl)methyl]-2-hydroxy-5-propan-2-ylbenzoic acid of NSP14/NSP10 exonuclease activity was determined as 86.33 μM. The fluorescent intensity signal during the time course of the reaction across increasing concentrations (μM) of 3-[(3-Carboxy-2-hydroxy-5-propan-2-ylphenyl)methyl]-2-hydroxy-5-propan-2-ylbenzoic acid is shown in FIG. 31B.

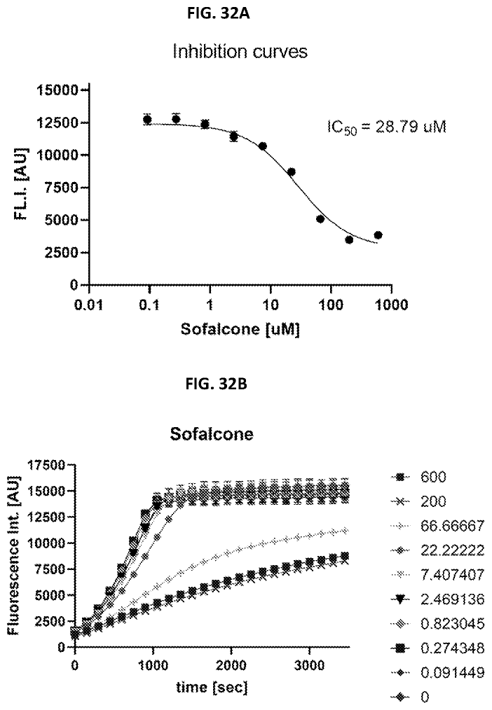

Sofalcone was also tested for in vitro inhibition of SARS-CoV2 NSP14/NSP10 exonuclease by FRET-based activity assay. The FRET-based activity assay was performed as described above. A description of a dose-response curve for Sofalcone is presented in FIG. 32A. The half maximal inhibitory concentration (IC₅₀) value for Sofalcone of NSP14/NSP10 exonuclease activity was determined as 28.79 μM. The fluorescent intensity signal during the time course of the reaction across increasing concentrations (μM) of Sofalcone is shown in FIG. 32B.

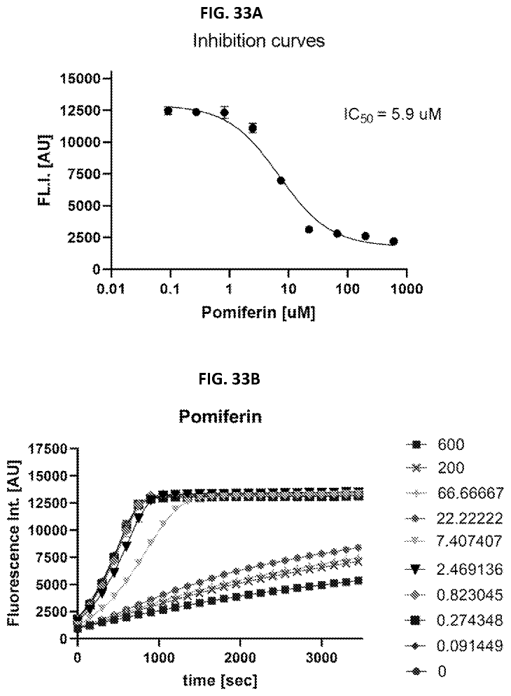

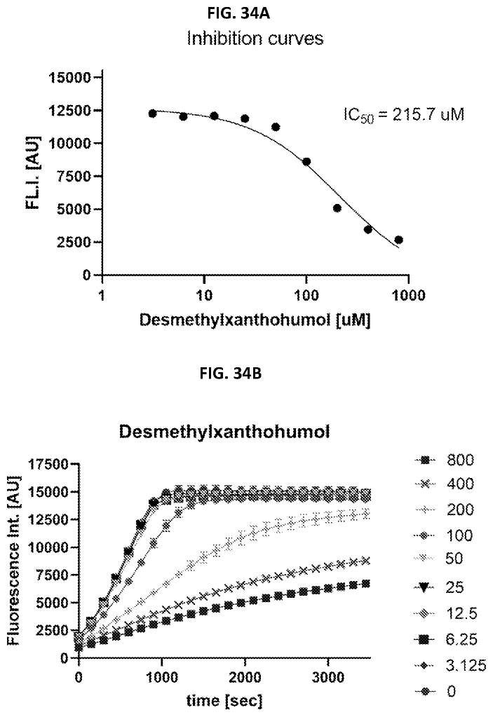

Pomiferin was also tested for in vitro inhibition of SARS-CoV2 NSP14/NSP10 exonuclease by FRET-based activity assay. The FRET-based activity assay was performed as described above. A description of a dose-response curve for Pomiferin is presented in FIG. 33A. The half maximal inhibitory concentration (IC₅₀) value for Pomiferin of NSP14/NSP10 exonuclease activity was determined as 5.9 μM. The fluorescent intensity signal during the time course of the reaction across increasing concentrations (μM) of Pomiferin is shown in FIG. 33B.

Desmethylxanthohumol was also tested for in vitro inhibition of SARS-CoV2 NSP14/NSP10 exonuclease by FRET-based activity assay. The FRET-based activity assay was performed as described above. A description of a dose-response curve for Desmethylxanthohumol is presented in FIG. 34A. The half maximal inhibitory concentration (IC₅₀) value for Desmethylxanthohumol of NSP14/NSP10 exonuclease activity was determined as 215.7 μM. The fluorescent intensity signal during the time course of the reaction across increasing concentrations (μM) of Desmethylxanthohumol is shown in FIG. 34BX.

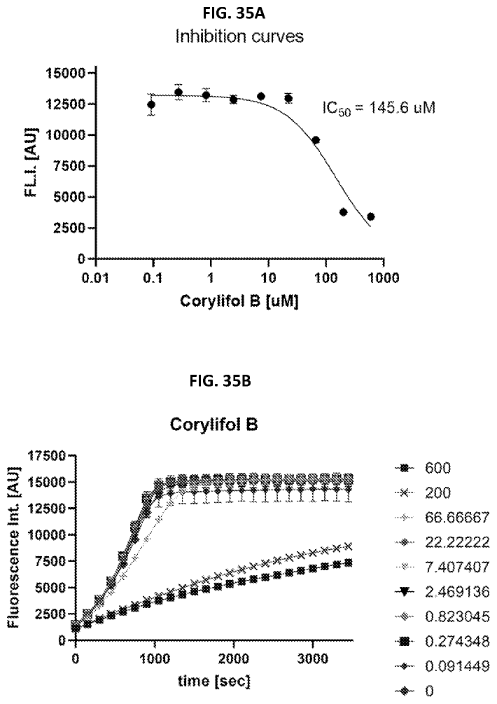

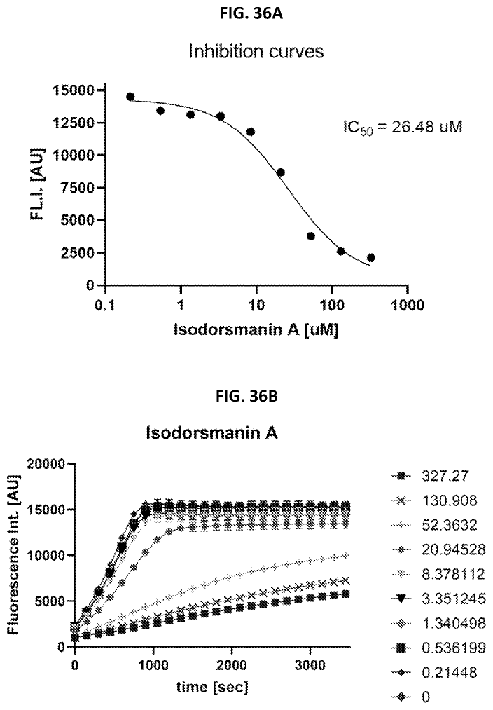

Corylifol B was also tested for in vitro inhibition of SARS-CoV2 NSP14/NSP10 exonuclease by FRET-based activity assay. The FRET-based activity assay was performed as described above. A description of a dose-response curve for Corylifol B is presented in FIG. 35A. The half maximal inhibitory concentration (IC₅₀) value for Corylifol B of NSP14/NSP10 exonuclease activity was determined as 145.6 μM. The fluorescent intensity signal during the time course of the reaction across increasing concentrations (μM) of Corylifol B is shown in FIG. 35B.

Isodorsmanin A was also tested for in vitro inhibition of SARS-CoV2 NSP14/NSP10 exonuclease by FRET-based activity assay. The FRET-based activity assay was performed as described above. A description of a dose-response curve for Isodorsmanin A is presented in FIG. 36A. The half maximal inhibitory concentration (IC₅₀) value for Isodorsmanin A of NSP14/NSP10 exonuclease activity was determined as 26.48 μM. The fluorescent intensity signal during the time course of the reaction across increasing concentrations (μM) of Isodorsmanin A is shown in FIG. 36B.

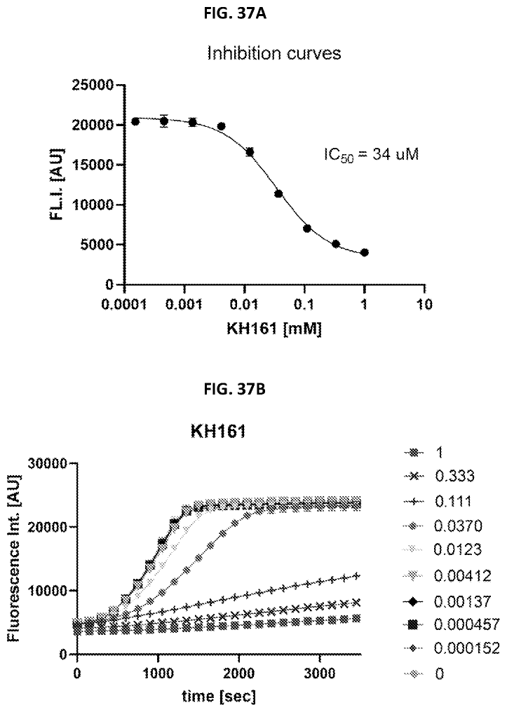

2-(3,4-dihydroxystyryl)-8-hydroxyquinoline-7-carboxylic acid (KH161) was also tested for in vitro inhibition of SARS-CoV2 NSP14/NSP10 exonuclease by FRET-based activity assay. The FRET-based activity assay was performed as described above. A description of a dose-response curve for KH161 is presented in FIG. 37A. The dose-response curve for KH161 shows half maximal inhibitory concentration (IC₅₀) of 34 μM. FIG. 37B shows a plot of the fluorescent intensity signal during the time course of NSP14/NSP10 inhibition across increasing concentrations of KH161 (mM), as detected by FRET-based activity assay.

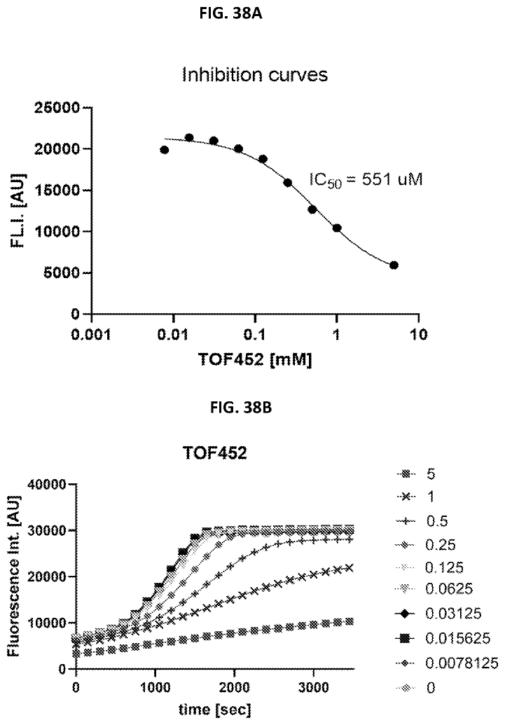

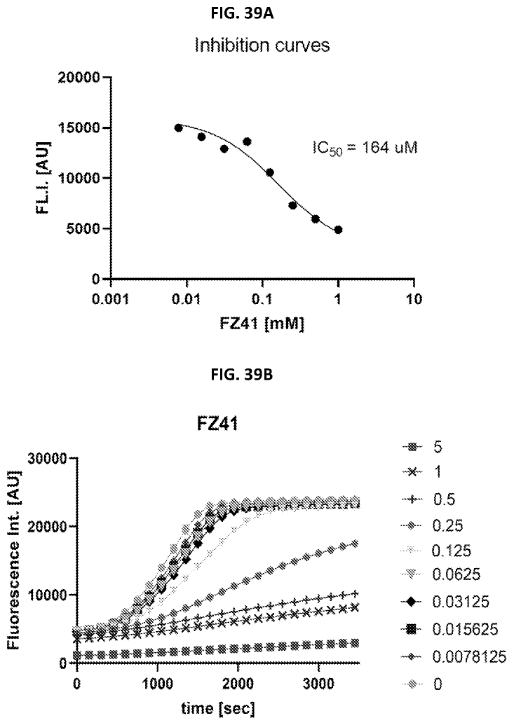

2-(3,4-dihydroxybenzylcarbamoyl)-8-hydroxyquinoline-7-carboxylic acid (TOF452) was also tested for in vitro inhibition of SARS-CoV2 NSP14/NSP10 exonuclease by FRET-based activity assay. The FRET-based activity assay was performed as described above. A description of a dose-response curve for TOF452 is presented in FIG. 38A. The dose-response curve for TOF452 shows half maximal inhibitory concentration (IC₅₀) of 551 μM. FIG. 38B shows a plot of the fluorescent intensity signal during the time course of NSP14/NSP10 inhibition across increasing concentrations of TOF452 (mM), as detected by FRET-based activity assay.

2-((3-methoxy-4,5-dihydroxy)styryl)-8-hydroxyquinoline-7-carboxylic acid (FZ41) was also tested for in vitro inhibition of SARS-CoV2 NSP14/NSP10 exonuclease by FRET-based activity assay. The FRET-based activity assay was performed as described above. A description of a dose-response curve for FZ41 is presented in FIG. 39A. The dose-response curve for FZ41 shows half maximal inhibitory concentration (IC₅₀) of 164 μM. FIG. 39B shows a plot of the fluorescent intensity signal during the time course of NSP14/NSP10 inhibition across increasing concentrations of FZ41 (mM), as detected by FRET-based activity assay.

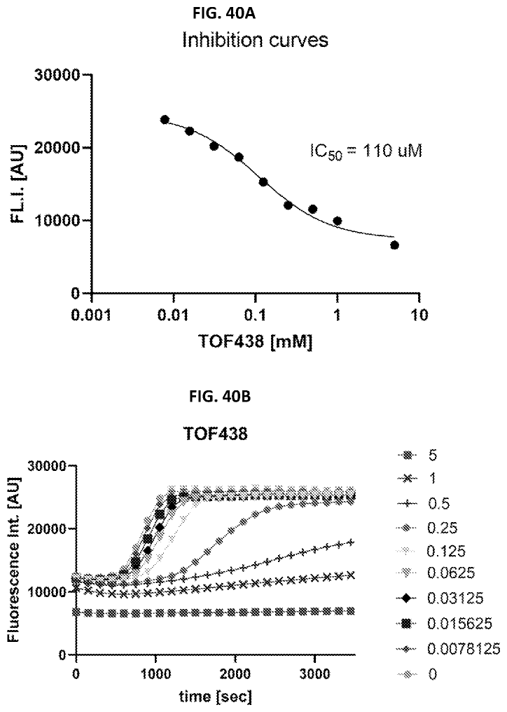

2-(2,3-dihydroxybenzylcarbamoyl)-8-hydroxyquinoline-7-carboxylic acid (TOF438) was also tested for in vitro inhibition of SARS-CoV2 NSP14/NSP10 exonuclease by FRET-based activity assay. The FRET-based activity assay was performed as described above. A description of a dose-response curve for TOF438 is presented in FIG. 40A. The dose-response curve for TOF438 shows half maximal inhibitory concentration (IC₅₀) of 110 μM. FIG. 40B shows a plot of the fluorescent intensity signal during the time course of NSP14/NSP10 inhibition across increasing concentrations of TOF438 (mM), as detected by FRET-based activity assay.

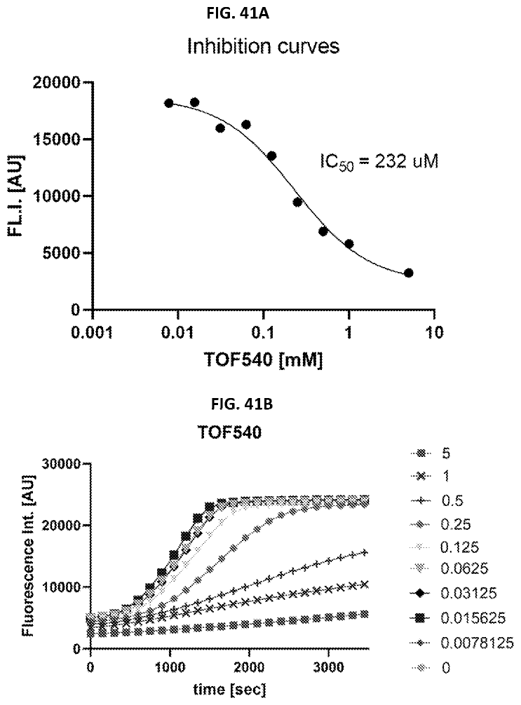

2-(3,5-dihydroxybenzylcarbamoyl)-8-hydroxyquinoline-7-carboxylic acid (TOF540) was also tested for in vitro inhibition of SARS-CoV2 NSP14/NSP10 exonuclease by FRET-based activity assay. The FRET-based activity assay was performed as described above. A description of a dose-response curve for TOF540 is presented in FIG. 41A. The dose-response curve for TOF540 shows half maximal inhibitory concentration (IC₅₀) of 232 μM. FIG. 41B shows a plot of the fluorescent intensity signal during the time course of NSP14/NSP10 inhibition across increasing concentrations of TOF540 (mM), as detected by FRET-based activity assay.

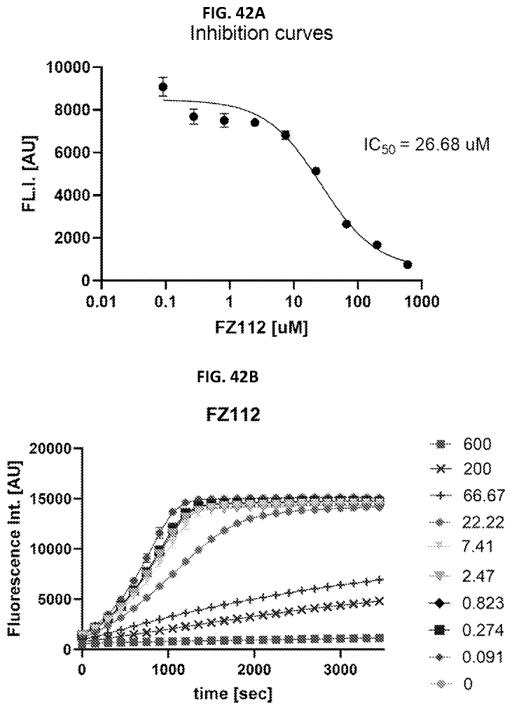

2-(3,5-dibromo-4-hydroxystyryl)-8-hydroxyquinoline-7-carboxylic acid (FZ112) was also tested for in vitro inhibition of SARS-CoV2 NSP14/NSP10 exonuclease by FRET-based activity assay. The FRET-based activity assay was performed as described above. A description of a dose-response curve for FZ112 is presented in FIG. 42A. The dose-response curve for FZ112 shows half maximal inhibitory concentration (IC₅₀) of 26.68 μM. FIG. 42B shows a plot of the fluorescent intensity signal during the time course of NSP14/NSP10 inhibition across increasing concentrations of FZ112 (μM), as detected by FRET-based activity assay.

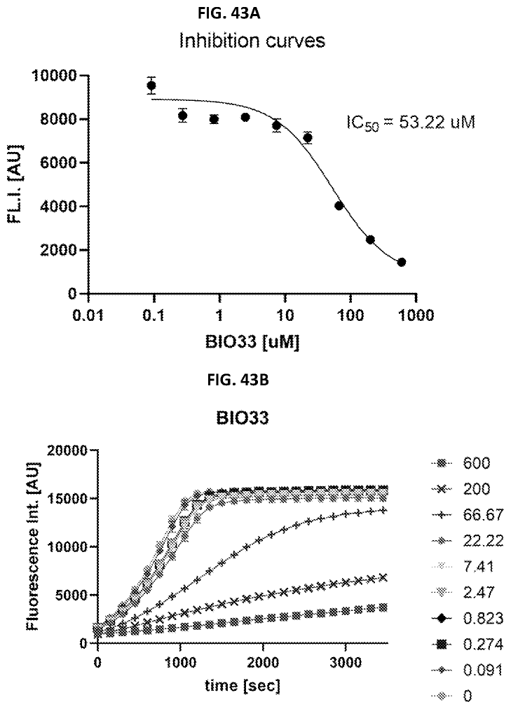

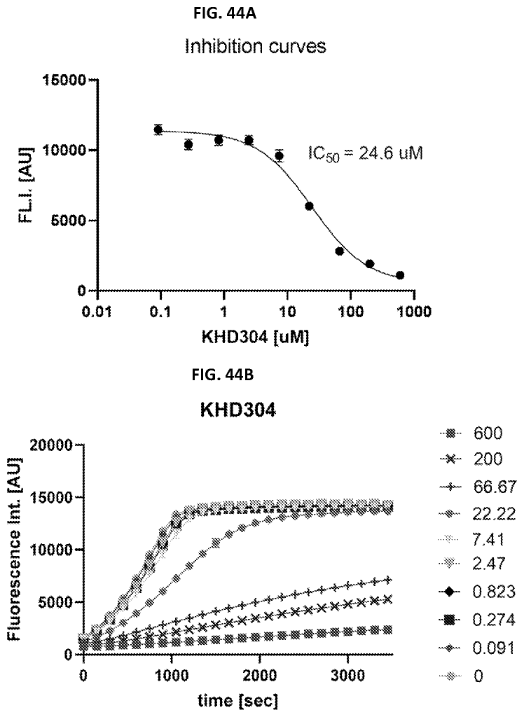

2-(3,4-difluorostyryl)-8-hydroxyquinoline-7-carboxylic acid (BI033) was also tested for in vitro inhibition of SARS-CoV2 NSP14/NSP10 exonuclease by FRET-based activity assay. The FRET-based activity assay was performed as described above. A description of a dose-response curve for BI033 is presented in FIG. 43A. The dose-response curve for BI033 shows half maximal inhibitory concentration (IC₅₀) of 53.22 μM. FIG. 43B shows a plot of the fluorescent intensity signal during the time course of NSP14/NSP10 inhibition across increasing concentrations of BI033 (μM), as detected by FRET-based activity assay.

2-(3-carboxy-4-hydroxystyryl)-8-hydroxyquinoline-7-carboxylic acid (KHD304) was also tested for in vitro inhibition of SARS-CoV2 NSP14/NSP10 exonuclease by FRET-based activity assay. The FRET-based activity assay was performed as described above. A description of a dose-response curve for KHD304 is presented in FIG. 44A. The dose-response curve for KHD304 shows half maximal inhibitory concentration (IC₅₀) of 24.6 μM. FIG. 44B shows a plot of the fluorescent intensity signal during the time course of NSP14/NSP10 inhibition across increasing concentrations of KHD304 (μM), as detected by FRET-based activity assay.

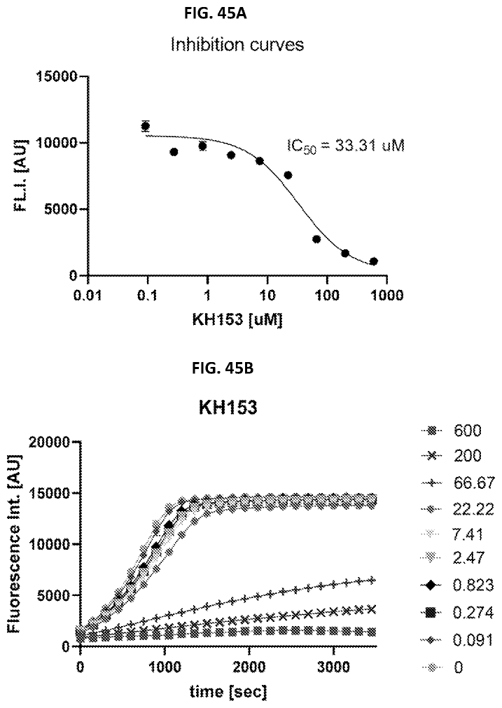

2-(3,4-dihydroxystyryl)-8-hydroxyquinoline (KH153) was also tested for in vitro inhibition of SARS-CoV2 NSP14/NSP10 exonuclease by FRET-based activity assay. The FRET-based activity assay was performed as described above. A description of a dose-response curve for KH153 is presented in FIG. 45A. The dose-response curve for KH153 shows half maximal inhibitory concentration (IC₅₀) of 33.31 μM. FIG. 45B shows a plot of the fluorescent intensity signal during the time course of NSP14/NSP10 inhibition across increasing concentrations of KH153 (μM), as detected by FRET-based activity assay.

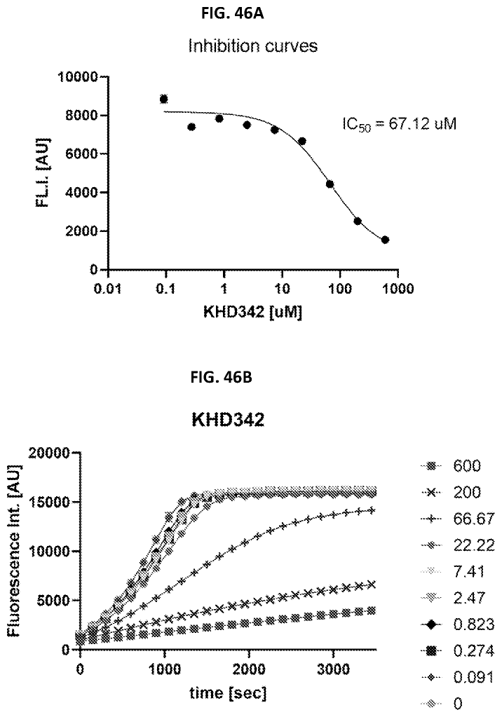

2-(3,4-dihydroxyphenethyl)-8-hydroxyquinoline-7-carboxylic acid (KHD342) was also tested for in vitro inhibition of SARS-CoV2 NSP14/NSP10 exonuclease by FRET-based activity assay. The FRET-based activity assay was performed as described above. A description of a dose-response curve for KHD342 is presented in FIG. 46A. The dose-response curve for KHD342 shows half maximal inhibitory concentration (IC₅₀) of 67.12 μM. FIG. 46B shows a plot of the fluorescent intensity signal during the time course of NSP14/NSP10 inhibition across increasing concentrations of KHD342 (μM), as detected by FRET-based activity assay.

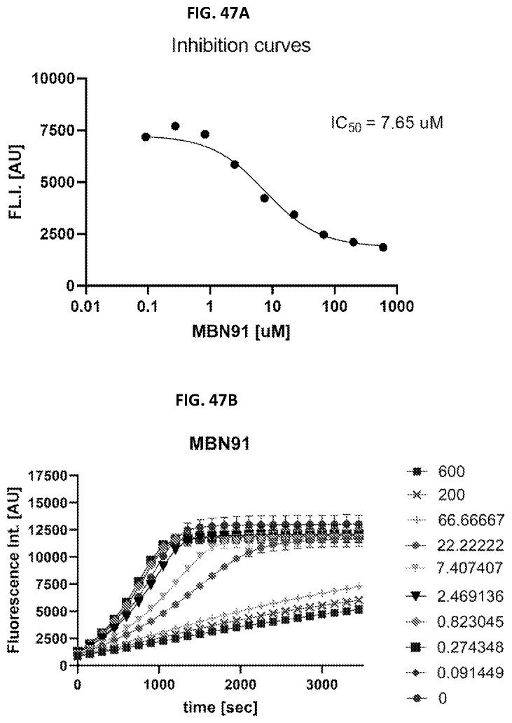

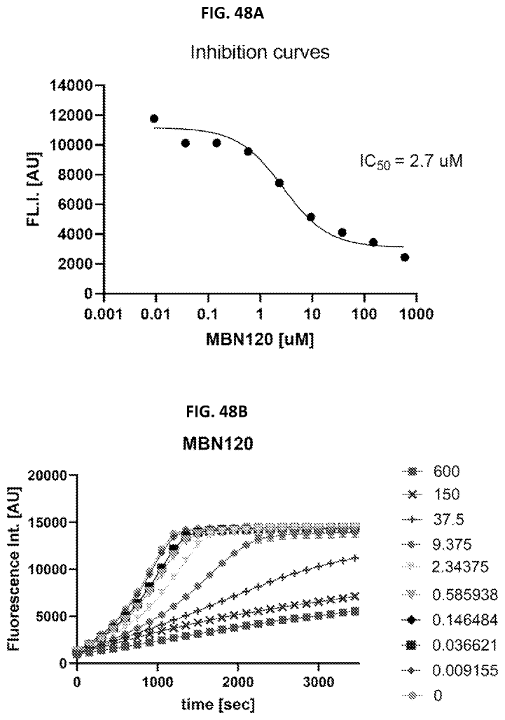

7-(3,4-difluorobenzoyl)-2-(3,4-dihydroxyvinyl)-8-hydroxyquinoline (MBN91) was also tested for in vitro inhibition of SARS-CoV2 NSP14/NSP10 exonuclease by FRET-based activity assay. The FRET-based activity assay was performed as described above. A description of a dose-response curve for MBN91 is presented in FIG. 47A. The dose-response curve for MBN91 shows half maximal inhibitory concentration (IC₅₀) of 7.65 μM. FIG. 47B shows a plot of the fluorescent intensity signal during the time course of NSP14/NSP10 inhibition across increasing concentrations of MBN91 (μM), as detected by FRET-based activity assay.

7-benzoyl-2-(3,4-dihydroxyvinyl)-8-hydroxyquinoline (MBN120) was also tested for in vitro inhibition of SARS-CoV2 NSP14/NSP10 exonuclease by FRET-based activity assay. The FRET-based activity assay was performed as described above. A description of a dose-response curve for MBN120 is presented in FIG. 48A. The dose-response curve for MBN120 shows half maximal inhibitory concentration (IC₅₀) of 2.70 μM. FIG. 48B shows a plot of the fluorescent intensity signal during the time course of NSP14/NSP10 inhibition across increasing concentrations of MBN120 (μM), as detected by FRET-based activity assay.

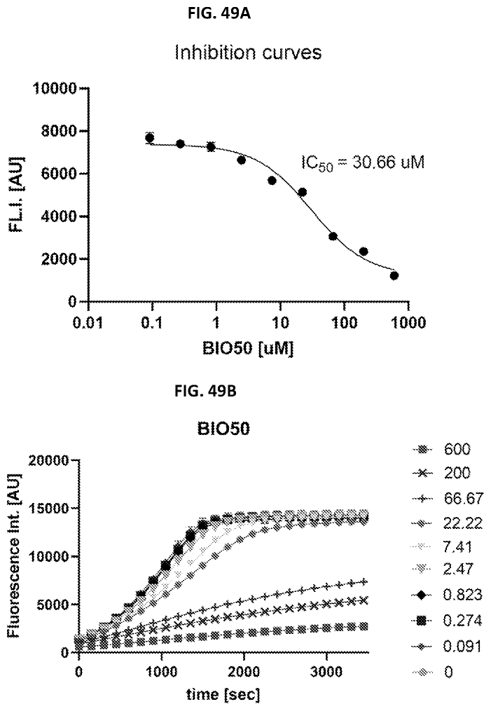

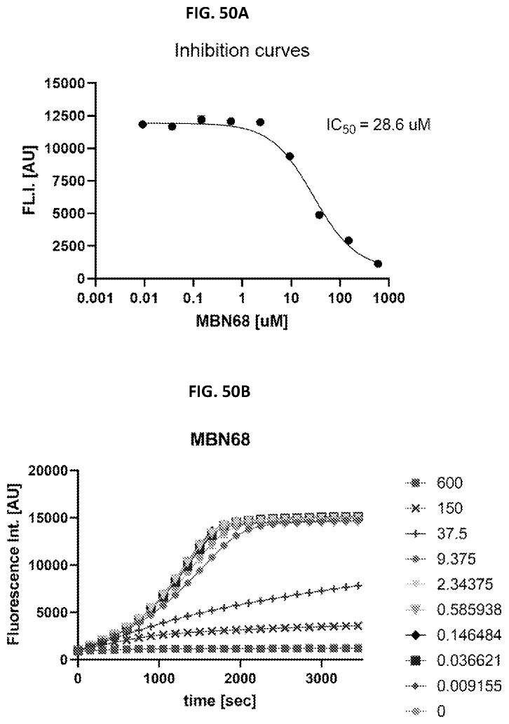

2-(3,4-dihydroxystyryl)-5-hydroxyquinoline-6-carboxylic acid (BI050) was also tested for in vitro inhibition of SARS-CoV2 NSP14/NSP10 exonuclease by FRET-based activity assay. The FRET-based activity assay was performed as described above. A description of a dose-response curve for BIO50 is presented in FIG. 49A. The dose-response curve for BIO50 shows half maximal inhibitory concentration (IC₅₀) of 30.66 μM. FIG. 49B shows a plot of the fluorescent intensity signal during the time course of NSP14/NSP10 inhibition across increasing concentrations of BIO50 (μM), as detected by FRET-based activity assay.

2-(3,4-dihydroxyphenethyl)-8-hydroxyquinoline-7-phosphonic acid (MBN68) was also tested for in vitro inhibition of SARS-CoV2 NSP14/NSP10 exonuclease by FRET-based activity assay. The FRET-based activity assay was performed as described above. A description of a dose-response curve for MBN68 is presented in FIG. 50A. The dose-response curve for MBN68 shows half maximal inhibitory concentration (IC₅₀) of 28.60 μM. FIG. 50B shows a plot of the fluorescent intensity signal during the time course of NSP14/NSP10 inhibition across increasing concentrations of MBN68 (μM), as detected by FRET-based activity assay.

### Example 6. AlamarBlue Viability Assay

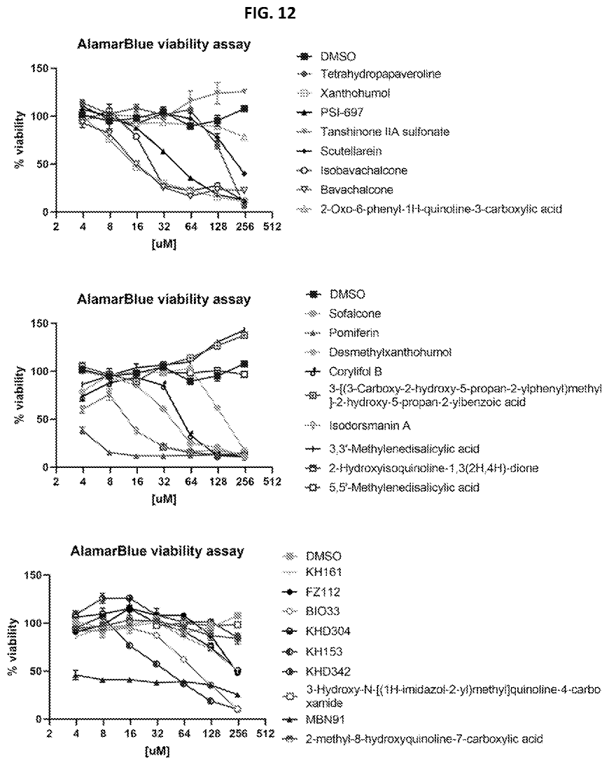

AlamarBlue viability assay was carried out on A549 cells. Ideally the inhibitors should not show toxicity on their own around their IC₅₀ value. This is true for several of the tested compounds. The results are shown on the four graphs in FIG. 12.

### Example 7. Thermal Stability of the NSP14/NSP10 Complex in the Presence of 2-Hydroxyisoquinoline-1,3(2H,4H)-Dione

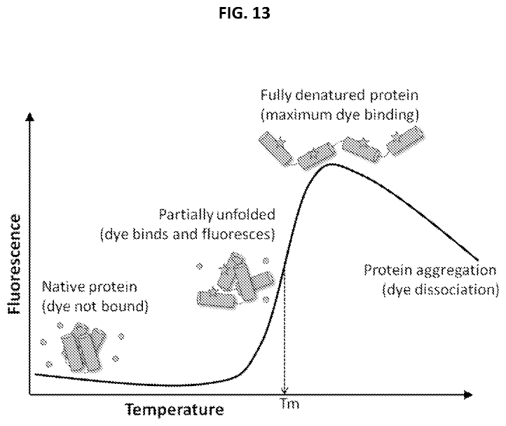

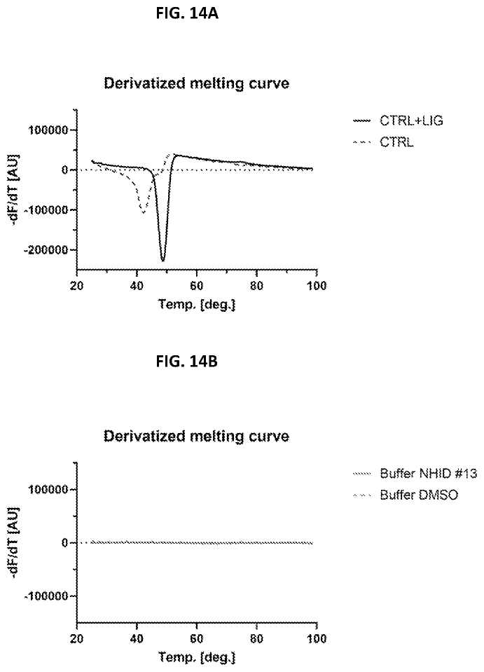

The thermal stability of the NSP14/NSP10 complex in the presence of 2-Hydroxyisoquinoline-1,3(2H,4H)-dione was tested by Thermofluor (thermal shift) assay, as schematically represented in FIG. 13. In this assay, an environmentally sensitive dye increasingly fluoresces upon binding to hydrophobic regions of unfolded proteins, maximally binding the fully denatured protein at its melting temperature (Tm), followed by dissociation of the dye thereby decreasing fluorescence signal upon protein aggregation. Protein Thermal Shift Starter Kit was used for the assay following the recommendation of the manufacturer. In brief 20 ul reaction volumes were prepared containing the indicated amount on NSP14, NSP10, DMSO or 2-Hydroxyisoquinoline-1,3(2H,4H)-dione in Protein Thermal Shift Buffer containing diluted Protein Thermal Shift Dye (SYPRO Orange).

As an initial evaluation of assay performance, the thermal stability of a control protein was first tested in the presence or absence of its cognate ligand. Reaction mixtures containing 1× Protein Thermal Shift Buffer containing 1× diluted Protein Thermal Shift Dye (SYPRO Orange) and 2 ul of Protein Thermal Shift Control protein w/wo 0.5 mM Protein Thermal Shift Control Ligand were prepared in quantitative polymerase chain reaction tubes. The samples were then briefly centrifuged and placed in a real-time PCR thermocycler. A melting curve profile was applied with a ramp rate of 0.2° C./s. Data were acquired in the ROX channel. The first derivative (slope) of the fluorescent curve (−dF/dT) was plotted against temperature to generate derivatized melting curves for the control protein in the presence or absence of its cognate ligand, as presented in FIG. 14A. Results showed a rightward shift (+6.6° C.) in melting temperature of the control protein plus the ligand versus the control protein alone condition, demonstrating enhanced stability of the control protein in its ligand-bound, as compared to its unbound state, as well as validating assay conditions. To confirm lack of signal from either the reaction buffer or the inhibitor compound, reaction buffer was prepared with Dimethyl Sulfoxide (DMSO) or with Hydroxyisoqunoline-1,3(2H,4H)-dione using methods similar to those described above. Derivatize melting curves for the reaction buffer plus DMSO and reaction buffer plus Hydroxyisoqunoline-1,3(2H,4H)-dione are shown in FIG. 14B. Results confirmed that neither the buffer nor the inhibitor contributed to signal detected by the assay.

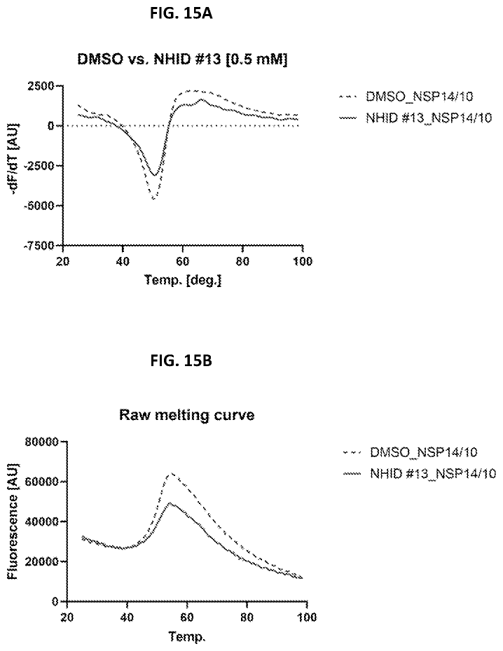

To determine whether Hydroxyisoquinoline-1,3(2H,4H)-dione affected the thermal stability of the NSP14/NSP10 complex, reaction mixtures were prepared containing either NSP14/10 (5 μM) complex plus DMSO (0.5%, equivalent vehicle control), or NSP14/10 complex (5 μM) in the presence of Hydroxyisoqunoline-1,3(2H,4H)-dione (NHID #13; 500 μM), using methods similar to those described above. The derivatize melting curve for the NSP14/10 complex plus DMSO, and the NSP14/10 complex in the presence of Hydroxyisoqunoline-1,3(2H,4H)-dione are shown in FIG. 15A. Raw melting curve data for the same conditions are presented in FIG. 15B. In each instance, no shift in melting curve was observed for the NSP14/10 complex in the presence of Hydroxyisoqunoline-1,3(2H,4H)-dione versus the NSP14/10 complex plus DMSO control condition. Together, these data demonstrated that Hydroxyisoqunoline-1,3(2H,4H)-dione did not abolish the structure of the NSP14/10 complex and thus, inhibition of the NSP14/NSP10 complex exonuclease activity did not arise from protein denaturation in the presence of the inhibitor.

### Example 8. Thermal Stability of the NSP14/NSP10 Complex in the Presence of 7-Trifluoromethyl-N-(4-Fluorobenzyl)-2-Hydroxy-1,3-Dioxo-4H-Isoquinoline-4-Carboxamide (VS59), Isobavachalcone and Solfacone

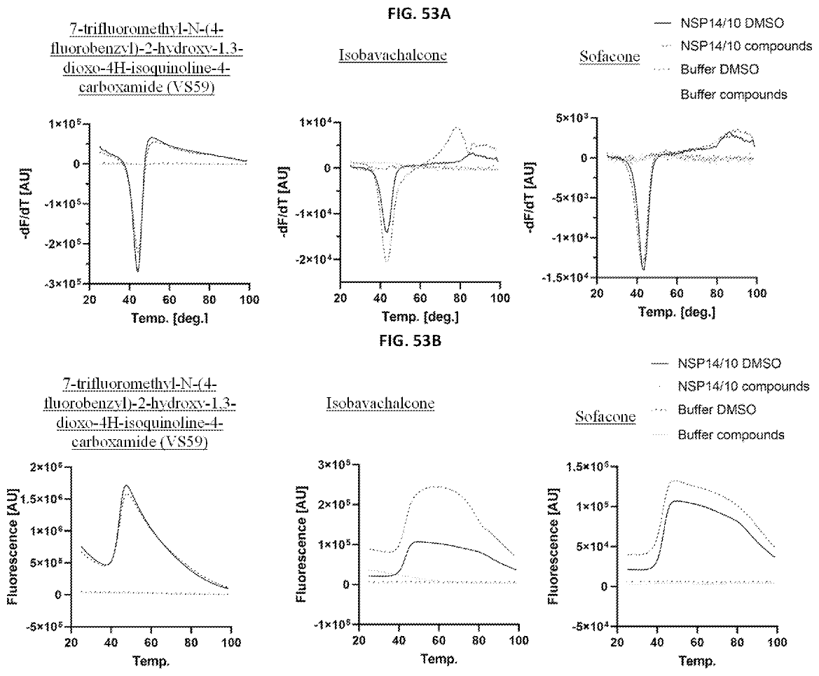

To determine whether 7-trifluoromethyl-N-(4-fluorobenzyl)-2-hydroxy-1,3-dioxo-4H-isoquinoline-4-carboxamide (VS59), Isobavachalcone and Solfacone affected the thermal stability of the NSP14/NSP10 complex, reaction mixtures were prepared containing either NSP14/10 (5 μM) complex plus DMSO (0.5%, equivalent vehicle control), or NSP14/10 complex (5 μM) in the presence of 7-trifluoromethyl-N-(4-fluorobenzyl)-2-hydroxy-1,3-dioxo-4H-isoquinoline-4-carboxamide (VS59), Isobavachalcone and Solfacone (50 M each), using methods similar to those described above. The derivatize melting curve for the NSP14/10 complex FIG. 53A. Raw melting curve data for the same conditions are presented in FIG. 53B. In each instance, no shift in melting curve was observed for the NSP14/10 complex in the presence of 7-trifluoromethyl-N-(4-fluorobenzyl)-2-hydroxy-1,3-dioxo-4H-isoquinoline-4-carboxamide (VS59), Isobavachalcone and Solfacone versus the NSP14/10 complex plus DMSO control condition. Together, these data demonstrated that 7-trifluoromethyl-N-(4-fluorobenzyl)-2-hydroxy-1,3-dioxo-4H-isoquinoline-4-carboxamide (VS59), Isobavachalcone and Solfacone did not abolish the structure of the NSP14/10 complex and thus, inhibition of the NSP14/NSP10 complex exonuclease activity did not arise from protein denaturation in the presence of the inhibitor.

### Example 9. Evaluation of Hydroxyisoquinoline-1,3(2H,4H)-Dione Effects on FRET Fluorescence Quenching Following Reaction Completion

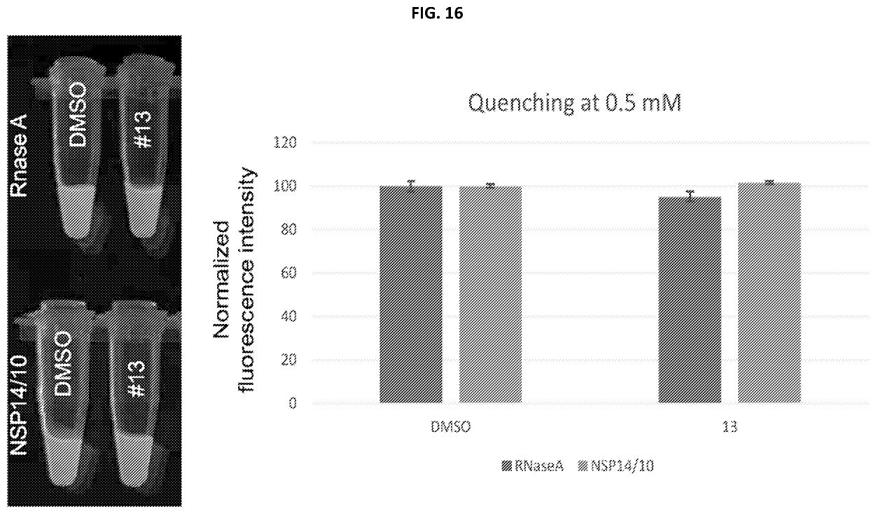

The present experiment was performed as a continuation of proof-of-principle studies aimed at evaluating inhibitory actions of Hydroxyisoquinoline-1,3(2H,4H)-dione on SARS-CoV2 NSP14/NSP10 exonuclease activity by FRET-based activity assay. Briefly, reaction buffer (50 mM Tris-HCl, 2 mM MgCl₂, 2 mM DTT, pH 7.5) containing dsRNA substrate Oligo D (1 μM) was treated with either RNase A (100 μg/ml) or 200 nM NSP14 and 600 nM. Following preparation, samples were incubated at 37° C. for 1 h. Either DMSO (0.5%), equivalent vehicle control) or Hydroxyisoquinoline-1,3(2H,4H)-dione (500 μM) was then added to the reaction mixtures. Reactions were visualized and imaged in 200 μl microtubes for side-by-side comparison using a blue light transilluminator and fluorescence intensity of the reactions quantified. The results are displayed in FIG. 16. The FRET signal obtained after digestion of Oligo D by either RNase A or the NSP14/10 complex was not quenched with addition of Hydroxyisoquinoline-1,3(2H,4H)-dione after reaction completion. The absence of signal observed when Hydroxyisoquinoline-1,3(2H,4H)-dione was added prior to reaction completion as above (see Example 5) could thus be attributed to the inhibitory effect of the compound on NSP14/10 complex activity.

### Example 10. Evaluation of Compounds #96, #112, #54, #60, #77, #68, #69 and #78 from Table 2 Effects on FRET Fluorescence Quenching Following Reaction Completion

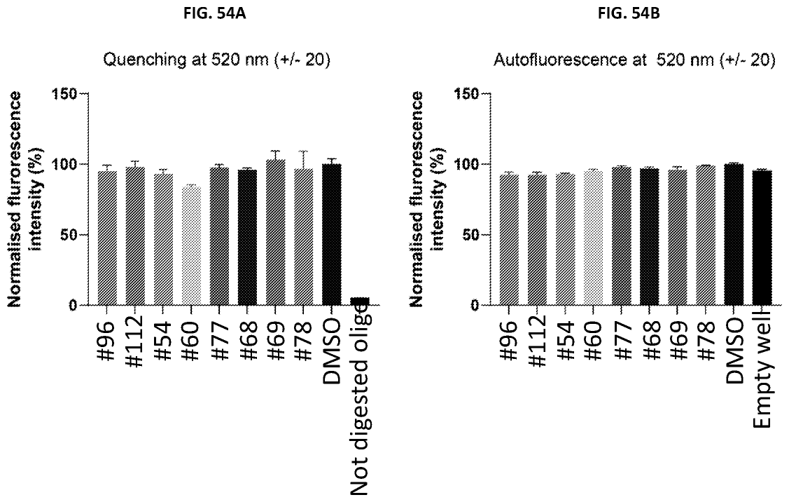

The present experiment was performed as a continuation of proof-of-principle studies aimed at evaluating inhibitory actions of compounds #96, #112, #54, #60, #77, #68, #69 and #78 from Table 2 on SARS-CoV2 NSP14/NSP10 exonuclease activity by FRET-based activity assay. Briefly, reaction buffer (50 mM Tris-HCl, 2 mM MgCl₂, 2 mM DTT, pH 7.5) containing dsRNA substrate Oligo D (1 μM) was treated with RNase A (100 μg/ml). Following preparation, samples were incubated at 37° C. for 1 h. Either DMSO (0.5% equivalent vehicle control) or compounds #96, #112, #54, #60, #77, #68, #69 and #78 from Table 2 (at 250 M concentration) were then added to the reaction mixtures. The results are displayed in FIG. 54A. The FRET signal obtained after digestion of Oligo D by either RNase A was not quenched with addition of compounds #96, #112, #54, #60, #77, #68, #69 and #78 after reaction completion. Compounds #96, #112, #54, #60, #77, #68, #69 and #78 were also tested against autofluorescence at the indicated wavelength at 250 μM final concentration. Results on FIG. 54B show none of these compounds have autofluorescence. Graphs were normalized to DMSO. Error bars represent SD from one experiment using technical triplicates.

### Example 11. Design and Assessment of a Substrate with a TexasRed-BHQ2 FRET Pair

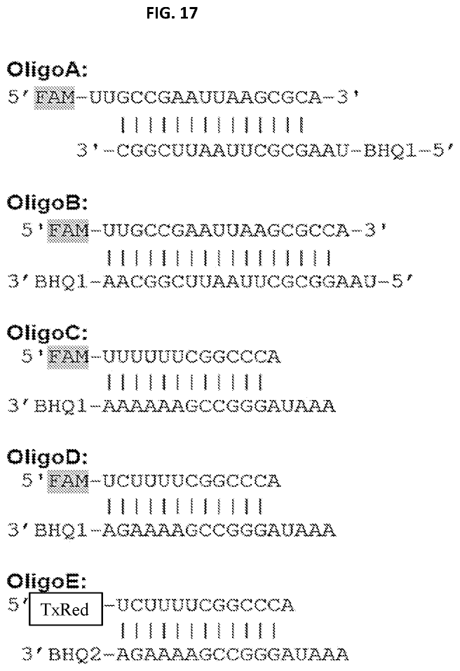

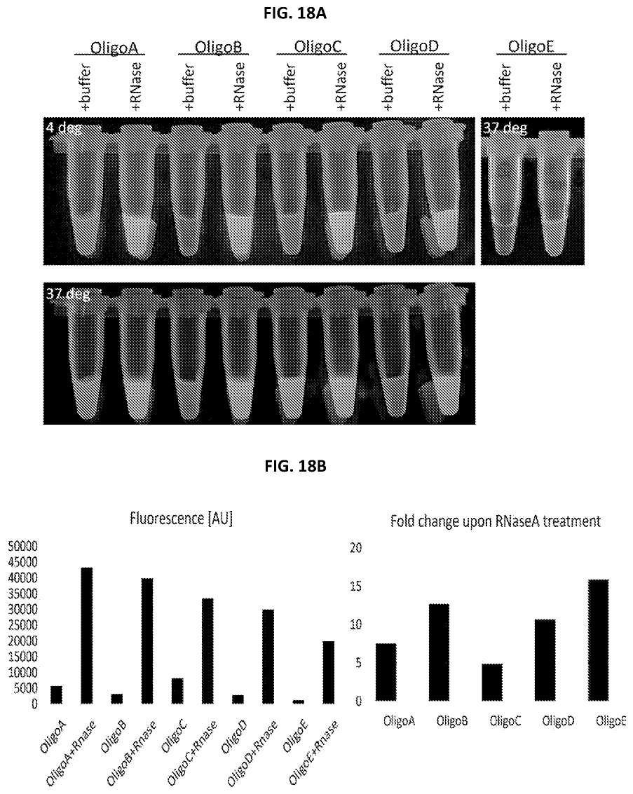

To enhance the flexibility and versatility of the SARS-CoV2 NSP14/NSP10 exonuclease FRET-based activity assay disclosed herein, a TexasRed-BHQ2 FRET pair was constructed in accordance with various aspects of reporter-quencher and dsRNA substrate design parameters described in detail above (see Example 1). Briefly, a fluorophore reporter TexasRed (TxRed) was attached to the 5′ end of the sense (5′-to-3′) strand of the dsRNA substrate design. TexasRed fluorescent dye is an attractive reporter as it can be acquired with excitation/emission of approximately 586/603 nm with a Tetramethylrhodamine (TRITC) or TexasRed filter common to most fluorescence detection equipment. To form the dsRNA substrate, the RNA sense strand was annealed to a complementary antisense strand (3′-to-5′) attached to a non-fluorescent Black Hole Quencher-2 (BHQ2) quencher molecule, which was positioned at the 3′ end of the antisense strand. BHQ2 is a dark quencher molecule exhibiting strong absorption from 560-670 nm, a wavelength spectrally optimal for TexasRed. The dsRNA substrate was identical to that of Oligo D, and as such differed from Oligo C by a single base pair (a guanine versus an adenine in the second base pair position 5′ to the quencher, respectively), exhibiting better signal-to-noise ratio due to a higher melting temperature. By comparison to Oligo B, Oligo E (as with Oligo D) was shorter in length, and thus by comparison had a slightly higher background signal and lower signal-to-noise. The reaction time for Oligo E was also faster than Oligo B, sufficient for complete digestion by RNase A. For an improved melting point, the 3′ end of the sequence of Oligo E contained a GC rich segment. Further, a single cytosine placed second from the TexasRed at the 5′ end stabilized the intact substrate at 37° C., resulting in lower background signal versus Oligo C. The uracil placed directly proximal to the TexasRed reporter minimized fluorescence quenching from the neighboring base for maximal assay sensitivity. The design for optimized Oligo E in the context of optimized Oligo A-D is presented in FIG. 17.

As an initial evaluation of the TexasRed-BHQ2 design (Oligo E) versus the 6FAM-BHQ1 designs (Oligo A-D), as described in detail above (see Example 1 and Example 2), annealed dsRNA substrate Oligo E (1 μM) was diluted in either: (1) reaction buffer (50 mM Tris-HCl, 2 mM MgCl₂, 2 mM DTT, pH 7.5) alone; or, (2) reaction buffer treated with RNase A (100 μg/ml). Following dilution, the sample was incubated at 37° C. for 1 h, and the reaction visualized and imaged for side-by-side comparison of fluorescence intensities across the different substrate designs. Images of the reactions are displayed in FIG. 18A.

For quantification of fluorescence intensity, the reaction for Oligo E (as with reactions for Oligo A-C, previously), prepared and incubated at 37° C. for 1 h as described above, was transferred to individual wells of a black, flat-well, low binding 96-well microplate. The microplate was then placed in a plate reader with excitation set at 590 nm (+/−9 nm) and emission detected at 620 nm (+/−20 nm). Fluorescence was measured and plotted in arbitrary units (AU), as shown in FIG. 18B (left). Fold change of fluorescence intensity upon RNase A treatment was calculated as the amount of fluorescence measured for the RNase A treatment group (AU) divided by the amount of fluorescence measured for the reaction buffer alone group (AU) for each dsRNA substrate, as presented in FIG. 18B (right). Together, these data confirm that Oligo E, as with Oligo A-D designs, is sufficient for use in the FRET-based activity assay, with Oligos B, D and E together displaying the best signal-to-noise ratio.

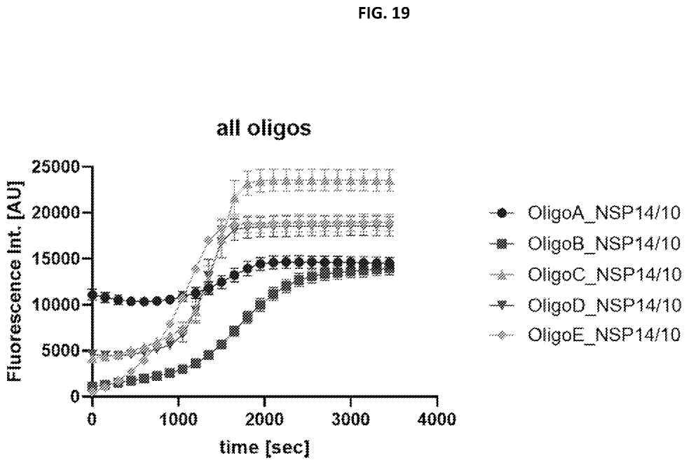

For kinetic profiling of NSP14/NSP10 complex activity on Oligo E, the FRET-based activity assay was used to quantify the time course of fluorescence intensity to reaction completion using methods as described above (see Example 4). As shown in FIG. 19, the time course to reaction completion for Oligo E aligned with those previously determined for Oligo A-C, reaching reaction completion by approximately 30 min (1800 sec).

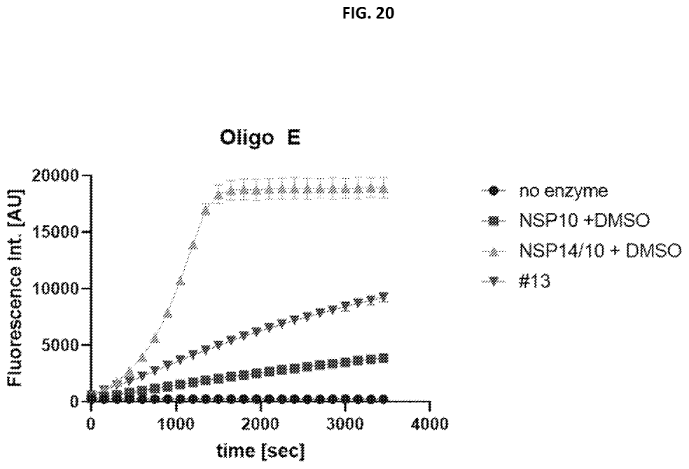

A description of the individual kinetic profiles for the activity of unbound NSP10, NSP14/NSP10 complex alone, and NSP14/NSP10 complex plus Hydroxyisoquinoline-1,3(2H,4H)-dione (labeled: #13; 500 μM) on Oligo E is presented in FIG. 20. Annealed double-stranded RNA substrate design Oligo E (1 μM) was diluted in reaction buffer (50 mM Tris-HCl, 2 mM MgCl₂, 2 mM DTT, pH 7.5) containing either unbound NSP10 (600 nM); NSP14 (200 nM) plus NSP10 (600 nM) alone (NSP14/10); or NSP14/10 plus Hydroxyisoquinoline-1,3(2H,4H)-dione (#13; 500 μM). These results demonstrated that as with Oligo A-D with the FAM-6-BHQ1 FRET pair, Oligo E with the TexasRed-BHQ2 FRET pair can be used for high-throughput screening of NSP14/NSP10 complex exonuclease activity. Hydroxyisoquinoline-1,3(2H,4H)-dione (Compound #13) at 500 M showed inhibition of NSP14/NSP10 complex exonuclease activity, similar to previous observations (see Example 5).

### Example 12. Gel Electrophoresis Assay

The same Oligo B as in the FRET based activity assay disclosed herein was used in the present gel electrophoresis assay. However, while the fluorophore reporter 6FAM was attached to the 5′ end of the sense (5′-to-3′) strand, the non-fluorescent Black Hole Quencher-1 (BHQ1) quencher molecule was absent from the annealed complementary antisense strand (3′-to-5′) at its 3′ end, as shown below. In the absence of the quencher molecule, Oligo B was thus constitutively fluorescent.

```
5′ FAM-UUGCCGAAUUAAGCGCCA (SEQ ID NO: 3)
              |||||||||||||||||
        3′-AACGGCUUAAUUCGCGGAAU (SEQ ID NO: 19)
```

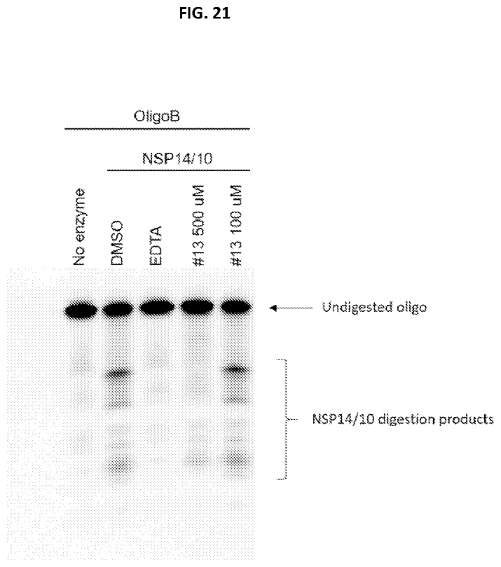

Reaction conditions were identical to the FRET assay, apart from the concentration of Oligo B, which was higher in this approach (5 μM) as compared to the FRET activity assay (1 μM). After an incubation period of 1 hour (37° C.), the reaction was stopped by adding 5× High-Density TBE-Sample Buffer. One fifth of the reaction volume was then loaded onto Novex TBE 20% Gels, and run for 60 minutes at 180V. Gels were visualized by a fluorescent gel documentation system (BioRad), as shown in FIG. 21. Degradation of Oligo B was observed as faster migrating bands versus inhibition of the NSP14/10 complex (Oligo B digestion), which resulted in a slower migration pattern. Inhibition of NSP14/10 by Hydroxyisoquinoline-1,3(2H,4H)-dione (#13) was greater at higher (500 μM) versus lower (100 μM) concentrations. EDTA was used as a control to show successful inhibition of the reaction. An additional control where no enzyme was added to the reaction mixture was used to show the undigested running pattern of the Oligo B. DMSO was used as a vehicle control for the compound, showing digestion of Oligo B by the NSP14/10 complex.

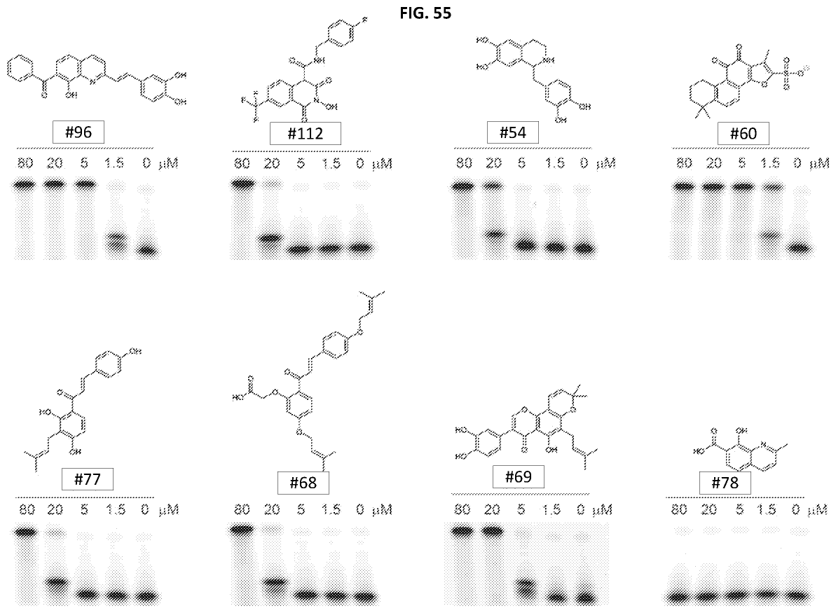

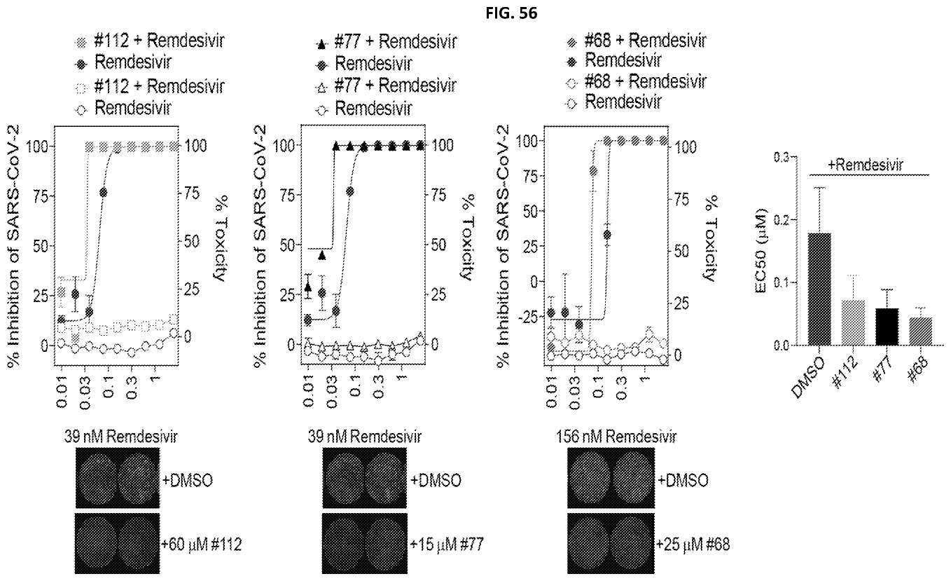

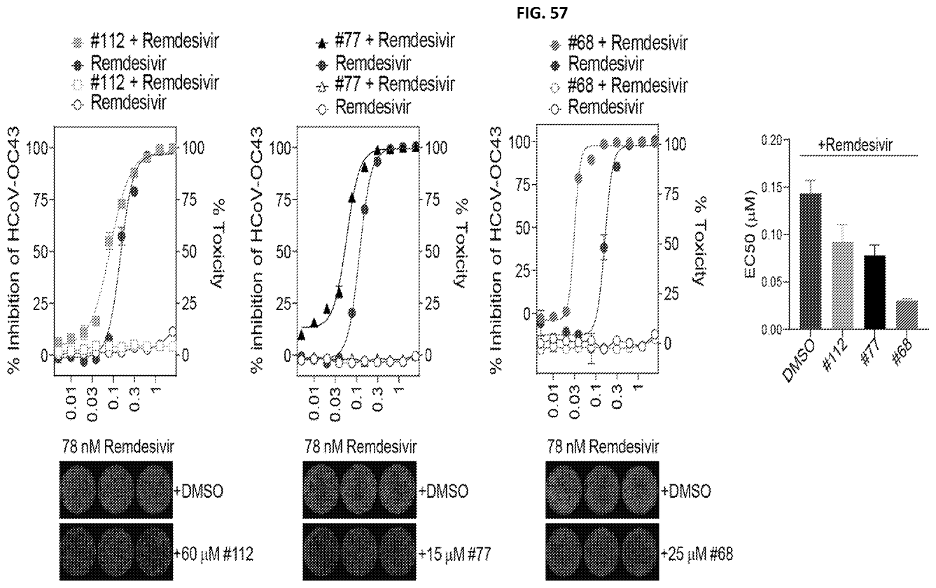

Identical gel electrophoresis assays were performed as above with compounds #96, #112, #54, #60, #77, #68, #69 and #78 (negative control) from Table 2 to measure their NSP14/NSP10 complex inhibitory potential. Inhibition of the NSP14/10 exonuclease activity results in the reduction of the full-length dsRNA oligo and increase in faster migrating bands. Compounds were used at the indicated concentrations as shown in FIG. 55. Results of the gel electrophoresis assay confirm the FRET-based assay, proving that these compounds (#96, #112, #54, #60, #77, #68, #69) are indeed novel NSP14 inhibitors.

### Example 13. Cultivation of SARS-CoV2

SARS-CoV2 (USA-WA1/2020, Cat. No.: NR-52281, BEI Resources) are cultivated on VeroE6 cells (ATCC). Culture supernatants are collected three days post infection and clarified by centrifugation. Titer is calculated by serially diluting virus on VeroE6 cells and performing a focus forming unit assay for virus infection by immunofluorescence detection of virus nucleoprotein (N).

### Example 14. Coronavirus Infection of A549-ACE2 Cell Lines

A549 cells with stable expression of the human ACE2 receptor, are plated onto tissue culture treated 96 well plates in DMEM with 10% FBS, 1% Penicillin-Streptomycin and 1% NEAA, 24 hours before infection. Cells are pre-treated for 2 hours with the compounds to be tested and their respective vehicle controls. Cells are infected for two hours with SARS-CoV2 with a multiplicity of infection (MOI) resulting in ˜50% infection efficiency allowing for multiple rounds of viral replication, and then are incubated for 48 hours at 37° C. in the presence of the compounds to be tested and their respective vehicle controls. After fixation with PFA, samples are assessed for infection efficiency via immunofluorescence detection of virus nucleoprotein (N). Mock infected wells are used to ensure the specificity of the immunofluorescence staining. Samples are imaged on an automated cell imaging multi-mode reader and are evaluated using CellProfiler or ImageJ. Infection efficiencies are calculated from the percentage of viral nucleoprotein positive-cells in each sample. Any defects in SARS-CoV2 infection or replication is observed as a decrease in N staining. Cell nuclei are counter stained with Hoechst 33342. Duplicate plates are generated for cell viability measurements to ensure that the concentration range used for the compounds is not toxic on their own for the cells. Cell viability is assessed with CellTiter-Glo® or alamarBlue™ following the recommendation of the manufacturer.

Representative graphs are shown of the antiviral activity (full symbols) and cytotoxicity (empty symbols) of compounds #112, #77 and #68 in A549-ACE2 cells infected with SARS-CoV-2. Compounds were applied at the following concentrations: #112: 60 μM, #77:15 μM, #68:25 μM. Remdesivir concentrations are indicated on the X-axis in μM. Error bars represents SEM. IF images of representative wells show anti-N staining (red) and DAPI signal (blue) at indicated drug concentrations. Graph shows the EC50 values from three independent experiments using technical triplicates. Compounds from Table 2 #112 (7-trifluoromethyl-N-(4-fluorobenzyl)-2-hydroxy-1,3-dioxo-4H-isoquinoline-4-carboxamide), #77 (Isobavachalcone), and #68 (Solfacone) showed synergistic effect with remdesivir, lowering the EC₅₀ values of remdesivir by ˜5 fold (FIG. 56).

### Example 15. Cultivation of HCoV-OC43

HMC3 cells were used for HCoV-OC43 infection assays. HCoV-OC43 viral stocks (ATCC VR-1558) were propagated/isolated as detailed below. MRC-5 cells were seeded at a density of 2×10⁶ cells in 100 mm dish. The next day, the cells were infected with 3×10⁶ pfu/ml of HCoV-OC43 and incubated at 33° C. for four days until 90-100% cells have cytopathic effects. The culture supernatant and infected cells were harvested, centrifuged at 1000×g for 5 min and the supernatant was stored at −80° C.

### Example 16. Coronavirus Infection of HMC3 Cell Line

Representative graphs are shown of the antiviral activity (full symbols) and cytotoxicity (empty symbols) of compounds #112, #77 and #68 in HMC3 cells infected with HCoV-OC43. Compounds were applied at the following concentrations: #112: 60 μM, #77:15 μM, #68:25 μM. Remdesivir concentrations are indicated on the X-axis in μM. Error bars represents SEM. IF images of representative wells show anti-N staining (red) and DAPI signal (blue) at indicated drug concentrations. Graph shows the EC₅₀ values from three independent experiments using technical triplicates. Compounds from Table 2 #112 (7-trifluoromethyl-N-(4-fluorobenzyl)-2-hydroxy-1,3-dioxo-4H-isoquinoline-4-carboxamide), #77 (Isobavachalcone), and #68 (Solfacone) showed synergistic effect with remdesivir, lowering the EC₅₀ values of remdesivir by ˜5 fold (FIG. 56).

### REFERENCES

1. Agostini et al., Coronavirus Susceptibility to the Antiviral Remdesivir (GS-5734) Is Mediated by the Viral Polymerase and the Proofreading Exoribonuclease. mBio. 2018 Mar. 6; 9(2):e00221-18.
2. Becares et al., Mutagenesis of Coronavirus nsp14 Reveals Its Potential Role in Modulation of the Innate Immune Response. J Virol. 2016 May 12; 90(11):5399-5414.
3. Bouvet et al., RNA 3′-end mismatch excision by the severe acute respiratory syndrome coronavirus nonstructural protein nsp10/nsp14 exoribonuclease complex. Proc Natl Acad Sci USA. 2012 May 24; 109(24):9372-9377.
4. Case et al., Murine Hepatitis Virus nsp14 Exoribonuclease Activity Is Required for Resistance to Innate Immunity. J Virol. 2017 Dec. 14; 92(1):e01531-17.
5. Eckerle et al., High fidelity of murine hepatitis virus replication is decreased in nsp14 exoribonuclease mutants. J Virol. 2007 November; 81(22):12135-44.
6. Ferron et al., Structural and molecular basis of mismatch correction and ribavirin excision from coronavirus RNA. Proc Natl Acad Sci USA. 2018 Jan. 9; 115(2):E162-E171.
7. Goyal et al., Inhibition of in vitro Ebola infection by anti-parasitic quinoline derivatives. F1000Res. 2020 Apr. 17; 9:268.
8. Graepel at al., Proofreading-Deficient Coronaviruses Adapt for Increased Fitness over Long-Term Passage without Reversion of Exoribonuclease-Inactivating Mutations. mBio. 2017 Nov. 7; 8(6):e01503-17.
9. Graham et al., A live, impaired-fidelity coronavirus vaccine protects in an aged, immunocompromised mouse model of lethal disease. Nat Med. 2012 December; 18(12):1820-6.
10. Ma et al., Structural basis and functional analysis of the SARS coronavirus nsp14-nsp10 complex. Proc Natl Acad Sci USA. 2015 Jul. 28; 112(30):9436-41.
11. Minkskaia et al., Discovery of an RNA virus 3′->5′ exoribonuclease that is critically involved in coronavirus RNA synthesis. Proc Natl Acad Sci USA. 2006 Mar. 28; 103(13):5108-13.
12. Mousnier et al., Nuclear import of HIV-1 integrase is inhibited in vitro by styrylquinoline derivatives. Mol Pharmacol. 2004 October; 66(4):783-8.
13. Orgando et al., The Curious Case of the Nidovirus Exoribonuclease: Its Role in RNA Synthesis and Replication Fidelity. Front Microbiol. 2019 Aug. 7; 10:1813.
14. Orgando et al., The Enzymatic Activity of the nsp14 Exoribonuclease Is Critical for Replication of MERS-CoV and SARS-CoV-2. J Virol. 2020 Nov. 9; 94(23):e01246-20.
15. Robson et al., Coronavirus RNA Proofreading: Molecular Basis and Therapeutic Targeting. Mol Cell. 2020 Sep. 3; 79(5):710-727.
16. Shannon et al., Remdesivir and SARS-CoV-2: Structural requirements at both nsp12 RdRp and nsp14 Exonuclease active-sites. Antiviral Res. 2020 June; 178:104793.
17. Silman et al., Identification and structure-activity relationship of 8-hydroxy-quinoline-7-carboxylic acid derivatives as inhibitors of Pim-1 kinase. Bioorg Med Chem Lett. 2010 May 1; 20(9):2801-5.
18. Smith et al., Coronaviruses lacking exoribonuclease activity are susceptible to lethal mutagenesis: evidence for proofreading and potential therapeutics. PLoS Pathog. 2013 August; 9(8):e1003565.
19. Yang. Nucleases: Diversity of Structure, Function, and Mechanism. Q Rev Biophys. 2011 February; 44(1)1-93.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1.-110. (canceled)

111. A fluorescence resonance energy transfer (FRET)-based method for measuring a 3′ to 5′ exonuclease activity in a sample, comprising:
(a) contacting the sample with a fluorescently labeled double-stranded RNA (dsRNA) substrate to create a test reaction mixture, wherein said dsRNA substrate comprises (i) at least one free 3′ OH group, and (ii) a pair of FRET probes comprising a fluorophore and a quencher, wherein one probe is located at the 5′ end of the strand comprising the free 3′ OH group and the other probe is located either at the 5′ end or at the 3′ end of the other strand of said dsRNA substrate, and when the substrate is uncleaved, the quencher quenches the fluorescence signal of the fluorophore;
(b) incubating said test reaction mixture under conditions and for a time sufficient for cleavage of the substrate by the 3′ to 5′ exonuclease, wherein the cleavage of the substrate by the 3′ to 5′ exonuclease causes sufficient separation of the fluorophore and the quencher to reduce quenching of the fluorescence signal of the fluorophore, and
(c) measuring the fluorescence signal emitted from the test reaction mixture.

112. A fluorescence resonance energy transfer (FRET)-based method for identifying and/or assessing a modulator of a 3′ to 5′ exonuclease, comprising:
(a) in a test reaction mixture, contacting the exonuclease with a test compound and a fluorescently labeled double-stranded RNA (dsRNA) substrate, wherein said dsRNA substrate comprises (i) at least one free 3′ OH group, and (ii) a pair of FRET probes comprising a fluorophore and a quencher, wherein one probe is located at the 5′ end of the strand comprising the free 3′ OH group and the other probe is located either at the 5′ end or at the 3′ end of the other strand of said dsRNA substrate, and when the substrate is uncleaved, the quencher quenches the fluorescence signal of the fluorophore;
(b) incubating said test reaction mixture under conditions and for a time sufficient for cleavage of the substrate by the exonuclease in the absence of the test compound, wherein the cleavage of the substrate by the exonuclease causes sufficient separation of the fluorophore and the quencher to reduce quenching of the fluorescence signal of the fluorophore;
(c) determining the fluorescence signal emitted from the test reaction mixture;
(d) comparing the fluorescence signal determined in step (c) to a control fluorescence signal, wherein the control fluorescence signal is the fluorescence signal determined under the same conditions in a control sample comprising the same amounts of exonuclease and dsRNA substrate but in the absence of the test compound, and
(e) (i) determining that the test compound is an inhibitor of the exonuclease if the fluorescence signal in the test reaction mixture is lower than in the control reaction mixture, or (ii) determining that the test compound is not an inhibitor of the exonuclease if the fluorescence signal in the test reaction mixture is not lower than in the control reaction mixture, or (iii) determining that the test compound is an activator of the exonuclease if the fluorescence signal in the test reaction mixture is higher than in the control reaction mixture.

113. A fluorescence resonance energy transfer (FRET)-based method for measuring processivity of a 3′ to 5′ exonuclease, comprising:
(a) contacting the exonuclease with a first fluorescently labeled double-stranded RNA (dsRNA) substrate to create a first reaction mixture, wherein said first dsRNA substrate comprises (i) at least one free 3′ OH group, and (ii) a pair of FRET probes comprising a fluorophore and a quencher, wherein one probe is located at the 5′ end of the strand comprising the free 3′ OH group and the other probe is located either at the 5′ end or at the 3′ end of the other strand of said first dsRNA substrate, and when the substrate is uncleaved, the quencher quenches the fluorescence signal of the fluorophore;
(b) contacting the exonuclease with a second dsRNA substrate to create a second reaction mixture, wherein the second dsRNA substrate differs from the first dsRNA substrate in that it is longer than the first substrate;
(c) incubating said first reaction mixture and said second reaction mixture under conditions and for a time allowing for cleavage of both substrates by the exonuclease, wherein the cleavage of the substrates by the exonuclease causes sufficient separation of the fluorophore and the quencher to reduce quenching of the fluorescence signal of the fluorophore, and
(d) determining the time required for the first reaction mixture and the second reaction mixture to reach the same level of fluorescence signal;
wherein the processivity of the exonuclease is measured as the difference in the length between the first and second substrate divided by the difference in the time required for the first reaction mixture and second reaction mixture to reach the same level of fluorescence signal.

114. A method of treating a viral infection in a subject comprising administering to the subject a compound capable of inhibiting enzymatic activity of the NSP14-NSP10 complex having the structure of Formula (I): 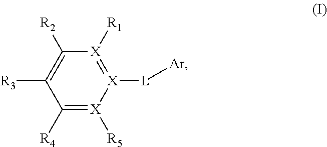
or a pharmaceutically acceptable salt thereof,
wherein X is independently at each occurrence selected from C, O, N and S;
L is a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar is phenyl or a 5- or 6-membered heterocycle comprising from 1 to 3 heteroatoms independently selected from N, O, and S, wherein Ar is optionally substituted with one or more groups R′;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least two of R₁, R₂, R₃, R₄ and R₅ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

115. A method of treating a viral infection in a subject comprising administering to the subject a compound capable of inhibiting enzymatic activity of the NSP14-NSP10 complex having the structure of Formula (II): 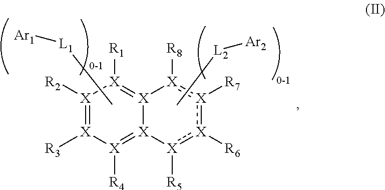
or a pharmaceutically acceptable salt thereof,
wherein X is independently at each occurrence selected from C, O, N, and S;
L₁ and L₂ are independently a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar₁ and Ar₂ are independently a phenyl, a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, R₇ and R₈ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

116. The method of claim 115, wherein the compound has the structure of Formula (IIA): 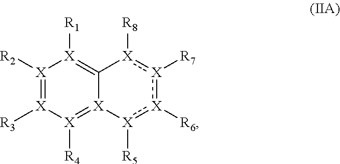
or a pharmaceutically acceptable salt thereof,
wherein X is independently at each occurrence selected from C, O, N, and S;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —F, —Cl, —Br, —I, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form one or more fused rings, and wherein at least two of R₁, R₂, R₃, R₄, R₅, R₆, R₇ and R₈ are not H;
R* is independently selected at each occurrence from hydrogen, C₁₋₁₂ alkyl, C₁₋₁₂ alkenyl, C₆₋₁₂ aryl, and C₁₋₁₂ aralkyl, each of which optionally contains 1-8 heteroatoms selected from halogen, O, N, and S and combinations thereof.

117. The method of claim 116, wherein the compound having structure of Formula (IIA) is selected from the group consisting of: 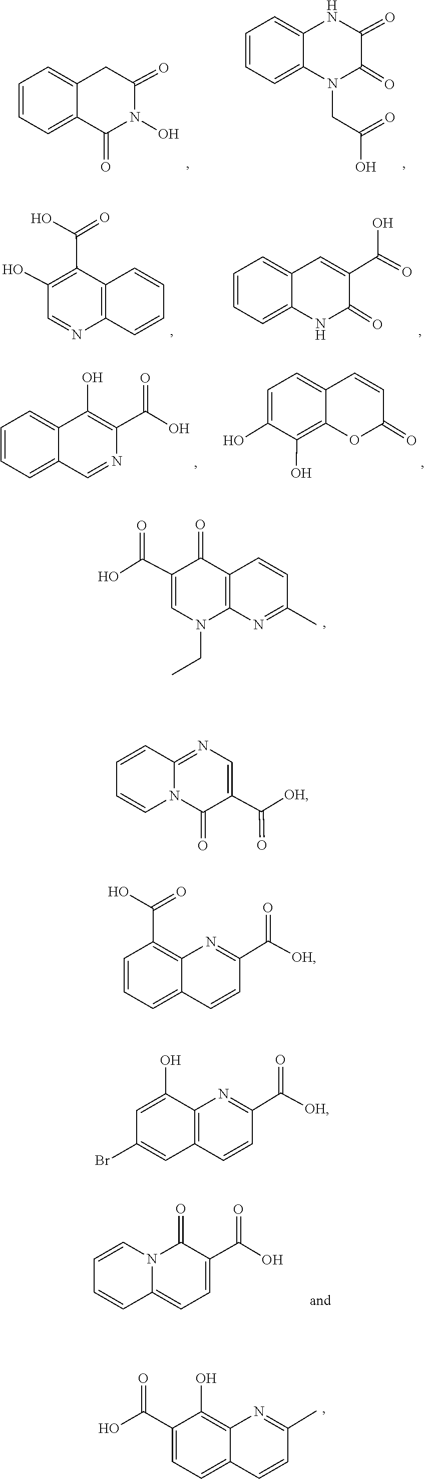
or a pharmaceutically acceptable salt thereof.

118. The method of claim 115, wherein the compound has the structure of Formula (IIB): 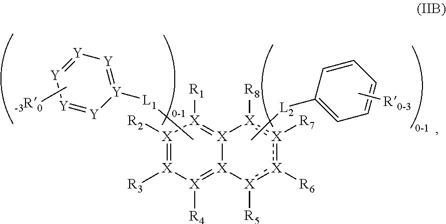
or a pharmaceutically acceptable salt thereof,
wherein X is independently at each occurrence selected from C, O, and N;
wherein Y is independently at each occurrence selected from a bond, C, O, and N
L₁ and L₂ are independently a linker selected from a bond and a C₁₋₃ alkyl optionally containing 1-2 heteroatoms selected from O, N, and S;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

119. The method of claim 118, wherein the compound having the structure of Formula (IIB) is selected from the group consisting of: 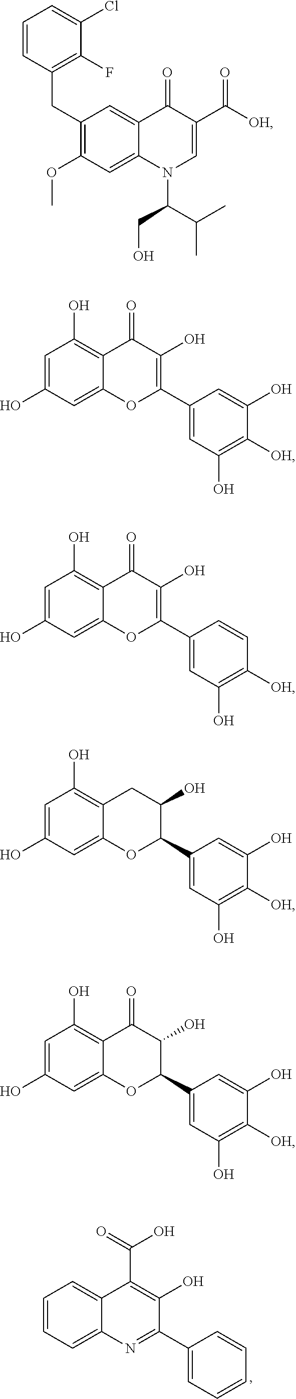 [SMILES: COc1cc2c(cc1Cc1cccc(Cl)c1F)c(=O)c(C(=O)O)cn2[C@H](CO)C(C)C ; O=c1c(O)c(-c2cc(O)c(O)c(O)c2)oc2cc(O)cc(O)c12 ; O=c1c(O)c(-c2ccc(O)c(O)c2)oc2cc(O)cc(O)c12 ; Oc1cc(O)c2c(c1)O[C@H](c1cc(O)c(O)c(O)c1)[C@H](O)C2 ; O=C1c2c(O)cc(O)cc2O[C@H](c2cc(O)c(O)c(O)c2)[C@H]1O ; O=C(O)c1c(O)c(-c2ccccc2)nc2ccccc12] 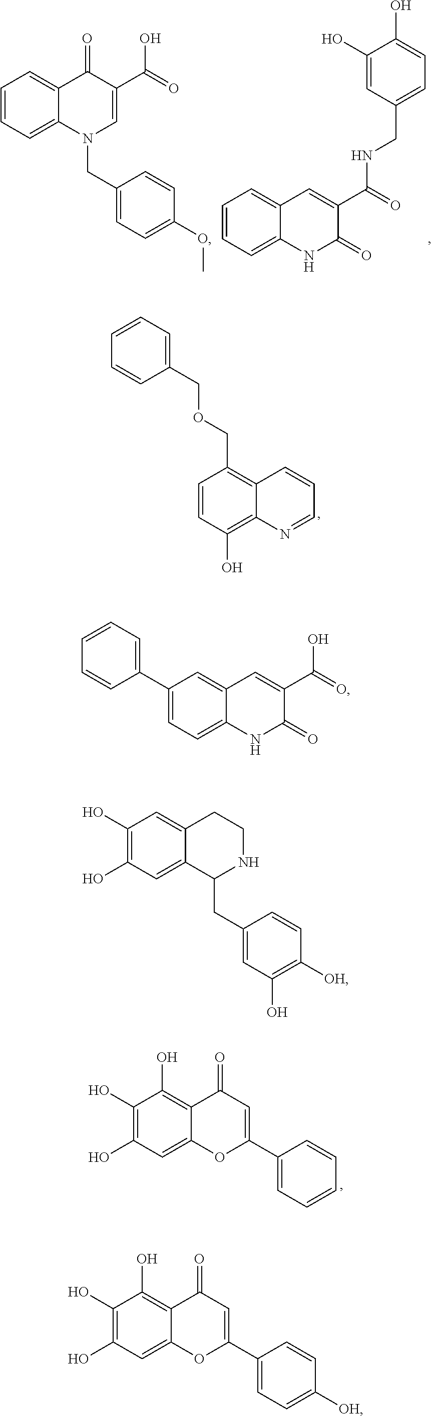 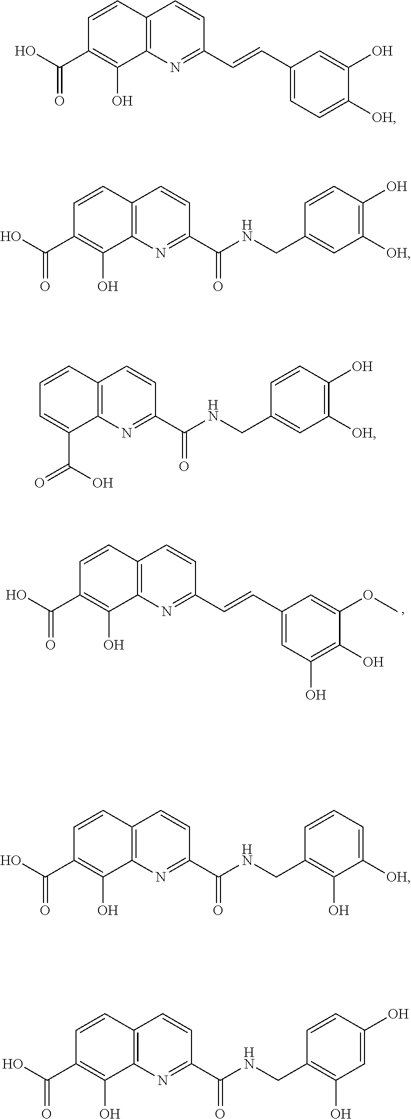 [SMILES: O=C(O)c1ccc2ccc(/C=C/c3ccc(O)c(O)c3)nc2c1O ; O=C(NCc1ccc(O)c(O)c1)c1ccc2ccc(C(=O)O)c(O)c2n1 ; O=C(NCc1ccc(O)c(O)c1)c1ccc2cccc(C(=O)O)c2n1 ; COc1cc(/C=C/c2ccc3ccc(C(=O)O)c(O)c3n2)cc(O)c1O ; O=C(NCc1cccc(O)c1O)c1ccc2ccc(C(=O)O)c(O)c2n1 ; O=C(NCc1ccc(O)cc1O)c1ccc2ccc(C(=O)O)c(O)c2n1] 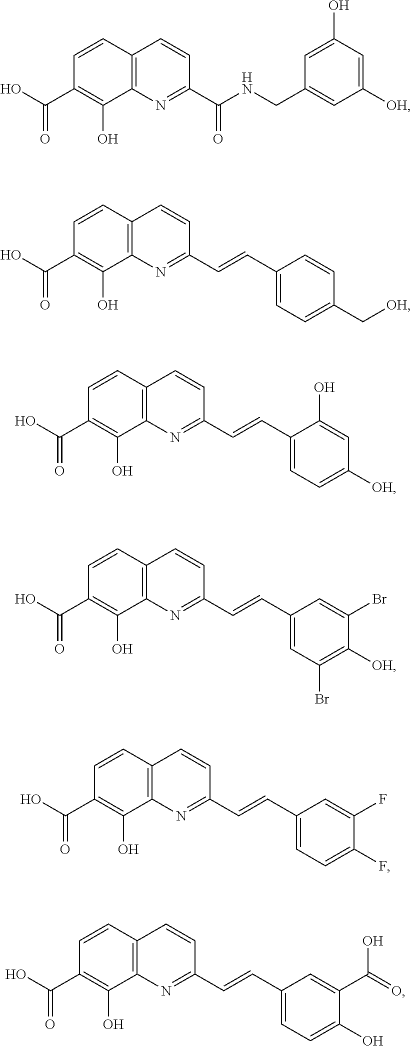 [SMILES: O=C(NCc1cc(O)cc(O)c1)c1ccc2ccc(C(=O)O)c(O)c2n1 ; O=C(O)c1ccc2ccc(/C=C/c3ccc(CO)cc3)nc2c1O ; O=C(O)c1ccc2ccc(/C=C/c3ccc(O)cc3O)nc2c1O ; O=C(O)c1ccc2ccc(/C=C/c3cc(Br)c(O)c(Br)c3)nc2c1O ; O=C(O)c1ccc2ccc(/C=C/c3ccc(F)c(F)c3)nc2c1O ; O=C(O)c1cc(/C=C/c2ccc3ccc(C(=O)O)c(O)c3n2)ccc1O] 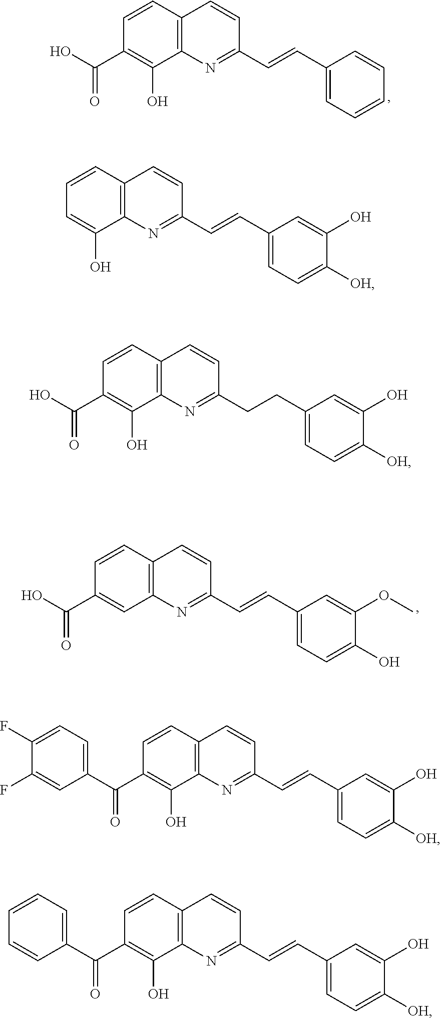 [SMILES: O=C(O)c1ccc2ccc(/C=C/c3ccccc3)nc2c1O ; Oc1ccc(/C=C/c2ccc3cccc(O)c3n2)cc1O ; O=C(O)c1ccc2ccc(CCc3ccc(O)c(O)c3)nc2c1O ; COc1cc(/C=C/c2ccc3ccc(C(=O)O)cc3n2)ccc1O ; O=C(c1ccc(F)c(F)c1)c1ccc2ccc(/C=C/c3ccc(O)c(O)c3)nc2c1O ; O=C(c1ccccc1)c1ccc2ccc(/C=C/c3ccc(O)c(O)c3)nc2c1O] 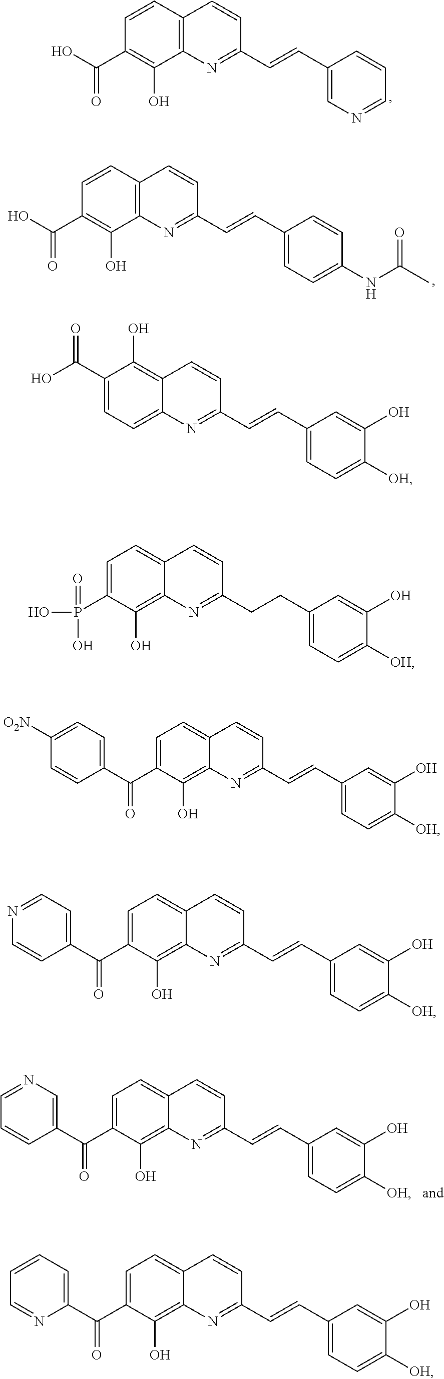
or a pharmaceutically acceptable salt thereof.

120. The method of claim 118, wherein the compound of Formula (IIB) has the structure selected from the group consisting of: 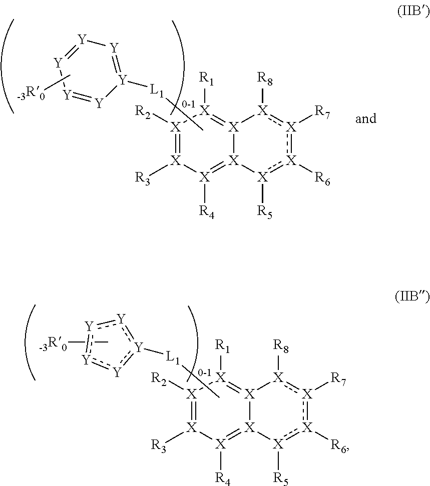
or a pharmaceutically acceptable salt thereof.

121. The method of claim 120, wherein the compound having the structure of Formula (IIB′) is selected from the group consisting of: 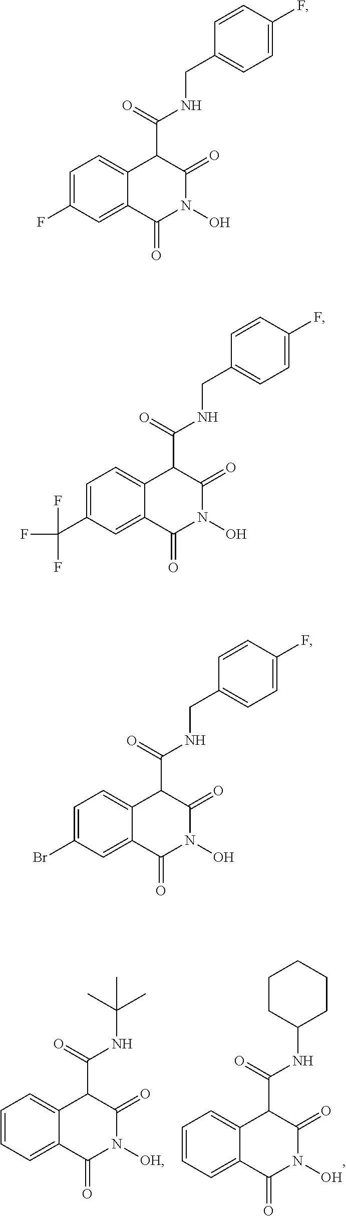 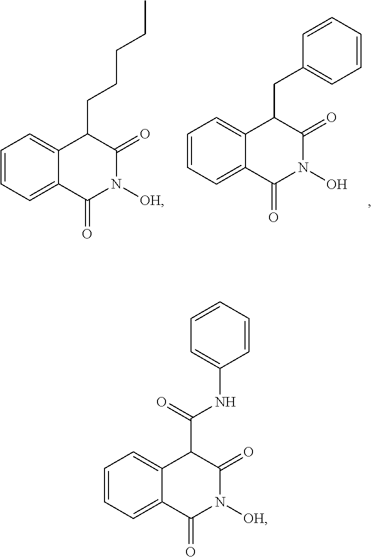
or a pharmaceutically acceptable salt thereof, or wherein the compound having the structure of Formula (IIB″) is selected from the group consisting of: 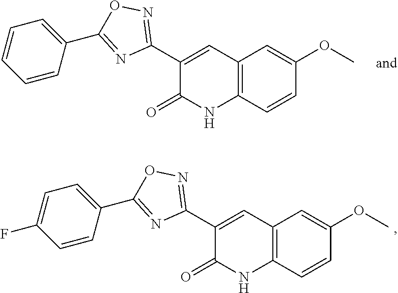 [SMILES: COc1ccc2[nH]c(=O)c(-c3noc(-c4ccccc4)n3)cc2c1 ; COc1ccc2[nH]c(=O)c(-c3noc(-c4ccc(F)cc4)n3)cc2c1]
or a pharmaceutically acceptable salt thereof.

122. The method of claim 115, wherein the compound has the structure of Formula (IIC): 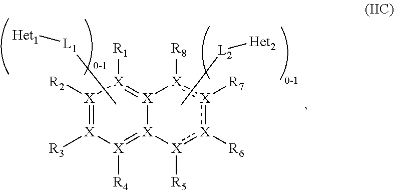
or a pharmaceutically acceptable salt thereof,
wherein X is independently at each occurrence selected from C, O, and N;
L₁ and L₂ are independently a linker selected from a bond and a C₁₋₃ alkyl optionally containing 1-2 heteroatoms selected from O, N, and S;
Het₁ and Het₂ are independently a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, R₇ and R₈ are independently at each occurrence H, optionally substituted C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NI—H₂; —O—(C═O)—NI—H₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

123. The method of claim 122, wherein the compound having the structure of Formula (IIC) is selected from the group consisting of: 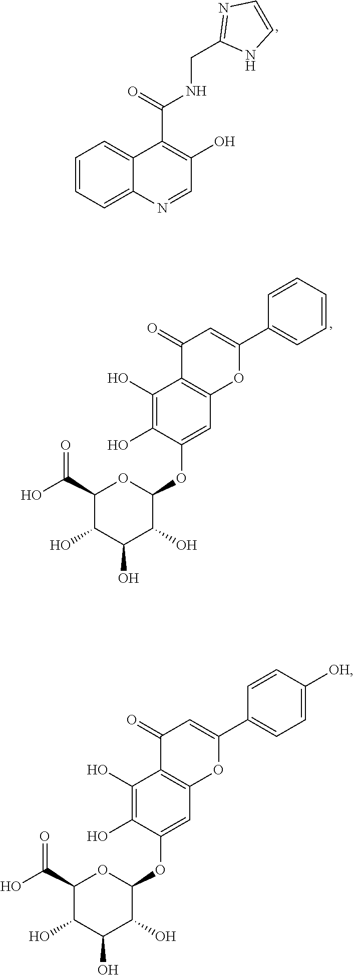 [SMILES: O=C(NCc1ncc[nH]1)c1c(O)cnc2ccccc12 ; O=C(O)[C@H]1O[C@@H](Oc2cc3oc(-c4ccccc4)cc(=O)c3c(O)c2O)[C@H](O)[C@@H](O)[C@@H]1O ; O=C(O)[C@H]1O[C@@H](Oc2cc3oc(-c4ccc(O)cc4)cc(=O)c3c(O)c2O)[C@H](O)[C@@H](O)[C@@H]1O] 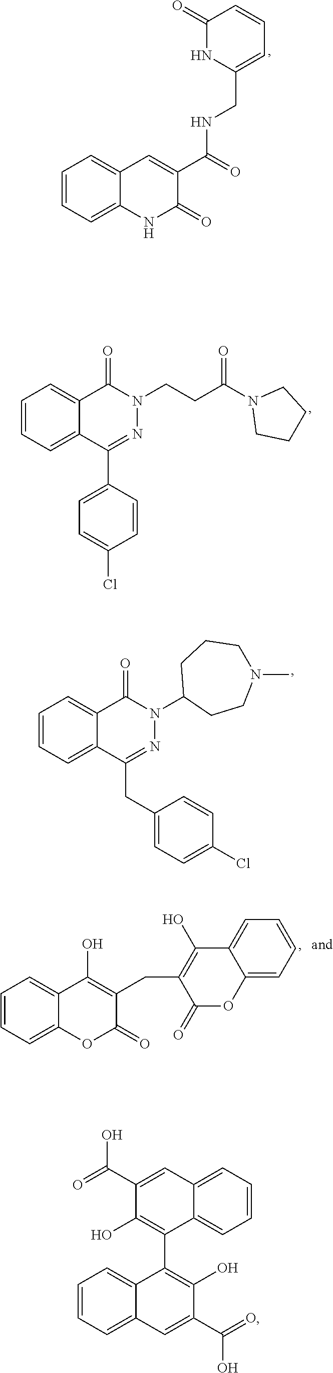 [SMILES: O=C(NCc1cccc(=O)[nH]1)c1cc2ccccc2[nH]c1=O ; O=C(CCn1nc(-c2ccc(Cl)cc2)c2ccccc2c1=O)N1CCCC1 ; CN1CCCC(n2nc(Cc3ccc(Cl)cc3)c3ccccc3c2=O)CC1 ; O=c1oc2ccccc2c(O)c1Cc1c(O)c2ccccc2oc1=O ; O=C(O)c1cc2ccccc2c(-c2c(O)c(C(=O)O)cc3ccccc23)c1O]
or a pharmaceutically acceptable salt thereof.

124. The method of claim 115, wherein the adjacent two or more of R₁, R₂, R₃, R₄, R₅, R₆, R₇ and R₈ combine to form one or more fused rings, which may be further substituted with one or more substituents to form a fused polycyclic ring system.

125. A method of treating a viral infection in a subject comprising administering to the subject a compound capable of inhibiting enzymatic activity of the NSP14-NSP10 complex having the structure of Formula (II′): 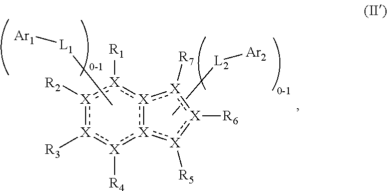
or a pharmaceutically acceptable salt thereof,
wherein X is independently at each occurrence selected from C, O, N, and S;
L₁ and L₂ are independently a linker selected from a bond, a C₁₋₃ alkyl, C₂₋₄ alkenyl, —CO—, —CO—NH—, —CO—(C₁₋₃ alkyl)-, and —CO—(C₂₋₄ alkenyl)-, wherein the C₁₋₃ alkyl optionally contains 1-2 heteroatoms selected from O, N, and S;
Ar₁ and Ar₂ are independently a phenyl, a 5-, 6-, or 7-membered heterocycle comprising from 1 to 3 heteroatoms independently selected from N, O, and S, or a fused bicyclic ring system optionally comprising from 1 to 3 heteroatoms independently selected from N, O, and S, wherein Ar₁ or Ar₂ is optionally substituted with one or more groups R′;
R₁, R₂, R₃, R₄, R₅, R₆, and R₇ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, or adjacent two moieties combine to form a fused ring, and wherein at least two of R₁, R₂, R₃, R₄, R₅, R₆, R₇ and R₈ are not H;
R′ is independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

126. A method of treating a viral infection in a subject comprising administering to the subject a compound capable of inhibiting enzymatic activity of the NSP14-NSP10 complex having the structure of Formula (IIIA): 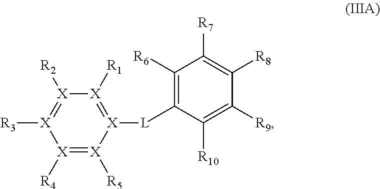
or a pharmaceutically acceptable salt thereof,
wherein X is independently at each occurrence selected from C, N, O, and S;
L is a linker selected from a bond, a C₁₋₁₂ alkyl, C₂₋₁₂ alkenyl, —CO—, —CO—NH—, —CO—(C₁₋₃ alkyl)-, and —CO—(C₂₋₄ alkenyl)-, and combinations thereof, wherein the C₁₋₁₂ alkyl or the C₂₋₁₂ alkenyl optionally contains 1-5 heteroatoms selected from O, N, and S;
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least one of R₁, R₂, R₃, R₄ and R₅ is not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

127. A method of treating a viral infection in a subject comprising administering to the subject a compound capable of inhibiting enzymatic activity of the NSP14-NSP10 complex having the structure of Formula (IIIB): 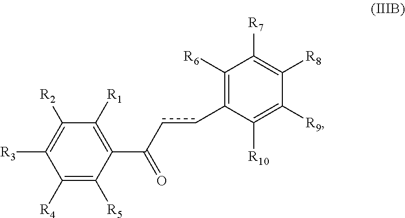
or a pharmaceutically acceptable salt thereof,
R₁, R₂, R₃, R₄ and R₅ are independently at each occurrence H, C₁₋₁₂ alkyl, C₁₋₁₂ alkenyl, C₆₋₁₂ aryl, C₁₋₁₂ aralkyl, C₁₋₄ haloalkyl, —OH, ═O, —CO₂H, —NO₂, —N—OH, —HSO₃, —H₂PO₃, —OR*, —(C═O)—R*, —CO₂R*, —CO—NHR*, —SO₂—NHR*, and wherein at least one of R₁, R₂, R₃, R₄ and R₅ is not H;
R₆, R₇, R₈, R₉ and Rio are independently at each occurrence C₁₋₁₂ alkyl, C₁₋₁₂ alkenyl, C₆₋₁₂ aryl, C₁₋₁₂ aralkyl, C₁₋₄ haloalkyl, a heteroaryl C₁-C₁₂ hydrocarbon, a C₁-C₁₂ perfluorocarbon, or a combination thereof, each of which optionally contains 1-8 heteroatoms selected from halogen, O, N, and S, and each of which is optionally substituted with one or more of —F; —Cl; —Br; —I; —OH, —OR*; —NO; —NO₂; —NO₃; —O—NO; —N₃; —NH₂; —NHR*; —N(R*)₂; —N(R*)₃+; —N(R*)—OH; —O—N(R*)₂; —N(R*)—O—R*; —CN; —NC; —(C═O)—R*; —CHO; —CO₂H; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NI—H₂; —O—(C═O)—NHNH₂; —(C═S)—NH₂; —(C═S)—N(R*)₂; —N(R*)—CHO; —N(R*)—(C═O)—R*; —SCN; —NCS; —NSO; —SSR*; —SO₂R*; —SO₂—N(R*)₂; —S(═O)—OR*; —S(═O)—R*; —Si(R*)₃; —CF₃; —O—CF₃; —P(R*)₂; —O—P(═O)(OR*)₂; —P(═O)(OR*)₂ and combinations thereof, and
R* is independently selected at each occurrence from hydrogen or C₁-C₁₂ hydrocarbons each of which optionally contains 1-8 heteroatoms selected from halogen, O, N, and S and combinations thereof.

128. A method of treating a viral infection in a subject comprising administering to the subject a compound capable of inhibiting enzymatic activity of the NSP14-NSP10 complex having the structure selected from the group presented in Table 2, or a pharmaceutically acceptable salt thereof.

129. The method of claim 115, wherein the viral infection is a SARS-CoV, a SARS-CoV2, a MERS-CoV, a HCoV-229E, a HCoV-NL63, a HCoV-HKU1 or a HCoV-OC43 infection or involves a coronavirus strain of animal or zoonotic origins.

130. The method of claim 115 wherein the method further comprises administering a ribonucleotide analog to the subject.


SEQUENCE LISTING (27 sequences)

SEQ ID NO: 1 - RNA, 17 nt, Artificial Sequence
  Description of Artificial Sequence Synthetic oligonucleotide
uugccgaauuaagcgca

SEQ ID NO: 2 - RNA, 17 nt, Artificial Sequence
  Description of Artificial Sequence Synthetic oligonucleotide
uaagcgcuuaauucggc

SEQ ID NO: 3 - RNA, 18 nt, Artificial Sequence
  Description of Artificial Sequence Synthetic oligonucleotide
uugccgaauuaagcgcca

SEQ ID NO: 4 - RNA, 20 nt, Artificial Sequence
  Description of Artificial Sequence Synthetic oligonucleotide
uaaggcgcuuaauucggcaa

SEQ ID NO: 5 - RNA, 13 nt, Artificial Sequence
  Description of Artificial Sequence Synthetic oligonucleotide
uuuuuucggccca

SEQ ID NO: 6 - RNA, 17 nt, Artificial Sequence
  Description of Artificial Sequence Synthetic oligonucleotide
aaauagggccgaaaaaa

SEQ ID NO: 7 - RNA, 13 nt, Artificial Sequence
  Description of Artificial Sequence Synthetic oligonucleotide
ucuuuucggccca

SEQ ID NO: 8 - RNA, 17 nt, Artificial Sequence
  Description of Artificial Sequence Synthetic oligonucleotide
aaauagggccgaaaaga

SEQ ID NO: 9 - RNA, 13 nt, Artificial Sequence
  Description of Artificial Sequence Synthetic oligonucleotide
ucuuuucggccca

SEQ ID NO: 10 - RNA, 17 nt, Artificial Sequence
  Description of Artificial Sequence Synthetic oligonucleotide
aaauagggccgaaaaga

SEQ ID NO: 11 - RNA, 13 nt, Artificial Sequence
  Description of Artificial Sequence Synthetic oligonucleotide
ucuuuucggccca

SEQ ID NO: 12 - RNA, 13 nt, Artificial Sequence
  Description of Artificial Sequence Synthetic oligonucleotide
ucuuuucggccca

SEQ ID NO: 13 - RNA, 13 nt, Artificial Sequence
  Description of Artificial Sequence Synthetic oligonucleotide
ucuuuucggccca

SEQ ID NO: 14 - RNA, 17 nt, Artificial Sequence
  Description of Artificial Sequence Synthetic oligonucleotide
aaauagggccgaaaaga

SEQ ID NO: 15 - DNA, 1629 bp, Artificial Sequence
  Description of Artificial Sequence Synthetic polynucleotide
atggccgaaaatgtcacgggtctgttcaaagattgttccaaagttatcacgggtctgcatccgacgcaagcaccgacgcacctgtctgtcgataccaaatttaaaacggaaggcctgtgcgtggatattccgggtatcccgaaagacatgacgtatcgtcgcctgattagcatgatgggcttcaaaatgaactatcaagtgaatggttacccgaacatgtttattacccgtgaagaagccatccgtcatgtgcgcgcatggatcggcttcgatgttgaaggttgtcacgcaacgcgcgaagctgttggcaccaatctgccgctgcagctgggctttagcacgggtgtgaacctggttgcggtgccgaccggttatgttgataccccgaacaatacggacttcagtcgcgtgtccgcaaaaccgccgccgggtgatcagtttaaacatctgattccgctgatgtacaaaggtctgccgtggaatgtggttcgtattaaaatcgttcaaatgctgtcagataccctgaaaaacctgtcggaccgcgtcgtgttcgttctgtgggcacacggctttgaactgacgtctatgaaatatttcgtcaaaatcggtccggaacgcacctgctgtctgtgcgatcgtcgcgcgacctgtttttcaacggcgtcggacacctacgcctgctggcatcacagtattggcttcgattatgtctacaatccgtttatgatcgacgtgcagcaatggggcttcaccggtaatctgcagtcgaaccatgatctgtattgccaagtccatggtaacgcgcacgtggccagctgtgacgccattatgacgcgttgcctggcagtgcacgaatgttttgttaaacgcgtcgattggaccattgaatatccgattatcggcgacgaactgaaaatcaatgcggcctgtcgtaaagtgcagcacatggttgtcaaagcagctctgctggccgataaatttccggttctgcacgacattggtaacccgaaagctatcaaatgcgtgccgcaggcggatgttgaatggaaattctatgatgcccaaccgtgttctgacaaagcatacaaaatcgaagaactgttttatagttacgctacgcattccgataaattcaccgacggcgtttgcctgttttggaactgtaatgtcgatcgctatccggccaatagtatcgtttgccgttttgacacccgcgtcctgtccaacctgaatctgccgggttgtgatggcggttcactgtacgtcaataaacatgcatttcacaccccggctttcgacaaatccgcgtttgttaacctgaaacagctgccgtttttctattacagcgattctccgtgcgaaagccatggcaaacaagtggtttctgatattgactatgtgccgctgaaaagtgcgacctgcatcacgcgttgtaacctgggcggtgctgtttgtcgtcatcacgcgaatgaatatcgcctgtacctggatgcttacaacatgatgattagcgcgggtttctctctgtgggtgtataaacagtttgatacctacaacctgtggaatacgtttacccgtctgcaaggcggcggttctggcggcggctcccaccaccaccatcaccattaa

SEQ ID NO: 16 - protein, 542 aa, Artificial Sequence
  Description of Artificial Sequence Synthetic polypeptide
MAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQGGGSGGGSHHHHHH

SEQ ID NO: 17 - DNA, 465 bp, Artificial Sequence
  Description of Artificial Sequence Synthetic polynucleotide
atggctggtaatgctacggaagtcccggcaaatagtacggtgctgtcgttctgtgcgttcgctgttgatgcggcaaaagcctacaaagattatctggccagtggcggtcagccgattacgaactgcgttaaaatgctgtgtacccataccggtaccggtcaggccatcaccgtcaccccggaagccaatatggaccaagaatcttttggcggtgcatcttgctgtctgtattgccgttgtcatattgatcacccgaacccgaaaggcttctgcgacctgaagggtaaatacgtccagatcccgaccacgtgtgctaacgatccggtgggctttaccctgaaaaatacggtgtgcaccgtttgtggcatgtggaaaggctacggttgcagctgtgaccaactgcgcgaaccgatgctgcaaggcggcggctcaggcggcggctcacaccaccaccaccatcattaa

SEQ ID NO: 18 - protein, 154 aa, Artificial Sequence
  Description of Artificial Sequence Synthetic polypeptide
MAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQGGGSGGGSHHHHHH

SEQ ID NO: 19 - RNA, 20 nt, Artificial Sequence
  Description of Artificial Sequence Synthetic oligonucleotide
uaaggcgcuuaauucggcaa

SEQ ID NO: 20 - DNA, 1629 bp, Artificial Sequence
  Description of Artificial Sequence Synthetic polynucleotide
atggctgaaaatgtaacaggactatttaaagactgttccaaaatcatcaccggcctgcatccgacccaggcaccgactcacctgtccgtggatatcaaatttaaaaccgagggtctgtgcgtggatattccgggtattccgaaagatatgacctaccgtcgtctgatttctatgatgggtttcaagatgaattatcaggttaatggttacccgaatatgttcatcacgcgtgaagaggcaattcgtcatgttcgtgcttggattggattcgacgttgaaggctgccatgctaccagagatgctgtgggtacaaaccttccactgcaactgggtttctcgaccggggtgaacttggttgctgtcccaacgggctacgtggacactgagaacaacaccgagttcacccgagttaatgcgaaaccgcctccgggcgaccagtttaaacacctgatcccgttgatgtataaaggtctgccgtggaatgttgtgcgtattaagatcgtacaaatgctgtccgacaccctgaagggtcttagcgaccgcgttgtctttgtgctgtgggcacatggcttcgaattgaccagcatgaaatacttcgtgaaaatcggtccggagcgtacctgctgtctgtgtgataagcgcgcgacgtgctttagcacctctagcgacacttacgcctgttggaaccacagcgtaggcttcgactacgtgtataacccatttatgatcgatgtgcagcagtggggtttcaccggtaacctgcaaagcaaccacgatcagcattgtcaggttcacggcaacgcgcatgttgccagctgtgatgcgatcatgactcgttgcctggcggtccacgagtgctttgttaaacgtgttgactggtccgtcgagtacccgattatcggcgacgaactgcgtgtgaacagcgcgtgccgtaaagtccagcacatggttgtgaagtccgcgctcctggccgataagttcccggttctgcacgacatcggcaacccgaaagcaatcaagtgcgtaccgcaggcggaagttgagtggaaattttacgatgcgcagccgtgcagcgacaaggcgtacaagattgaagagttgttctacagttatgctacgcatcacgataagtttaccgatggtgtgtgcttgttttggaactgcaacgtcgaccgttatccggcgaatgccattgtttgtcgctttgatacccgcgtgttgtctaatttgaatttacccgggtgcgacggcggttcgctgtatgtgaacaaacatgcgttccataccccggcattcgataagtcagccttcaccaatctcaagcaactgccgtttttttattatagcgacagcccgtgcgaaagccatggtaaacaagtcgttagcgacatcgactacgtgccgttgaagagcgcgacatgcatcacccgttgtaacttgggcggtgccgtgtgccgccatcacgcgaatgaataccgccagtatctggatgcgtataacatgatgattagcgcgggtttctctctgtggatttataagcaattcgacacgtacaacttatggaacacctttacccgcctgcaaggtggcggctctggtggcggctcccaccaccaccaccaccactaa

SEQ ID NO: 21 - protein, 542 aa, Artificial Sequence
  Description of Artificial Sequence Synthetic polypeptide
MAENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSDSPCESHGKQVVSDIDYVPLKSATCITRCNLGGAVCRHHANEYRQYLDAYNMMISAGFSLWIYKQFDTYNLWNTFTRLQGGGSGGGSHHHHHH

SEQ ID NO: 22 - DNA, 465 bp, Artificial Sequence
  Description of Artificial Sequence Synthetic polynucleotide
atggcaggaaatgcgacagaagtacccgctaacagcaccgtcctgagcttctgcgcgtttgcagttgatccagcgaaggcgtataaagattacttggcttccggcggccaaccgattaccaattgcgtgaagatgctgtgtacccatactggtacaggccaagcgatcacggttaccccggaagccaacatggatcaagagtcgttcggcggcgcatcttgttgcctgtactgccgttgtcatatcgaccatccgaatccgaaaggcttttgcgacttaaagggtaaatacgtgcagattccgaccacgtgcgctaacgacccggttggtttcaccttgcgcaacaccgtatgcaccgtgtgcggtatgtggaaaggttatggttgtagctgcgaccagctgcgtgagccgctgatgcagggtggcggttccggtggtggcagccatcaccaccaccaccactaa

SEQ ID NO: 23 - protein, 154 aa, Artificial Sequence
  Description of Artificial Sequence Synthetic polypeptide
MAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQGGGSGGGSHHHHHH

SEQ ID NO: 24 - DNA, 1620 bp, Artificial Sequence
  Description of Artificial Sequence Synthetic polynucleotide
atgtcacaaatagtaacaggactatttaaagattgtagccgtgagaccagcggcttgtccccggcgtacgcaccgacctatgtgagcgttgatgataaatacaagacctctgatgaactttgcgtgaacttgaatttaccggctaatgtcccgtacagtcgtgttattagccgcatgggcttcaaattagacgctacggttccgggctacccgaaactattcatcacccgggaggaagcggttcgtcaggttcgtagctggatcggtttcgatgtggaaggtgcccacgcctcgcgcaacgcctgcggtacaaatgttccgctgcagctgggtttcagcaccggtgttaattttgttgtccaaccagtgggcgtggtcgacaccgagtggggtaacatgctgacgggtatcgccgctcgcccacccccgggtgaacagtttaagcacctggttccgctgatgcataaaggtgcggcgtggcctattgtgcgccgtcgtattgtacaaatgttgtcggacacgctggataagttgtccgactactgtaccttcgtgtgctgggcacacggcttcgagctgacctctgcgtcctacttctgcaaaatcggcaaagagcagaaatgttgcatgtgtaaccgtcgcgctgctgcgtacagcagcccgctgcaaagctatgcgtgctggacccactcttgcggttacgattatgtctataacccgttttttgtcgacgttcaacagtggggttacgtaggcaacctggcgactaaccacgatcgttattgcagcgtgcaccagggtgcgcacgtggcctcaaatgatgcgatcatgacccgttgcctggcgattcatagctgctttatcgagcgcgtggactgggatatcgaatacccgtatatcagccatgaaaaaaaactgaacagctgctgccgtattgtcgaacgcaatgtggtacgtgcagcgctgctggcgggtagctttgacaaggtctacgatatcggtaacccgaagggcatcccgattgttgacgatccggttgtggactggcattattttgacgcccagccgttgacccgcaaagtgcaacaactgttctacactgaggatatggcaagccgttttgcggatggtctctgcctgttctggaattgtaacgttccgaagtacccaaataacgcaattgtttgccgttttgacactcgcgttcacagcgaattcaacctgccggggtgcgacggcggttcgctgtatgtgaataagcatgcgtttcataccccggcgtacgacgtgtctgcgtttcgtgatctgaagccgctcccgtttttctattactccacgaccccgtgtgaagttcatggtaacggttctatgattgaggacatcgactacgtgccgcttaagagcgcggtttgcattaccgcatgcaacttaggaggcgcggtgtgtcgtaagcacgcaaccgagtatcgtgagtatatggaagcgtacaacttggtttccgcttccggcttccgcctgtggtgttacaagacgttcgacatctataacctgtggtccaccttcaccaaagttcagggcggcggctcgggtggcggcagccatcaccaccatcaccactaa

SEQ ID NO: 25 - protein, 539 aa, Artificial Sequence
  Description of Artificial Sequence Synthetic polypeptide
MSQIVTGLFKDCSRETSGLSPAYAPTYVSVDDKYKTSDELCVNLNLPANVPYSRVISRMGFKLDATVPGYPKLFITREEAVRQVRSWIGFDVEGAHASRNACGTNVPLQLGFSTGVNFVVQPVGVVDTEWGNMLTGIAARPPPGEQFKHLVPLMHKGAAWPIVRRRIVQMLSDTLDKLSDYCTFVCWAHGFELTSASYFCKIGKEQKCCMCNRRAAAYSSPLQSYACWTHSCGYDYVYNPFFVDVQQWGYVGNLATNHDRYCSVHQGAHVASNDAIMTRCLAIHSCFIERVDWDIEYPYISHEKKLNSCCRIVERNVVRAALLAGSFDKVYDIGNPKGIPIVDDPVVDWHYFDAQPLTRKVQQLFYTEDMASRFADGLCLFWNCNVPKYPNNAIVCRFDTRVHSEFNLPGCDGGSLYVNKHAFHTPAYDVSAFRDLKPLPFFYYSTTPCEVHGNGSMIEDIDYVPLKSAVCITACNLGGAVCRKHATEYREYMEAYNLVSASGFRLWCYKTFDIYNLWSTFTKVQGGGSGGGSHHHHHH

SEQ ID NO: 26 - DNA, 468 bp, Artificial Sequence
  Description of Artificial Sequence Synthetic polynucleotide
atggctggatcaaatacagaatttgcaagtaacagctcggtgctttctttggttaacttcaccgtagatccgcaaaaagcgtatctggacttcgtgaacgccggtggtgctccgctgaccaattgcgtgaagatgctgacgccaaagaccggcaccggtattgcgatcagcgtgaagccggaaagcaccgctgaccaagagacgtacggcggcgcgagcgtctgcctgtactgccgtgcacatatcgagcatccggatgttagcggggtgtgcaaatacaaaggtaaatttgtccagatcccggcgcagtgtgttcgtgatcctgttggtttttgcctgagcaatactccgtgtaatgtttgccagtattggattggttatggttgtaactgcgactctttgcgccaggcggcactgccgcaaggtggcggctccggcggtggctcccatcaccaccaccaccactaa

SEQ ID NO: 27 - protein, 155 aa, Artificial Sequence
  Description of Artificial Sequence Synthetic polypeptide
MAGSNTEFASNSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQGGGSGGGSHHHHHH